(19) United States
(12) Patent Application Publication
(10) Pub. No.: US 2023/0203026 A1
(43) Pub. Date: Jun. 29, 2023

(54) PESTICIDAL COMPOSITIONS AND METHODS

(71) Applicant: CORTEVA AGRISCIENCE LLC, INDIANAPOLIS, IN (US)

(72) Inventors: NATALIE C. GIAMPIETRO, CARMEL, IN (US); THOMAS BARTON, INDIANAPOLIS, IN (US); THOMAS C. SPARKS, GREENFIELD, IN (US); DAVID A. DEMETER, FISHERS, IN (US); LINDSEY G. HORTY, INDIANAPOLIS, IN (US); JEFFERY D. WEBSTER, NEW PALESTINE, IN (US)

(73) Assignee: CORTEVA AGRISCIENCE LLC, INDIANAPOLIS, IN (US)

(21) Appl. No.: 18/170,600

(22) Filed: Feb. 17, 2023

Related U.S. Application Data
(63) Continuation of application No. 16/767,611, filed on May 28, 2020.
(60) Provisional application No. 62/594,723, filed on Dec. 5, 2017.

(51) Int. Cl. C07D 417/12; A01N 47/24; A01N 47/36

(52) U.S. Cl. CPC C07D 417/12; A01N 47/24; A01N 47/36

(57) ABSTRACT

This disclosure relates to the field of molecules having pesticidal utility against pests in phyla Nematoda, Arthropoda, and/or Mollusca, processes to produce such molecules and intermediates used in such processes, compositions containing such molecules, and processes of using such molecules against such pests. These molecules may be used, for example, as nematicides, acaricides, insecticides, miticides, and/or molluscicides. This document discloses molecules having the structure of Formula A.

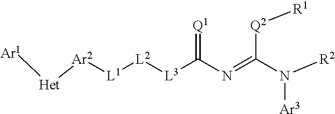

### CROSS REFERENCE TO RELATED APPLICATIONS

This application is a continuation of and claims the benefit of U.S. patent application Ser. No. 16/797,611 filed May 28, 2020, currently pending, which is a National Stage Entry of PCT Application Serial Number PCT/US18/63756 filed Dec. 4, 2018, which claims the benefit of and priority from U.S. Provisional Patent Application Ser. No. 62/594,723 filed Dec. 5, 2017, which is expressly incorporated by reference herein.

### FIELD OF THE INVENTION

The invention disclosed in this document is related to the field of pesticides and their use in controlling pests.

### BACKGROUND OF THE INVENTION

Pests cause millions of human deaths around the world each year. Furthermore, there are more than ten thousand species of pests that cause losses in agriculture. These agricultural losses amount to billions of U.S. dollars each year. Termites cause damage to various structures such as homes. These termite damage losses amount to billions of U.S. dollars each year. As a final note, many stored food pests eat and adulterate stored food. These stored food losses amount to billions of U.S. dollars each year, but more importantly, deprive people of needed food.

There is an acute need for new pesticides. Insects are developing resistance to pesticides in current use. Hundreds of insect species are resistant to one or more pesticides.

The development of resistance to some of the older pesticides, such as DDT, the carbamates, and the organophosphates, is well known. But resistance has even developed to some of the newer pesticides. Therefore, a need exists for new pesticides and particularly for pesticides that have new modes of action.

### SUMMARY OF THE INVENTION

In one aspect, provided are molecules having the structure of Formula A:

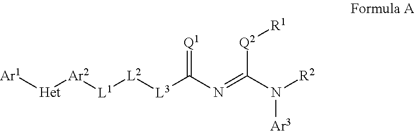

wherein:

(A) Ar¹ is selected from

(1) furanyl, phenyl, pyridazinyl, pyridyl, pyrimidinyl, thienyl, or

(2) substituted furanyl, substituted phenyl, substituted pyridazinyl, substituted pyridyl, substituted pyrimidinyl, or substituted thienyl,
wherein said substituted furanyl, substituted phenyl, substituted pyridazinyl, substituted pyridyl, substituted pyrimidinyl, and substituted thienyl have one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO²(C₁-C₈ haloalkyl), C(═O)NR^{x}R^{y}, (C₁-C₈ alkyl)NR^{x}R^{y}, C(═O)C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, or S(O)ₙNR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)OC₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1);
   (B) Het is a 5- or 6-membered, saturated or unsaturated, heterocyclic ring, containing one or more heteroatoms independently selected from nitrogen, sulfur, or oxygen, and where Ar¹ and Ar² are not ortho to each other (but may be meta or para, such as, for a five-membered ring they are 1,3 and for a 6-membered ring they are either 1,3 or 1,4) and where said heterocyclic ring may also be substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, or S(O)ₙNR^{x}R^{y},
wherein each alkyl, haloalkyl, cycloalkyl, halocycloalkyl, alkoxy, haloalkoxy, alkenyl, cycloalkenyl, haloalkenyl, alkynyl, phenyl, and phenoxy substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, or S(O)ₙNR^{x}R^{y};
   (C) Ar² is selected from

(1) furanyl, phenyl, pyridazinyl, pyridyl, pyrimidinyl, thienyl, or

(2) substituted furanyl, substituted phenyl, substituted pyridazinyl, substituted pyridyl, substituted pyrimidinyl, or substituted thienyl,
wherein said substituted furanyl, substituted phenyl, substituted pyridazinyl, substituted pyridyl, substituted pyrimidinyl, and substituted thienyl, have one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁⁻C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)NR^{x}R^{y}, or (Het-1);
   (D) L¹ is linker selected from

(1) a saturated, substituted or unsubstituted, one carbon linker,

(2) a saturated or unsaturated, substituted or unsubstituted, linear C₂-C₄ hydrocarbyl linker, or

(3) a saturated or unsaturated, substituted or unsubstituted, cyclic C₃-C₈ hydrocarbyl group linker,

wherein said substituted one carbon linker, substituted linear C₂-C₄ hydrocarbyl linker, and substituted cyclic C₃-C₈ hydrocarbyl linker has one or more substituents independently selected from R³, R⁴, R⁵, R⁶, and R⁷, wherein each R³, R⁴, R⁵, R⁶, and R⁷ is selected from H, F, Cl, Br, I, CN, OH, SH, NO₂, oxo, thioxo, NR^{x}R^{y}, C₁-C₈ alkyl, C₁-C₈ haloalkyl, C₁-C₈ alkoxy, C₁-C₈ haloalkoxy, C₂-C₈ alkenyl, C₂-C₈ haloalkenyl, C₂-C₈ alkynyl, C₂-C₈ haloalkynyl, C₃-C₈ cycloalkyl, C₃-C₈ halocycloalkyl, C₃-C₈ cycloalkenyl, C₃-C₈ halocycloalkenyl, S(C₁-C₈ alkyl), S(C₃-C₈ cycloalkyl), S(C₁-C₈ haloalkyl), S(C₃-C₈ halocycloalkyl), phenyl, or phenoxy;

(E) Each of L² and L³ is a linker independently selected from —O—, ═N—, or —N(R⁸)—,

wherein each R⁸ is independently selected from H, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁⁻C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, and (C₁-C₈ alkyl)-O-phenyl, phenyl, phenoxy, Si(C₁-C₈ alkyl)₃, S(O)ₙNR^{x}R^{y}, or (Het-1),

wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, and (C₁-C₈ alkyl)-O-phenyl, phenyl, phenoxy, Si(C₁-C₈ alkyl)₃, S(O)ₙNR^{x}R^{y}, or (Het-1);

(F) Q¹ is selected from 0 or S;
(G) Q² is selected from 0 or S;
(H) R¹ is selected from (J), H, C₁-C₈ alkyl, C₃-C₈ cycloalkyl, C₂-C₈ alkenyl, C₂-C₈ alkynyl, C(═O)(C₁-C₈ alkyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-O—C(═O)NR^{x}R^{y}, (C₁-C₈ alkyl)-C(═O)N(R^{x})(C₁-C₈ alkyl)-(Het-1), (C₁-C₈ alkyl)-C(═O)(Het-1), (C₁-C₈ alkyl)-C(═O)N(R^{x})(C₁-C₈ alkyl)N(R^{y})C(═O)OH, (C₁-C₈ alkyl)-C(═O)N(R^{x})(C₁-C₈ alkyl)N(R^{x})(R^{y}), (C₁-C₈ alkyl)-C(═O)N(R^{x})(C₁-C₈ alkyl)N(R^{y})C(═O)O—(C₁-C₈ alkyl), (C₁-C₈ alkyl)-C(═O)N(R^{x})(C₁-C₈ alkyl)(N(R^{y})C(═O)O—(C₁-C₈ alkyl)C(═O)OH, (C₁-C₈ alkyl)-C(═O)(Het-1)C(═O)O—(C₁-C₈ alkyl), (C₁-C₈alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-OC(═O)—(C₁-C₈ alkyl)N(R^{x})C(═O)O—(C₁-C₈ alkyl), (C₁-C₈ alkyl)-NR^{x}R^{y}, (C₁-C₈ alkyl)-S-(Het-1), (C₁-C₈ alkyl)S(O)ₙ(Het-1), or (C₁-C₈ alkyl)-O-(Het-1),

wherein each alkyl, cycloalkyl, phenyl, and (Het-1) are optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)OH, C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1);

(I) R² is selected from (J), H, OH, SH, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO₂(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-O—C(═O)NR^{x}R^{y}, (C₁-C₈alkyl)-C(═O)N(R^{x})(C₁-C₈ alkyl)-(Het-1), (C₁-C₈alkyl)-C(═O)(Het-1), (C₁-C₈alkyl)-C(═O)N(R^{x})(C₁-C₈ alkyl)N(R^{y})C(═O)OH, (C₁-C₈alkyl)-C(═O)N(R^{x})(C₁-C₈ alkyl)N(R^{x})(R^{y}), (C₁-C₈alkyl)-C(═O)N(R^{x})(C₁-C₈ alkyl)N(R^{y})C(═O)O—(C₁-C₈ alkyl), (C₁-C₈alkyl)-C(═O)N(R^{x})(C₁-C₈ alkyl)(N(R^{y})C(═O)O—(C₁-C₈ alkyl)C(═O)OH, (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-OC(═O)—(C₁-C₈ alkyl)N(R^{x})C(═O)O—(C₁-C₈ alkyl), (C₁-C₈ alkyl)-NR^{x}R^{y}, (C₁-C₈ alkyl)-S-(Het-1), (C₁-C₈ alkyl)S(O)ₙ(Het-1), or (C₁-C₈ alkyl)-O-(Het-1),

wherein each alkyl, haloalkyl, cycloalkyl, halocycloalkyl, cycloalkoxy, halocycloalkoxy, alkoxy, haloalkoxy, alkenyl, cycloalkenyl, haloalkenyl, alkynyl, phenyl, and (Het-1), are optionally substituted with one or more substituents independently selected from H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO₂(C₁-C₈ haloalkyl), C(═O)H, C(═O)OH, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, halophenyl, phenoxy, and (Het-1);

(J) R¹ and R² may be a 1- to 4-membered saturated or unsaturated, hydrocarbyl link, which may contain one or more heteroatoms selected from nitrogen, sulfur, and oxygen, and together with (Q²)(C)(N) forms a 4- to 7-membered cyclic structure, wherein said hydrocarbyl link may optionally be substituted with one or more substituents independently selected from R⁹, R¹⁰, and R¹¹, wherein each R⁹, R₁₀, and RH is selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, substituted phenyl, phenoxy, or (Het-1);
(K) Ara is selected from C₃-C₈ cycloalkyl, phenyl, (C₁-C₈ alkyl)phenyl, (C₁-C₈ phenyl, (C₂-C₈ alkenyl)-O-phenyl, (Het-1), (C₁-C₈ alkyl)-(Het-1), (C₁-C₈ alkyl)-O-(Het-1),

wherein the C₃-C₈ cycloalkyl, phenyl, (C₁-C₈ alkyl)phenyl, (C₁-C₈ alkyl)-O-phenyl, (C₂-C₈ alkenyl)-O-phenyl, (Het-1), (C₁-C₈ alkyl)-(Het-1), or (C₁-C₈ alkyl)-O-(Het-1) may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO₂(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1);

(L) R^{x} and R^{y} are independently selected from H, OH, SH, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈alkyl)-C(═O)(Het-1), (C₁-C₈alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-S-(Het-1), (C₁-C₈ alkyl)S(O)ₙ(Het-1), or (C₁-C₈ alkyl)-O-(Het-1),

wherein each alkyl, haloalkyl, cycloalkyl, halocycloalkyl, cycloalkoxy, halocycloalkoxy, alkoxy, haloalkoxy, alkenyl, cycloalkenyl, haloalkenyl, alkynyl, phenyl, and (Het-1), are optionally substituted with one or more substituents independently selected from H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)OH, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, halophenyl, phenoxy, and (Het-1),

or R^{x} and R^{y} together can optionally form a 5- to 7-membered saturated or unsaturated cyclic group which may contain one or more heteroatoms selected from nitrogen, sulfur, and oxygen, and where said cyclic group may be substituted with H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, substituted phenyl, phenoxy, and (Het-1);

(M) (Het-1) is a 5- or 6-membered, saturated or unsaturated, heterocyclic ring, containing one or more heteroatoms independently selected from nitrogen, sulfur or oxygen, wherein said heterocyclic ring may also be substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, and phenoxy,

wherein each alkyl, cycloalkyl, alkoxy, alkenyl, alkynyl, phenyl, and phenoxy may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO₂(C₁-C₈ haloalkyl), C(═O)H, C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, and phenoxy; and

(N) n is each individually 0, 1, or 2.

In one embodiment, the molecules of Formula A have the proviso that L² and L³ cannot be both —O—.

In another embodiment, the molecules of Formula A have the proviso that L³ cannot be —N(R⁸)— when L² is ═N—.

In another embodiment, Het and L¹ are not ortho to each other, but may be meta or para, such as, for a five membered ring they are 1,3, and for a 6 membered ring they are either 1,3 or 1,4.

In another embodiment, the molecules provided have the structure of Formula One or Formula Two:

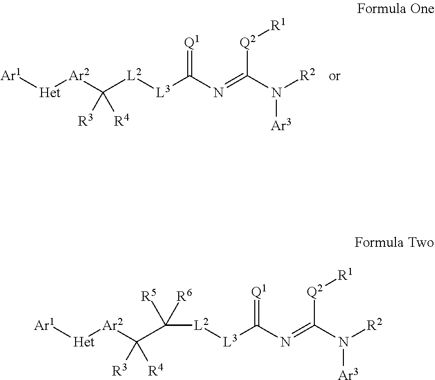

wherein:
(A) Ar¹ is a phenyl or substituted phenyl having one or more substituents independently selected from C₁-C₆ alkyl, C₁-C₆ haloalkyl, C₁-C₆ alkoxy and C₁-C₆ haloalkoxy;
(B) Het is triazolyl;
(C) Are is a phenyl or a substituted phenyl having one or more substituents independently selected from F, Cl, Br, I, CN, NO₂, NR^{x}R^{y}, C₁-C₆ alkyl, and C₁-C₆ haloalkyl;
(D) Each R³, R⁴, R⁵, and R⁶ is selected from a bond, H, F, Cl, Br, I, CN, oxo, C₁-C₆ alkyl, C₂-C₆ alkenyl, C₂-C₆ haloalkenyl, C₂-C₆ alkynyl, C₁-C₆ haloalkyl, C₃-C₆ cycloalkyl, C₃-C₆ cycloalkenyl, C₃-C₆ halocycloalkyl, and phenyl;
(E) Each of L² and L³ is a linker independently selected from —O—, ═N—, or —N(R⁸)—,

wherein each R⁸ is independently selected from H, CN, OH, SH, C₁-C₆ alkyl or C₂-C₆ alkenyl, wherein said alkyl or alkenyl is optionally substituted with a C₃-C₆ cycloalkyl or C₁-C₆ alkoxy, with the proviso that L³ cannot be —N(R⁸)— when L² is ═N— in Formula One;

(F) Q¹ is selected from O or S;
(G) Q² is selected from O or S;
(H) R¹ is selected from (J), H, F, Cl, Br, I, CN, OH, SH, C₁-C₆ alkyl or C₂-C₆ alkenyl, wherein said alkyl or alkenyl is optionally substituted with a C₃-C₆ cycloalkyl or C₁-C₆ alkoxy;
(I) R² is selected from (J), H, F, Cl, Br, I, CN, OH, SH, C₁-C₆ alkyl or C₂-C₆ alkenyl, wherein said alkyl or alkenyl is optionally substituted with a C₃-C₆ cycloalkyl or C₁-C₆ alkoxy;
(J) R¹ and R² may be a 1- to 4-membered saturated or unsaturated, hydrocarbyl link, which may contain one or more heteroatoms selected from nitrogen, sulfur, and oxygen, and together with (Q²)(C)(N) forms a 4- to 7-membered cyclic structure, wherein said hydrocarbyl link may optionally be substituted with one or more substituents independently selected from R⁹, R¹⁰, and R¹¹, wherein each R⁹, R¹⁰, and R¹¹ is selected from H, F, Cl, Br, I, CN, OH, SH, NO₂, NR^{x}R^{y}, C₁-C₆ alkyl, C₁-C₆ haloalkyl, C₁-C₆ alkoxy, C₁-C₆ haloalkoxy, S(C₁-C₆ alkyl), S(C₁-C₆ haloalkyl), phenyl, and oxo;
(K) Ara is phenyl or (Het-1), wherein the phenyl or (Het-1) may be optionally substituted with one or more substituents independently selected from F, Cl, Br, I, CN, OH, SH, NO₂, NR^{x}R^{y}, C₁-C₆ alkyl, C₁-C₆ haloalkyl, C₁-C₆ alkoxy, C₁-C₆ haloalkoxy, S(C₁-C₆ alkyl), S(C₁-C₆ haloalkyl), phenyl, and oxo;
(L) R^{x} and R^{y} are independently selected from H, C₁-C₆ alkyl, C₁-C₆ haloalkyl, C₃-C₆ cycloalkyl, C₃-C₆ halocycloalkyl, C₂-C₆ alkenyl, C₂-C₆ alkynyl, and phenyl; and
(M) (Het-1) is a 5- or 6-membered, saturated or unsaturated, heterocyclic ring, containing one or more heteroatoms independently selected from nitrogen, sulfur or oxygen, wherein said heterocyclic ring may also be substituted with one or more substituents independently selected from F, Cl, Br, I, CN, OH, SH, NO₂, NR^{x}R^{y}, C₁-C₆ alkyl, C₁-C₆ haloalkyl, C₁-C₆ alkoxy, C₁-C₆ haloalkoxy, S(C₁-C₆ alkyl), S(C₁-C₆ haloalkyl), phenyl, and oxo.

In one embodiment, Ar¹ is substituted phenyl having one or more substituents independently selected from OCF₃, OCF₂CF₃, and CF₃. In another embodiment, Het is 1,2,4-triazolyl. In another embodiment, Are is phenyl.

In another embodiment, the molecules of Formula One or Two have the proviso that L² and L³ cannot be both —O—.

In another embodiment, R¹ and R² together form a 5-membered ring containing one or two C═O, and such ring is optionally substituted with OH, F, Cl, Br, I, CN, C₁-C₆ alkyl, C₁-C₆ haloalkyl, C₁-C₆ alkoxy, C₁-C₆ haloalkoxy, phenyl or phenoxy. In another embodiment, each of R⁸ is independently H or a C₁-C₆ alkyl. In another embodiment, Ara is substituted phenyl with one or more substituents independently selected from OH, F, Cl, Br, I, C₁-C₆ alkyl, C₁-C₆ haloalkyl, C₁-C₆ hydroxyalkyl, C₁-C₆ alkoxy, or C₁-C₆ haloalkoxy.

In another aspect, provided is a process to apply a molecule provided herein. The process comprises applying a molecule provided herein, to an area to control a pest, in an amount sufficient to control such pest. In one embodiment, the pest is beet armyworm (BAW), corn earworm (CEW), or green peach aphid (GPA).

In another aspect, provided is a molecule that is a pesticidally acceptable acid addition salt, a salt derivative, a solvate, or an ester derivative, of a molecule provided herein. In another aspect, provided is a molecule provided herein wherein at least one H is ²H or at least one C is ¹⁴C. In another aspect, provided is a composition comprising a molecule provided herein and at least one other compound having insecticidal, herbicidal, acaricidal, nematicidal, or fungicidal activity. In another aspect, provided is a composition comprising a molecule provided herein and a seed.

In another aspect, provided is a process comprising applying a molecule provided herein to a genetically modified plant or a genetically-modified seed, which has been genetically modified to express one or more specialized traits. In another aspect, provided is a process comprising: orally administering or topically applying a molecule provided herein, to a non-human animal, to control endoparasites, ectoparasites, or both.

### DETAILED DESCRIPTION OF THE INVENTION

The examples given for the substituents are (except for halo) non-exhaustive and must not be construed as limiting the invention disclosed in this document.

### Definitions

“Alkenyl” means an acyclic, unsaturated (at least one carbon-carbon double bond), branched or unbranched, substituent consisting of carbon and hydrogen, for example, vinyl, allyl, butenyl, pentenyl, hexenyl, heptenyl, octenyl, nonenyl, and decenyl.

“Alkenyloxy” means an alkenyl further consisting of a carbon-oxygen single bond, for example, allyloxy, butenyloxy, pentenyloxy, hexenyloxy, heptenyloxy, octenyloxy, nonenyloxy, and decenyloxy.

“Alkoxy” means an alkyl further consisting of a carbon-oxygen single bond, for example, methoxy, ethoxy, propoxy, isopropoxy, 1-butoxy, 2-butoxy, isobutoxy, tert-butoxy, pentoxy, 2-methylbutoxy, 1,1-dimethylpropoxy, hexoxy, heptoxy, octoxy, nonoxy, and decoxy.

“Alkyl” means an acyclic, saturated, branched or unbranched, substituent consisting of carbon and hydrogen, for example, methyl, ethyl, propyl, isopropyl, 1-butyl, 2-butyl, isobutyl, tert-butyl, pentyl, 2-methylbutyl, 1,1-dimethylpropyl, hexyl, heptyl, octyl, nonyl, and decyl.

“Alkynyl” means an acyclic, unsaturated (at least one carbon-carbon triple bond, and any double bonds), branched or unbranched, substituent consisting of carbon and hydrogen, for example, ethynyl, propargyl, butynyl, pentynyl, hexynyl, heptynyl, octynyl, nonynyl, and decynyl.

“Alkynyloxy” means an alkynyl further consisting of a carbon-oxygen single bond, for example, pentynyloxy, hexynyloxy, heptynyloxy, octynyloxy, nonynyloxy, and decynyloxy.

“Aryl” means a cyclic, aromatic substituent consisting of hydrogen and carbon, for example, phenyl, naphthyl, and biphenyl.

“Cycloalkenyl” means a monocyclic or polycyclic, unsaturated (at least one carbon-carbon double bond) substituent consisting of carbon and hydrogen, for example, cyclobutenyl, cyclopentenyl, cyclohexenyl, cycloheptenyl, cyclooctenyl, cyclodecenyl, norbornenyl, bicyclo[2.2.2]octenyl, tetrahydronaphthyl, hexahydronaphthyl, and octahydronaphthyl.

“Cycloalkenyloxy” means a cycloalkenyl further consisting of a carbon-oxygen single bond, for example, cyclobutenyloxy, cyclopentenyloxy, cyclohexenyloxy, cycloheptenyloxy, cyclooctenyloxy, cyclodecenyloxy, norbornenyloxy, and bicyclo[2.2.2]octenyloxy.

“Cycloalkyl” means a monocyclic or polycyclic, saturated substituent consisting of carbon and hydrogen, for example, cyclopropyl, cyclobutyl, cyclopentyl, cyclohexyl, cycloheptyl, cyclooctyl, cyclodecyl, norbornyl, bicyclo[2.2.2]octyl, and decahydronaphthyl.

“Cycloalkoxy” means a cycloalkyl further consisting of a carbon-oxygen single bond, for example, cyclopropyloxy, cyclobutyloxy, cyclopentyloxy, cyclohexyloxy, cycloheptyloxy, cyclooctyloxy, cyclodecyloxy, norbornyloxy, and bicyclo[2.2.2]octyloxy.

“Halo” means fluoro, chloro, bromo, and iodo.

“Haloalkyl” means an alkyl further consisting of, from one to the maximum possible number of, identical or different, halos, for example, fluoromethyl, difluoromethyl, trifluoromethyl, 1-fluoromethyl, 2-fluoroethyl, 2,2,2-trifluoroethyl, chloromethyl, trichloromethyl, and 1,1,2,2-tetrafluoroethyl.

“Heterocyclyl” means a cyclic substituent that may be fully saturated, partially unsaturated, or fully unsaturated, where the cyclic structure contains at least one carbon and at least one heteroatom, where said heteroatom is nitrogen, sulfur, or oxygen, for example, benzofuranyl, benzoisothiazolyl, benzoisoxazolyl, benzoxazolyl, benzothienyl, benzothiazolyl cinnolinyl, furanyl, indazolyl, indolyl, imidazolyl, isoindolyl, isoquinolinyl, isothiazolyl, isoxazolyl, 1,3,4-oxadiazolyl, oxazolinyl, oxazolyl, phthalazinyl, pyrazinyl, pyrazolinyl, pyrazolyl, pyridazinyl, pyridyl, pyrimidinyl, pyrrolyl, quinazolinyl, quinolinyl, quinoxalinyl, 1,2,3,4-tetrazolyl, thiazolinyl, thiazolyl, thienyl, 1,2,3-triazinyl, 1,2,4-triazinyl, 1,3,5-triazinyl, 1,2,3-triazolyl, and 1,2,4-triazolyl.

### Compounds

The compounds of this invention have the structure of Formula A:

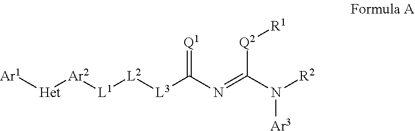

wherein:
(A) Ar¹ is selected from

(1) furanyl, phenyl, pyridazinyl, pyridyl, pyrimidinyl, thienyl, or

(2) substituted furanyl, substituted phenyl, substituted pyridazinyl, substituted pyridyl, substituted pyrimidinyl, or substituted thienyl,
wherein said substituted furanyl, substituted phenyl, substituted pyridazinyl, substituted pyridyl, substituted pyrimidinyl, and substituted thienyl have one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, or S(O)ₙNR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)OC₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1);
   (B) Het is a 5- or 6-membered, saturated or unsaturated, heterocyclic ring, containing one or more heteroatoms independently selected from nitrogen, sulfur, or oxygen, and where Ar¹ and Ar² are not ortho to each other (but may be meta or para, such as, for a five-membered ring they are 1,3 and for a 6-membered ring they are either 1,3 or 1,4) and where said heterocyclic ring may also be substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, or S(O)ₙNR^{x}R^{y},

wherein each alkyl, haloalkyl, cycloalkyl, halocycloalkyl, alkoxy, haloalkoxy, alkenyl, cycloalkenyl, haloalkenyl, alkynyl, phenyl, and phenoxy substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, or S(O)ₙNR^{x}R^{y};

(C) Are is selected from

(1) furanyl, phenyl, pyridazinyl, pyridyl, pyrimidinyl, thienyl, or

(2) substituted furanyl, substituted phenyl, substituted pyridazinyl, substituted pyridyl, substituted pyrimidinyl, or substituted thienyl,
wherein said substituted furanyl, substituted phenyl, substituted pyridazinyl, substituted pyridyl, substituted pyrimidinyl, and substituted thienyl, have one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1);
   (D) L¹ is linker selected from

(1) a saturated, substituted or unsubstituted, one carbon linker,

(2) a saturated or unsaturated, substituted or unsubstituted, linear C₂-C₄ hydrocarbyl linker, or

(3) a saturated or unsaturated, substituted or unsubstituted, cyclic C₃-C₈ hydrocarbyl group linker,

wherein said substituted one carbon linker, substituted linear C₂-C₄ hydrocarbyl linker, and substituted cyclic C₃-C₈ hydrocarbyl linker has one or more substituents independently selected from R³, R⁴, R⁵, R⁶, and R⁷, wherein each R³, R⁴, R⁵, R⁶, and R⁷ is selected from H, F, Cl, Br, I, CN, OH, SH, NO₂, oxo, thioxo, NR^{x}R^{y}, C₁-C₈ alkyl, C₁-C₈ haloalkyl, C₁-C₈ alkoxy, C₁-C₈ haloalkoxy, C₂-C₈ alkenyl, C₂-C₈ haloalkenyl, C₂-C₈ alkynyl, C₂-C₈ haloalkynyl, C₃-C₈ cycloalkyl, C₃-C₈ halocycloalkyl, C₃-C₈ cycloalkenyl, C₃-C₈ halocycloalkenyl, S(C₁-C₈ alkyl), S(C₃-C₈ cycloalkyl), S(C₁-C₈ haloalkyl), S(C₃-C₈ halocycloalkyl), phenyl, or phenoxy;

(E) Each of L² and L³ is a linker independently selected from —O—, ═N—, or —N(R⁸)—,

wherein each R⁸ is independently selected from H, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, and (C₁-C₈ alkyl)-O-phenyl, phenyl, phenoxy, Si(C₁-C₈ alkyl)₃, S(O)ₙNR^{x}R^{y}, or (Het-1),

wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, and (C₁-C₈ alkyl)-O-phenyl, phenyl, phenoxy, Si(C₁-C₈ alkyl)₃, S(O)ₙNR^{x}R^{y}, or (Het-1);

(F) Q¹ is selected from O or S;
(G) Q² is selected from O or S;
(H) R¹ is selected from (J), H, C₁-C₈ alkyl, C₃-C₈ cycloalkyl, C₂-C₈ alkenyl, C₂-C₈ alkynyl, C(═O)(C₁-C₈ alkyl), (C₁-C₈ alkyl)O(C₁-C₈ alkyl), (C₁-C₈ alkyl)S(C₁-C₈ alkyl), (C₁-C₈alkyl)S(O)ₙ(C₁-C₈ alkyl), (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-O—C(═O)NR^{x}R^{y}, (C₁-C₈ alkyl)-C(═O)N(R^{x})(C₁-C₈ alkyl)-(Het-1), (C₁-C₈ alkyl)-C(═O)(Het-1), (C₁-C₈ alkyl)-C(═O)N(R^{x})(C₁-C₈ alkyl)N(R^{y})C(═O)OH, (C₁-C₈ alkyl)-C(═O)N(R^{x})(C₁-C₈ alkyl)N(R^{x})(R^{y}), (C₁-C₈ alkyl)-C(═O)N(R^{x})(C₁-C₈ alkyl)N(R^{y})C(═O)O—(C₁-C₈ alkyl), (C₁-C₈ alkyl)-C(═O)N(R^{x})(C₁-C₈ alkyl)(N(R^{y})C(═O)O—(C₁-C₈ alkyl)C(═O)OH, (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-OC(═O)—(C₁-C₈ alkyl)N(R^{x})C(═O)O—(C₁-C₈ alkyl), (C₁-C₈ alkyl)-NR^{x}R^{y}, (C₁-C₈ alkyl)-S-(Het-1), (C₁-C₈ alkyl)S(O)(Het-1), or (C₁-C₈ alkyl)-O-(Het-1), wherein each alkyl, cycloalkyl, phenyl, and (Het-1) are optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO₂(C₁-C₈ haloalkyl), C(═O)H, C(═O)OH, C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1);
(I) R² is selected from (J), H, OH, SH, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-O—C(═O)NR^{x}R^{y}, (C₁-C₈alkyl)-C(═O)N(R^{x})(C₁-C₈ alkyl)-(Het-1), (C₁-C₈alkyl)-C(═O)(Het-1), (C₁-C₈alkyl)-C(═O)N(R^{x})(C₁-C₈ alkyl)N(R^{y})C(═O)OH, (C₁-C₈alkyl)-C(═O)N(R^{x})(C₁-C₈ alkyl)N(R^{x})(R^{y}), (C₁-C₈alkyl)-C(═O)N(R^{x})(C₁-C₈ alkyl)N(R^{y})C(═O)O—(C₁-C₈ alkyl), (C₁-C₈alkyl)-C(═O)N(R^{x})(C₁-C₈ alkyl)(N(R^{y})C(═O)O—(C₁-C₈ alkyl)C(═O)OH, (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-OC(═O)—(C₁-C₈ alkyl)N(R^{x})C(═O)O—(C₁-C₈ alkyl), (C₁-C₈ alkyl)-NR^{x}R^{y}, (C₁-C₈ alkyl)-S-(Het-1), (C₁-C₈ alkyl)S(O)ₙ(Het-1), or (C₁-C₈ alkyl)-O-(Het-1),

wherein each alkyl, haloalkyl, cycloalkyl, halocycloalkyl, cycloalkoxy, halocycloalkoxy, alkoxy, haloalkoxy, alkenyl, cycloalkenyl, haloalkenyl, alkynyl, phenyl, and (Het-1), are optionally substituted with one or more substituents independently selected from H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)OH, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, halophenyl, phenoxy, and (Het-1);

(J) R¹ and R² may be a 1-to 4-membered saturated or unsaturated, hydrocarbyl link, which may contain one or more heteroatoms selected from nitrogen, sulfur, and oxygen, and together with (Q²)(C)(N) forms a 4- to 7-membered cyclic structure, wherein said hydrocarbyl link may optionally be substituted with one or more substituents independently selected from R⁹, R¹⁰, and R¹¹ wherein each R⁹, R¹⁰, and R¹¹ is selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, substituted phenyl, phenoxy, or (Het-1);
(K) Ar³ is selected from C₃-C₈ cycloalkyl, phenyl, (C₁-C₈ alkyl)phenyl, (C₁-C₈ alkyl)-O-phenyl, (C₂-C₈ alkenyl)-O-phenyl, (Het-1), (C₁-C₈ alkyl)-(Het-1), (C₁-C₈ alkyl)-O-(Het-1), wherein the C₃-C₈ cycloalkyl, phenyl, (C₁-C₈ alkyl)phenyl, (C₁-C₈ alkyl)-O-phenyl, (C₂-C₈ alkenyl)-O-phenyl, (Het-1), (C₁-C₈ alkyl)-(Het-1), or (C₁-C₈ alkyl)-O-(Het-1) may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, S(O)ₙNR^{x}R^{y}, or (Het-1);
(L) R^{x} and R^{y} are independently selected from H, OH, SH, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO₂(C₁-C₈ alkyl), OSO₂(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-C(═O)(Het-1), (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-S-(Het-1), (C₁-C₈ alkyl)S(O)ₙ(Het-1), or (C₁-C₈ alkyl)-O-(Het-1),

wherein each alkyl, haloalkyl, cycloalkyl, halocycloalkyl, cycloalkoxy, halocycloalkoxy, alkoxy, haloalkoxy, alkenyl, cycloalkenyl, haloalkenyl, alkynyl, phenyl, and (Het-1), are optionally substituted with one or more substituents independently selected from H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)OH, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, halophenyl, phenoxy, and (Het-1),

or R^{x} and R^{y} together can optionally form a 5- to 7-membered saturated or unsaturated cyclic group which may contain one or more heteroatoms selected from nitrogen, sulfur, and oxygen, and where said cyclic group may be substituted with H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, substituted phenyl, phenoxy, and (Het-1);

(M) (Het-1) is a 5- or 6-membered, saturated or unsaturated, heterocyclic ring, containing one or more heteroatoms independently selected from nitrogen, sulfur or oxygen, wherein said heterocyclic ring may also be substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, and phenoxy,

wherein each alkyl, cycloalkyl, alkoxy, alkenyl, alkynyl, phenyl, and phenoxy may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(O)ₙ(C₁-C₈ alkyl), S(O)ₙ(C₁-C₈ haloalkyl), OSO²(C₁-C₈ alkyl), OSO²(C₁-C₈ haloalkyl), C(═O)H, C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(O)ₙ(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, and phenoxy; and

(N) n is each individually 0, 1, or 2.

In one embodiment, the compounds provided have a proviso where L³ cannot be —N(R⁸)— when L² is ═N—.

In one embodiment, Ar¹ is phenyl or substituted phenyl having one or more substituents independently selected from C₁-C₆ alkyl, C₁-C₆ haloalkyl, and C₁-C₆ haloalkoxy.

In another embodiment, Het is a triazolyl, imidazolyl, pyrrolyl, or pyrazolyl.

In another embodiment, Ar² is phenyl or a substituted phenyl having one or more substituents independently selected from C₁-C₆ alkyl, C₁-C₆ haloalkyl, and C₁-C₆ haloalkoxy.

In another embodiment, R¹, R², and each of R⁸ are independently selected from H, C₁-C₆ alkyl, C₁-C₆ alkoxy, C₁-C₆ haloalkyl, C₁-C₆ haloalkoxy, phenyl, or phenoxy; wherein R¹ and R² together can optionally form a 5- to 7-membered ring and is optionally substituted with OH, F, Cl, Br, I, CN, NO₂, C₁-C₆ alkyl, C₁-C₆ alkoxy, C₁-C₆ haloalkyl, C₁-C₆ haloalkoxy, phenyl, phenoxy, or (Het-1),

wherein (Het-1) is a 5- or 6-membered, saturated or unsaturated, heterocyclic ring, containing one or more heteroatoms independently selected from nitrogen, sulfur and oxygen.

In another embodiment, R¹ and R² together form a 5- to 7-membered ring containing one or more C═O, C═S, N, S or O, and such ring is optionally substituted with OH, F, Cl, Br, I, C₁-C₆ alkyl, C₁-C₆ haloalkyl, C₁-C₆ hydroxyalkyl, C₁-C₆ alkoxy, C₁-C₆ haloalkoxy, phenyl, or phenoxy,

wherein said phenyl or phenoxy is optionally substituted with one or more OH, F, Cl, Br, I, C₁-C₆ alkyl, C₁-C₆ haloalkyl, C₁-C₆ hydroxyalkyl, C₁-C₆ alkoxy, C₁-C₆ haloalkoxy, or phenyl.

In another embodiment, 10 and R² together form a 5- to 7-membered ring which contains one or more C═O, C═S, N, S or O.

In another embodiment, Ar³ is phenyl optionally substituted with one or more substituents independently selected from OH, F, Cl, Br, I, C₁-C₆ alkyl, C₁-C₆ haloalkyl, C₁-C₆ hydroxyalkyl, C₁-C₆ alkoxy, C₁-C₆ haloalkoxy, phenyl, or phenoxy.

In another embodiment, Ar¹ is substituted phenyl having one or more substituents independently selected from OCF₃, OCF₂CF₃, and CF₃.

In another embodiment, Het is substituted pyrazolyl wherein said substituted pyrazolyl has one or more substituents independently selected from H, C(═O)O(C₁-C₆ alkyl), or C(═O)NR^{x}R^{y}.

In another embodiment, Het is 1,2,4-triazolyl.

In another embodiment, Ar² is phenyl.

In another embodiment, Ar² is substituted phenyl having one or more substituents independently selected from OCF₃, OCF₂CF₃, and CF₃.

In another embodiment, R¹ is H or C₁-C₆ alkyl.

In another embodiment, R² is H or C₁-C₆ alkyl.

In another embodiment, each of R⁸ is independently H or C₁-C₆ alkyl.

In another embodiment, each of R¹ and R² is independently H or a C₁-C₆ alkyl.

In another embodiment, R¹ and R² together form a 5-membered ring containing one or two C═O, and such ring is optionally substituted with OH, F, Cl, Br, I, CN, C₁-C₆ alkyl, C₁-C₆ haloalkyl, C₁-C₆ alkoxy, C₁-C₆ haloalkoxy, phenyl or phenoxy.

In another embodiment, Ar³ is substituted phenyl with one or more OH, F, Cl, Br, I, C₁-C₆ alkyl, C₁-C₆ haloalkyl, C₁-C₆ hydroxyalkyl, C₁-C₆ alkoxy, or C₁-C₆ haloalkoxy.

In another embodiment, Ar³ is substituted phenyl having one or more substituents independently selected from OCF₃, OCF₂CF₃, and CF₃.

In another embodiment, the molecule has a structure selected from compounds listed in Table 1 below:

TABLE 1
Structures for Compounds

| | |
|---|---|
| A3 | 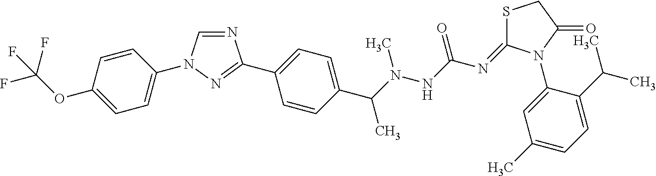 [SMILES: Cc1ccc(C(C)C)c(N2C(=O)CS/C2=N\C(=O)NN(C)C(C)c2ccc(-c3ncn(-c4ccc(OC(F)(F)F)cc4)n3)cc2)c1] |
| A4 | 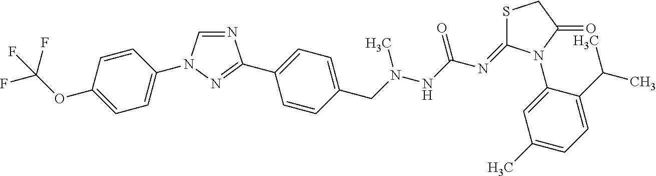 [SMILES: Cc1ccc(C(C)C)c(N2C(=O)CS/C2=N\C(=O)NN(C)Cc2ccc(-c3ncn(-c4ccc(OC(F)(F)F)cc4)n3)cc2)c1] |
| A5 | 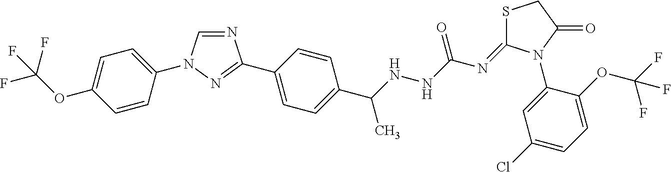 [SMILES: CC(NNC(=O)/N=C1\SCC(=O)N1c1cc(Cl)ccc1OC(F)(F)F)c1ccc(-c2ncn(-c3ccc(OC(F)(F)F)cc3)n2)cc1] |
| A6 | 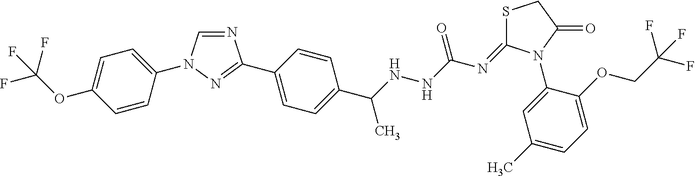 [SMILES: Cc1ccc(OCC(F)(F)F)c(N2C(=O)CS/C2=N\C(=O)NNC(C)c2ccc(-c3ncn(-c4ccc(OC(F)(F)F)cc4)n3)cc2)c1] |
| A7 | 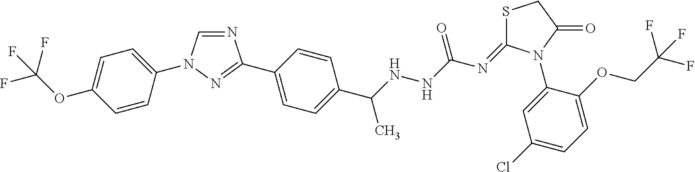 [SMILES: CC(NNC(=O)/N=C1\SCC(=O)N1c1cc(Cl)ccc1OCC(F)(F)F)c1ccc(-c2ncn(-c3ccc(OC(F)(F)F)cc3)n2)cc1] |
| A8 | 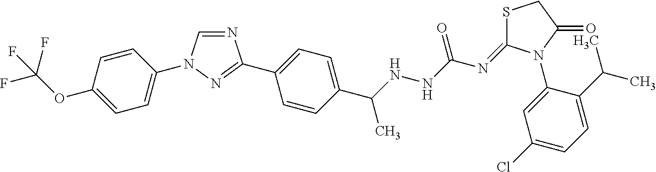 [SMILES: CC(C)c1ccc(Cl)cc1N1C(=O)CS/C1=N\C(=O)NNC(C)c1ccc(-c2ncn(-c3ccc(OC(F)(F)F)cc3)n2)cc1] |
| A9 | 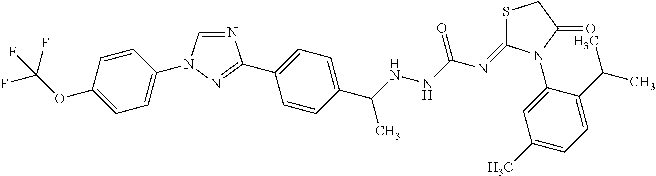 [SMILES: Cc1ccc(C(C)C)c(N2C(=O)CS/C2=N\C(=O)NNC(C)c2ccc(-c3ncn(-c4ccc(OC(F)(F)F)cc4)n3)cc2)c1] |
| A10 | 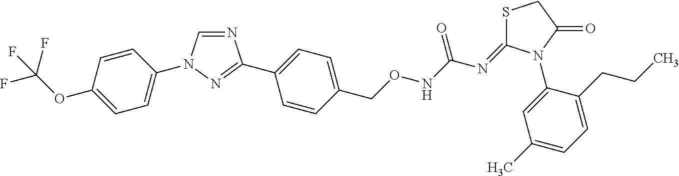 [SMILES: CCCc1ccc(C)cc1N1C(=O)CS/C1=N\C(=O)NOCc1ccc(-c2ncn(-c3ccc(OC(F)(F)F)cc3)n2)cc1] |
| A11 | 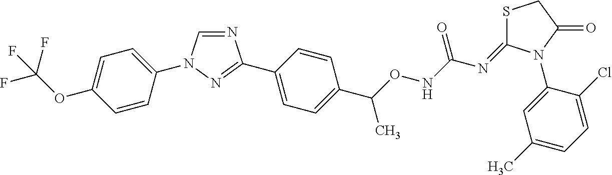 [SMILES: Cc1ccc(Cl)c(N2C(=O)CS/C2=N\C(=O)NOC(C)c2ccc(-c3ncn(-c4ccc(OC(F)(F)F)cc4)n3)cc2)c1] |
| A12 | 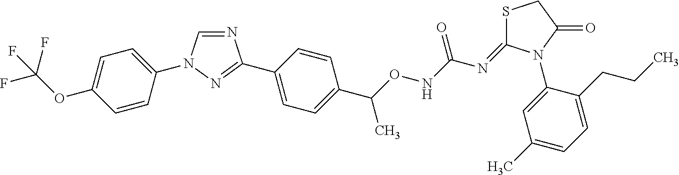 [SMILES: CCCc1ccc(C)cc1N1C(=O)CS/C1=N\C(=O)NOC(C)c1ccc(-c2ncn(-c3ccc(OC(F)(F)F)cc3)n2)cc1] |
| A13 | 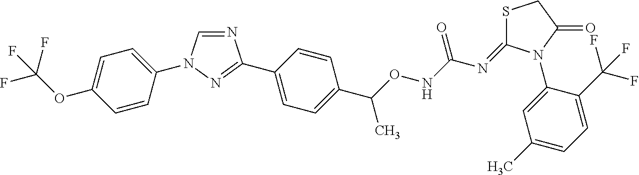 [SMILES: Cc1ccc(C(F)(F)F)c(N2C(=O)CS/C2=N\C(=O)NOC(C)c2ccc(-c3ncn(-c4ccc(OC(F)(F)F)cc4)n3)cc2)c1] |
| A14 | 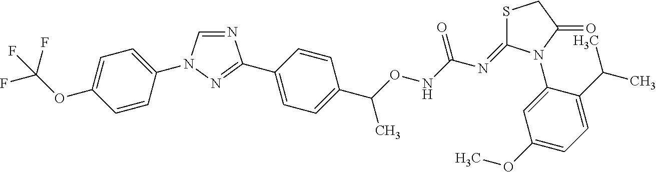 [SMILES: COc1ccc(C(C)C)c(N2C(=O)CS/C2=N\C(=O)NOC(C)c2ccc(-c3ncn(-c4ccc(OC(F)(F)F)cc4)n3)cc2)c1] |
| A15 | 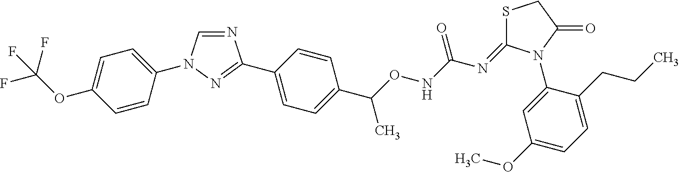 [SMILES: CCCc1ccc(OC)cc1N1C(=O)CS/C1=N\C(=O)NOC(C)c1ccc(-c2ncn(-c3ccc(OC(F)(F)F)cc3)n2)cc1] |
| A16 | 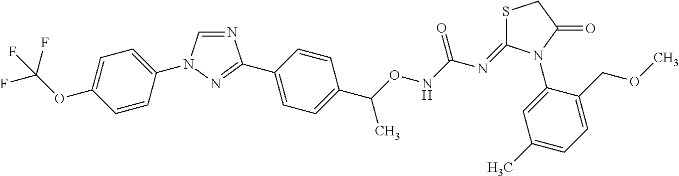 [SMILES: COCc1ccc(C)cc1N1C(=O)CS/C1=N\C(=O)NOC(C)c1ccc(-c2ncn(-c3ccc(OC(F)(F)F)cc3)n2)cc1] |
| A17 | 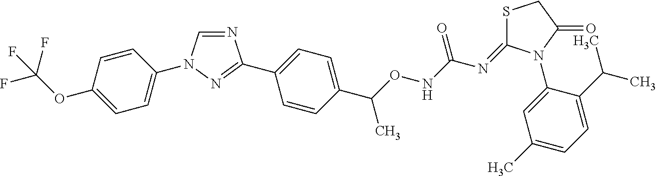 [SMILES: Cc1ccc(C(C)C)c(N2C(=O)CS/C2=N\C(=O)NOC(C)c2ccc(-c3ncn(-c4ccc(OC(F)(F)F)cc4)n3)cc2)c1] |
| A18 | 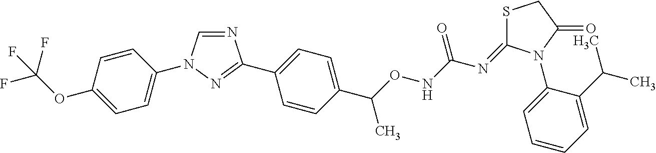 [SMILES: CC(C)c1ccccc1N1C(=O)CS/C1=N\C(=O)NOC(C)c1ccc(-c2ncn(-c3ccc(OC(F)(F)F)cc3)n2)cc1] |
| A19 | 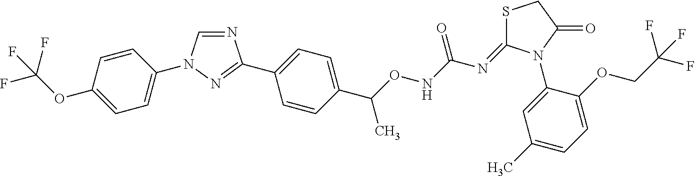 [SMILES: Cc1ccc(OCC(F)(F)F)c(N2C(=O)CS/C2=N\C(=O)NOC(C)c2ccc(-c3ncn(-c4ccc(OC(F)(F)F)cc4)n3)cc2)c1] |
| A20 | 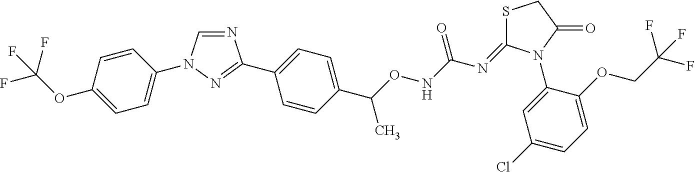 [SMILES: CC(ONC(=O)/N=C1\SCC(=O)N1c1cc(Cl)ccc1OCC(F)(F)F)c1ccc(-c2ncn(-c3ccc(OC(F)(F)F)cc3)n2)cc1] |
| A21 | 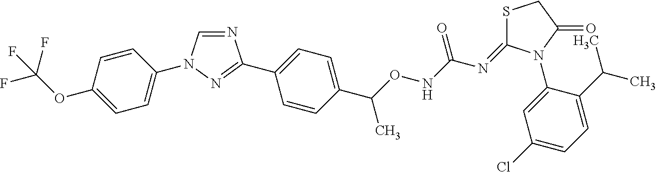 [SMILES: CC(C)c1ccc(Cl)cc1N1C(=O)CS/C1=N\C(=O)NOC(C)c1ccc(-c2ncn(-c3ccc(OC(F)(F)F)cc3)n2)cc1] |
| A22 | 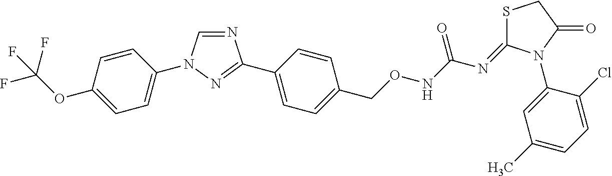 [SMILES: Cc1ccc(Cl)c(N2C(=O)CS/C2=N\C(=O)NOCc2ccc(-c3ncn(-c4ccc(OC(F)(F)F)cc4)n3)cc2)c1] |
| A23 | 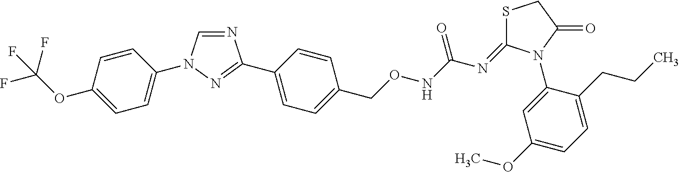 [SMILES: CCCc1ccc(OC)cc1N1C(=O)CS/C1=N\C(=O)NOCc1ccc(-c2ncn(-c3ccc(OC(F)(F)F)cc3)n2)cc1] |
| A24 | 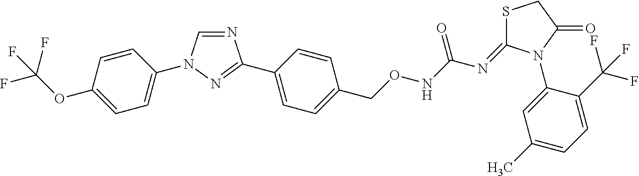 [SMILES: Cc1ccc(C(F)(F)F)c(N2C(=O)CS/C2=N\C(=O)NOCc2ccc(-c3ncn(-c4ccc(OC(F)(F)F)cc4)n3)cc2)c1] |
| A25 | 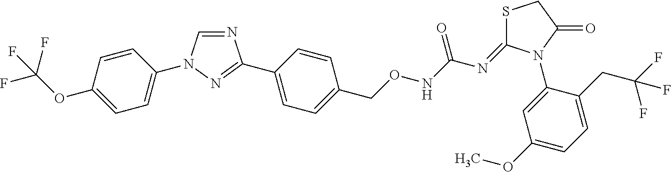 [SMILES: COc1ccc(CC(F)(F)F)c(N2C(=O)CS/C2=N\C(=O)NOCc2ccc(-c3ncn(-c4ccc(OC(F)(F)F)cc4)n3)cc2)c1] |
| A26 | 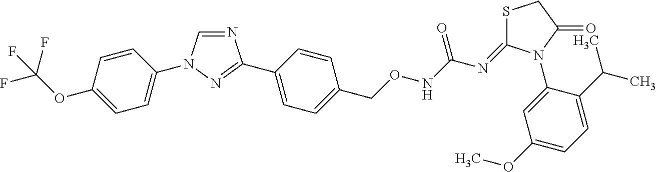 [SMILES: COc1ccc(C(C)C)c(N2C(=O)CS/C2=N\C(=O)NOCc2ccc(-c3ncn(-c4ccc(OC(F)(F)F)cc4)n3)cc2)c1] |
| A27 | 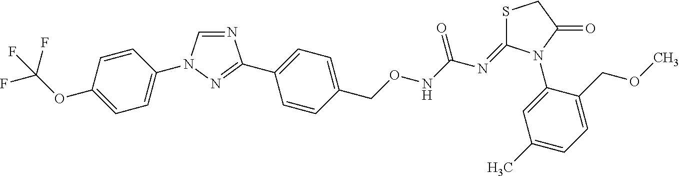 [SMILES: COCc1ccc(C)cc1N1C(=O)CS/C1=N\C(=O)NOCc1ccc(-c2ncn(-c3ccc(OC(F)(F)F)cc3)n2)cc1] |
| A28 | 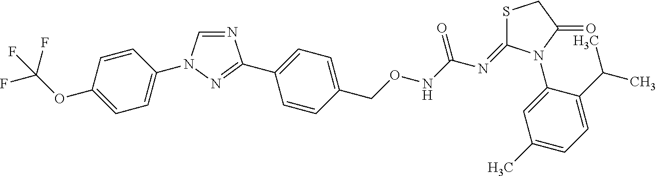 [SMILES: Cc1ccc(C(C)C)c(N2C(=O)CS/C2=N\C(=O)NOCc2ccc(-c3ncn(-c4ccc(OC(F)(F)F)cc4)n3)cc2)c1] |
| A29 | 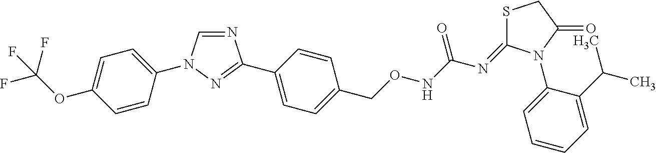 [SMILES: CC(C)c1ccccc1N1C(=O)CS/C1=N\C(=O)NOCc1ccc(-c2ncn(-c3ccc(OC(F)(F)F)cc3)n2)cc1] |
| A30 | 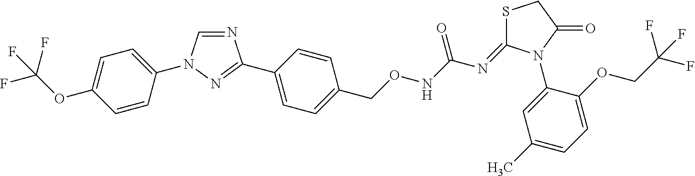 [SMILES: Cc1ccc(OCC(F)(F)F)c(N2C(=O)CS/C2=N\C(=O)NOCc2ccc(-c3ncn(-c4ccc(OC(F)(F)F)cc4)n3)cc2)c1] |
| A31 | 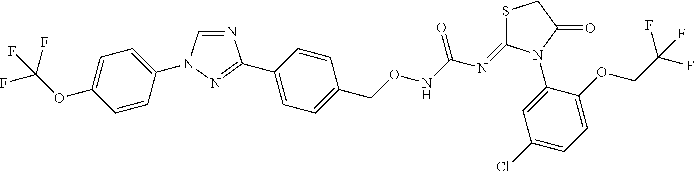 [SMILES: O=C(/N=C1\SCC(=O)N1c1cc(Cl)ccc1OCC(F)(F)F)NOCc1ccc(-c2ncn(-c3ccc(OC(F)(F)F)cc3)n2)cc1] |
| A32 | 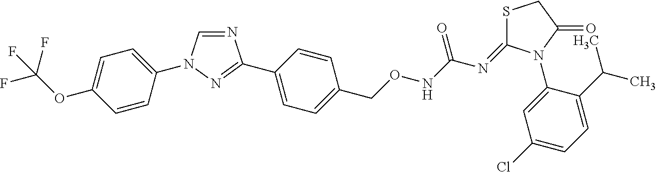 [SMILES: CC(C)c1ccc(Cl)cc1N1C(=O)CS/C1=N\C(=O)NOCc1ccc(-c2ncn(-c3ccc(OC(F)(F)F)cc3)n2)cc1] |
| A33 | 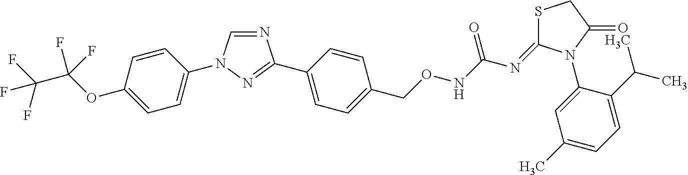 [SMILES: Cc1ccc(C(C)C)c(N2C(=O)CS/C2=N\C(=O)NOCc2ccc(-c3ncn(-c4ccc(OC(F)(F)C(F)(F)F)cc4)n3)cc2)c1] |
| A34 | 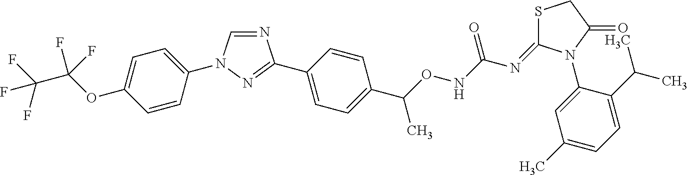 [SMILES: Cc1ccc(C(C)C)c(N2C(=O)CS/C2=N\C(=O)NOC(C)c2ccc(-c3ncn(-c4ccc(OC(F)(F)C(F)(F)F)cc4)n3)cc2)c1] |
| A35 | 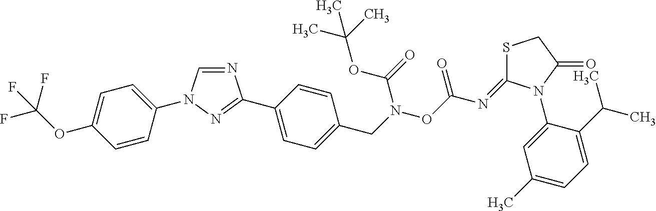 [SMILES: Cc1ccc(C(C)C)c(N2C(=O)CS/C2=N\C(=O)ON(Cc2ccc(-c3ncn(-c4ccc(OC(F)(F)F)cc4)n3)cc2)C(=O)OC(C)(C)C)c1] |
| A36 | 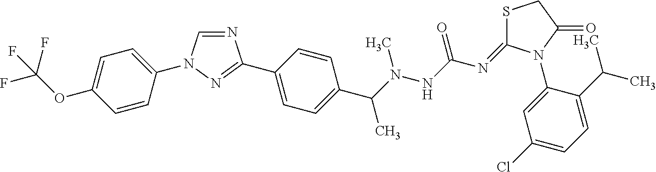 [SMILES: CC(C)c1ccc(Cl)cc1N1C(=O)CS/C1=N\C(=O)NN(C)C(C)c1ccc(-c2ncn(-c3ccc(OC(F)(F)F)cc3)n2)cc1] |
| A37 | 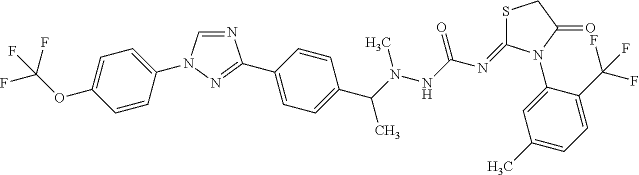 [SMILES: Cc1ccc(C(F)(F)F)c(N2C(=O)CS/C2=N\C(=O)NN(C)C(C)c2ccc(-c3ncn(-c4ccc(OC(F)(F)F)cc4)n3)cc2)c1] |
| A38 | 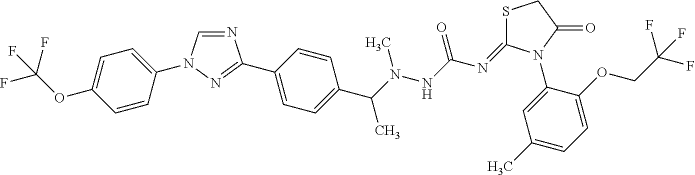 [SMILES: Cc1ccc(OCC(F)(F)F)c(N2C(=O)CS/C2=N\C(=O)NN(C)C(C)c2ccc(-c3ncn(-c4ccc(OC(F)(F)F)cc4)n3)cc2)c1] |
| A39 | 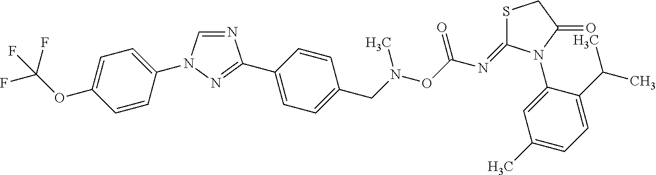 [SMILES: Cc1ccc(C(C)C)c(N2C(=O)CS/C2=N\C(=O)ON(C)Cc2ccc(-c3ncn(-c4ccc(OC(F)(F)F)cc4)n3)cc2)c1] |
| A40 | 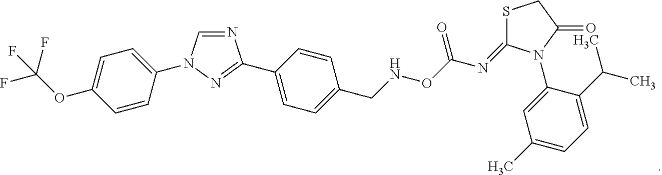 [SMILES: Cc1ccc(C(C)C)c(N2C(=O)CS/C2=N\C(=O)ONCc2ccc(-c3ncn(-c4ccc(OC(F)(F)F)cc4)n3)cc2)c1] |

### Preparation of Triaryl Hydrazine Ureas

Hydrazines 1-2 wherein Het, Aid, and Ar² are as previously disclosed, and R³ is H or (C₁-C₆)alkyl may be prepared by condensing a triaryl aldehyde or ketone 1-1 wherein Het, Ar¹, Ar², and R³ are as disclosed above with tert-butyl hydrazinecarboxylate in refluxing ethanol (Scheme 1, step a). The resultant hydrazone intermediate is immediately reduced with a reducing agent such as sodium cyanoborohydride in the presence of an acid such as glacial acetic acid in refluxing ethanol (Scheme 1, step b). Triaryl intermediates 1-1 can be prepared by methods previously described in the chemical literature. Several of these methods are described below.

Intermediates wherein ‘Het’ is a disubstituted pyridine, pyrimidine, pyrazine or pyridazine can be made by coupling of a halo- or alkylthio-substituted pyridine, pyrimidine or pyrazine with an aryl boronic acid or borate ester, under Suzuki arylation conditions. See, for example, the following.

For pyridines: Couve-Bonnaire et al. Tetrahedron 2003, 59, 2793 and Puglisi et al. Eur. J. Org. Chem. 2003, 1552.

For pyrazines: Schultheiss and Bosch Heterocycles 2003, 60, 1891.

For pyrimidines: Qing et al. J. Fluorine Chem. 2003, 120, 21 and Ceide and Montalban Tetrahedron Lett. 2006, 47, 4415.

For 2,4-diaryl pyrimidines: Schomaker and Delia, J. Org. Chem. 2001, 66, 7125.

Triaryl hydrazine ureas 1-5, wherein Het, Ar¹, Ar², Ar³, R³, and R⁴ are as previously disclosed, can be prepared by treating a triaryl hydrazine 1-2, wherein Het, Ar¹, Ar², R³, and R⁴ are as previously disclosed, via the hydrochloride salt of the hydrazine 1-4 in either one (Scheme 1, step d) or two steps (Scheme 1, steps c and d). Optionally, reductive amination using 37% aqueous formaldehyde in the presence of an acid such as glacial acetic acid in ethanol provides the alkylated intermediate, wherein R¹² is CH₃ (Scheme 1, step c). Removal of the tert-butyl oxycarbonyl group can be accomplished with acid, such as 4 molar (M) hydrogen chloride in 1,4-dioxane at ambient temperature (Scheme 1, step d). The hydrochloride salt of the hydrazine 1-4, wherein Het, Ar¹, Ar², Ar³, R³, and R⁴ are as previously disclosed and R¹² is CH₃, can be treated with a cyclic thiourea 1-4a, wherein Ar³, R¹ and R² are as previously disclosed and A is p-nitrophenyl carbamate, in a polar solvent, such as acetonitrile, and in the presence of a base, such as diisopropylethylamine, to afford a triaryl hydrazine urea 1-5, wherein Het, Ar¹, Ar², Ar³, R³, R², R³, R⁴, and R¹² are as previously disclosed (Scheme 1, step e).

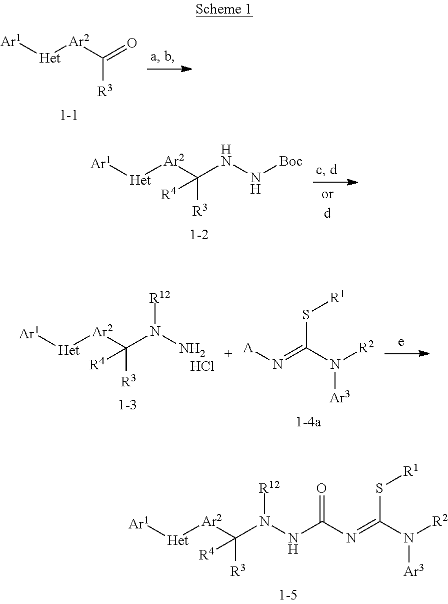

### Preparation of Hydroxylamine Analogs

Hydroxylamine analogs can be prepared as shown in Scheme 2 below. A triaryl aldehyde 2-1, wherein Het, Ar¹, Ar² are as disclosed above and R³ is H may be treated with a reducing agent such as lithium aluminum hydride in a polar, aprotic solvent such as tetrahydrofuran (THF) at a temperature of about −50° C. to about −35° C. to provide the alcohol 2-2, wherein Het, Ar¹, Ar², and R³ are as disclosed above and R⁴ is H. Alternatively, a triaryl aldehyde 2-1, wherein Het, Ar¹, Ar² are as disclosed above and R³ is H may be treated with a Grignard reagent such as methyl magnesium chloride in a polar, aprotic solvent such as THF at a temperature of about −10° C. to about 10° C. to provide the alcohol 2-2, wherein Het, Ar¹, Ar², and R³ are as disclosed above and R⁴ is CH₃ (Scheme 2, steps a and b, respectively). The alcohol 2-2, wherein Het, Ar¹, Ar², R³ and R⁴ are as disclosed above, can be converted to the corresponding bromides 2-3, wherein Het, Ar¹, Ar², and R³ are as disclosed above and R⁴ is H or CH₃, by reaction with carbon tetrabromide and triphenylphosphine in a polar, aprotic solvent such as THF at ambient temperature (Scheme 2, step c). The triaryl hydroxylamines 2-4 can be prepared in two steps. Reaction of a triaryl bromide 2-3, wherein Het, Ar¹, Ar², and R³ are as disclosed above and R⁴ is H or CH₃, with 2-hydroxyisoindoline-1,3-dione in a polar, aprotic solvent, such as N,N-dimethylformamide, and in the presence of a base, such as 1,8-diazabicyclo[5.4.0]undec-7-ene at a temperature of about −10° C. to about 10° C. provides the phthalimide-protected hydroxylamine (not shown, Scheme 2, step d). Removal of the phthalimide group with hydrazine monohydrate in an aprotic solvent such as dichloromethane (DCM) at ambient temperature affords the triaryl hydroxylamine 2-4, wherein Het, Ar¹, Ar², and R³ are as disclosed above and R⁴ is H or CH₃, (Scheme 2, step e).

Triaryl hydroxylamine ureas 2-5, wherein Het, Ar¹, Ar², and R³ are as disclosed above and R⁴ is H or CH₃, can be prepared by reacting an activated triaryl hydroxylamine 2-4a, wherein Het, Ar¹, Ar², R³, and R⁴ are as previously disclosed, with a cyclic thiourea 1-4b, wherein Ar³, R¹, and R² are as previously disclosed, in an aprotic solvent, such as dichloromethane, and in the presence of a base, such as sodium bicarbonate (Scheme 2, step g). The activated triaryl hydroxylamine 2-4a, wherein Het, Ar¹, Ar², R³ and R⁴ are as previously disclosed, can be generated by treatment of the triaryl hydroxylamine 2-4 (Scheme 2) with an activating agent such as N,N′-disuccinimidyl carbonate in a polar, aprotic solvent, such as acetonitrile, and in the presence of a base, such as pyridine, (Scheme 2, step f). The activated intermediate is then allowed to react with a cyclic thiourea 1-4b, wherein Ar³, R¹, and R² are as previously disclosed, in an aprotic solvent, such as dichloromethane, and in the presence of a base, such as sodium bicarbonate (Scheme 2, step g) to afford the urea 2-5.

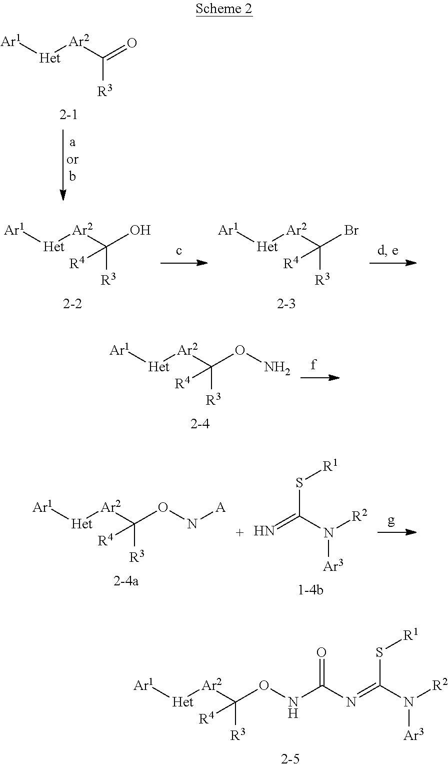

Hydroxylamine analogs can be prepared as shown in Scheme 3 below. A triaryl aldehyde 2-1, wherein Het, Ar¹, Ar² are as disclosed above and R³ is H may be treated with a nucleophile such as hydroxylamine hydrochloride in the presence of a base such as triethylamine and in a polar, protic solvent such as ethanol at the reflux temperature to provide the oxime 3-2, wherein Het, Ar¹, Ar², and R³ are as disclosed above. The oxime 3-2, wherein Het, Ar¹, Ar², R³ and R³ are as disclosed above, can be reduced to the corresponding hydroxylamine 3-3, wherein Het, Ar¹, Ar², and R³ are as disclosed above and R⁸ is H, by reaction with sodium cyanoborohydride in a polar, protic solvent such as glacial acetic acid at ambient temperature (Scheme 2, step b). The triaryl hydroxylamines 3-4 can be prepared in two steps. Reaction of a triaryl hydroxylamine 3-3, wherein Het, Ar¹, Ar², and R³ are as disclosed above and R⁸ is H, with di-tert-butyl dicarbonate (Boc₂O) in a polar, protic solvent, such as water, and in the presence of a base, such as sodium bicarbonate at ambient temperature provides the fully Boc-protected hydroxylamine (not shown, Scheme 3, step c). Removal of one of the Boc groups with a solution of ammonia in methanol in a polar, protic solvent such as methanol at ambient temperature affords the mono-Boc-protected triaryl hydroxylamine 2-4, wherein Het, Ar¹, Ar², and R³ are as disclosed above (Scheme 3, step d). Triaryl carbamate 3-5, wherein Het, Ar¹, Ar², and R³ are as disclosed above, can be prepared by reacting an activated triaryl hydroxylamine 3-4, wherein Het, Ar¹, Ar², and R³ are as previously disclosed, with an activating agent such as N,N′-disuccinimidyl carbonate in a polar, aprotic solvent such as acetonitrile and in the presence of a base such as pyridine to generate the succinimidyl-activated intermediate (not shown). This intermediate is then allowed to react with a cyclic thiourea 1-4b, wherein Ara, R¹, and R² are as previously disclosed, in an aprotic solvent, such as dichloromethane, and in the presence of a base, such as sodium bicarbonate (Scheme 3, step e) to afford the carbamate 3-5.

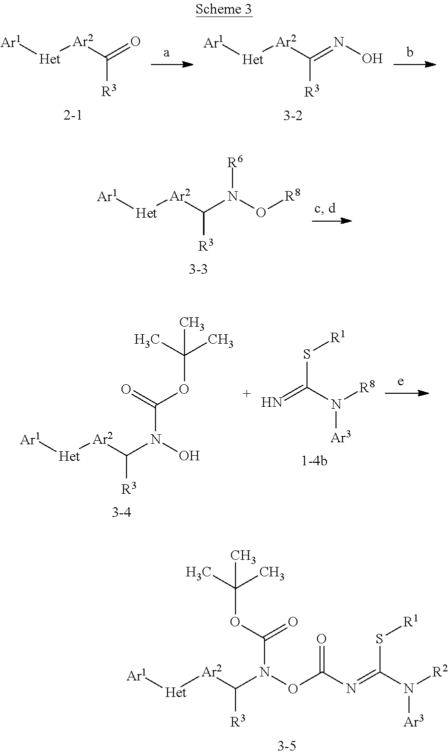

Cyclic thiourea 1-4b, wherein Ar³, R¹, and R² are as previously disclosed, can be transformed to the corresponding succinimidyl carbamate 1-4c by the addition of bis(2,5-dioxopyrrolidin-1-yl) carbonate in the presence of a polar, aprotic solvent such as acetonitrile and a base such as pyridine at ambient temperature as in Scheme 4 step a. The succinimidyl carbamate 1-4c can be reacted with 2-hydroxyisoindoline-1,3-dione in an aprotic solvent such as dichloromethane and in the presence of a base such as triethylamine to provide the phthalimide carbamate 1-4d, wherein Ar³, R¹, and R² are as previously disclosed (Scheme 4, step b). Removal of the phthalimide group from 1-4d can be accomplished using hydrazine monohydrate in an aprotic solvent such as dichloromethane at ambient temperature to afford the amino carbamate 4-1, wherein Ar³, R¹, and R² are as previously disclosed (Scheme 4, step c). A triaryl aldehyde 4-2, wherein Het, Ar¹, Ar² are as disclosed above and R³ is H may be treated with a nucleophile such as the amino carbamate 4-1 in an aprotic solvent such as dichloromethane. The intermediate imine is dried dissolved in a polar, protic solvent such as ethanol and reacted with a reducing agent such as sodium cyanoborohydride in the presence of an acid such as 1.25 molar (M) hydrogen chloride in ethanol at ambient temperature (Scheme 4, step d) to provide the carbamate 4-3.

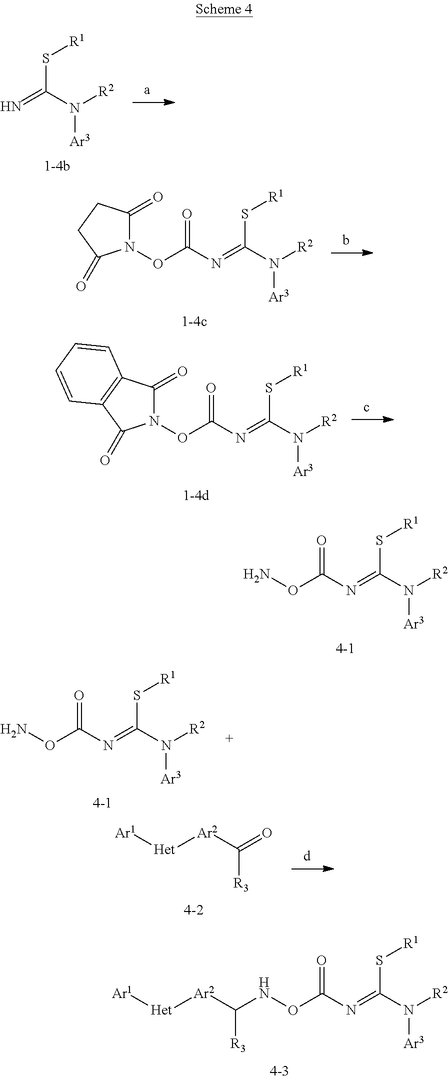

The bromide 2-3, wherein Het, Ar¹, Ar², and R³ are as disclosed above and R⁴ is H or CH₃, can be transformed into the corresponding tetrahydropyranyl (THP)-protected hydroxylamine 5-1, wherein Het, Ar¹, Ar², and R³ are as disclosed above and R⁴ is H or CH₃, by reaction with THP-hydroxylamine in a polar, aprotic solvent such as acetonitrile and in the presence of a base such as potassium carbonate at a temperature of about 60° C. to about 70° C. as in Scheme 5, step a. Methylation of 5-1 can be accomplished using a methylating agent such as iodomethane in a polar, aprotic solvent such as THF in the presence of a base such as potassium carbonate at ambient temperature to provide 5-2, wherein Het, Ar¹, Ar², and R³ are as disclosed above and R⁴ is H or CH₃ (Scheme 5, step b). Removal of the THP protecting group is effected by reaction with an acid such as 2 normal (N) hydrochloric acid in a polar, aprotic solvent such as THF at ambient temperature to afford the N-methylated oxime 5-3 as in Scheme 5, step c. The N-methylated oxime 5-3, wherein Het, Ar¹, Ar², R³ and R⁴ are as previously disclosed, can be reacted with the succinimidyl carbamate 1-4c, wherein R¹, R², and Ar³ are as previously disclosed, in an aprotic solvent, such as dichloromethane, and in the presence of a base, such as triethylamine (Scheme 5, step d) to provide the N-methylated carbamate 5-4.

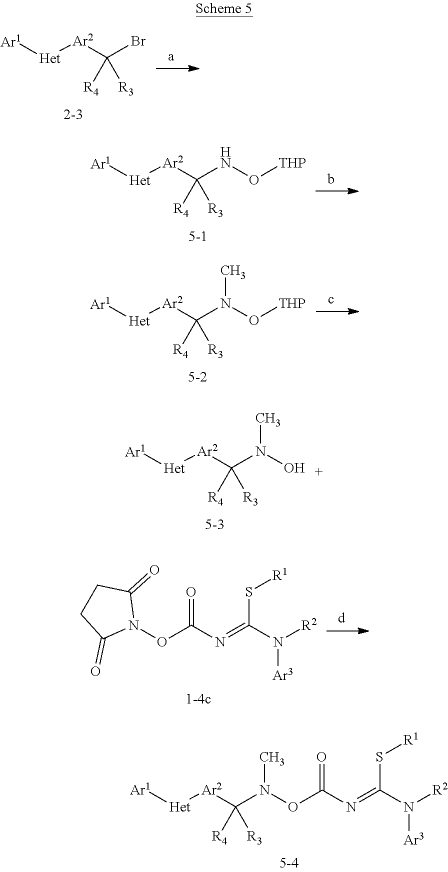

### Acid and Salt Derivatives and Solvates

The compounds disclosed in this invention can be in the form of pesticidally acceptable acid addition salts.

By way of non-limiting example, an amine function can form salts with hydrochloric, hydrobromic, sulfuric, phosphoric, acetic, benzoic, citric, malonic, salicylic, malic, fumaric, oxalic, succinic, tartaric, lactic, gluconic, ascorbic, maleic, aspartic, benzenesulfonic, methanesulfonic, ethanesulfonic, hydroxymethanesulfonic, and hydroxyethanesulfonic acids.

Additionally, by way of non-limiting example, an acid function can form salts including those derived from alkali or alkaline earth metals and those derived from ammonia and amines. Examples of preferred cations include sodium, potassium, magnesium, and aminium cations.

The salts are prepared by contacting the free base form with a sufficient amount of the desired acid to produce a salt. The free base forms may be regenerated by treating the salt with a suitable dilute aqueous base solution such as dilute aqueous sodium hydroxide (NaOH), potassium carbonate, ammonia, and sodium bicarbonate. As an example, in many cases, a pesticide is modified to a more water soluble form e.g. 2,4-dichlorophenoxy acetic acid dimethyl amine salt is a more water soluble form of 2,4-dichlorophenoxy acetic acid, a well-known herbicide.

The compounds disclosed in this invention can also form stable complexes with solvent molecules that remain intact after the non-complexed solvent molecules are removed from the compounds. These complexes are often referred to as “solvates.”

### Stereoisomers

Certain compounds disclosed in this document can exist as one or more stereoisomers. The various stereoisomers include geometric isomers, diastereomers, and enantiomers. Thus, the compounds disclosed in this invention include racemic mixtures, individual stereoisomers, and optically active mixtures. It will be appreciated by those skilled in the art that one stereoisomer may be more active than the others. Individual stereoisomers and optically active mixtures may be obtained by selective synthetic procedures, by conventional synthetic procedures using resolved starting materials, or by conventional resolution procedures.

### Pests

In another embodiment, the invention disclosed in this document can be used to control pests.

In another embodiment, the invention disclosed in this document can be used to control pests of the Phylum Nematoda.

In another embodiment, the invention disclosed in this document can be used to control pests of the Phylum Arthropoda.

In another embodiment, the invention disclosed in this document can be used to control pests of the Subphylum Chelicerata.

In another embodiment, the invention disclosed in this document can be used to control pests of the Class Arachnida.

In another embodiment, the invention disclosed in this document can be used to control pests of the Subphylum Myriapoda.

In another embodiment, the invention disclosed in this document can be used to control pests of the Class Symphyla.

In another embodiment, the invention disclosed in this document can be used to control pests of the Subphylum Hexapoda.

In another embodiment, the invention disclosed in this document can be used to control pests of the Class Insecta.

In another embodiment, the invention disclosed in this document can be used to control Coleoptera (beetles). A non-exhaustive list of these pests includes, but is not limited to, Acanthoscelides spp. (weevils), Acanthoscelides obtectus (common bean weevil), Agrilus planipennis (emerald ash borer), Agriotes spp. (wireworms), Anoplophora glabripennis (Asian longhorned beetle), Anthonomus spp. (weevils), Anthonomus grandis (boll weevil), Aphidius spp., Apion spp. (weevils), Apogonia spp. (grubs), Ataenius spretulus (Black Turfgrass Ataenius), Atomaria linearis (pygmy mangold beetle), Aulacophora spp., Bothynoderes punctiventris (beet root weevil), Bruchus spp. (weevils), Bruchus pisorum (pea weevil), Cacoesia spp., Callosobruchus maculatus (southern cow pea weevil), Carpophilus hemipteras (dried fruit beetle), Cassida vittata, Cerosterna spp Cerotoma spp. (chrysomeids), Cerotoma trifurcata (bean leaf beetle), Ceutorhynchus spp. (weevils), Ceutorhynchus assimilis (cabbage seedpod weevil), Ceutorhynchus napi (cabbage curculio), Chaetocnema spp. (chrysomelids), Colaspis spp. (soil beetles), Conoderus scalaris, Conoderus stigmosus, Conotrachelus nenuphar (plum curculio), Cotinus nitidis (Green June beetle), Crioceris asparagi (asparagus beetle), Cryptolestes ferrugineus (rusty grain beetle), Cryptolestes pusillus (flat grain beetle), Cryptolestes turcicus (Turkish grain beetle), Ctenicera spp. (wireworms), Curculio spp. (weevils), Cyclocephala spp. (grubs), Cylindrocpturus adspersus (sunflower stem weevil), Deporaus marginatus (mango leaf-cutting weevil), Dermestes lardarius (larder beetle), Dermestes maculates (hide beetle), Diabrotica spp. (chrysolemids), Epilachna varivestis (Mexican bean beetle), Faustinus cubae, Hylobius pales (pales weevil), Hypera spp. (weevils), Hypera postica (alfalfa weevil), Hyperdoes spp. (Hyperodes weevil), Hypothenemus hampei (coffee berry beetle), Ips spp. (engravers), Lasioderma serricorne (cigarette beetle), Leptinotarsa decemlineata (Colorado potato beetle), Liogenys fuscus, Liogenys suturalis, Lissorhoptrus oryzophilus (rice water weevil), Lyctus spp. (wood beetles/powder post beetles), Maecolaspis joliveti, Megascelis spp., Melanotus communis, Meligethes spp Meligethes aeneus (blossom beetle), Melolontha melolontha (common European cockchafer), Oberea brevis, Oberea linearis, Oryctes rhinoceros (date palm beetle), Oryzaephilus mercator (merchant grain beetle), Oryzaephilus surinamensis (sawtoothed grain beetle), Otiorhynchus spp. (weevils), Oulema melanopus (cereal leaf beetle), Oulema oryzae, Pantomorus spp. (weevils), Phyllophaga spp. (May/June beetle), Phyllophaga cuyabana, Phyllotreta spp. (chrysomelids), Phynchites spp Popillia japonica (Japanese beetle), Prostephanus truncates (larger grain borer), Rhizopertha dominica (lesser grain borer), Rhizotrogus spp. (European chafer), Rhynchophorus spp. (weevils), Scolytus spp. (wood beetles), Shenophorus spp. (Billbug), Sitona lineatus (pea leaf weevil), Sitophilus spp. (grain weevils), Sitophilus granaries (granary weevil), Sitophilus oryzae (rice weevil), Stegobium paniceum (drugstore beetle), Tribolium spp. (flour beetles), Tribolium castaneum (red flour beetle), Tribolium confusum (confused flour beetle), Trogoderma variabile (warehouse beetle), and Zabrus tenebioides.

In another embodiment, the invention disclosed in this document can be used to control dermaptera (earwigs).

In another embodiment, the invention disclosed in this document can be used to control Dictyoptera (cockroaches). A non-exhaustive list of these pests includes, but is not limited to, Blattella germanica (German cockroach), Blatta orientalis (oriental cockroach), Parcoblatta pennylvanica, Periplaneta americana (American cockroach), Periplaneta australoasiae (Australian cockroach), Periplaneta brunnea (brown cockroach), Periplaneta fuliginosa (smokybrown cockroach), Pyncoselus suninamensis (Surinam cockroach), and Supella longipalpa (brownbanded cockroach).

In another embodiment, the invention disclosed in this document can be used to control Diptera (true flies). A non-exhaustive list of these pests includes, but is not limited to, Aedes spp. (mosquitoes), Agromyza frontella (alfalfa blotch leafminer), Agromyza spp. (leaf miner flies), Anastrepha spp. (fruit flies), Anastrepha suspensa (Caribbean fruit fly), Anopheles spp. (mosquitoes), Batrocera spp. (fruit flies), Bactrocera cucurbitae (melon fly), Bactrocera dorsalis (oriental fruit fly), Ceratitis spp. (fruit flies), Ceratitis capitata (Mediterranean fruit fly), Chrysops spp. (deer flies), Cochliomyia spp. (screwworms), Contarinia spp. (gall midges), Culex spp. (mosquitoes), Dasineura spp. (gall midges), Dasineura brassicae (cabbage gall midge), Delia spp., Delia platura (seedcorn maggot), Drosophila spp. (vinegar flies), Fannia spp. (filth flies), Fannia canicularis (little house fly), Fannia scalaris (latrine fly), Gasterophilus intestinalis (horse bot fly), Gracillia perseae, Haematobia irritans (horn fly), Hylemyia spp. (root maggots), Hypoderma lineatum (common cattle grub), Liriomyza spp. (leafminer flies), Liriomyza brassica (serpentine leafminer), Melophagus ovinus (sheep ked), Musca spp. (muscid flies), Musca autumnalis (face fly), Musca domestica (housefly), Oestrus ovis (sheep bot fly), Oscinella frit (frit fly), Pegomyia betae (beet leafminer), Phorbia spp., Psila rosae (carrot rust fly), Rhagoletis cerasi (cherry fruit fly), Rhagoletis pomonella (apple maggot), Sitodiplosis mosellana (orange wheat blossom midge), Stomoxys calcitrans (stable fly), Tabanus spp. (horse flies), and Tipula spp. (crane flies).

In another embodiment, the invention disclosed in this document can be used to control Hemiptera (true bugs). A non-exhaustive list of these pests includes, but is not limited to, Acrosternum hilare (green stink bug), Blissus leucopterus (chinch bug), Calocoris norvegicus (potato mirid), Cimex hemipterus (tropical bed bug), Cimex lectularius (bed bug), Dagbertus fasciatus, Dichelops furcatus, Dysdercus suturellus (cotton stainer), Edessa meditabunda, Eurygaster maura (cereal bug), Euschistus heros, Euschistus servus (brown stink bug), Helopeltis antonii, Helopeltis theivora (tea blight plantbug), Lagynotomus spp. (stink bugs), Leptocorisa oratorius, Leptocorisa varicornis, Lygus spp. (plant bugs), Lygus hesperus (western tarnished plant bug), Maconellicoccus hirsutus, Neurocolpus longirostris, Nezara viridula (southern green stink bug), Phytocoris spp. (plant bugs), Phytocoris californicus, Phytocoris relativus, Piezodorus gilding, Poecilocapsus lineatus (fourlined plant bug), Psallus vaccinicola, Pseudacysta perseae, Scaptocoris castanea, and Triatoma spp. (bloodsucking conenose bugs/kissing bugs).

In another embodiment, the invention disclosed in this document can be used to control homoptera (aphids, scales, whiteflies, leafhoppers). A non-exhaustive list of these pests includes, but is not limited to, Acrythosiphon pisum (pea aphid), Adelges spp. (adelgids), Aleurodes proletella (cabbage whitefly), Aleurodicus disperses, Aleurothrixus floccosus (woolly whitefly), Aluacaspis spp., Amrasca bigutella bigutella, Aphrophora spp. (leafhoppers), Aonidiella aurantii (California red scale), Aphis spp. (aphids), Aphis gossypii (cotton aphid), Aphis pomi (apple aphid), Aulacorthum solani (foxglove aphid), Bemisia spp. (whiteflies), Bemisia argentifolii, Bemisia tabaci (sweetpotato whitefly), Brachycolus noxius (Russian aphid), Brachycorynella asparagi (asparagus aphid), Brevennia rehi, Brevicoryne brassicae (cabbage aphid), Ceroplastes spp. (scales), Ceroplastes rubens (red wax scale), Chionaspis spp. (scales), Chrysomphalus spp. (scales), Coccus spp. (scales), Dysaphis plantaginea (rosy apple aphid), Empoasca spp. (leafhoppers), Eriosoma lanigerum (woolly apple aphid), Kerya purchasi (cottony cushion scale), Idioscopus nitidulus (mango leafhopper), Laodelphax striatellus (smaller brown planthopper), Lepidosaphes spp., Macrosiphum spp., Macrosiphum euphorbiae (potato aphid), Macrosiphum granarium (English grain aphid), Macrosiphum rosae (rose aphid), Macrosteles quadrilineatus (aster leafhopper), Mahanarva frimbiolata, Metopolophium dirhodum (rose grain aphid), Mictis longicornis, Myzus persicae (green peach aphid), Nephotettix spp. (leafhoppers), Nephotettix cinctipes (green leafhopper), Nilaparvata lugens (brown planthopper), Parlatoria pergandii (chaff scale), Parlatoria ziziphi (ebony scale), Peregrinus maidis (corn delphacid), Philaenus spp. (spittlebugs), Phylloxera vitifoliae (grape phylloxera), Physokermes piceae (spruce bud scale), Planococcus spp. (mealybugs), Pseudococcus spp. (mealybugs), Pseudococcus brevipes (pineapple mealybug), Quadraspidiotus perniciosus (San Jose scale), Rhapalosiphum spp. (aphids), Rhapalosiphum maida (corn leaf aphid), Rhapalosiphum padi (oat bird-cherry aphid), Saissetia spp. (scales), Saissetia oleae (black scale), Schizaphis graminum (greenbug), Sitobion avenae (English grain aphid), Sogatella furcifera (white-backed planthopper), Therioaphis spp. (aphids), Toumeyella spp. (scales), Toxoptera spp. (aphids), Trialeurodes spp. (whiteflies), Trialeurodes vaporariorum (greenhouse whitefly), Trialeurodes abutiloneus (bandedwing whitefly), Unaspis spp. (scales), Unaspis yanonensis (arrowhead scale), and Zulia entreriana.

In another embodiment, the invention disclosed in this document can be used to control Hymenoptera (ants, wasps, and bees). A non-exhaustive list of these pests includes, but is not limited to, Acromyrrmex spp., Athalia rosae, Atta spp. (leafcutting ants), Camponotus spp. (carpenter ants), Diprion spp. (sawflies), Formica spp. (ants), Iridomyrmex humilis (Argentine ant), Monomorium ssp., Monomorium minumum (little black ant), Monomorium pharaonis (Pharaoh ant), Neodiprion spp. (sawflies), Pogonomyrmex spp. (harvester ants), Polistes spp. (paper wasps), Solenopsis spp. (fire ants), Tapoinoma sessile (odorous house ant), Tetranomorium spp. (pavement ants), Vespula spp. (yellow jackets), and Xylocopa spp. (carpenter bees).

In another embodiment, the invention disclosed in this document can be used to control Isoptera (termites). A non-exhaustive list of these pests includes, but is not limited to, Coptotermes spp., Coptotermes curvignathus, Coptotermes frenchii, Coptotermes formosanus (Formosan subterranean termite), Cornitermes spp. (nasute termites), Cryptotermes spp. (drywood termites), Heterotermes spp. (desert subterranean termites), Heterotermes aureus, Kalotermes spp. (drywood termites), Incistitermes spp. (drywood termites), Macrotermes spp. (fungus growing termites), Marginitermes spp. (drywood termites), Microcerotermes spp. (harvester termites), Microtermes obesi, Procornitermes spp., Reticulitermes spp. (subterranean termites), Reticulitermes banyulensis, Reticulitermes grassei, Reticulitermes flavipes (eastern subterranean termite), Reticulitermes hageni, Reticulitermes hesperus (western subterranean termite), Reticulitermes santonensis, Reticulitermes speratus, Reticulitermes tibialis, Reticulitermes virginicus, Schedorhinotermes spp., and Zootermopsis spp. (rotten-wood termites).

In another embodiment, the invention disclosed in this document can be used to control Lepidoptera (moths and butterflies). A non-exhaustive list of these pests includes, but is not limited to, Achoea janata, Adoxophyes spp., Adoxophyes orana, Agrotis spp. (cutworms), Agrotis ipsilon (black cutworm), Alabama argillacea (cotton leafworm), Amorbia cuneana, Amyelosis transitella (navel orangeworm), Anacamptodes defectaria, Anarsia lineatella (peach twig borer), Anomis sabulifera (jute looper), Anticarsia gemmatalis (velvetbean caterpillar), Archips argyrospila (fruit tree leafroller), Archips rosana (rose leaf roller), Argyrotaenia spp. (tortricid moths), Argyrotaenia citrana (orange tortrix), Autographa gamma, Bonagota cranaodes, Borbo cinnara (rice leaf folder), Bucculatrix thurberiella (cotton leaf perforator), Caloptilia spp. (leaf miners), Capua reticulana, Carposina niponensis (peach fruit moth), Chilo spp., Chlumetia transversa (mango shoot borer), Choristoneura rosaceana (oblique banded leaf roller), Chrysodeixis spp., Cnaphalocerus medinalis (grass leafroller), Colias spp., Conpomorpha cramerella, Cossus cossus (carpenter moth), Crambus spp. (Sod webworms), Cydia funebrana (plum fruit moth), Cydia molesta (oriental fruit moth), Cydia nignicana (pea moth), Cydia pomonella (codling moth), Darna diducta, Diaphania spp. (stem borers), Diatraea spp. (stalk borers), Diatraea saccharalis (sugarcane borer), Diatraea graniosella (southwestern corn borer), Earias spp. (bollworms), Earias insulata (Egyptian bollworm), Earias vitella (rough northern bollworm), Ecdytopopha aurantianum, Elasmopalpus lignosellus (lesser cornstalk borer), Epiphysias postruttana (light brown apple moth), Ephestia spp. (flour moths), Ephestia cautella (almond moth), Ephestia elutella (tobacco moth), Ephestia kuehniella (Mediterranean flour moth), Epimeces spp., Epinotia aporema, Erionota thrax (banana skipper), Eupoecilia ambiguella (grape berry moth), Euxoa auxiliaris (army cutworm), Feltia spp. (cutworms), Gortyna spp. (stemborers), Grapholita molesta (oriental fruit moth), Hedylepta indicata (bean leaf webber), Helicoverpa spp. (noctuid moths), Helicoverpa armigera (cotton bollworm), Helicoverpa zea (bollworm/corn earworm), Heliothis spp. (noctuid moths), Heliothis virescens (tobacco budworm), Hellula undalis (cabbage webworm), Indarbela spp. (root borers), Keiferia lycopersicella (tomato pinworm), Leucinodes orbonalis (eggplant fruit borer), Leucoptera malifoliella, Lithocollectis spp., Lobesia botrana (grape fruit moth), Loxagrotis spp. (noctuid moths), Loxagrotis albicosta (western bean cutworm), Lymantria dispar (gypsy moth), Lyonetia clerkella (apple leaf miner), Mahasena corbetti (oil palm bagworm), Malacosoma spp. (tent caterpillars), Mamestra brassicae (cabbage armyworm), Maruca testulalis (bean pod borer), Metisa plana (bagworm), Mythimna unipuncta (true armyworm), Neoleucinodes elegantalis (small tomato borer), Nymphula depunctalis (rice caseworm), Operophthera brumata (winter moth), Ostrinia nubilalis (European corn borer), Oxydia vesulia, Pandemis cerasana (common currant tortrix), Pandemis heparana (brown apple tortrix), Papilio demodocus, Pectinophora gossypiella (pink bollworm), Peridroma spp. (cutworms), Peridroma saucia (variegated cutworm), Perileucoptera coffeella (white coffee leafminer), Phthorimaea operculella (potato tuber moth), Phyllocnisitis citrella, Phyllonorycter spp. (leafminers), Pieris rapae (imported cabbageworm), Plathypena scabra, Plodia interpunctella (Indian meal moth), Plutella xylostella (diamondback moth), Polychrosis viteana (grape berry moth), Prays endocarpa, Prays oleae (olive moth), Pseudaletia spp. (noctuid moths), Pseudaletia unipunctata (armyworm), Pseudoplusia includens (soybean looper), Rachiplusia nu, Scirpophaga incertulas, Sesamia spp. (stemborers), Sesamia inferens (pink rice stem borer), Sesamia nonagrioides, Setora nitens, Sitotroga cerealella (Angoumois grain moth), Sparganothis pilleriana, Spodoptera spp. (armyworms), Spodoptera exigua (beet armyworm), Spodoptera frugiperda (fall armyworm), Spodoptera oridania (southern armyworm), Synanthedon spp. (root borers), Thecla basilides, Thermisia gemmatalis, Tineola bisselliella (webbing clothes moth), Trichoplusia ni (cabbage looper), Tuta absoluta, Yponomeuta spp., Zeuzera coffeae (red branch borer), and Zeuzera pyrina (leopard moth).

In another embodiment, the invention disclosed in this document can be used to control Mallophaga (chewing lice). A non-exhaustive list of these pests includes, but is not limited to, Bovicola ovis (sheep biting louse), Menacanthus stramineus (chicken body louse), and Menopon gallinea (common hen louse).

In another embodiment, the invention disclosed in this document can be used to control Orthoptera (grasshoppers, locusts, and crickets). A non-exhaustive list of these pests includes, but is not limited to, Anabrus simplex (Mormon cricket), Gryllotalpidae (mole crickets), Locusta migratoria, Melanoplus spp. (grasshoppers), Microcentrum retinerve (angular winged katydid), Pterophylla spp. (katydids), chistocerca gregaria, Scudderia furcata (fork tailed bush katydid), and Valanga nigricorni.

In another embodiment, the invention disclosed in this document can be used to control Phthiraptera (sucking lice). A non-exhaustive list of these pests includes, but is not limited to, Haematopinus spp. (cattle and hog lice), Linognathus ovillus (sheep louse), Pediculus humanus capitis (human body louse), Pediculus humanus humanus (human body lice), and Pthirus pubis (crab louse),

In another embodiment, the invention disclosed in this document can be used to control Siphonaptera (fleas). A non-exhaustive list of these pests includes, but is not limited to, Ctenocephalides canis (dog flea), Ctenocephalides felis (cat flea), and Pulex irritans (human flea).

In another embodiment, the invention disclosed in this document can be used to control Thysanoptera (thrips). A non-exhaustive list of these pests includes, but is not limited to, Frankliniella fusca (tobacco thrips), Frankliniella occidentalis (western flower thrips), Frankliniella shultzei Frankliniella williamsi (corn thrips), Heliothrips haemorrhaidalis (greenhouse thrips), Riphiphorothrips cruentatus, Scirtothrips spp., Scirtothrips citri (citrus thrips), Scirtothrips dorsalis (yellow tea thrips), Taeniothrips rhopalantennalis, and Thrips spp.

In another embodiment, the invention disclosed in this document can be used to control Thysanura (bristletails). A non-exhaustive list of these pests includes, but is not limited to, Lepisma spp. (silverfish) and Thermobia spp. (firebrats).

In another embodiment, the invention disclosed in this document can be used to control Acarina (mites and ticks). A non-exhaustive list of these pests includes, but is not limited to, Acarapsis woodi (tracheal mite of honeybees), Acarus spp. (food mites), Acarus siro (grain mite), Aceria mangiferae (mango bud mite), Aculops spp., Aculops lycopersici (tomato russet mite), Aculops pelekasi, Aculus pelekassi, Aculus schlechtendali (apple rust mite), Amblyomma americanum (lone star tick), Boophilus spp. (ticks), Brevipalpus obovatus (privet mite), Brevipalpus phoenicis (red and black flat mite), Demodex spp. (mange mites), Dermacentor spp. (hard ticks), Dermacentor variabilis (American dog tick), Dermatophagoides pteronyssinus (house dust mite), Eotetranycus spp., Eotetranychus carpini (yellow spider mite), Epitimerus spp., Eriophyes spp., Ixodes spp. (ticks), Metatetranycus spp., Notoedres cati, Oligonychus spp Oligonychus coffee, Oligonychus ilicus (southern red mite), Panonychus spp., Panonychus citri (citrus red mite), Panonychus ulmi (European red mite), Phyllocoptruta oleivora (citrus rust mite), Polyphagotarsonemun latus (broad mite), Rhipicephalus sanguineus (brown dog tick), Rhizoglyphus spp. (bulb mites), Sarcoptes scabiei (itch mite), Tegolophus perseaflorae, Tetranychus spp., Tetranychus urticae (two-spotted spider mite), and Varroa destructor (honey bee mite).

In another embodiment, the invention disclosed in this document can be used to control Nematoda (nematodes). A non-exhaustive list of these pests includes, but is not limited to, Aphelenchoides spp. (bud and leaf & pine wood nematodes), Belonolaimus spp. (sting nematodes), Criconemella spp. (ring nematodes), Dirofilaria immitis (dog heartworm), Ditylenchus spp. (stem and bulb nematodes), Heterodera spp. (cyst nematodes), Heterodera zeae (corn cyst nematode), Hirschmanniella spp. (root nematodes), Hoplolaimus spp. (lance nematodes), Meloidogyne spp. (root knot nematodes), Meloidogyne incognita (root knot nematode), Onchocerca volvulus (hook-tail worm), Pratylenchus spp. (lesion nematodes), Radopholus spp. (burrowing nematodes), and Rotylenchus reniformis (kidney-shaped nematode).

In another embodiment, the invention disclosed in this document can be used to control Symphyla (symphylans). A non-exhaustive list of these pests includes, but is not limited to, Scutigerella immaculata.

### Mixtures

The invention disclosed in this document can also be used with various insecticides, both for reasons of economy and synergy. Such insecticides include, but are not limited to, antibiotic insecticides, macrocyclic lactone insecticides (for example, avermectin insecticides, milbemycin insecticides, and spinosyn insecticides), arsenical insecticides, botanical insecticides, carbamate insecticides (for example, benzofuranyl methylcarbamate insecticides, dimethylcarbamate insecticides, oxime carbamate insecticides, and phenyl methylcarbamate insecticides), diamide insecticides, desiccant insecticides, dinitrophenol insecticides, fluorine insecticides, formamidine insecticides, fumigant insecticides, inorganic insecticides, insect growth regulators (for example, chitin synthesis inhibitors, juvenile hormone mimics, juvenile hormones, moulting hormone agonists, moulting hormones, moulting inhibitors, precocenes, and other unclassified insect growth regulators), nereistoxin analogue insecticides, nicotinoid insecticides (for example, nitroguanidine insecticides, nitromethylene insecticides, and pyridylmethylamine insecticides), organochlorine insecticides, organophosphorus insecticides, oxadiazine insecticides, oxadiazolone insecticides, phthalimide insecticides, pyrazole insecticides, pyrethroid insecticides, pyrimidinamine insecticides, pyrrole insecticides, tetramic acid insecticides, tetronic acid insecticides, thiazole insecticides, thiazolidine insecticides, thiourea insecticides, urea insecticides, as well as, other unclassified insecticides.

Some of the particular insecticides that can be employed beneficially in combination with the invention disclosed in this document include, but are not limited to, the following 1,2-dichloropropane, 1,3-dichloropropene, abamectin, acephate, acetamiprid, acethion, acetoprole, acrinathrin, acrylonitrile, acynonapyr, afidopyropen, afoxolaner, alanycarb, aldicarb, aldoxycarb, aldrin, allethrin, allosamidin, allyxycarb, alpha-cypermethrin, alpha-endosulfan, amidithion, aminocarb, amiton, amitraz, anabasine, athidathion, azadirachtin, azamethiphos, azinphos-ethyl, azinphos-methyl, azothoate, barium hexafluorosilicate, barthrin, bendiocarb, benfuracarb, bensultap, benzpyrimoxan, beta-cyfluthrin, beta-cypermethrin, bifenthrin, bioallethrin, bioethanomethrin, biopermethrin, bioresmethrin, bistrifluron, borax, boric acid, broflanilide, bromfenvinfos, bromocyclen, bromo-DDT, bromophos, bromophos-ethyl, bufencarb, buprofezin, butacarb, butathiofos, butocarboxim, butonate, butoxycarboxim, cadusafos, calcium arsenate, calcium polysulfide, camphechlor, carbanolate, carbaryl, carbofuran, carbon disulfide, carbon tetrachloride, carbophenothion, carbosulfan, cartap, chlorantraniliprole, chlorbicyclen, chlordane, chlordecone, chlordimeform, chlorethoxyfos, chlorfenapyr, chlorfenvinphos, chlorfluazuron, chlormephos, chloroform, chloropicrin, chloroprallethrin, chlorphoxim, chlorprazophos, chlorpyrifos, chlorpyrifos-methyl, chlorthiophos, chromafenozide, cinerin I, cinerin II, cismethrin, cloethocarb, closantel, clothianidin, copper acetoarsenite, copper arsenate, copper naphthenate, copper oleate, coumaphos, coumithoate, crotamiton, crotoxyphos, crufomate, cryolite, cyanofenphos, cyanophos, cyanthoate, cyantraniliprole, cyclaniliprole, cyclethrin, cycloprothrin, cycloxaprid, cyfluthrin, cyhalothrin, cyhalodiamide, cypermethrin, cyphenothrin, cyromazine, cythioate, DDT, decarbofuran, deltamethrin, demephion, demephion-O, demephion-S, demeton, demeton-methyl, demeton-O, demeton-O-methyl, demeton-S, demeton-S-methyl, demeton-S-methylsulphon, diafenthiuron, dialifos, diatomaceous earth, diazinon, dicapthon, dichlofenthion, dichlorvos, dicloromezotiaz, dicresyl, dicrotophos, dicyclanil, dieldrin, diflubenzuron, dilor, dimefluthrin, dimefox, dimetan, dimethoate, dimethrin, dimethylvinphos, dimetilan, dinex, dinoprop, dinosam, dinotefuran, diofenolan, dioxabenzofos, dioxacarb, dioxathion, di sulfoton, dithicrofos, d-limonene, DNOC, doramectin, ecdysterone, emamectin, ElVIPC, empenthrin, endosulfan, endothion, endrin, EPN, epofenonane, eprinomectin, epsilon-metofluthrin, epsilon-momfluorothrin, esfenvalerate, etaphos, ethiofencarb, ethion, ethiprole, ethoate-methyl, ethoprophos, ethyl formate, ethyl-DDD, ethylene dibromide, ethylene dichloride, ethylene oxide, etofenprox, etrimfos, EXD, famphur, fenamiphos, fenazaflor, fenchlorphos, fenethacarb, fenfluthrin, fenitrothion, fenobucarb, fenoxacrim, fenoxycarb, fenpirithrin, fenpropathrin, fensulfothion, fenthion, fenthion-ethyl, fenvalerate, fipronil, flonicamid, florylpicoxamid, flubendiamide, flucofuron, flucycloxuron, flucythrinate, flufenerim, flufenoxuron, flufenprox, flufiprole, fluhexafon, flupyradifurone, flupyrimin, fluralaner, fluvalinate, fluxametamide, fonofos, formetanate, formothion, formparanate, fosmethilan, fospirate, fosthietan, furathiocarb, furethrin, gamma-cyhalothrin, gamma-HCH, halfenprox, halofenozide, HCH, HEOD, heptachlor, heptenophos, heterophos, hexaflumuron, HHDN, hydramethylnon, hydrogen cyanide, hydroprene, hyquincarb, imidacloprid, imidaclothiz, imiprothrin, indoxacarb, iodomethane, IPSP, isazofos, isobenzan, isocarbophos, isocycloseram, isodrin, isofenphos, isoprocarb, isoprothiolane, isothioate, isoxathion, ivermectin, jasmolin I, jasmolin II, jodfenphos, juvenile hormone I, juvenile hormone II, juvenile hormone III, kappa-bifenthrin, kappa-tefluthrin, kelevan, kinoprene, lambda-cyhalothrin, lead arsenate, lepimectin, leptophos, lindane, lirimfos, lotilaner lufenuron, lythidathion, malathion, malonoben, mazidox, mecarbam, mecarphon, menazon, meperfluthrin, mephosfolan, mercurous chloride, mesulfenfos, metaflumizone, methacrifos, methamidophos, methidathion, methiocarb, methocrotophos, methomyl, methoprene, methoxychlor, methoxyfenozide, methyl bromide, methylchloroform, methylene chloride, metofluthrin, metolcarb, metoxadiazone, mevinphos, mexacarbate, milbemectin, milbemycin oxime, mipafox, mirex, momfluorothrin, monocrotophos, morphothion, moxidectin, naftalofos, naled, naphthalene, nicotine, nifluridide, nitenpyram, nithiazine, nitrilacarb, novaluron, noviflumuron, omethoate, oxamyl, oxazosulfyl, oxydemeton-methyl, oxydeprofos, oxydisulfoton, paichongding, para-dichlorobenzene, parathion, parathion-methyl, penfluron, pentachlorophenol, permethrin, phenkapton, phenothrin, phenthoate, phorate, phosalone, phosfolan, phosmet, phosnichlor, phosphamidon, phosphine, phoxim, phoxim-methyl, pirimetaphos, pirimicarb, pirimiphos-ethyl, pirimiphos-methyl, potassium arsenite, potassium thiocyanate, pp′-DDT, prallethrin, precocene I, precocene II, precocene III, primidophos, profenofos, profluthrin, promacyl, promecarb, propaphos, propetamphos, propoxur, prothidathion, prothiofos, prothoate, protrifenbute, pyraclofos, pyrafluprole, pyrazophos, pyresmethrin, pyrethrin I, pyrethrin II, pyridaben, pyridalyl, pyridaphenthion, pyrifluquinazon, pyrimidifen, pyrimitate, pyriprole, pyriproxyfen, quassia, quinalphos, quinalphos-methyl, quinothion, rafoxanide, resmethrin, rotenone, ryania, sabadilla, sarolaner, schradan, selamectin, silafluofen, silica gel, sodium arsenite, sodium fluoride, sodium hexafluorosilicate, sodium thiocyanate, sophamide, spinetoram, spinosad, spiromesifen, spiropidion, spirotetramat, sulcofuron, sulfoxaflor, sulfluramid, sulfotep, sulfuryl fluoride, sulprofos, tau-fluvalinate, tazimcarb, TDE, tebufenozide, tebufenpyrad, tebupirimfos, teflubenzuron, tefluthrin, temephos, TEPP, terallethrin, terbufos, tetrachlorantraniliprole, tetrachloroethane, tetrachlorvinphos, tetramethrin, tetramethylfluthrin, tetraniliprole, theta-cypermethrin, thiacloprid, thiamethoxam, thicrofos, thiocarboxime, thiocyclam, thiodicarb, thiofanox, thiometon, thiosultap, thuringiensin, tolfenpyrad, tralomethrin, transfluthrin, transpermethrin, triarathene, triazamate, triazophos, trichlorfon, trichlormetaphos-3, trichloronat, trifenofos, triflumezopyrim, triflumuron, trimethacarb, triprene, tyclopyrazoflor, vamidothion, vaniliprole, XMC, xylylcarb, zeta-cypermethrin, zolaprofos, and α-ecdysone.

Additionally, any combination of the above insecticides can be used.

The invention disclosed in this document can also be used, for reasons of economy and synergy, with acaricides, algicides, antifeedants, avicides, bactericides, bird repellents, chemosterilants, fungicides, herbicide safeners, herbicides, insect attractants, insect repellents, mammal repellents, mating disrupters, molluscicides, plant activators, plant growth regulators, rodenticides, synergists, defoliants, desiccants, disinfectants, semiochemicals, and virucides (these categories not necessarily mutually exclusive).

### Synergistic Mixtures

The invention disclosed in this document can be used with other compounds such as the ones mentioned under the heading “Mixtures” to form synergistic mixtures where the mode of action of the compounds in the mixtures are the same, similar, or different.

Examples of mode of actions include, but are not limited to: acetylcholinesterase inhibitor; sodium channel modulator; chitin biosynthesis inhibitor; GABA-gated chloride channel antagonist; GABA- and glutamate-gated chloride channel agonist; acetylcholine receptor agonist; MET I inhibitor; Mg-stimulated ATPase inhibitor; nicotinic acetylcholine receptor; Midgut membrane disrupter; oxidative phosphorylation disrupter; and ryanodine receptor (RyRs).

Additionally, the following compounds are known as synergists and can be used with the invention disclosed in this document: piperonyl butoxide, piprotal, propyl isome, sesamex, sesamolin, and sulfoxide.

### Formulations

A pesticide is rarely suitable for application in its pure form. It is usually necessary to add other substances so that the pesticide can be used at the required concentration and in an appropriate form, permitting ease of application, handling, transportation, storage, and maximum pesticide activity. Thus, pesticides are formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra low volume solutions.

Pesticides are applied most often as aqueous suspensions or emulsions prepared from concentrated formulations of such pesticides. Such water-soluble, water-suspendable, or emulsifiable formulations, are either solids, usually known as wettable powders, or water dispersible granules, or liquids usually known as emulsifiable concentrates, or aqueous suspensions. Wettable powders, which may be compacted to form water dispersible granules, comprise an intimate mixture of the pesticide, a carrier, and surfactants. The concentration of the pesticide is usually from about 10% to about 90% by weight. The carrier is usually chosen from among the attapulgite clays, the montmorillonite clays, the diatomaceous earths, or the purified silicates. Effective surfactants, comprising from about 0.5% to about 10% of the wettable powder, are found among sulfonated lignins, condensed naphthalenesulfonates, naphthalenesulfonates, alkylbenzenesulfonates, alkyl sulfates, and nonionic surfactants such as ethylene oxide adducts of alkyl phenols.

Emulsifiable concentrates of pesticides comprise a convenient concentration of a pesticide, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are chosen from conventional anionic and nonionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums, may also be added, to increase the density and viscosity of the aqueous carrier. It is often most effective to grind and mix the pesticide at the same time by preparing the aqueous mixture and homogenizing it in an implement such as a sand mill, ball mill, or piston-type homogenizer.

Pesticides may also be applied as granular compositions that are particularly useful for applications to the soil. Granular compositions usually contain from about 0.5% to about 10% by weight of the pesticide, dispersed in a carrier that comprises clay or a similar substance. Such compositions are usually prepared by dissolving the pesticide in a suitable solvent and applying it to a granular carrier which has been pre-formed to the appropriate particle size, in the range of from about 0.5 to about 3 mm. Such compositions may also be formulated by making a dough or paste of the carrier and compound and crushing and drying to obtain the desired granular particle size.

Dusts containing a pesticide are prepared by intimately mixing the pesticide in powdered form with a suitable dusty agricultural carrier, such as kaolin clay, ground volcanic rock, and the like. Dusts can suitably contain from about 1% to about 10% of the pesticide. They can be applied as a seed dressing or as a foliage application with a dust blower machine.

It is equally practical to apply a pesticide in the form of a solution in an appropriate organic solvent, usually petroleum oil, such as the spray oils, which are widely used in agricultural chemistry.

Pesticides can also be applied in the form of an aerosol composition. In such compositions the pesticide is dissolved or dispersed in a carrier, which is a pressure-generating propellant mixture. The aerosol composition is packaged in a container from which the mixture is dispensed through an atomizing valve.

Pesticide baits are formed when the pesticide is mixed with food or an attractant or both. When the pests eat the bait they also consume the pesticide. Baits may take the form of granules, gels, flowable powders, liquids, or solids. They are used in pest harborages.

Fumigants are pesticides that have a relatively high vapor pressure and hence can exist as a gas in sufficient concentrations to kill pests in soil or enclosed spaces. The toxicity of the fumigant is proportional to its concentration and the exposure time. They are characterized by a good capacity for diffusion and act by penetrating the pest's respiratory system or being absorbed through the pest's cuticle. Fumigants are applied to control stored product pests under gas proof sheets, in gas sealed rooms or buildings or in special chambers.

Pesticides can be microencapsulated by suspending the pesticide particles or droplets in plastic polymers of various types. By altering the chemistry of the polymer or by changing factors in the processing, microcapsules can be formed of various sizes, solubility, wall thicknesses, and degrees of penetrability. These factors govern the speed with which the active ingredient within is released, which in turn, affects the residual performance, speed of action, and odor of the product.

Oil solution concentrates are made by dissolving pesticide in a solvent that will hold the pesticide in solution. Oil solutions of a pesticide usually provide faster knockdown and kill of pests than other formulations due to the solvents themselves having pesticidal action and the dissolution of the waxy covering of the integument increasing the speed of uptake of the pesticide. Other advantages of oil solutions include better storage stability, better penetration of crevices, and better adhesion to greasy surfaces.

Another embodiment is an oil-in-water emulsion, wherein the emulsion comprises oily globules which are each provided with a lamellar liquid crystal coating and are dispersed in an aqueous phase, wherein each oily globule comprises at least one compound which is agriculturally active, and is individually coated with a monolamellar or oligolamellar layer comprising: (1) at least one nonionic lipophilic surface-active agent, (2) at least one nonionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007, having patent application Ser. No. 11/495,228. For ease of use this embodiment will be referred to as “OIWE”.

### Other Formulation Components

Generally, the invention disclosed in this document when used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of a particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, nonionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates. Tristyrylphenol ethoxylate phosphate esters are also used. Nonionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO block copolymers; and graft copolymers.

An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The type of surfactants usually used for solubilization are nonionics: sorbitan monooleates; sorbitan monooleate ethoxylates; and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often nonionics such as: alkyl ethoxylates; linear aliphatic alcohol ethoxylates; aliphatic amine ethoxylates.

A carrier or diluent in an agricultural formulation is a material added to the pesticide to give a product of the required strength. Carriers arc usually materials with high absorptive capacities, while diluents are usually materials with low absorptive capacities. Carriers and diluents are used in the formulation of dusts, wettable powders, granules and water-dispersible granules.

Organic solvents are used mainly in the formulation of emulsifiable concentrates, ULV (ultra low volume) formulations, and to a lesser extent granular formulations. Sometimes mixtures of solvents are used. The first main groups of solvents are aliphatic paraffinic oils such as kerosene or refined paraffins. The second main group and the most common comprises the aromatic solvents such as xylene and higher molecular weight fractions of C₉ and C₁₀ aromatic solvents. Chlorinated hydrocarbons are useful as cosolvents to prevent crystallization of pesticides when the formulation is emulsified into water. Alcohols are sometimes used as cosolvents to increase solvent power.

Thickeners or gelling agents are used mainly in the formulation of suspension concentrates, emulsions and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are not limited to, montmorillonite, e.g. bentonite; magnesium aluminum silicate; and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenam; alginates; methyl cellulose; sodium carboxymethyl cellulose (SCMC); hydroxyethyl cellulose (HEC). Other types of anti-settling agents are based on modified starches, polyacrylates, polyvinyl alcohol and polyethylene oxide. Another good anti-settling agent is xanthan gum.

Microorganisms cause spoilage of formulated products. Therefore preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazalin-3-one (BIT).

The presence of surfactants, which lower interfacial tension, often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane while the non-silicone anti-foam agents are water-insoluble oils, such as octanol and nonanol, or silica. In both cases, the function of the anti-foam agent is to displace the surfactant from the air-water interface.

### Applications

The actual amount of pesticide to be applied to loci of pests is generally not critical and can readily be determined by those skilled in the art. In general, concentrations from about 0.01 grams of pesticide per hectare to about 5000 grams of pesticide per hectare are expected to provide good control.

The locus to which a pesticide is applied can be any locus inhabited by any pest, for example, vegetable crops, fruit and nut trees, grapevines, ornamental plants, domesticated animals, the interior or exterior surfaces of buildings, and the soil around buildings. Controlling pests generally means that pest populations, activity, or both, are reduced in a locus. This can come about when: pest populations are repulsed from a locus; when pests are incapacitated in or around a locus; or pests are exterminated, in whole or in part, in or around a locus. Of course a combination of these results can occur. Generally, pest populations, activity, or both are desirably reduced more than fifty percent, preferably more than 90 percent.

Generally, with baits, the baits are placed in the ground where, for example, termites can come into contact with the bait. Baits can also be applied to a surface of a building, (horizontal, vertical, or slant surface) where, for example, ants, termites, cockroaches, and flies, can come into contact with the bait.

Because of the unique ability of the eggs of some pests to resist pesticides repeated applications may be desirable to control newly emerged larvae.

Systemic movement of pesticides in plants may be utilized to control pests on one portion of the plant by applying the pesticides to a different portion of the plant. For example, control of foliar-feeding insects can be controlled by drip irrigation or furrow application, or by treating the seed before planting. Seed treatment can be applied to all types of seeds, including those from which plants genetically transformed to express specialized traits will germinate. Representative examples include those expressing proteins toxic to invertebrate pests, such as Bacillus thuringiensis or other insecticidal toxins, those expressing herbicide resistance, such as “Roundup Ready” seed, or those with “stacked” foreign genes expressing insecticidal toxins, herbicide resistance, nutrition-enhancement or any other beneficial traits. Furthermore, such seed treatments with the invention disclosed in this document can further enhance the ability of a plant to better withstand stressful growing conditions. This results in a healthier, more vigorous plant, which can lead to higher yields at harvest time.

It should be readily apparent that the invention can be used with plants genetically transformed to express specialized traits, such as Bacillus thuringiensis or other insecticidal toxins, or those expressing herbicide resistance, or those with “stacked” foreign genes expressing insecticidal toxins, herbicide resistance, nutrition-enhancement or any other beneficial traits.

The invention disclosed in this document is suitable for controlling endoparasites and ectoparasites in the veterinary medicine sector or in the field of animal keeping. Compounds are applied in a known manner, such as by oral administration in the form of, for example, tablets, capsules, drinks, granules, by dermal application in the form of, for example, dipping, spraying, pouring on, spotting on, and dusting, and by parenteral administration in the form of, for example, an injection.

The invention disclosed in this document can also be employed advantageously in livestock keeping, for example, cattle, sheep, pigs, chickens, and geese. Suitable formulations are administered orally to the animals with the drinking water or feed. The dosages and formulations that are suitable depend on the species.

Before a pesticide can be used or sold commercially, such pesticide undergoes lengthy evaluation processes by various governmental authorities (local, regional, state, national, international). Voluminous data requirements are specified by regulatory authorities and must be addressed through data generation and submission by the product registrant or by another on the product registrant's behalf. These governmental authorities then review such data and if a determination of safety is concluded, provide the potential user or seller with product registration approval. Thereafter, in that locality where the product registration is granted and supported, such user or seller may use or sell such pesticide.

### Combinations

In another embodiment of this invention, molecules of Formula A, Formula One or Formula Two may be used in combination (such as, in a compositional mixture, or a simultaneous or sequential application) with one or more active ingredients.

In another embodiment of this invention, molecules of Formula A, Formula One or Formula Two may be used in combination (such as, in a compositional mixture, or a simultaneous or sequential application) with one or more active ingredients each having a MoA that is the same as, similar to, but more likely—different from, the MoA of the molecules of Formula A, Formula One or Formula Two.

In another embodiment, molecules of Formula A, Formula One or Formula Two may be used in combination (such as, in a compositional mixture, or a simultaneous or sequential application) with one or more molecules having acaricidal, algicidal, avicidal, bactericidal, fungicidal, herbicidal, insecticidal, molluscicidal, nematicidal, rodenticidal, and/or virucidal properties.

In another embodiment, the molecules of Formula A, Formula One or Formula Two may be used in combination (such as, in a compositional mixture, or a simultaneous or sequential application) with one or more molecules that are antifeedants, bird repellents, chemosterilants, herbicide safeners, insect attractants, insect repellents, mammal repellents, mating disrupters, plant activators, plant growth regulators, and/or synergists.

In another embodiment, molecules of Formula A, Formula One or Formula Two may also be used in combination (such as in a compositional mixture, or a simultaneous or sequential application) with one or more biopesticides.

TABLE A
Weight Ratios
Molecule of the Formula A, Formula
One or Formula Two: active ingredient
100:1 to 1:100
50:1 to 1:50
20:1 to 1:20
10:1 to 1:10
5:1 to 1:5
3:1 to 1:3
2:1 to 1:2
1:1

In another embodiment, in a pesticidal composition combinations of a molecule of Formula A, Formula One or Formula Two and an active ingredient may be used in a wide variety of weight ratios. For example, in a two-component mixture, the weight ratio of a molecule of Formula A, Formula One or Formula Two to an active ingredient, the weight ratios in Table A may be used. However, in general, weight ratios less than about 10:1 to about 1:10 are preferred. It is also preferred sometimes to use a three, four, five, six, seven, or more, component mixture comprising a molecule of Formula A, Formula One or Formula Two and an additional two or more active ingredients.

Weight ratios of a molecule of Formula A, Formula One or Formula Two to an active ingredient may also be depicted as X:Y; wherein X is the parts by weight of a molecule of Formula A, Formula One or Formula Two and Y is the parts by weight of active ingredient. The numerical range of the parts by weight for X is 0<X≤100 and the parts by weight for Y is 0<Y≤100 and is shown graphically in Table B. By way of non-limiting example, the weight ratio of a molecule of Formula A, Formula One or Formula Two to an active ingredient may be 20:1.

Ranges of weight ratios of a molecule of Formula A, Formula One or Formula Two to an active ingredient may be depicted as X₁:Y₁ to X₂:Y₂, wherein X and Y are defined as above.

In one embodiment, the range of weight ratios may be X₁:Y₁ to X₂:Y₂, wherein X₁>Y₁ and X₂<Y₂. By way of non-limiting example, the range of a weight ratio of a molecule of Formula A, Formula One or Formula Two to an active ingredient may be between 3:1 and 1:3, inclusive of the endpoints.

TABLE B

| | | | | | | | | | | |
|---|---|---|---|---|---|---|---|---|---|---|
| active | 100 | X, Y | | X, Y | | | X, Y | | | |
| ingredient | 50 | X, Y | X, Y | X, Y | | | X, Y | X, Y | | |
| (Y) Parts | 20 | X, Y | | X, Y | X, Y | | X, Y | | X, Y | |
| by weight | 15 | X, Y | X, Y | | | | | X, Y | X, Y | X, Y |
| | 10 | X, Y | | X, Y | | | | | | |
| | 5 | X, Y | X, Y | X, Y | | | | X, Y | | |
| | 3 | X, Y | X, Y | | X, Y | X, Y | | X, Y | X, Y | X, Y |
| | 2 | X, Y | | X, Y | X, Y | | X, Y | | X, Y | |
| | 1 | X, Y | X, Y | X, Y | X, Y | X, Y | X, Y | X, Y | X, Y | X, Y |
| | | 1 | 2 | 3 | 5 | 10 | 15 | 20 | 50 | 100 |

molecule of Formula A, Formula One or Formula Two
(X) Parts by weight

In another embodiment, the range of weight ratios may be X₁:Y₁ to X₂:Y₂, wherein X₁>Y₁ and X₂>Y₂. By way of non-limiting example, the range of weight ratio of a molecule of Formula A, Formula One or Formula Two to an active ingredient may be between 15:1 and 3:1, inclusive of the endpoints.

In another embodiment, the range of weight ratios may be X₁:Y₁ to X₂:Y₂, wherein X₁ <Y₁ and X₂<Y₂. By way of non-limiting example, the range of weight ratios of a molecule of Formula A, Formula One or Formula Two to an active ingredient may be between about 1:3 and about 1:20, inclusive of the endpoints.

It is envisioned that certain weight ratios of a molecule of Formula A, Formula One or Formula Two to an active ingredient, as presented in Table A and B, may be synergistic.

### EXAMPLES

The examples are for illustration purposes and are not to be construed as limiting the invention disclosed in this document to only the embodiments disclosed in these examples.

Starting materials, reagents and solvents which are obtained from commercial sources are used without further purification. Anhydrous solvents are purchased as Sure/Seal™ from Aldrich and are used as received. Melting points are obtained on a Thomas Hoover Unimelt capillary melting point apparatus or an OptiMelt Automated Melting Point System from Sanford Research Systems and are uncorrected. Examples using “room temperature” were conducted in climate controlled laboratories with temperatures ranging from about 20° C. to about 24° C. Molecules are given their known names, named according to naming programs within ISIS Draw, ChemDraw, or ACD Name Pro. If such programs are unable to name a molecule, such molecule is named using conventional naming rules. 41 nuclear magnetic resonance (NMR) spectral data are in parts per million (ppm, δ) and were recorded at 300, 400, or 500 MHz; ¹³C NMR spectral data are in ppm (δ) and were recorded at 75, 100, or 150 MHz, and ¹⁹F NMR spectral data are in ppm (δ) and were recorded at 376 MHz, unless otherwise stated.

### Example 1: Preparation of (Z)—N-(3-(2-isopropyl-5-methylphenyl)-4-oxothiazolidin-2-ylidene)-2-methyl-2-(1-(4-(1-(4-(trifluoromethoxy)phenyl)-1H-1,2,4-triazol-3-yl)phenyl)ethyl)hydrazine-1-carboxamide (A3)

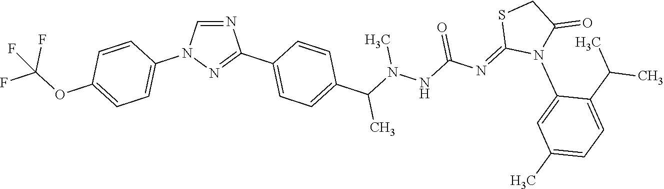 [SMILES: Cc1ccc(C(C)C)c(N2C(=O)CS/C2=N\C(=O)NN(C)C(C)c2ccc(-c3ncn(-c4ccc(OC(F)(F)F)cc4)n3)cc2)c1]

3-(4-(1-(1-Methylhydrazinyl)ethyl)phenyl)-1-(4-(trifluoromethoxy)phenyl)-1H-1,2,4-triazole hydrochloride (C1; 0.100 g, 0.242 mmol) and bis(2,5-dioxopyrrolidin-1-yl) carbonate (0.077 g, 0.302 mmol) were combined in acetonitrile (0.967 mL), and N,N-diisopropylethylamine (0.127 mL, 0.725 mmol) was added. 2-Imino-3-(2-isopropyl-5-methylphenyl)thiazolidin-4-one (0.066 g, 0.266 mmol) was added, and the mixture was stirred for 30 min. The reaction mixture was concentrated. Purification via silica gel chromatography with a gradient of 0-80% ethyl acetate (EtOAc) in hexanes yielded the title compound as a yellow foamy glass (42 mg, 27%).

The following compounds were prepared in like manner to the procedure outlined in Example 1:

### (Z)—N-(3-(5-Chloro-2-isopropylphenyl)-4-oxothiazolidin-2-ylidene)-2-methyl-2-(1-(4-(1-(4-(trifluoromethoxy)phenyl)-1H-1,2,4-triazol-3-yl)phenyl)ethyl)hydrazine-1-carboxamide (A36)

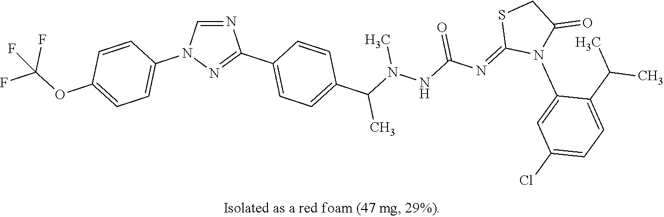 [SMILES: CC(C)c1ccc(Cl)cc1N1C(=O)CS/C1=N\C(=O)NN(C)C(C)c1ccc(-c2ncn(-c3ccc(OC(F)(F)F)cc3)n2)cc1]

Isolated as a red foam (47 mg, 29%).

### (Z)-2-Methyl-N-(3-(5-methyl-2-(trifluoromethyl)phenyl)-4-oxothiazolidin-2-ylidene)-2-(1-(4-(1-(4-(trifluoromethoxy)phenyl)-1H-1,2,4-triazol-3-yl)phenyl)ethyl)hydrazine-1-carboxamide (A37)

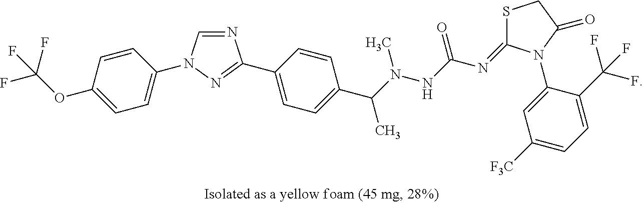 [SMILES: CC(c1ccc(-c2ncn(-c3ccc(OC(F)(F)F)cc3)n2)cc1)N(C)NC(=O)/N=C1\SCC(=O)N1c1cc(C(F)(F)F)ccc1C(F)(F)F]

Isolated as a yellow foam (45 mg, 28%).

### (Z)-2-Methyl-N-(3-(5-methyl-2-(2,2,2-trifluoroethoxy)phenyl)-4-oxothiazolidin ylidene)-2-(1-(4-(1-(4-(trifluoromethoxy)phenyl)-1H-1,2,4-triazol yl)phenyl)ethyl)hydrazine-1-carboxamide (A38)

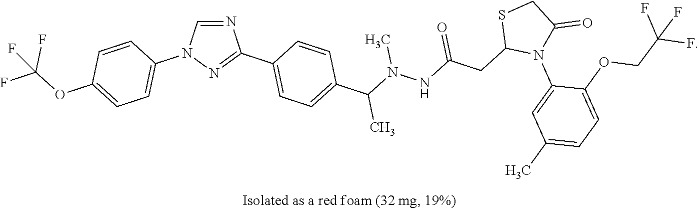 [SMILES: Cc1ccc(OCC(F)(F)F)c(N2C(=O)CSC2CC(=O)NN(C)C(C)c2ccc(-c3ncn(-c4ccc(OC(F)(F)F)cc4)n3)cc2)c1]

Isolated as a red foam (32 mg, 19%).

### Example 2: Preparation of (Z)—N-(3-(2-isopropyl-5-methylphenyl)-4-oxothiazolidin-2-ylidene)-2-methyl-2-(4-(1-(4-(trifluoromethoxy)phenyl)-1H-1,2,4-triazol-3-yl)benzyl)hydrazine-1-carboxamide (A4)

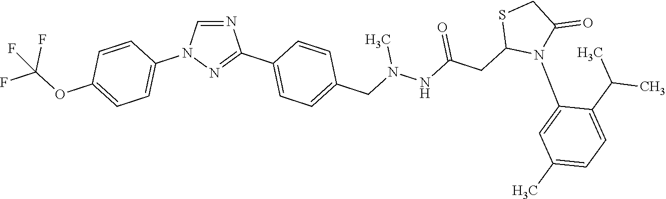 [SMILES: Cc1ccc(C(C)C)c(N2C(=O)CSC2CC(=O)NN(C)Cc2ccc(-c3ncn(-c4ccc(OC(F)(F)F)cc4)n3)cc2)c1]

3-(4-((1-Methylhydrazinyl)methyl)phenyl)-1-(4-(trifluoromethoxy)phenyl)-1H-1,2,4-triazole hydrochloride (C2; 0.050 g, 0.125 mmol) was combined with bis(2,5-dioxopyrrolidin-1-yl) carbonate (0.040 g, 0.156 mmol) in acetonitrile (1.251 mL), and N,N-diisopropylethylamine (0.066 mL, 0.375 mmol) was added. The reaction mixture was stirred for 30 min, and 2-imino-3-(2-isopropyl-5-methylphenyl)thiazolidin-4-one (0.037 g, 0.150 mmol) was added. The mixture was stirred at rt for 30 min and was concentrated. Purification via silica gel chromatography with a gradient of 0-80% EtOAc in hexanes provided the title compound as a foamy, sticky, semi-solid (45 mg, 56%).

### Example 3: Preparation of (Z)—N-(3-(5-chloro-2-(trifluoromethoxy)phenyl)-4-oxothiazolidin-2-ylidene)-2-(1-(4-(1-(4-(trifluoromethoxy)phenyl)-1H-1,2,4-triazol-3-yl)phenyl)ethyl)hydrazine-1-carboxamide (A5)

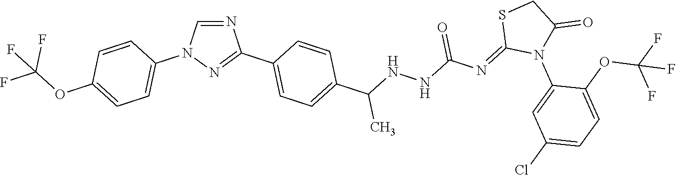 [SMILES: CC(NNC(=O)/N=C1\SCC(=O)N1c1cc(Cl)ccc1OC(F)(F)F)c1ccc(-c2ncn(-c3ccc(OC(F)(F)F)cc3)n2)cc1]

3-(4-(1-Hydrazinylethyl)phenyl)-1-(4-(trifluoromethoxy)phenyl)-1H-1,2,4-triazole hydrochloride (C3; 0.010 g, 0.025 mmol) was suspended in dry acetonitrile (0.250 mL) and 4-nitrophenyl (Z)-(3-(2-isopropyl-5-methylphenyl)-4-oxothiazolidin-2-ylidene)carbamate (0.012 g, 0.030 mmol) was added. To the pale yellow mixture was added N,N-diisopropylethylamine (0.013 mL, 0.075 mmol). The mixture was heated to 55° C. for 5 h. The reaction mixture was concentrated. Purification via silica gel chromatography with a gradient of 0-80% acetone in hexanes afforded the title compound as a clear, colorless oil observed as a mixture of rotamers by NMR spectroscopy (32 mg, 37%).

The following compounds were prepared in like manner to the procedure outlined in Example 3:

### (Z)—N-(3-(5-Methyl-2-(2,2,2-trifluoroethoxy)phenyl)-4-oxothiazolidin-2-ylidene)-2-(1-(4-(1-(4-(trifluoromethoxy)phenyl)-1H-1,2,4-triazol-3-yl)phenyl)ethyl)hydrazine-1-carboxamide (A6)

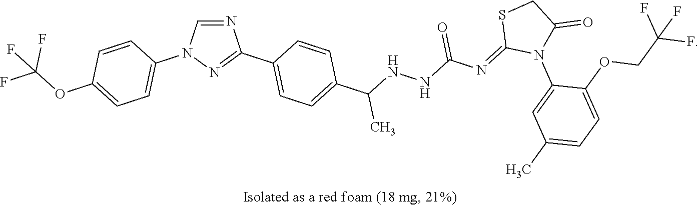 [SMILES: Cc1ccc(OCC(F)(F)F)c(N2C(=O)CS/C2=N\C(=O)NNC(C)c2ccc(-c3ncn(-c4ccc(OC(F)(F)F)cc4)n3)cc2)c1]

Isolated as a red foam (18 mg, 21%).

### (Z)—N-(3-(5-Chloro-2-(2,2,2-trifluoroethoxy)phenyl)-4-oxothiazolidin-2-ylidene)-2-(1-(4-(1-(4-(trifluoromethoxy)phenyl)-1H-1,2,4-triazol-3-yl)phenyl)ethyl)hydrazine-1-carboxamide (A7)

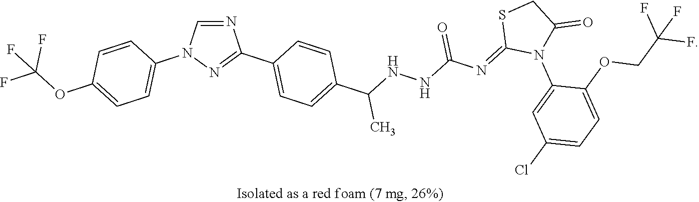 [SMILES: CC(NNC(=O)/N=C1\SCC(=O)N1c1cc(Cl)ccc1OCC(F)(F)F)c1ccc(-c2ncn(-c3ccc(OC(F)(F)F)cc3)n2)cc1]

Isolated as a red foam (7 mg, 26%).

### (Z)—N-(3-(5-Chloro-2-isopropylphenyl)-4-oxothiazolidin-2-ylidene)-2-(1-(4-(1-(4-(trifluoromethoxy)phenyl)-1H-1,2,4-triazol-3-yl)phenyl)ethyl)hydrazine-1-carboxamide (A8)

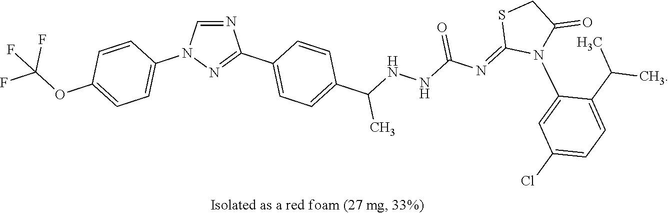 [SMILES: CC(C)c1ccc(Cl)cc1N1C(=O)CS/C1=N\C(=O)NNC(C)c1ccc(-c2ncn(-c3ccc(OC(F)(F)F)cc3)n2)cc1]

Isolated as a red foam (27 mg, 33%).

### (Z)—N-(3-(2-Isopropyl-5-methylphenyl)-4-oxothiazolidin-2-ylidene)-2-(1-(4-(1-(4-(trifluoromethoxy)phenyl)-1H-1,2,4-triazol-3-yl)phenyl)ethyl)hydrazine-1-carboxamide (A9)

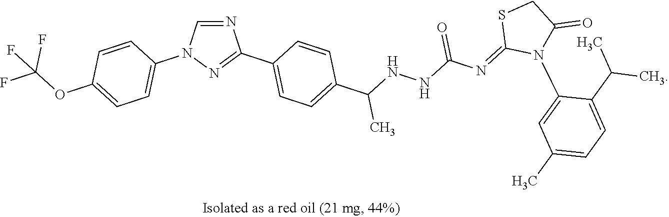 [SMILES: Cc1ccc(C(C)C)c(N2C(=O)CS/C2=N\C(=O)NNC(C)c2ccc(-c3ncn(-c4ccc(OC(F)(F)F)cc4)n3)cc2)c1]

Isolated as a red oil (21 mg, 44%).

### Example 4: Preparation of (Z)-1-(3-(5-methyl-2-propylphenyl)-4-oxothiazolidin-2-ylidene)-3-((4-(1-(4-(trifluoromethoxy)phenyl)-1H-1,2,4-triazol-3-yl)benzyl)oxy)urea (A10)

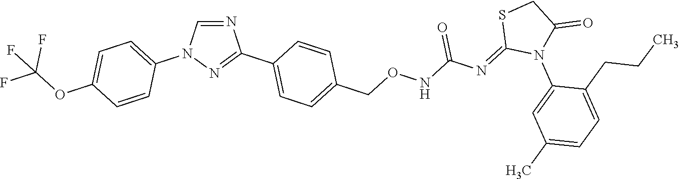 [SMILES: CCCc1ccc(C)cc1N1C(=O)CS/C1=N\C(=O)NOCc1ccc(-c2ncn(-c3ccc(OC(F)(F)F)cc3)n2)cc1]

To O-(4-(1-(4-(trifluoromethoxy)phenyl)-1H-1,2,4-triazol-3-yl)benzyl)hydroxylamine (C₄; 58 mg, 0.17 mmol) and N,N-disuccinimidyl carbonate (51 mg, 0.20 mmol) in acetonitrile (0.83 mL) was added pyridine (0.054 mL, 0.66 mmol). The reaction mixture was stirred at rt for 1 h, then concentrated and dissolved in DCM (0.8 mL). 2-Imino-3-(5-methyl-2-propylphenyl)thiazolidin-4-one (49 mg, 0.20 mmol), sodium bicarbonate (NaHCO₃; 139 mg, 1.66 mmol), and water (0.2 mL) were added. The reaction mixture was stirred at rt for 1 h and diluted with water and dichloromethane. The mixture was filtered through a phase separator directly onto a Celite® cartridge. Purification by flash chromatography (0-40% gradient, hold at 40%, 40-100% EtOAc/[1:1 DCM/hexanes] gradient) provided the title compound as a yellow oil (69 mg, 63% yield).

The following compounds were prepared in like manner to the procedure outlined in Example 4:

### (Z)-1-(3-(2-Chloro-5-methylphenyl)-4-oxothiazolidin-2-ylidene)-3-(1-(4-(1-(4-(trifluoromethoxy)phenyl)-1H-1,2,4-triazol-3-yl)phenyl)ethoxy)urea (A11)

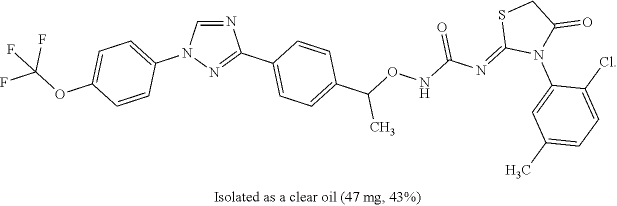 [SMILES: Cc1ccc(Cl)c(N2C(=O)CS/C2=N\C(=O)NOC(C)c2ccc(-c3ncn(-c4ccc(OC(F)(F)F)cc4)n3)cc2)c1]

Isolated as a clear oil (47 mg, 43%).

### (Z)-1-(3-(5-Methyl-2-propylphenyl)-4-oxothiazolidin-2-ylidene)-3-(1-(4-(1-(4-(trifluoromethoxy)phenyl)-1H-1,2,4-triazol-3-yl)phenyl)ethoxy)urea (A12)

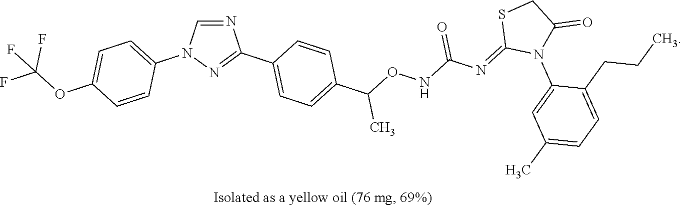 [SMILES: CCCc1ccc(C)cc1N1C(=O)CS/C1=N\C(=O)NOC(C)c1ccc(-c2ncn(-c3ccc(OC(F)(F)F)cc3)n2)cc1]

Isolated as a yellow oil (76 mg, 69%).

### (Z)-1-(3-(5-Methyl-2-(trifluoromethyl)phenyl)-4-oxothiazolidin-2-ylidene)-3-(1-(4-(1-(4-(trifluoromethoxy)phenyl)-1H-1,2,4-triazol-3-yl)phenyl)ethoxy)urea (A13)

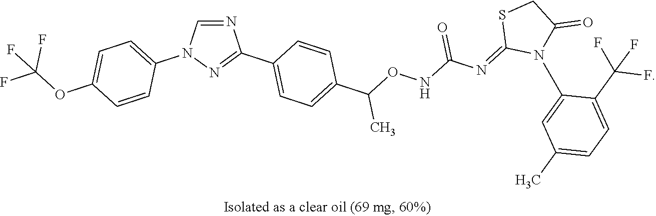 [SMILES: Cc1ccc(C(F)(F)F)c(N2C(=O)CS/C2=N\C(=O)NOC(C)c2ccc(-c3ncn(-c4ccc(OC(F)(F)F)cc4)n3)cc2)c1]

Isolated as a clear oil (69 mg, 60%).

### (Z)-1-(3-(2-Isopropyl-5-methoxyphenyl)-4-oxothiazolidin-2-ylidene)-3-(1-(4-(1-(4-(trifluoromethoxy)phenyl)-1H-1,2,4-triazol-3-yl)phenyl)ethoxy)urea (A14)

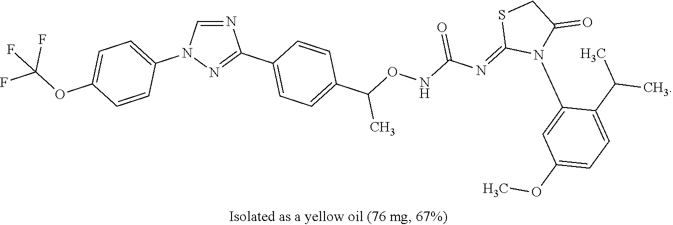 [SMILES: COc1ccc(C(C)C)c(N2C(=O)CS/C2=N\C(=O)NOC(C)c2ccc(-c3ncn(-c4ccc(OC(F)(F)F)cc4)n3)cc2)c1]

Isolated as a yellow oil (76 mg, 67%).

### (Z)-1-(3-(5-Methoxy-2-propylphenyl)-4-oxothiazolidin-2-ylidene)-3-(1-(4-(1-(4-(trifluoromethoxy)phenyl)-1H-1,2,4-triazol-3-yl)phenyl)ethoxy)urea (A15)

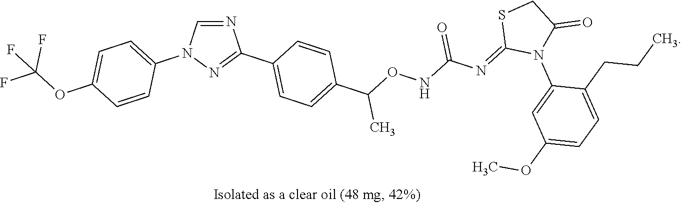 [SMILES: CCCc1ccc(OC)cc1N1C(=O)CS/C1=N\C(=O)NOC(C)c1ccc(-c2ncn(-c3ccc(OC(F)(F)F)cc3)n2)cc1]

Isolated as a clear oil (48 mg, 42%).

### (Z)-1-(3-(2-(Methoxymethyl)-5-methylphenyl)-4-oxothiazolidin-2-ylidene)-3-(1-(4-(1-(4-(trifluoromethoxy)phenyl)-1H-1,2,4-triazol-3-yl)phenyl)ethoxy)urea (A16)

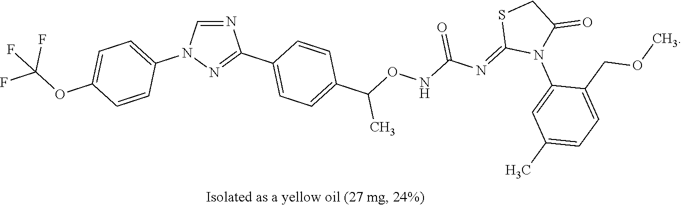 [SMILES: COCc1ccc(C)cc1N1C(=O)CS/C1=N\C(=O)NOC(C)c1ccc(-c2ncn(-c3ccc(OC(F)(F)F)cc3)n2)cc1]

Isolated as a yellow oil (27 mg, 24%).

### (Z)-1-(3-(2-Isopropyl-5-methylphenyl)-4-oxothiazolidin-2-ylidene)-3-(1-(4-(1-(4-(trifluoromethoxy)phenyl)-1H-1,2,4-triazol-3-yl)phenyl)ethoxy)urea (A17)

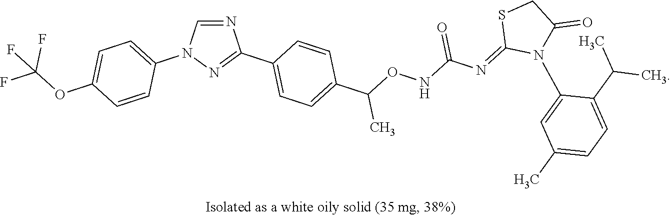 [SMILES: Cc1ccc(C(C)C)c(N2C(=O)CS/C2=N\C(=O)NOC(C)c2ccc(-c3ncn(-c4ccc(OC(F)(F)F)cc4)n3)cc2)c1]

Isolated as a white oily solid (35 mg, 38%).

### (Z)-1-(3-(2-Isopropylphenyl)-4-oxothiazolidin-2-ylidene)-3-(1-(4-(1-(4-(trifluoromethoxy)phenyl)-1H-1,2,4-triazol-3-yl)phenyl)ethoxy)urea (A18)

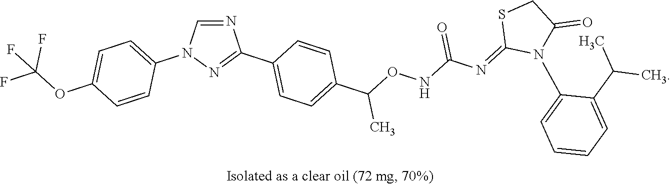 [SMILES: CC(C)c1ccccc1N1C(=O)CS/C1=N\C(=O)NOC(C)c1ccc(-c2ncn(-c3ccc(OC(F)(F)F)cc3)n2)cc1]

Isolated as a clear oil (72 mg, 70%).

### (Z)-1-(3-(5-Methyl-2-(2,2,2-trifluoroethoxy)phenyl)-4-oxothiazolidin-2-ylidene)-3-(1-(4-(1-(4-(trifluoromethoxy)phenyl)-1H-1,2,4-triazol-3-yl)phenyl)ethoxy)urea (A19)

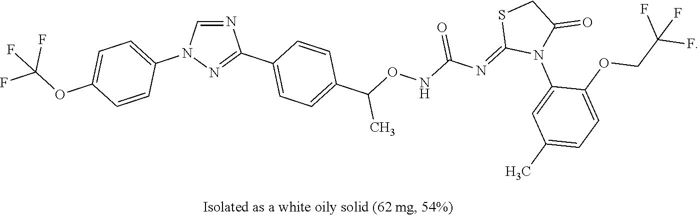 [SMILES: Cc1ccc(OCC(F)(F)F)c(N2C(=O)CS/C2=N\C(=O)NOC(C)c2ccc(-c3ncn(-c4ccc(OC(F)(F)F)cc4)n3)cc2)c1]

Isolated as a white oily solid (62 mg, 54%).

### (Z)-1-(3-(5-Chloro-2-(2,2,2-trifluoroethoxy)phenyl)-4-oxothiazolidin-2-ylidene)-3-(1-(4-(1-(4-(trifluoromethoxy)phenyl)-1H-1,2,4-triazol-3-yl)phenyl)ethoxy)urea (A20)

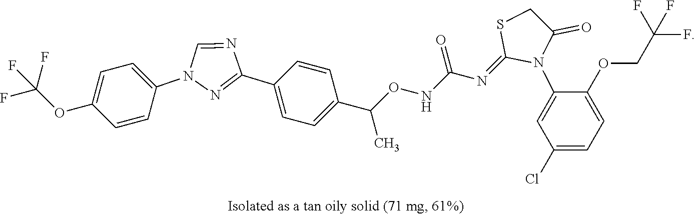 [SMILES: CC(ONC(=O)/N=C1\SCC(=O)N1c1cc(Cl)ccc1OCC(F)(F)F)c1ccc(-c2ncn(-c3ccc(OC(F)(F)F)cc3)n2)cc1]

Isolated as a tan oily solid (71 mg, 61%).

### (Z)-1-(3-(5-Chloro-2-isopropylphenyl)-4-oxothiazolidin-2-ylidene)-3-(1-(4-(1-(4-(trifluoromethoxy)phenyl)-1H-1,2,4-triazol-3-yl)phenyl)ethoxy)urea (A21)

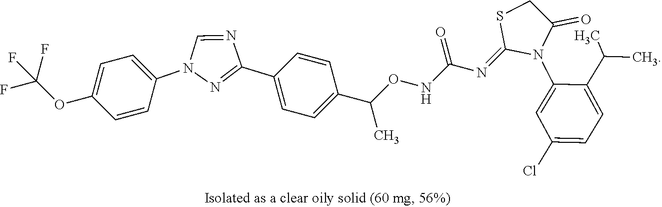 [SMILES: CC(C)c1ccc(Cl)cc1N1C(=O)CS/C1=N\C(=O)NOC(C)c1ccc(-c2ncn(-c3ccc(OC(F)(F)F)cc3)n2)cc1]

Isolated as a clear oily solid (60 mg, 56%).

### (Z)-1-(3-(2-Chloro-5-methylphenyl)-4-oxothiazolidin-2-ylidene)-3-((4-(1-(4-(trifluoromethoxy)phenyl)-1H-1,2,4-triazol-3-yl)benzyl)oxy)urea (A22)

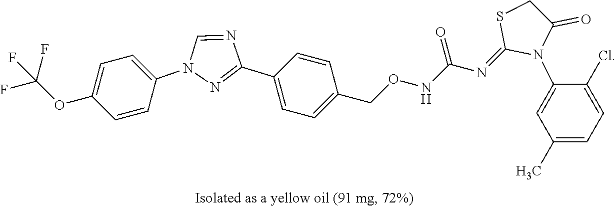 [SMILES: Cc1ccc(Cl)c(N2C(=O)CS/C2=N\C(=O)NOCc2ccc(-c3ncn(-c4ccc(OC(F)(F)F)cc4)n3)cc2)c1]

Isolated as a yellow oil (91 mg, 72%).

### (Z)-1-(3-(5-Methoxy-2-propylphenyl)-4-oxothiazolidin-2-ylidene)-3-((4-(1-(4-(trifluoromethoxy)phenyl)-1H-1,2,4-triazol-3-yl)benzyl)oxy)urea (A23)

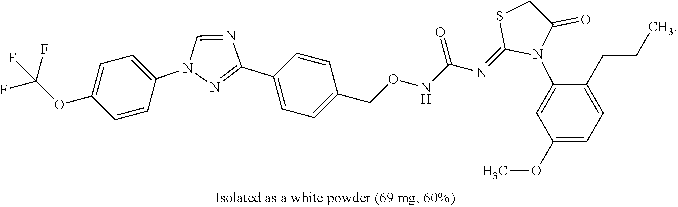 [SMILES: CCCc1ccc(OC)cc1N1C(=O)CS/C1=N\C(=O)NOCc1ccc(-c2ncn(-c3ccc(OC(F)(F)F)cc3)n2)cc1]

Isolated as a white powder (69 mg, 60%).

### (Z)-1-(3-(5-Methyl-2-(trifluoromethyl)phenyl)-4-oxothiazolidin-2-ylidene)-3-((4-(1-(4-(trifluoromethoxy)phenyl)-1H-1,2,4-triazol-3-yl)benzyl)oxy)urea (A24)

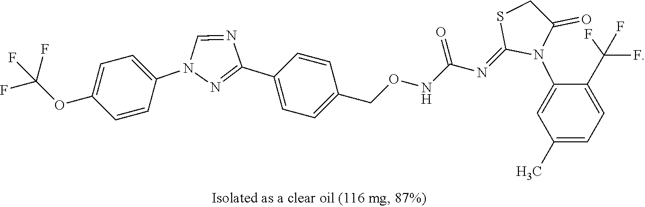 [SMILES: Cc1ccc(C(F)(F)F)c(N2C(=O)CS/C2=N\C(=O)NOCc2ccc(-c3ncn(-c4ccc(OC(F)(F)F)cc4)n3)cc2)c1]

Isolated as a clear oil (116 mg, 87%).

### (Z)-1-(3-(5-Methoxy-2-(2,2,2-trifluoroethyl)phenyl)-4-oxothiazolidin-2-ylidene)-3-((4-(1-(4-(trifluoromethoxy)phenyl)-1H-1,2,4-triazol-3-yl)benzyl)oxy)urea (A25)

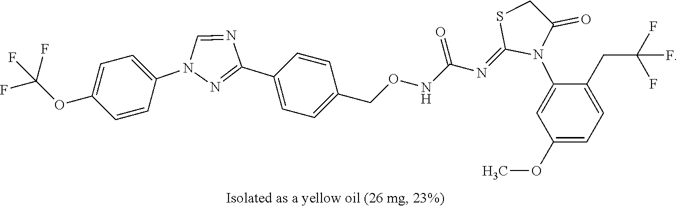 [SMILES: COc1ccc(CC(F)(F)F)c(N2C(=O)CS/C2=N\C(=O)NOCc2ccc(-c3ncn(-c4ccc(OC(F)(F)F)cc4)n3)cc2)c1]

Isolated as a yellow oil (26 mg, 23%).

### (Z)-1-(3-(2-Isopropyl-5-methoxyphenyl)-4-oxothiazolidin-2-ylidene)-3-((4-(1-(4-(trifluoromethoxy)phenyl)-1H-1,2,4-triazol-3-yl)benzyl)oxy)urea (A26)

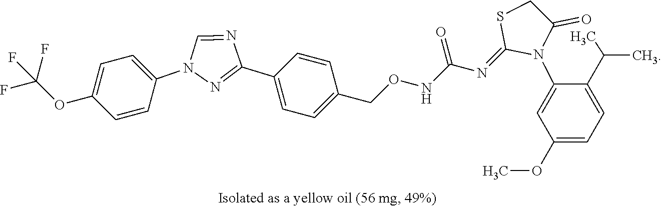 [SMILES: COc1ccc(C(C)C)c(N2C(=O)CS/C2=N\C(=O)NOCc2ccc(-c3ncn(-c4ccc(OC(F)(F)F)cc4)n3)cc2)c1]

Isolated as a yellow oil (56 mg, 49%).

### (Z)-1-(3-(2-(Methoxymethyl)-5-methylphenyl)-4-oxothiazolidin-2-ylidene)-3-((4-(1-(4-(trifluoromethoxy)phenyl)-1H-1,2,4-triazol-3-yl)benzyl)oxy)urea (A27)

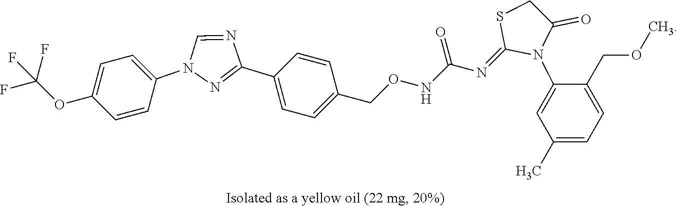 [SMILES: COCc1ccc(C)cc1N1C(=O)CS/C1=N\C(=O)NOCc1ccc(-c2ncn(-c3ccc(OC(F)(F)F)cc3)n2)cc1]

Isolated as a yellow oil (22 mg, 20%).

### (Z)-1-(3-(2-Isopropyl-5-methylphenyl)-4-oxothiazolidin-2-ylidene)-3-((4-(1-(4-(trifluoromethoxy)phenyl)-1H-1,2,4-triazol-3-yl)benzyl)oxy)urea (A28)

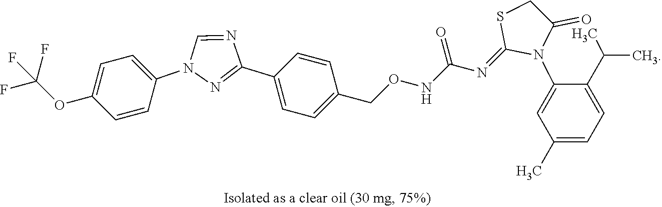 [SMILES: Cc1ccc(C(C)C)c(N2C(=O)CS/C2=N\C(=O)NOCc2ccc(-c3ncn(-c4ccc(OC(F)(F)F)cc4)n3)cc2)c1]

Isolated as a clear oil (30 mg, 75%).

### (Z)-1-(3-(2-Isopropylphenyl)-4-oxothiazolidin-2-ylidene)-3-((4-(1-(4-(trifluoromethoxy)phenyl)-1H-1,2,4-triazol-3-yl)benzyl)oxy)urea (A29)

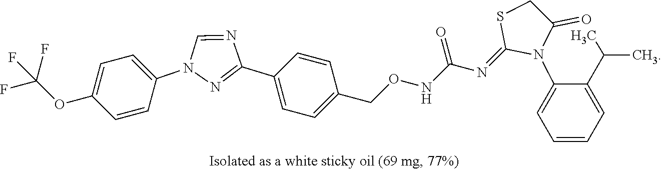 [SMILES: CC(C)c1ccccc1N1C(=O)CS/C1=N\C(=O)NOCc1ccc(-c2ncn(-c3ccc(OC(F)(F)F)cc3)n2)cc1]

Isolated as a white sticky oil (69 mg, 77%).

### (Z)-1-(3-(5-Methyl-2-(2,2,2-trifluoroethoxy)phenyl)-4-oxothiazolidin-2-ylidene)-3-((4-(1-(4-(trifluoromethoxy)phenyl)-1H-1,2,4-triazol-3-yl)benzyl)oxy)urea (A30)

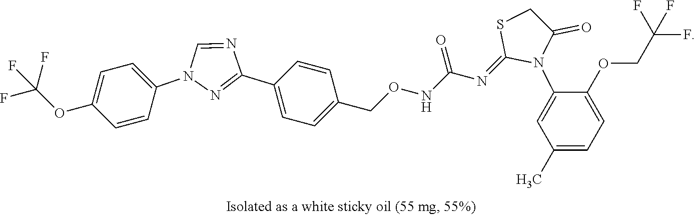 [SMILES: Cc1ccc(OCC(F)(F)F)c(N2C(=O)CS/C2=N\C(=O)NOCc2ccc(-c3ncn(-c4ccc(OC(F)(F)F)cc4)n3)cc2)c1]

Isolated as a white sticky oil (55 mg, 55%).

### (Z)-1-(3-(5-Chloro-2-(2,2,2-trifluoroethoxy)phenyl)-4-oxothiazolidin-2-ylidene)-3-((4-(1-(4-(trifluoromethoxy)phenyl)-1H-1,2,4-triazol-3-yl)benzyl)oxy)urea (A31)

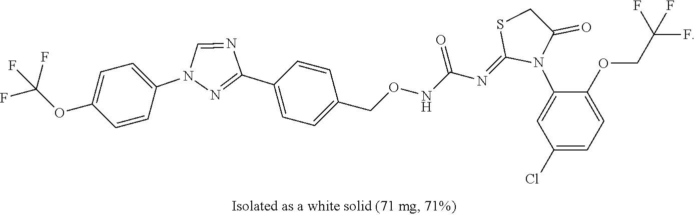 [SMILES: O=C(/N=C1\SCC(=O)N1c1cc(Cl)ccc1OCC(F)(F)F)NOCc1ccc(-c2ncn(-c3ccc(OC(F)(F)F)cc3)n2)cc1]

Isolated as a white solid (71 mg, 71%).

### (Z)-1-(3-(5-Chloro-2-isopropylphenyl)-4-oxothiazolidin-2-ylidene)-3-((4-(1-(4-(trifluoromethoxy)phenyl)-1H-1,2,4-triazol-3-yl)benzyl)oxy)urea (A32)

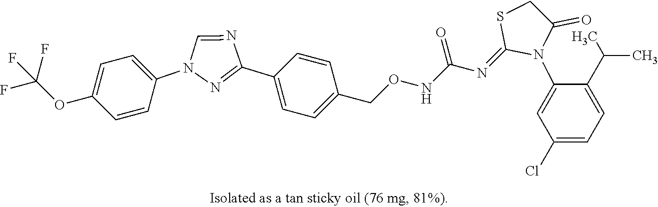 [SMILES: CC(C)c1ccc(Cl)cc1N1C(=O)CS/C1=N\C(=O)NOCc1ccc(-c2ncn(-c3ccc(OC(F)(F)F)cc3)n2)cc1]

Isolated as a tan sticky oil (76 mg, 81%).

### (Z)-1-(3-(2-Isopropyl-5-methylphenyl)-4-oxothiazolidin-2-ylidene)-3-((4-(1-(4-(perfluoroethoxy)phenyl)-1H-1,2,4-triazol-3-yl)benzyl)oxy)urea (A33)

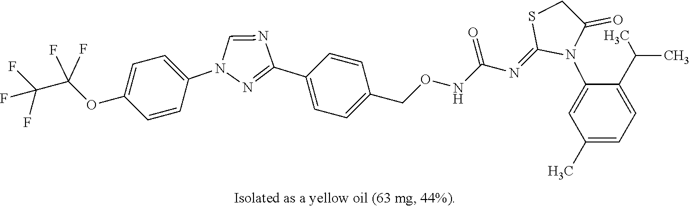 [SMILES: Cc1ccc(C(C)C)c(N2C(=O)CS/C2=N\C(=O)NOCc2ccc(-c3ncn(-c4ccc(OC(F)(F)C(F)(F)F)cc4)n3)cc2)c1]

Isolated as a yellow oil (63 mg, 44%).

### (Z)-1-(3-(2-Isopropyl-5-methylphenyl)-4-oxothiazolidin-2-ylidene)-3-(1-(4-(1-(4-(perfluoroethoxy)phenyl)-1H-1,2,4-triazol-3-yl)phenyl)ethoxy)urea (A34)

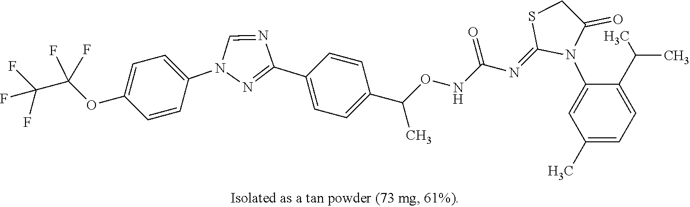 [SMILES: Cc1ccc(C(C)C)c(N2C(=O)CS/C2=N\C(=O)NOC(C)c2ccc(-c3ncn(-c4ccc(OC(F)(F)C(F)(F)F)cc4)n3)cc2)c1]

Isolated as a tan powder (73 mg, 61%).

### tert-Butyl (Z)-(((3-(2-isopropyl-5-methylphenyl)-4-oxothiazolidin-2-ylidene)carbamoyl)oxy)(4-(1-(4-(trifluoromethoxy)phenyl)-1H-1,2,4-triazol-3-yl)benzyl)carbamate (A35)

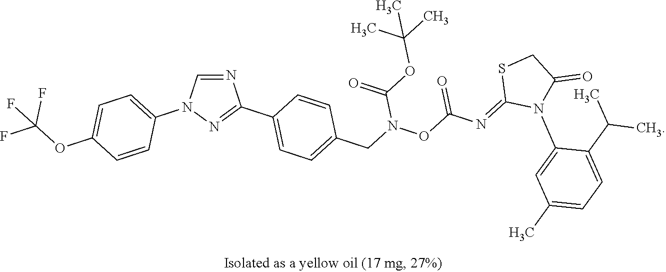 [SMILES: Cc1ccc(C(C)C)c(N2C(=O)CS/C2=N\C(=O)ON(Cc2ccc(-c3ncn(-c4ccc(OC(F)(F)F)cc4)n3)cc2)C(=O)OC(C)(C)C)c1]

Isolated as a yellow oil (17 mg, 27%).

### Example 5: Preparation of 3-(4-(1-hydrazinylethyl)phenyl)-1-(4-(trifluoromethoxy)phenyl)-1H-1,2,4-triazole hydrochloride (C₃)

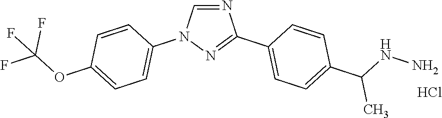 [SMILES: CC(NN)c1ccc(-c2ncn(-c3ccc(OC(F)(F)F)cc3)n2)cc1 ; Cl]

To tert-butyl 2-(1-(4-(1-(4-(trifluoromethoxy)phenyl)-1H-1,2,4-triazol-3-yl)phenyl)ethyl)hydrazine-1-carboxylate (C8; 0.100 g, 0.216 mmol) was added hydrochloric acid (4 M solution in dioxane; 0.270 mL, 1.079 mmol). The reaction mixture was stirred overnight, and the solvent was removed by a stream of N₂. The resulting white solid (81 mg, 94%) was used in the next reaction without further manipulation: mp 180-183° C.; ¹H NMR (400 MHz, DMSO-d₆) δ 9.45 (s, 1H), 8.13 (d, J=8.4 Hz, 2H), 8.09 (d, J=8.8 Hz, 2H), 7.63 (d, J=8.8 Hz, 2H), 7.59 (d, J=8.4 Hz, 2H), 4.30-4.25 (m, 1H), 1.44 (d, J=6.8 Hz, 3H); ¹⁹F NMR (376 MHz, DMSO-d₆) δ −57.00; ESIMS m/z 364.5 ([M+H]⁺).

The following compounds were prepared in like manner to the procedure outlined in Example 5:

### 3-(4-(1-(1-Methylhydrazinyl)ethyl)phenyl)-1-(4-(trifluoromethoxy)phenyl)-1H-1,2,4-triazole hydrochloride (C₁)

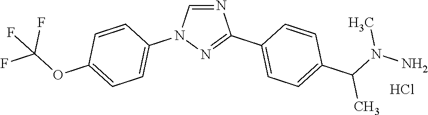 [SMILES: CC(c1ccc(-c2ncn(-c3ccc(OC(F)(F)F)cc3)n2)cc1)N(C)N ; Cl]

Isolated as a white solid (471 mg, 86%): ¹H NMR (300 MHz, methanol-d₄) δ 9.39 (s, 1H), 8.25-8.21 (m, 2H), 8.09-8.02 (m, 2H), 7.66-7.59 (m, 2H), 7.57-7.49 (m, 2H), 4.34-4.22 (m, 1H), 2.79 (s, 3H), 1.65 (d, J=6.8 Hz, 3H); ESIMS m/z 378.2 ([M+H]⁺).

### 3-(44(1-Methylhydrazinyl)methyl)phenyl)-1-(4-(trifluoromethoxy)phenyl)-1H-1,2,4-triazole hydrochloride (C₂)

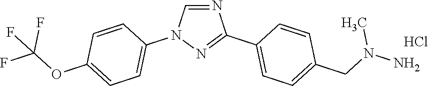 [SMILES: CN(N)Cc1ccc(-c2ncn(-c3ccc(OC(F)(F)F)cc3)n2)cc1 ; Cl]

Isolated as a white solid (36 mg, 95%): ¹H NMR (400 MHz, methanol-d₄) δ 9.24 (s, 1H), 8.22 (d, J=8.0 Hz, 2H), 8.10-8.01 (m, 2H), 7.58 (d, J=8.1 Hz, 2H), 7.52 (d, J=8.5 Hz, 2H), 4.17 (s, 2H), 2.79 (s, 3H); ¹⁹F NMR (376 MHz, methanol-d₄) δ −59.68; ESIMS m/z 364.2 ([M+H]+).

### Example 6: Preparation of tert-butyl 2-(1-(4-(1-(4-(trifluoromethoxy)phenyl)-1H-1,2,4-triazol-3-yl)phenyl)ethyl)hydrazine-1-carboxylate (C₈)

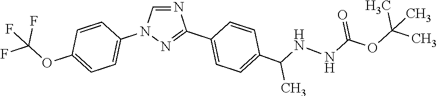 [SMILES: CC(NNC(=O)OC(C)(C)C)c1ccc(-c2ncn(-c3ccc(OC(F)(F)F)cc3)n2)cc1]

tert-Butyl hydrazinecarboxylate (0.188 g, 1.42 mmol) and 1-(4-(1-(4-(trifluoromethoxy)phenyl)-1H-1,2,4-triazol-3-yl)phenyl)ethan-1-one (0.412 g, 1.19 mmol) (see for example WO 2011/017504 A1) were dissolved in ethanol (7.91 mL). The turbid solution was heated to 80° C. for 1 h, at which point conversion to the desired hydrazone was judged complete by thin layer chromatography. The reaction mixture was cooled, and the hydrazone precipitated from solution. Acetic acid (0.204 mL, 3.56 mmol) and sodium cyanoborohydride (0.112 g, 1.78 mmol) were added, and the reaction mixture was heated to 80° C. for 45 min. The reaction mixture was poured into water and extracted with EtOAc. The organic extracts were washed with brine, dried, and concentrated to a pale yellow oil. Purification via silica gel chromatography with a gradient of 0-40% acetone in hexanes provided the title compound as a clear oil that foamed and became a white gum upon drying under high vacuum (0.103 g, 77%): 41 NMR (400 MHz, CDCl₃) δ 8.56 (s, 1H), 8.18-8.14 (m, 2H), 7.84-7.78 (m, 2H), 7.47 (d, J=8.0 Hz, 2H), 7.39 (d, J=8.7 Hz, 2H), 5.97 (s, 1H), 4.28 (s, 1H), 1.44 (s, 9H), 1.37 (d, J=6.6 Hz, 3H); ¹⁹F NMR (376 MHz, CDCl₃) δ −58.03; IR (thin film) 3285, 2977, 1697, 1515 cm⁻¹; ESIMS m/z 464.2 ([M+H]+).

### Example 7: Preparation of tert-butyl 2-methyl-2-(1-(4-(1-(4-(trifluoromethoxy)phenyl)-1H-1,2,4-triazol-3-yl)phenyl)ethyl)hydrazine-1-carboxylate (C₆)

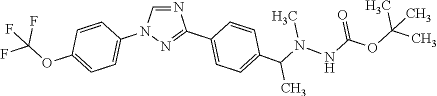 [SMILES: CC(c1ccc(-c2ncn(-c3ccc(OC(F)(F)F)cc3)n2)cc1)N(C)NC(=O)OC(C)(C)C]

1-(4-(1-(4-(Trifluoromethoxy)phenyl)-1H-1,2,4-triazol-3-yl)phenyl)ethan-1-one (1 g, 2.88 mmol) (see for example WO 2011/017504 A1) and tert-butyl hydrazinecarboxylate (0.438 g, 3.31 mmol) were dissolved in ethanol (11.5 mL). The reaction mixture was heated to 80° C. After 30 min, the mixture was cooled, and acetic acid (0.495 mL, 8.64 mmol) and sodium cyanoborohydride (0.543 g, 8.64 mmol) were added. The reaction mixture was stirred for 30 min at rt, and the mixture was briefly heated to 80° C. (with proper venting) and cooled again. Formaldehyde (37% aqueous; 0.268 mL, 3.60 mmol) and sodium cyanoborohydride (0.543 g, 8.64 mmol) were both added, and the reaction mixture was stirred for 30 min. The solvent was removed, and the residue was partitioned between water and DCM. The phases were separated and the organic layer was concentrated. Purification via silica gel chromatography with a gradient of 0-50% EtOAc in hexanes provided the title compound as a white, amorphous solid (1.202 g, 87%): ¹H NMR (300 MHz, CDCl₃) δ 8.58 (s, 1H), 8.21-8.10 (m, 2H), 7.86-7.75 (m, 2H), 7.48-7.43 (m, 2H), 7.43-7.36 (m, 2H), 5.44 (s, 1H), 3.97 (s, 1H), 2.51 (s, 3H), 1.50-1.37 (m, 12H); ESIMS m/z 478.3 ([M+H]⁺).

The following compounds were prepared in like manner to the procedure outlined in Example 7:

### tert-Butyl 2-methyl-2-(4-(1-(4-(trifluoromethoxy)phenyl)-1H-1,2,4-triazol yl)benzyl)hydrazine-1-carboxylate (C₇)

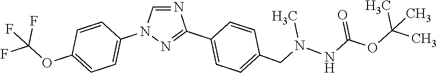 [SMILES: CN(Cc1ccc(-c2ncn(-c3ccc(OC(F)(F)F)cc3)n2)cc1)NC(=O)OC(C)(C)C]

Starting from 4-(1-(4-(trifluoromethoxy)phenyl)-1H-1,2,4-triazol-3-yl)benzaldehyde (see for example WO 2009/102736 A1), the title compound was prepared and isolated as a yellow oil (475 mg, 92%): ¹H NMR (400 MHz, CDCl₃) δ 8.62 (s, 1H), 8.19-8.11 (m, 2H), 7.84-7.76 (m, 2H), 7.46 (d, J=8.0 Hz, 2H), 7.38 (d, J=8.5 Hz, 2H), 5.86 (d, J=26.2 Hz, 1H), 3.99 (s, 2H), 2.67 (s, 3H), 1.42 (s, 9H); ¹⁹F NMR (376 MHz, CDCl₃) δ −58.05; ESIMS m/z 464.3 ([M+H]⁺).

### Example 8: Preparation of tert-butyl 2-methyl-2-(4-(1-(4-(trifluoromethoxy)phenyl)-1H-1,2,4-triazol-3-yl)benzyl)hydrazine-1-carboxylate (C₇)

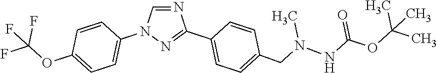 [SMILES: CN(Cc1ccc(-c2ncn(-c3ccc(OC(F)(F)F)cc3)n2)cc1)NC(=O)OC(C)(C)C]

tert-Butyl 2-(4-(1-(4-(trifluoromethoxy)phenyl)-1H-1,2,4-triazol-3-yl)benzyl)hydrazine-1-carboxylate (C₈; 0.050 g, 0.111 mmol) was dissolved in ethanol (1.11 mL). Formaldehyde (0.012 mL, 0.167 mmol) and acetic acid (0.013 mL, 0.223 mmol) were added. Solid sodium cyanoborohydride (0.014 g, 0.223 mmol) was added, and the mixture stirred for 60 min at rt. The reaction mixture was then poured into water and extracted with diethyl ether. The organic extracts were dried and concentrated. The title compound was isolated as a sticky, yellow oil, which was used without further purification (44 mg, 85%): ¹H NMR (400 MHz, CDCl₃) δ 8.62 (s, 1H), 8.19-8.11 (m, 2H), 7.84-7.76 (m, 2H), 7.46 (d, J=8.0 Hz, 2H), 7.38 (d, J=8.5 Hz, 2H), 5.86 (d, J=26.2 Hz, 1H), 3.99 (s, 2H), 2.67 (s, 3H), 1.42 (s, 9H); ¹⁹F NMR (376 MHz, CDCl₃) δ −58.05; ESIMS m/z 464.3 ([M+H]⁺).

### Example 9: Preparation of tert-butyl 2-(4-(1-(4-(trifluoromethoxy)phenyl)-1H-1,2,4-triazol-3-yl)benzyl)hydrazine-1-carboxylate (C₈)

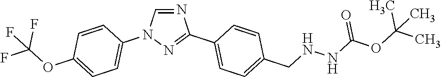 [SMILES: CC(C)(C)OC(=O)NNCc1ccc(-c2ncn(-c3ccc(OC(F)(F)F)cc3)n2)cc1]

tert-Butyl hydrazinecarboxylate (0.416 g, 3.15 mmol) was combined with 4-(1-(4-(trifluoromethoxy)phenyl)-1H-1,2,4-triazol-3-yl)benzaldehyde (1 g, 3.00 mmol) (see for example WO 2009/102736 A1) in ethanol (15 mL). The mixture was heated to 80° C. for 45 min, at which point the solution became homogeneous and yellow in color. The reaction mixture was cooled, which induced precipitation. Acetic acid (0.858 mL, 15 mmol) and sodium cyanoborohydride (0.566 g, 9 mmol) were added sequentially. After the initial off-gassing, the mixture was heated to 80° C. for 60 min. The reaction mixture was then cooled, poured into water, and extracted with ether. The organic extracts were washed with brine and dried over Na₂SO₄. The solvents were concentrated. The title compound was isolated as an off-white, amorphous solid that was used without further purification (1.30 g, 96%): ¹H NMR (400 MHz, CDCl₃) δ 8.59 (s, 1H), 8.19-8.14 (m, 2H), 7.84-7.79 (m, 2H), 7.47 (d, J=7.9 Hz, 2H), 7.39 (d, J=8.5 Hz, 2H), 6.23 (s, 1H), 4.07 (s, 2H), 1.48 (s, 9H); ¹⁹F NMR (376 MHz, CDCl₃) δ −58.03; ESIMS m/z 450.3 ([M+H]+).

### Example 10: Preparation of 0-(4-(1-(4-(trifluoromethoxy)phenyl)-1H-1,2,4-triazol-3-yl)benzyl)hydroxylamine (C₄)

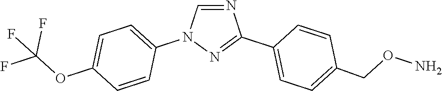 [SMILES: NOCc1ccc(-c2ncn(-c3ccc(OC(F)(F)F)cc3)n2)cc1]

To 2-((4-(1-(4-(trifluoromethoxy)phenyl)-1H-1,2,4-triazol-3-yl)benzyl)oxy)isoindoline-1,3-dione (C9; 348 mg, 0.72 mmol) in DCM (2.9 mL) was added hydrazine monohydrate (0.053 mL, 1.09 mmol). The reaction mixture was stirred at rt for 2 h. The reaction mixture was diluted with DCM and 1 Normal (N) sodium hydroxide (NaOH). The biphasic mixture was filtered through a phase separator and concentrated to provide the title compound as a white solid (257 mg, 100%): mp 92-94.5° C.; ¹H NMR (400 MHz, CDCl₃) δ 8.57 (s, 1H), 8.20 (d, J=8.2 Hz, 2H), 7.83-7.77 (m, 2H), 7.51-7.46 (m, 2H), 7.39 (dt, J=8.1, 1.1 Hz, 2H), 5.46 (s, 2H), 4.76 (s, 2H); ¹⁹F NMR (376 MHz, CDCl₃) δ −58.03; HRMS-ESI (m/z) [M+H]⁺ calcd for C₁₆H₁₃F₃N₄O₂, 351.1063; found, 351.1069.

The following compounds were prepared in like manner to the procedure outlined in Example 10:

### O-(1-(4-(1-(4-(Trifluoromethoxy)phenyl)-1H-1,2,4-triazol-3-yl)phenyl)ethyl)hydroxylamine (C₁₀)

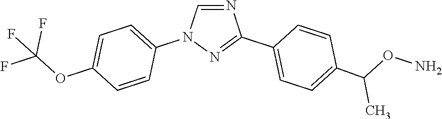 [SMILES: CC(ON)c1ccc(-c2ncn(-c3ccc(OC(F)(F)F)cc3)n2)cc1]

Isolated as a yellow oil (563 mg, 98%): ¹H NMR (400 MHz, CDCl₃) δ 8.57 (s, 1H), 8.28-8.15 (m, 2H), 7.84-7.76 (m, 2H), 7.46 (d, J=8.2 Hz, 2H), 7.39 (d, J=8.6 Hz, 2H), 5.28 (s, 2H), 4.79-4.65 (m, 1H), 1.47 (d, J=6.5 Hz, 3H); ¹⁹F NMR (376 MHz, CDCl₃) δ −58.03; HRMS-ESI (m/z) [M+H]⁺ calcd for C₁₇H₁₅F₃N₄O₂, 365.1220; found, 365.1220.

### O-(4-(1-(4-(Perfluoroethoxy)phenyl)-1H-1,2,4-triazol-3-yl)benzyl)hydroxylamine (C₁₁)

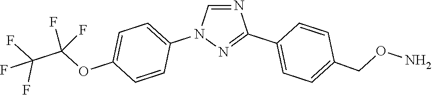 [SMILES: NOCc1ccc(-c2ncn(-c3ccc(OC(F)(F)C(F)(F)F)cc3)n2)cc1]

Isolated as a white solid (83 mg, 63%): mp 83-88° C.; IE NMR (400 MHz, CDCl₃) δ 8.58 (s, 1H), 8.23-8.16 (m, 2H), 7.85-7.77 (m, 2H), 7.48 (d, J=7.9 Hz, 2H), 7.40 (d, J=8.7 Hz, 3H), 5.46 (s, 2H), 4.76 (s, 2H); ¹⁹F NMR (376 MHz, CDCl₃) δ −85.90, −87.85; HRMS-ESI (m/z) [M+H]⁺ calcd for C₁₇H₁₅F₅N₄O₂, 401.1031; found, 401.1029.

### O-(1-(4-(1-(4-(Perfluoroethoxy)phenyl)-1H-1,2,4-triazol-3-yl)phenyl)ethyl)hydroxylamine (C₁₂)

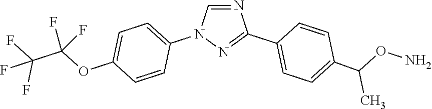 [SMILES: CC(ON)c1ccc(-c2ncn(-c3ccc(OC(F)(F)C(F)(F)F)cc3)n2)cc1]

Isolated as a clear oil (142 mg, 81%): ¹H NMR (400 MHz, CDCl₃) δ 8.58 (s, 1H), 8.24-8.15 (m, 2H), 7.87-7.77 (m, 2H), 7.49-7.43 (m, 2H), 7.40 (d, J=8.8 Hz, 2H), 5.28 (s, 2H), 4.73 (q, J=6.6 Hz, 1H), 1.47 (d, J=6.6 Hz, 3H); ¹⁹F NMR (376 MHz, CDCl₃) δ −85.89, −87.84; HRMS-ESI (m/z) [M+H]⁺ calcd for C₁₈H₁₅F₅N₄O₂, 415.1188; found, 415.1186.

### Example 11: Preparation of 2-((4-(1-(4-(trifluoromethoxy)phenyl)-1H-1,2,4-triazol-3-yl)benzyl)oxy)isoindoline-1,3-dione (C₉)

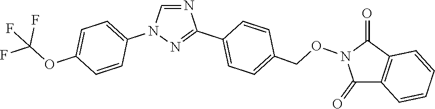 [SMILES: O=C1c2ccccc2C(=O)N1OCc1ccc(-c2ncn(-c3ccc(OC(F)(F)F)cc3)n2)cc1]

To 2-hydroxyisoindoline-1,3-dione (147 mg, 0.90 mmol) in N,N-dimethylformamide (DMF; 2.5 mL) at 0° C. was added 1,2-diazabicyclo[5.4.0]undec-7-ene (DBU; 135 microliters (μL), 0.90 mmol). The reaction mixture was stirred for 5 min, and 3-(4-(bromomethyl)phenyl)-1-(4-(trifluoromethoxy)phenyl)-1H-1,2,4-triazole (prepared as in WO2012027521 A1; 300 mg, 0.753 mmol) was added. The reaction mixture was stirred at rt for 1 h and then quenched with water and 1 N hydrochloric acid. The resultant white precipitate was collected by vacuum filtration and dried in the vacuum oven overnight. The title compound was isolated as a white solid (362 mg, 99%): mp 200-202° C.; ¹H NMR (400 MHz, CDCl₃) δ 8.57 (s, 1H), 8.22 (d, J=8.2 Hz, 2H), 7.84-7.77 (m, 4H), 7.74 (dd, J=5.5, 3.0 Hz, 2H), 7.66 (d, J=8.2 Hz, 2H), 7.39 (d, J=8.5 Hz, 2H), 5.28 (s, 2H); ¹⁹F NMR (376 MHz, CDCl₃) δ −58.02; HRMS-ESI (m/z) [M+H]⁺ calcd for C₂₄H¹⁵F³N₄O₄, 481.1118; found, 481.1122.

The following compounds were prepared in like manner to the procedure outlined in Example 11:

### 2-(1-(4-(1-(4-(Trifluoromethoxy)phenyl)-1H-1,2,4-triazol-3-yl)phenyl)ethoxy)isoindoline-1,3-dione (C₁₃)

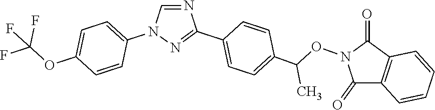 [SMILES: CC(ON1C(=O)c2ccccc2C1=O)c1ccc(-c2ncn(-c3ccc(OC(F)(F)F)cc3)n2)cc1]

Isolated as a white solid (791 mg, 92%): mp 150-155° C.; ¹H NMR (400 MHz, CDCl₃) δ 8.55 (s, 1H), 8.17 (d, J=8.0 Hz, 2H), 7.80-7.73 (m, 4H), 7.69 (dd, J=5.4, 3.1 Hz, 2H), 7.63 (d, J=8.1 Hz, 2H), 7.38 (d, J=8.5 Hz, 2H), 5.58 (q, J=6.5 Hz, 1H), 1.76 (d, J=6.5 Hz, 3H); ¹⁹F NMR (376 MHz, CDCl₃) δ −58.03; HRMS-ESI (m/z) [M+H]⁺ calcd for C₂₅H₁₇F₃N₄O₄, 495.1275; found, 495.1275.

### 2-((4-(1-(4-(Perfluoroethoxy)phenyl)-1H-1,2,4-triazol-3-yl)benzyl)oxy)isoindoline-1,3-dione (C₁₄)

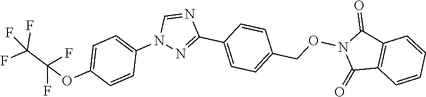 [SMILES: O=C1c2ccccc2C(=O)N1OCc1ccc(-c2ncn(-c3ccc(OC(F)(F)C(F)(F)F)cc3)n2)cc1]

Isolated as a white solid (194 mg, 76%): mp 181-186° C.; ¹H NMR (400 MHz, CDCl₃) δ 8.58 (s, 1H), 8.27-8.16 (m, 2H), 7.85-7.78 (m, 4H), 7.74 (dd, J=5.5, 3.1 Hz, 2H), 7.66 (d, J=8.0 Hz, 2H), 7.40 (d, J=8.6 Hz, 2H), 5.28 (s, 2H); ¹⁹F NMR (376 MHz, CDCl₃) δ −85.90, −87.85; HRMS-ESI (m/z) [M+H]⁺ calcd for C₂₅H₁₅F₅N₄O₄, 531.1086;

### 2-(1-(4-(1-(4-(Perfluoroethoxy)phenyl)-1H-1,2,4-triazol-3-yl)phenyl)ethoxy)isoindoline-1,3-dione (C₁₅)

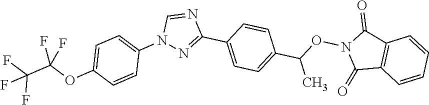 [SMILES: CC(ON1C(=O)c2ccccc2C1=O)c1ccc(-c2ncn(-c3ccc(OC(F)(F)C(F)(F)F)cc3)n2)cc1]

Isolated as a white solid (221 mg, 82%): mp 140-144° C.; ¹H NMR (400 MHz, CDCl₃) δ 8.56 (s, 1H), 8.19-8.13 (m, 2H), 7.82-7.77 (m, 2H), 7.75 (dd, J=5.5, 3.2 Hz, 2H), 7.69 (dd, J=5.5, 3.1 Hz, 2H), 7.63 (d, J=8.1 Hz, 2H), 7.39 (d, J=8.8 Hz, 2H), 5.58 (q, J=6.5 Hz, 1H), 1.76 (d, J=6.5 Hz, 3H); ¹⁹F NMR (376 MHz, CDCl₃) δ −85.90, −87.85; HRMS-ESI (m/z) [M+H]⁺ calcd for C₂₆H₁₇F₅N₄O₄, 545.1243; found, 545.1243.

### Example 12: Preparation of tert-butyl hydroxy(4-(1-(4-(trifluoromethoxy)phenyl)-1H-1,2,4-triazol-3-yl)benzyl)carbamate (C₁₆)

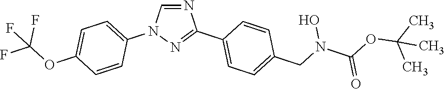 [SMILES: CC(C)(C)OC(=O)N(O)Cc1ccc(-c2ncn(-c3ccc(OC(F)(F)F)cc3)n2)cc1]

To tert-butyl ((tert-butoxycarbonyl)oxy)(4-(1-(4-(trifluoromethoxy)phenyl)-1H-1,2,4-triazol-3-yl)benzyl)carbamate (43 mg, 0.078 mmol) in methanol (0.16 mL) was added 2 N ammonia in methanol (0.039 mL, 0.078 mmol). The reaction mixture was stirred at room temperature overnight. Additional 2 N ammonia in methanol (0.02 mL) was added, and the reaction mixture was stirred at room temperature for 24 h. The mixture was concentrated under a stream of nitrogen to provide the title compound as a white solid (39 mg, 100%): ¹H NMR (400 MHz, CDCl₃) δ 8.57 (s, 1H), 8.17 (d, J=8.1 Hz, 2H), 7.80 (d, J=8.9 Hz, 2H), 7.44 (d, J=8.1 Hz, 2H), 7.39 (d, J=8.5 Hz, 2H), 5.91 (s, 1H), 4.71 (s, 2H), 1.51 (s, 9H); ¹⁹F NMR (376 MHz, CDCl₃) δ −58.03; ESIMS m/z 451 ([M+H]⁺).

### Example 13: Preparation of tert-butyl ((Cert-butoxycarbonyl)oxy)(4-(1-(4-(trifluoromethoxy)phenyl)-1H-1,2,4-triazol-3-yl)benzyl)carbamate (C₁₇)

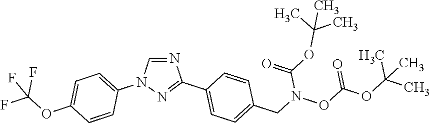 [SMILES: CC(C)(C)OC(=O)ON(Cc1ccc(-c2ncn(-c3ccc(OC(F)(F)F)cc3)n2)cc1)C(=O)OC(C)(C)C]

Step 1—To (E)-4-(1-(4-(trifluoromethoxy)phenyl)-1H-1,2,4-triazol-3-yl)benzaldehyde oxime (40 mg, 0.115 mmol) in acetic acid (0.38 mL) was added sodium cyanoborohydride (14 mg, 0.230 mmol). The reaction mixture was stirred at room temperature for 4 h, was diluted with water, was neutralized with 2 N NaOH, and was extracted with DCM. The biphasic layers were filtered through a phase separator into a tared vial and concentrated to provide a yellow that was used without purification (41 mg): ¹H NMR (400 MHz, CDCl₃) δ 8.57 (s, 1H), 8.21-8.11 (m, 2H), 7.83-7.75 (m, 2H), 7.45 (d, J=8.1 Hz, 2H), 7.38 (d, J=8.5 Hz, 3H), 4.14 (s, 2H); ¹⁹F NMR (376 MHz, CDCl₃) δ −58.04; ESIMS m/z 351 ([M+H]+).

Step 2—To N-(4-(1-(4-(trifluoromethoxy)phenyl)-1H-1,2,4-triazol-3-yl)benzyl)hydroxylamine (60 mg, 0.171 mmol) in THF (0.3 mL) and water (0.03 mL) were added di-tert-butyl dicarbonate (45 mg, 0.206 mmol) and sodium bicarbonate (29 mg, 0.343 mmol). The reaction mixture was stirred at room temperature for 4 h, was diluted with water and DCM, and was filtered through a phase separator directly onto a Celite® cartridge. Purification by flash chromatography (0-100% EtOAc in hexanes) provided the title compound as a clear oil (43 mg, 45%): ¹H NMR (400 MHz, CDCl₃) δ 8.56 (s, 1H), 8.19-8.13 (m, 2H), 7.84-7.78 (m, 2H), 7.46 (d, J=8.2 Hz, 2H), 7.41-7.36 (m, 2H), 4.82 (s, 2H), 1.50 (s, 9H), 1.47 (s, 9H); ¹⁹F NMR (376 MHz, CDCl₃) δ −58.03; ESIMS m/z 551 ([M+H]+).

### Example 14: Preparation of (E)-4-(1-(4-(trifluoromethoxy)phenyl)-1H-1,2,4-triazol-3-yl)benzaldehyde oxime (C₁₈)

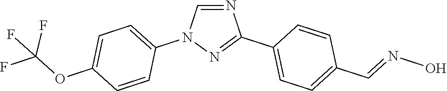 [SMILES: O/N=C/c1ccc(-c2ncn(-c3ccc(OC(F)(F)F)cc3)n2)cc1]

To 4-(1-(4-(trifluoromethoxy)phenyl)-1H-1,2,4-triazol-3-yl)benzaldehyde (665 mg, 2.0 mmol) in ethanol (5 mL) were added hydroxylamine hydrochloride (208 mg, 2.99 mmol) and triethylamine (0.56 mL, 4.0 mmol). The reaction mixture was stirred at reflux for 90 min. The ethanol was removed under a stream of nitrogen. The solid was dissolved in EtOAc and water. The biphasic mixture was filtered through a universal phase separator into a tared vial and the organic layer was concentrated. The title compound was isolated as a tan solid (678 mg, 96%): mp 162-172° C.; ¹H NMR (400 MHz, DMSO-d₆) δ 11.39 (s, 1H), 9.43 (s, 1H), 8.21 (s, 1H), 8.15-8.12 (m, 2H), 8.11-8.05 (m, 2H), 7.77-7.72 (m, 2H), 7.63 (d, J=8.6 Hz, 2H); ¹⁹F NMR (376 MHz, DMSO-d₆) δ −56.96; ESIMS m/z 349 ([M+H]⁺).

### Example 15: Preparation of (Z)-3-(2-isopropyl-5-methylphenyl)-2-((((methyl(4-(1-(4-(trifluoromethoxy)phenyl)-1H-1,2,4-triazol-3-yl)benzyl)amino)oxy)carbonyl)imino)thiazolidin-4-one (A39)

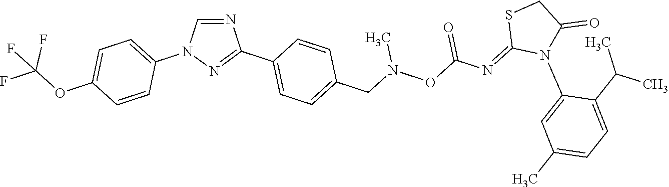 [SMILES: Cc1ccc(C(C)C)c(N2C(=O)CS/C2=N\C(=O)ON(C)Cc2ccc(-c3ncn(-c4ccc(OC(F)(F)F)cc4)n3)cc2)c1]

To N-methyl-N-(4-(1-(4-(trifluoromethoxy)phenyl)-1H-1,2,4-triazol-3-yl)benzyl)hydroxylamine (62 mg, 0.170 mmol) and 2,5-dioxopyrrolidin-1-yl (Z)-(3-(2-isopropyl-5-methylphenyl)-4-oxothiazolidin-2-ylidene)carbamate (66 mg, 0.170 mmol) in DCM (0.85 mL) was added triethylamine (23 μL, 0.170 mmol). The reaction mixture was stirred at room temperature overnight. The reaction mixture was loaded onto a Celite® cartridge with DCM. Purification by flash chromatography (0-100% EtOAc in hexanes) provided the title compound as a yellow oil (33 mg, 30%).

### Example 16: Preparation of N-methyl-N-(4-(1-(4-(trifluoromethoxy)phenyl)-1H-1,2,4-triazol-3-yl)benzyl)hydroxylamine (C₁₉)

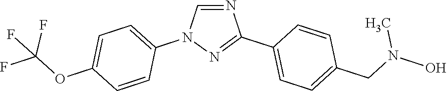 [SMILES: CN(O)Cc1ccc(-c2ncn(-c3ccc(OC(F)(F)F)cc3)n2)cc1]

To N-methyl-O-(tetrahydro-2H-pyran-2-yl)-N-(4-(1-(4-(trifluoromethoxy)phenyl)-1H-1,2,4-triazol-3-yl)benzyl)hydroxylamine (115 mg, 0.256 mmol) in THF (2.5 mL) was added 2 N HCl (2.5 mL, 5.13 mmol). The reaction mixture was stirred at room temperature for 4 h. The reaction mixture was diluted with water and extracted with EtOAc. The layers were filtered through a universal phase separator and dried under a stream of nitrogen to provide the title compound as a white solid (71 mg, 75%): ¹H NMR (400 MHz, CDCl₃) δ 8.63 (s, 1H), 8.22 (d, J=8.1 Hz, 2H), 7.79 (d, J=9.0 Hz, 2H), 7.68 (d, J=8.1 Hz, 2H), 7.39 (d, J=8.6 Hz, 2H), 4.57 (s, 2H), 3.04 (s, 3H); ¹⁹F NMR (376 MHz, CDCl₃) δ −58.04; ESIMS m/z 365 ([M+H]⁺).

### Example 17: Preparation of N-methyl-O-(tetrahydro-2H-pyran-2-yl)-N-(4-(1-(4-(trifluoromethoxy)phenyl)-1H-1,2,4-triazol-3-yl)benzyl)hydroxylamine (C₂₀)

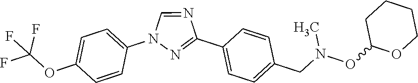 [SMILES: CN(Cc1ccc(-c2ncn(-c3ccc(OC(F)(F)F)cc3)n2)cc1)OC1CCCCO1]

To 0-(tetrahydro-2H-pyran-2-yl)-N-(4-(1-(4-(trifluoromethoxy)phenyl)-1H-1,2,4-triazol-3-yl)benzyl)hydroxylamine (169 mg, 0.389 mmol) and potassium carbonate (215 mg, 1.556 mmol) in THF (4 mL) was added iodomethane (0.24 mL, 3.89 mmol). The reaction mixture was stirred overnight at room temperature. Additional potassium carbonate (85 mg) and methyl iodide (0.1 mL) were added, and the reaction mixture was stirred another 24 h. The mixture was concentrated under a stream of nitrogen and dissolved in DCM. Water was added, and the biphasic mixture was filtered through a phase separator directly onto a Celite® cartridge. Purification by flash chromatography (0-80% EtOAc/hexanes) provided the title compound as a yellow oil (136 mg, 74%): ¹H NMR (400 MHz, CDCl₃) δ 8.57 (s, 1H), 8.17-8.12 (m, 2H), 7.84-7.77 (m, 2H), 7.51-7.47 (m, 2H), 7.39 (dq, J=9.0, 1.0 Hz, 2H), 4.52 (s, 1H), 3.97-3.82 (m, 3H), 3.48 (dt, J=11.1, 5.3 Hz, 1H), 2.78 (s, 3H), 1.74-1.62 (m, 1H), 1.42 (d, J=41.0 Hz, 5H); ¹⁹F NMR (376 MHz, CDCl₃) δ −58.03; ESIMS m/z 449 ([M+H]⁺).

### Example 18: Preparation of 0-(tetrahydro-2H-pyran-2-yl)-N-(4-1-(4-(trifluoromethoxy)phenyl)-1H-1,2,4-triazol-3-yl)benzyl)hydroxylamine (C₂₁)

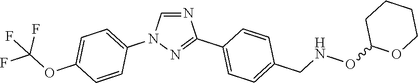 [SMILES: FC(F)(F)Oc1ccc(-n2cnc(-c3ccc(CNOC4CCCCO4)cc3)n2)cc1]

To 3-(4-(bromomethyl)phenyl)-1-(4-(trifluoromethoxy)phenyl)-1H-1,2,4-triazole (240 mg, 0.603 mmol) and potassium carbonate (100 mg, 0.723 mmol) in acetonitrile (4 mL) was added O-(tetrahydro-2H-pyran-2-yl)hydroxylamine (75 mg, 0.640 mmol). The reaction mixture was heated to 65° C. for 6 h, then cooled and diluted with EtOAc and water. The biphasic solution was filtered through a universal phase separator. The organic layer was concentrated to provide the title compound as a clear oil (196 mg, 71%): ¹H NMR (400 MHz, CDCl₃) δ 8.57 (d, J=1.5 Hz, 1H), 8.16 (dd, J=8.3, 2.0 Hz, 2H), 7.83-7.75 (m, 2H), 7.49 (d, J=8.3 Hz, 2H), 7.42-7.36 (m, 3H), 4.82-4.52 (m, 1H), 4.23-4.13 (m, 2H), 3.95-3.73 (m, 2H), 3.62-3.34 (m, 2H), 1.76-1.40 (m, 6H); ¹⁹F NMR (376 MHz, CDCl₃) δ −58.03; ESIMS m/z 435 ([M+H]+).

### Example 19: Preparation of (Z)-3-(2-isopropyl-5-methylphenyl)-2-(((((4-(1-(4-(trifluoromethoxy)phenyl)-1H-1,2,4-triazol-3-yl)benzyl)amino)oxy)carbonyl)imino)thiazolidin-4-one (A40)

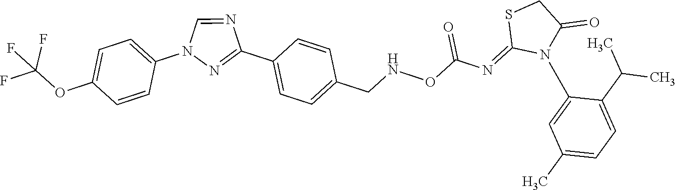 [SMILES: Cc1ccc(C(C)C)c(N2C(=O)CS/C2=N\C(=O)ONCc2ccc(-c3ncn(-c4ccc(OC(F)(F)F)cc4)n3)cc2)c1]

4-(1-(4-(Trifluoromethoxy)phenyl)-1H-1,2,4-triazol-3-yl)benzaldehyde (40 mg, 0.120 mmol) and (Z)-2-(((aminooxy)carbonyl)imino)-3-(2-isopropyl-5-methylphenyl)thiazolidin-4-one (50 mg, 0.163 mmol) in DCM (0.25 mL) was stirred at room temperature overnight. The reaction mixture was concentrated under a stream of nitrogen and was dissolved in ethanol (0.25 mL). To the reaction mixture was added sodium cyanoborohydride (23 mg, 0.36 mmol). After 1 h, 1.25 M HCl in ethanol (0.1 mL, 0.125 mmol) was added. The reaction mixture was stirred at room temperature overnight. Additional sodium cyanoborohydride (23 mg, 0.36 mmol) was added. After stirring at room temperature for 4 h, additional 1.25 M HCl in ethanol (0.1 mL, 0.125 mmol) and sodium cyanoborohydride (23 mg, 0.36 mmol) were added. The mixture was stirred for 3 days. The reaction was quenched with saturated aqueous NaHCO₃ and the mixture was extracted twice with EtOAc. The organic layer was separated and filtered through a Na₂SO₄ cartridge into a tared vial. The solvent was removed under a stream of nitrogen to provide a yellow oil. Purification by flash chromatography (0-100% EtOAc/[1:1 DCM/hexanes]) to provide the title compound as an off-white oil (17 mg, 22%).

### Example 20: Preparation of (Z)-2-(((aminooxy)carbonyl)imino)-3-(2-isopropyl-5-methylphenyl)thiazolidin-4-one (C₂₂)

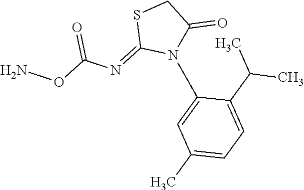 [SMILES: Cc1ccc(C(C)C)c(N2C(=O)CS/C2=N\C(=O)ON)c1]

To 1,3-dioxoisoindolin-2-yl (Z)-(3-(2-isopropyl-5-methylphenyl)-4-oxothiazolidin ylidene)carbamate (32 mg, 0.073 mmol) in DCM (0.3 mL) was added 1 drop of hydrazine monohydrate. The reaction mixture immediately turned bright orange-brown, then within 30 seconds turned cloudy white. The reaction mixture was diluted with DCM-water and was filtered through a phase separator into a tared vial. The solvent was removed under a stream of nitrogen. The title compound was isolated as yellow oil (24 mg, 96%): ¹H NMR (400 MHz, CDCl₃) δ 7.33 (d, J=8.0 Hz, 1H), 7.28 (d, J=1.8 Hz, 1H), 6.84 (d, J=1.7 Hz, 1H), 4.03-3.88 (m, 2H), 3.83 (s, 2H), 2.61 (p, J=6.9 Hz, 1H), 2.35 (s, 3H), 1.15 (dd, J=6.9, 2.9 Hz, 6H).

### Example 21: Preparation of 1,3-dioxoisoindolin-2-yl (Z)-(3-(2-isopropyl-5-methylphenyl)-4-oxothiazolidin-2-ylidene)carbamate (C₂₃)

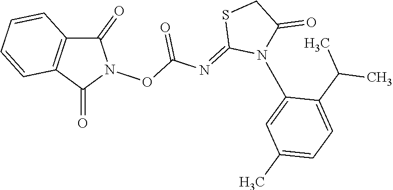 [SMILES: Cc1ccc(C(C)C)c(N2C(=O)CS/C2=N\C(=O)ON2C(=O)c3ccccc3C2=O)c1]

To 2,5-dioxopyrrolidin-1-yl (Z)-(3-(2-isopropyl-5-methylphenyl)-4-oxothiazolidin-2-ylidene)carbamate (95 mg, 0.244 mmol) and 2-hydroxyisoindoline-1,3-dione (60 mg, 0.368 mmol) in DCM (1.22 mL) was added triethylamine (34.0 μL, 0.244 mmol). The reaction mixture was stirred at room temperature for 2.5 h. The mixture was loaded onto a Celite® cartridge with DCM. Purification by flash chromatography (0-100% EtOAc in hexanes) provided the title compound as an off-white solid (36 mg, 33%): mp 233-235° C. (dec); ¹H NMR (400 MHz, CDCl₃) δ 7.91-7.69 (m, 4H), 7.26 (m, 2H), 6.81 (s, 1H), 4.07 (d, J=3.0 Hz, 2H), 2.54 (s, 1H), 2.40-2.22 (m, 3H), 1.23-1.09 (m, 6H); ESIMS m/z 438 ([M+H]+).

### Example 22: Preparation of 2,5-dioxopyrrolidin-1-yl (Z)-(3-(2-isopropyl-5-methylphenyl)-4-oxothiazolidin-2-ylidene)carbamate (C₂₄)

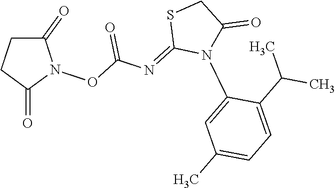 [SMILES: Cc1ccc(C(C)C)c(N2C(=O)CS/C2=N\C(=O)ON2C(=O)CCC2=O)c1]

To 2-imino-3-(2-isopropyl-5-methylphenyl)thiazolidin-4-one (971 mg, 3.91 mmol) and bis(2,5-dioxopyrrolidin-1-yl) carbonate (1.002 g, 3.91 mmol) in acetonitrile (13 mL) was added pyridine (0.32 mL, 3.91 mmol). The reaction mixture was stirred at room temperature. The acetonitrile was removed under a stream of nitrogen. The orange oil was dissolved in DCM and partitioned with water. The biphasic mixture was filtered through a phase separator directly onto a Celite® cartridge. Purification by flash chromatography (0-100% EtOAc in hexanes) provided the title compound as a tan solid (726 mg, 47%): mp 205-225° C.; NMR (300 MHz, CDCl₃) δ 7.37-7.26 (m, 2H), 6.85 (d, J=1.5 Hz, ¹H), 4.08 (d, J=1.6 Hz, 2H), 2.72 (s, 4H), 2.57 (h, J=7.1 Hz, 1H), 2.36 (d, J=0.7 Hz, 3H), 1.18 (dd, J=12.9, 6.8 Hz, 6H); ¹³C NMR (126 MHz, CDCl₃) δ 171.90, 168.94, 158.67, 143.13, 131.55, 128.27, 126.86, 33.37, 28.52, 25.39, 23.92, 23.51, 20.73; ESIMS m/z 390 ([M+H]⁺).

Using the procedures disclosed herein, the following lists of molecules are provided as examples (Table P and Table 1).

TABLE P
Structures of Prophetic Compounds

| | |
|---|---|
| P1 | 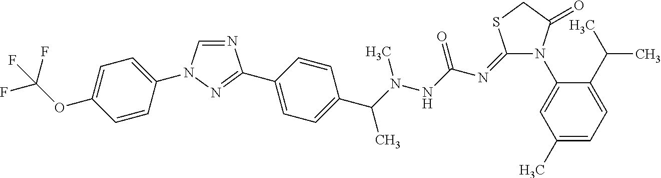 [SMILES: Cc1ccc(C(C)C)c(N2C(=O)CS/C2=N\C(=O)NN(C)C(C)c2ccc(-c3ncn(-c4ccc(OC(F)(F)F)cc4)n3)cc2)c1] |
| P2 | 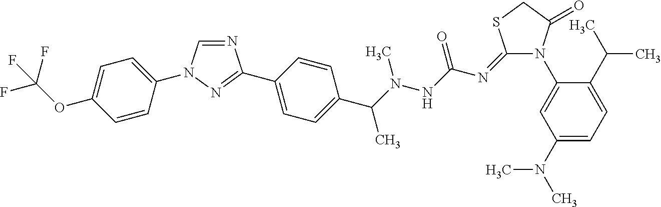 [SMILES: CC(C)c1ccc(N(C)C)cc1N1C(=O)CS/C1=N\C(=O)NN(C)C(C)c1ccc(-c2ncn(-c3ccc(OC(F)(F)F)cc3)n2)cc1] |
| P3 | 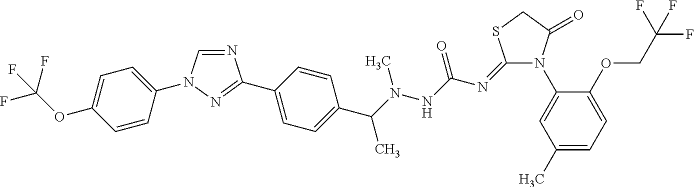 [SMILES: Cc1ccc(OCC(F)(F)F)c(N2C(=O)CS/C2=N\C(=O)NN(C)C(C)c2ccc(-c3ncn(-c4ccc(OC(F)(F)F)cc4)n3)cc2)c1] |
| P4 | 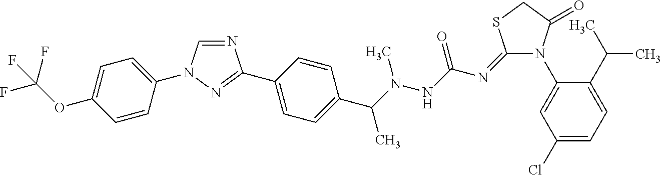 [SMILES: CC(C)c1ccc(Cl)cc1N1C(=O)CS/C1=N\C(=O)NN(C)C(C)c1ccc(-c2ncn(-c3ccc(OC(F)(F)F)cc3)n2)cc1] |
| P5 | 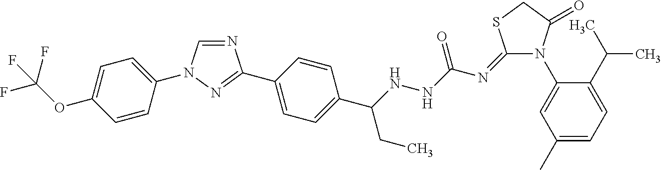 [SMILES: CCC(NNC(=O)/N=C1\SCC(=O)N1c1cc(C)ccc1C(C)C)c1ccc(-c2ncn(-c3ccc(OC(F)(F)F)cc3)n2)cc1] |
| P6 | 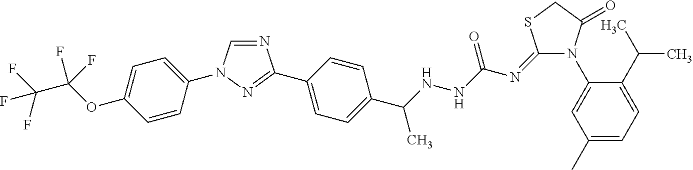 [SMILES: Cc1ccc(C(C)C)c(N2C(=O)CS/C2=N\C(=O)NNC(C)c2ccc(-c3ncn(-c4ccc(OC(F)(F)C(F)(F)F)cc4)n3)cc2)c1] |
| P7 | 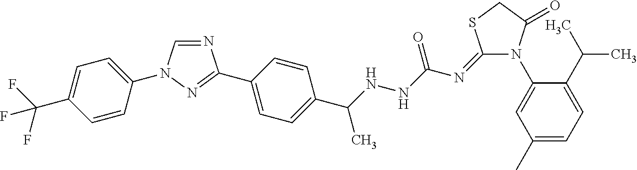 [SMILES: Cc1ccc(C(C)C)c(N2C(=O)CS/C2=N\C(=O)NNC(C)c2ccc(-c3ncn(-c4ccc(C(F)(F)F)cc4)n3)cc2)c1] |
| P8 | 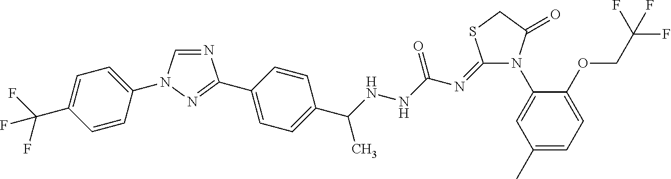 [SMILES: Cc1ccc(OCC(F)(F)F)c(N2C(=O)CS/C2=N\C(=O)NNC(C)c2ccc(-c3ncn(-c4ccc(C(F)(F)F)cc4)n3)cc2)c1] |
| P9 | 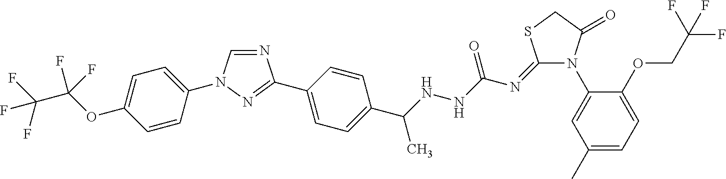 [SMILES: Cc1ccc(OCC(F)(F)F)c(N2C(=O)CS/C2=N\C(=O)NNC(C)c2ccc(-c3ncn(-c4ccc(OC(F)(F)C(F)(F)F)cc4)n3)cc2)c1] |
| P10 | 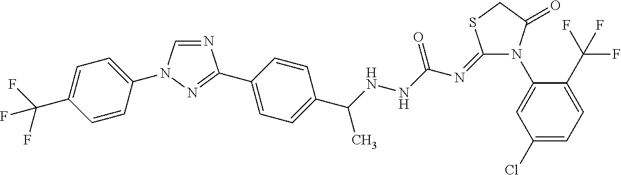 [SMILES: CC(NNC(=O)/N=C1\SCC(=O)N1c1cc(Cl)ccc1C(F)(F)F)c1ccc(-c2ncn(-c3ccc(C(F)(F)F)cc3)n2)cc1] |
| P11 | 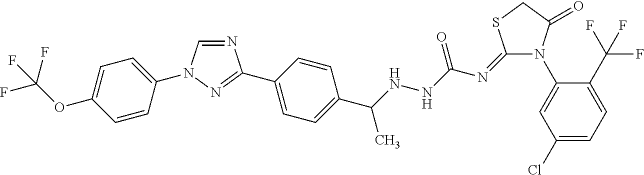 [SMILES: CC(NNC(=O)/N=C1\SCC(=O)N1c1cc(Cl)ccc1C(F)(F)F)c1ccc(-c2ncn(-c3ccc(OC(F)(F)F)cc3)n2)cc1] |
| P12 | 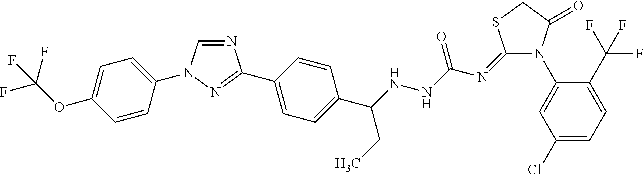 [SMILES: CCC(NNC(=O)/N=C1\SCC(=O)N1c1cc(Cl)ccc1C(F)(F)F)c1ccc(-c2ncn(-c3ccc(OC(F)(F)F)cc3)n2)cc1] |
| P13 | 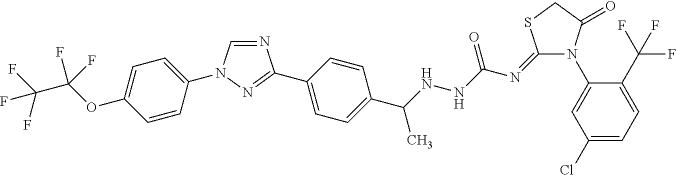 [SMILES: CC(NNC(=O)/N=C1\SCC(=O)N1c1cc(Cl)ccc1C(F)(F)F)c1ccc(-c2ncn(-c3ccc(OC(F)(F)C(F)(F)F)cc3)n2)cc1] |
| P14 | 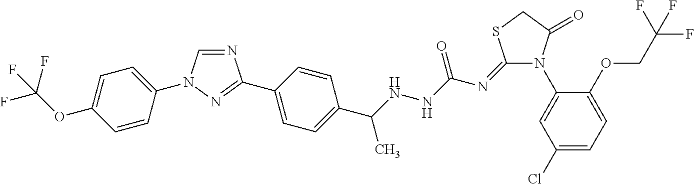 [SMILES: CC(NNC(=O)/N=C1\SCC(=O)N1c1cc(Cl)ccc1OCC(F)(F)F)c1ccc(-c2ncn(-c3ccc(OC(F)(F)F)cc3)n2)cc1] |
| P15 | 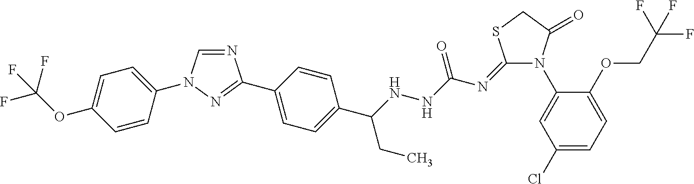 [SMILES: CCC(NNC(=O)/N=C1\SCC(=O)N1c1cc(Cl)ccc1OCC(F)(F)F)c1ccc(-c2ncn(-c3ccc(OC(F)(F)F)cc3)n2)cc1] |
| P16 | 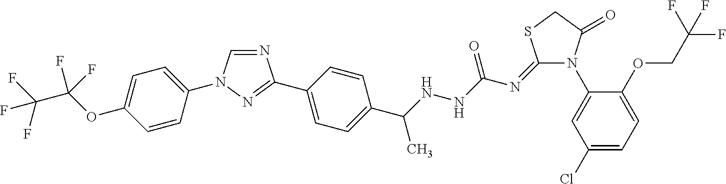 [SMILES: CC(NNC(=O)/N=C1\SCC(=O)N1c1cc(Cl)ccc1OCC(F)(F)F)c1ccc(-c2ncn(-c3ccc(OC(F)(F)C(F)(F)F)cc3)n2)cc1] |
| P17 | 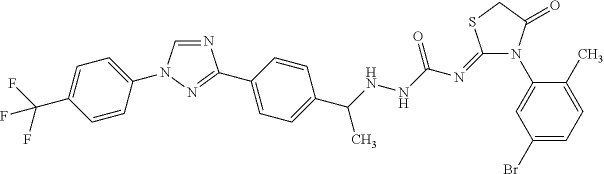 [SMILES: Cc1ccc(Br)cc1N1C(=O)CS/C1=N\C(=O)NNC(C)c1ccc(-c2ncn(-c3ccc(C(F)(F)F)cc3)n2)cc1] |
| P18 | 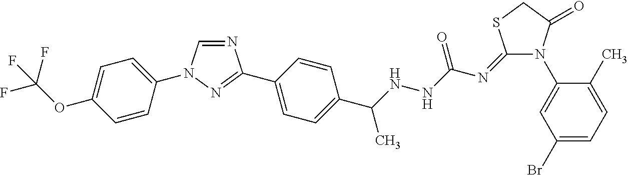 [SMILES: Cc1ccc(Br)cc1N1C(=O)CS/C1=N\C(=O)NNC(C)c1ccc(-c2ncn(-c3ccc(OC(F)(F)F)cc3)n2)cc1] |
| P19 | 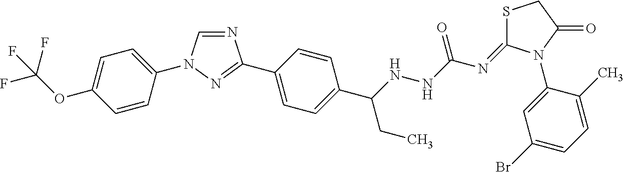 [SMILES: CCC(NNC(=O)/N=C1\SCC(=O)N1c1cc(Br)ccc1C)c1ccc(-c2ncn(-c3ccc(OC(F)(F)F)cc3)n2)cc1] |
| P20 | 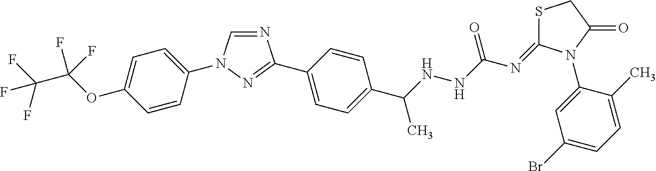 [SMILES: Cc1ccc(Br)cc1N1C(=O)CS/C1=N\C(=O)NNC(C)c1ccc(-c2ncn(-c3ccc(OC(F)(F)C(F)(F)F)cc3)n2)cc1] |
| P21 | 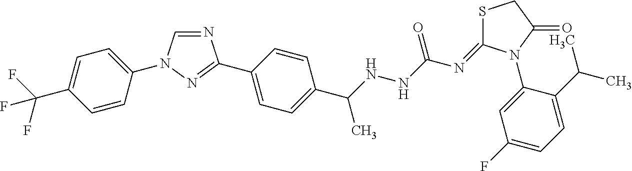 [SMILES: CC(C)c1ccc(F)cc1N1C(=O)CS/C1=N\C(=O)NNC(C)c1ccc(-c2ncn(-c3ccc(C(F)(F)F)cc3)n2)cc1] |
| P22 | 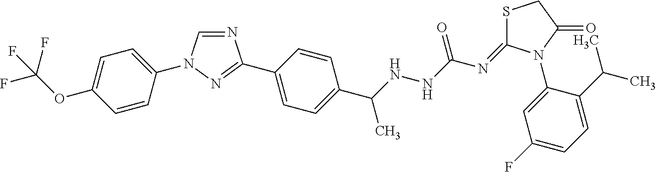 [SMILES: CC(C)c1ccc(F)cc1N1C(=O)CS/C1=N\C(=O)NNC(C)c1ccc(-c2ncn(-c3ccc(OC(F)(F)F)cc3)n2)cc1] |
| P23 | 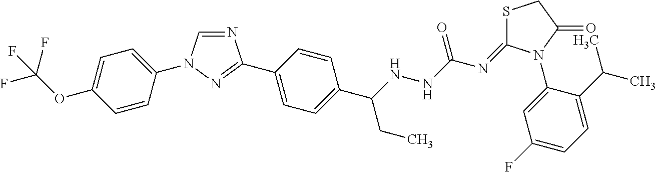 [SMILES: CCC(NNC(=O)/N=C1\SCC(=O)N1c1cc(F)ccc1C(C)C)c1ccc(-c2ncn(-c3ccc(OC(F)(F)F)cc3)n2)cc1] |
| P24 | 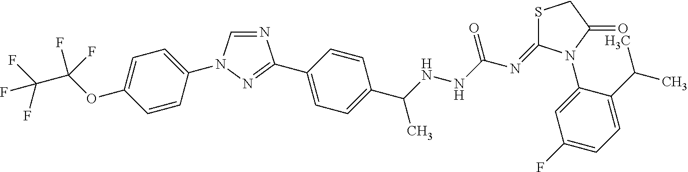 [SMILES: CC(C)c1ccc(F)cc1N1C(=O)CS/C1=N\C(=O)NNC(C)c1ccc(-c2ncn(-c3ccc(OC(F)(F)C(F)(F)F)cc3)n2)cc1] |
| P25 | 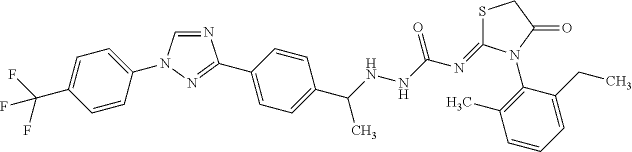 [SMILES: CCc1cccc(C)c1N1C(=O)CS/C1=N\C(=O)NNC(C)c1ccc(-c2ncn(-c3ccc(C(F)(F)F)cc3)n2)cc1] |
| P26 | 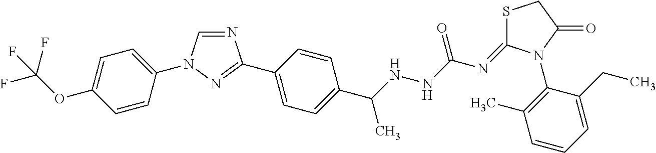 [SMILES: CCc1cccc(C)c1N1C(=O)CS/C1=N\C(=O)NNC(C)c1ccc(-c2ncn(-c3ccc(OC(F)(F)F)cc3)n2)cc1] |
| P27 | 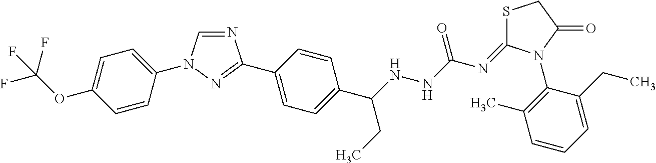 [SMILES: CCc1cccc(C)c1N1C(=O)CS/C1=N\C(=O)NNC(CC)c1ccc(-c2ncn(-c3ccc(OC(F)(F)F)cc3)n2)cc1] |
| P28 | 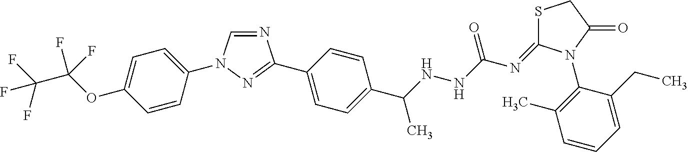 [SMILES: CCc1cccc(C)c1N1C(=O)CS/C1=N\C(=O)NNC(C)c1ccc(-c2ncn(-c3ccc(OC(F)(F)C(F)(F)F)cc3)n2)cc1] |
| P29 | 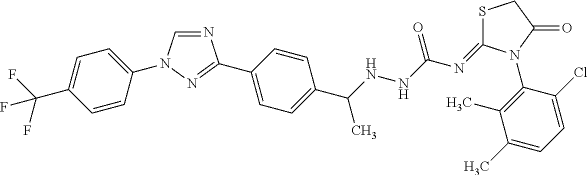 [SMILES: Cc1ccc(Cl)c(N2C(=O)CS/C2=N\C(=O)NNC(C)c2ccc(-c3ncn(-c4ccc(C(F)(F)F)cc4)n3)cc2)c1C] |
| P30 | 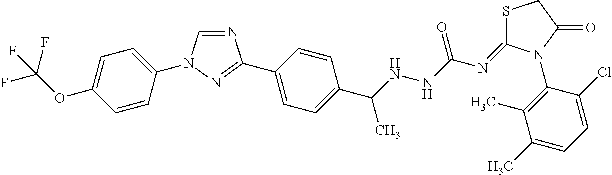 [SMILES: Cc1ccc(Cl)c(N2C(=O)CS/C2=N\C(=O)NNC(C)c2ccc(-c3ncn(-c4ccc(OC(F)(F)F)cc4)n3)cc2)c1C] |
| P31 | 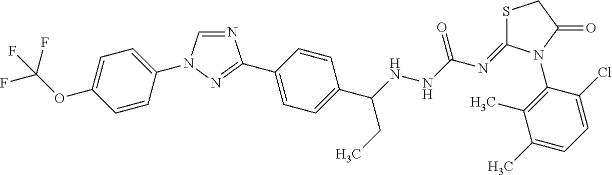 [SMILES: CCC(NNC(=O)/N=C1\SCC(=O)N1c1c(Cl)ccc(C)c1C)c1ccc(-c2ncn(-c3ccc(OC(F)(F)F)cc3)n2)cc1] |
| P32 | 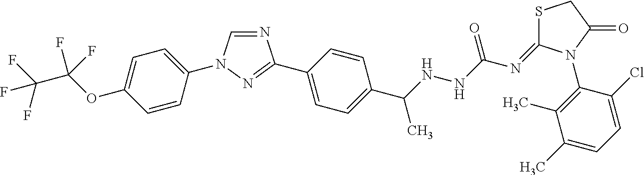 [SMILES: Cc1ccc(Cl)c(N2C(=O)CS/C2=N\C(=O)NNC(C)c2ccc(-c3ncn(-c4ccc(OC(F)(F)C(F)(F)F)cc4)n3)cc2)c1C] |
| P33 | 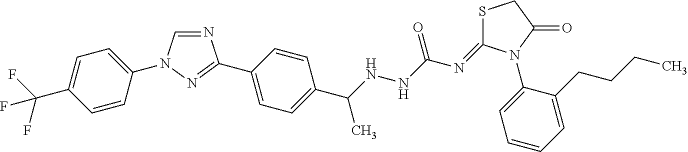 [SMILES: CCCCc1ccccc1N1C(=O)CS/C1=N\C(=O)NNC(C)c1ccc(-c2ncn(-c3ccc(C(F)(F)F)cc3)n2)cc1] |
| P34 | 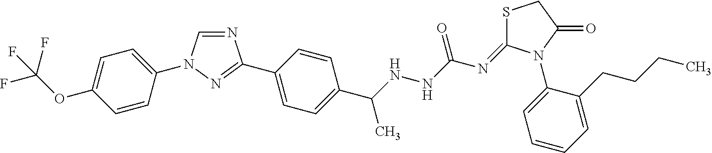 [SMILES: CCCCc1ccccc1N1C(=O)CS/C1=N\C(=O)NNC(C)c1ccc(-c2ncn(-c3ccc(OC(F)(F)F)cc3)n2)cc1] |
| P35 | 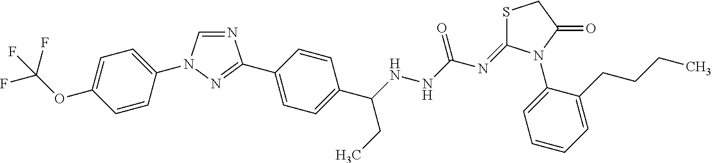 [SMILES: CCCCc1ccccc1N1C(=O)CS/C1=N\C(=O)NNC(CC)c1ccc(-c2ncn(-c3ccc(OC(F)(F)F)cc3)n2)cc1] |
| P36 | 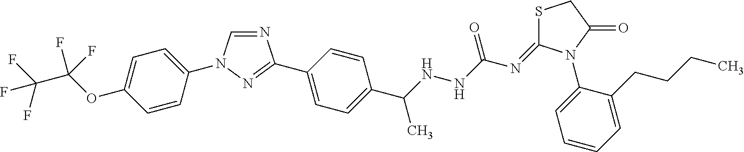 [SMILES: CCCCc1ccccc1N1C(=O)CS/C1=N\C(=O)NNC(C)c1ccc(-c2ncn(-c3ccc(OC(F)(F)C(F)(F)F)cc3)n2)cc1] |
| P37 | 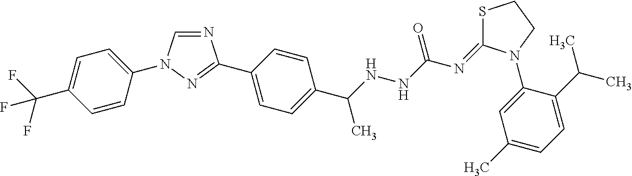 [SMILES: Cc1ccc(C(C)C)c(N2CCS/C2=N\C(=O)NNC(C)c2ccc(-c3ncn(-c4ccc(C(F)(F)F)cc4)n3)cc2)c1] |
| P38 | 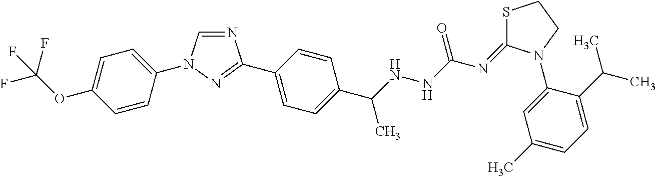 [SMILES: Cc1ccc(C(C)C)c(N2CCS/C2=N\C(=O)NNC(C)c2ccc(-c3ncn(-c4ccc(OC(F)(F)F)cc4)n3)cc2)c1] |
| P39 | 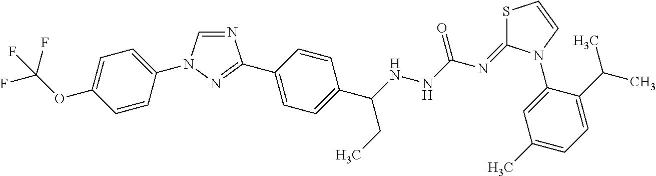 [SMILES: CCC(NNC(=O)/N=c1\sccn1-c1cc(C)ccc1C(C)C)c1ccc(-c2ncn(-c3ccc(OC(F)(F)F)cc3)n2)cc1] |
| P40 | 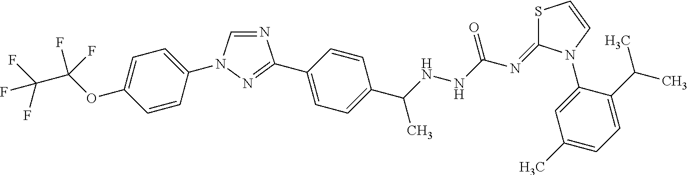 [SMILES: Cc1ccc(C(C)C)c(-n2ccs/c2=N\C(=O)NNC(C)c2ccc(-c3ncn(-c4ccc(OC(F)(F)C(F)(F)F)cc4)n3)cc2)c1] |
| P41 | 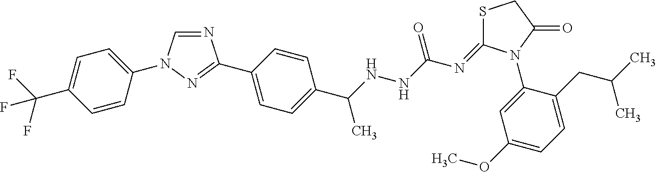 [SMILES: COc1ccc(CC(C)C)c(N2C(=O)CS/C2=N\C(=O)NNC(C)c2ccc(-c3ncn(-c4ccc(C(F)(F)F)cc4)n3)cc2)c1] |
| P42 | 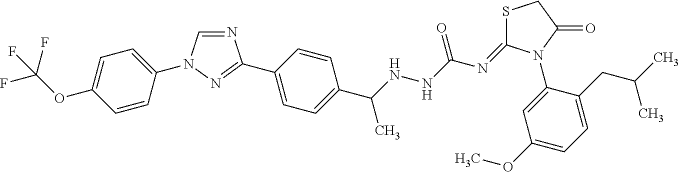 [SMILES: COc1ccc(CC(C)C)c(N2C(=O)CS/C2=N\C(=O)NNC(C)c2ccc(-c3ncn(-c4ccc(OC(F)(F)F)cc4)n3)cc2)c1] |
| P43 | 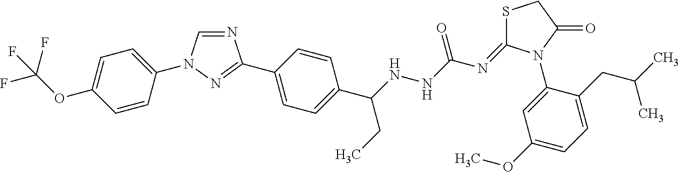 [SMILES: CCC(NNC(=O)/N=C1\SCC(=O)N1c1cc(OC)ccc1CC(C)C)c1ccc(-c2ncn(-c3ccc(OC(F)(F)F)cc3)n2)cc1] |
| P44 | 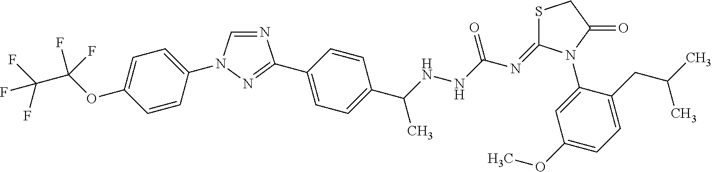 [SMILES: COc1ccc(CC(C)C)c(N2C(=O)CS/C2=N\C(=O)NNC(C)c2ccc(-c3ncn(-c4ccc(OC(F)(F)C(F)(F)F)cc4)n3)cc2)c1] |
| P45 | 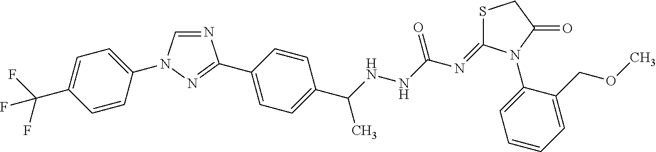 [SMILES: COCc1ccccc1N1C(=O)CS/C1=N\C(=O)NNC(C)c1ccc(-c2ncn(-c3ccc(C(F)(F)F)cc3)n2)cc1] |
| P46 | 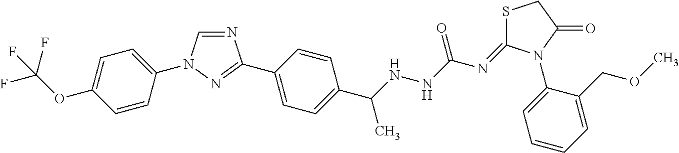 [SMILES: COCc1ccccc1N1C(=O)CS/C1=N\C(=O)NNC(C)c1ccc(-c2ncn(-c3ccc(OC(F)(F)F)cc3)n2)cc1] |
| P47 | 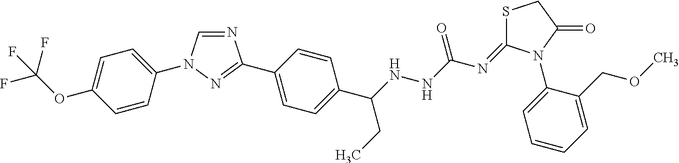 [SMILES: CCC(NNC(=O)/N=C1\SCC(=O)N1c1ccccc1COC)c1ccc(-c2ncn(-c3ccc(OC(F)(F)F)cc3)n2)cc1] |
| P48 | 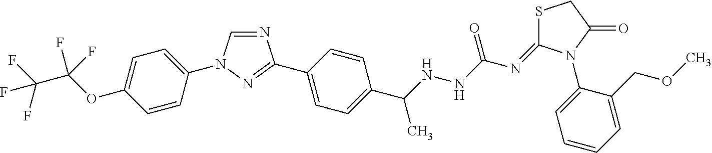 [SMILES: COCc1ccccc1N1C(=O)CS/C1=N\C(=O)NNC(C)c1ccc(-c2ncn(-c3ccc(OC(F)(F)C(F)(F)F)cc3)n2)cc1] |
| P49 | 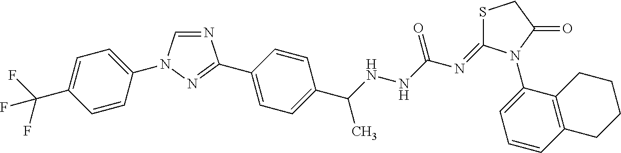 [SMILES: CC(NNC(=O)/N=C1\SCC(=O)N1c1cccc2c1CCCC2)c1ccc(-c2ncn(-c3ccc(C(F)(F)F)cc3)n2)cc1] |
| P50 | 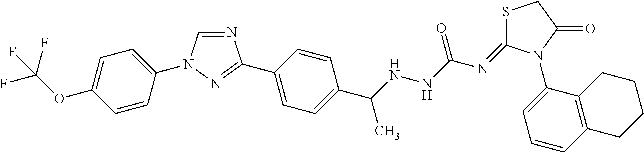 [SMILES: CC(NNC(=O)/N=C1\SCC(=O)N1c1cccc2c1CCCC2)c1ccc(-c2ncn(-c3ccc(OC(F)(F)F)cc3)n2)cc1] |
| P51 | 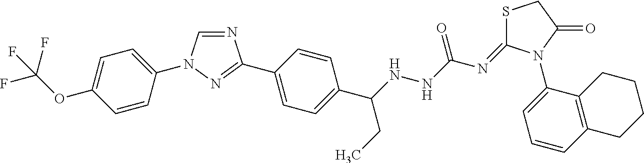 [SMILES: CCC(NNC(=O)/N=C1\SCC(=O)N1c1cccc2c1CCCC2)c1ccc(-c2ncn(-c3ccc(OC(F)(F)F)cc3)n2)cc1] |
| P52 | 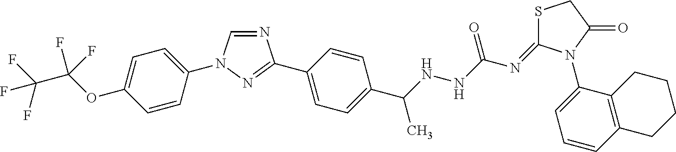 [SMILES: CC(NNC(=O)/N=C1\SCC(=O)N1c1cccc2c1CCCC2)c1ccc(-c2ncn(-c3ccc(OC(F)(F)C(F)(F)F)cc3)n2)cc1] |
| P53 | 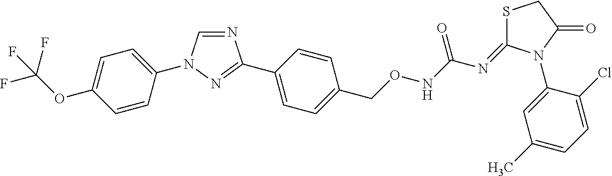 [SMILES: Cc1ccc(Cl)c(N2C(=O)CS/C2=N\C(=O)NOCc2ccc(-c3ncn(-c4ccc(OC(F)(F)F)cc4)n3)cc2)c1] |
| P54 | 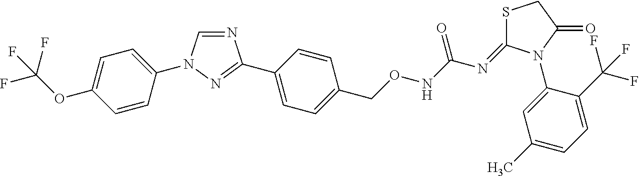 [SMILES: Cc1ccc(C(F)(F)F)c(N2C(=O)CS/C2=N\C(=O)NOCc2ccc(-c3ncn(-c4ccc(OC(F)(F)F)cc4)n3)cc2)c1] |
| P55 | 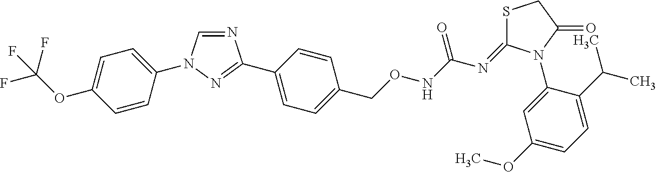 [SMILES: COc1ccc(C(C)C)c(N2C(=O)CS/C2=N\C(=O)NOCc2ccc(-c3ncn(-c4ccc(OC(F)(F)F)cc4)n3)cc2)c1] |
| P56 | 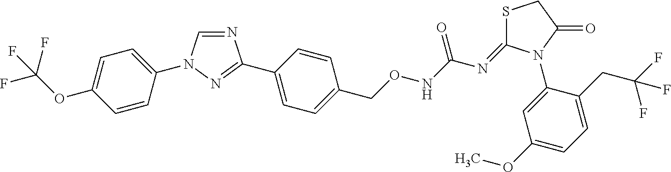 [SMILES: COc1ccc(CC(F)(F)F)c(N2C(=O)CS/C2=N\C(=O)NOCc2ccc(-c3ncn(-c4ccc(OC(F)(F)F)cc4)n3)cc2)c1] |
| P57 | 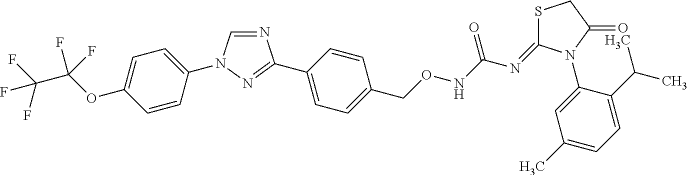 [SMILES: Cc1ccc(C(C)C)c(N2C(=O)CS/C2=N\C(=O)NOCc2ccc(-c3ncn(-c4ccc(OC(F)(F)C(F)(F)F)cc4)n3)cc2)c1] |
| P58 | 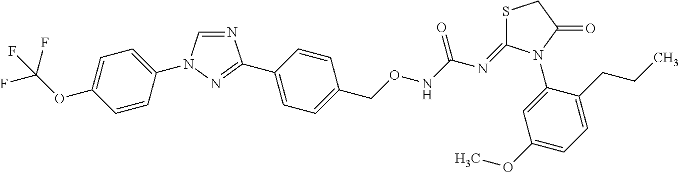 [SMILES: CCCc1ccc(OC)cc1N1C(=O)CS/C1=N\C(=O)NOCc1ccc(-c2ncn(-c3ccc(OC(F)(F)F)cc3)n2)cc1] |
| P59 | 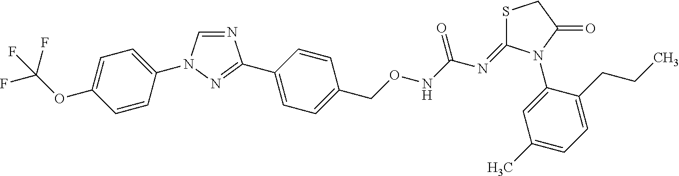 [SMILES: CCCc1ccc(C)cc1N1C(=O)CS/C1=N\C(=O)NOCc1ccc(-c2ncn(-c3ccc(OC(F)(F)F)cc3)n2)cc1] |
| P60 | 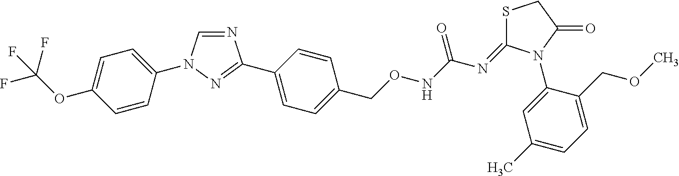 [SMILES: COCc1ccc(C)cc1N1C(=O)CS/C1=N\C(=O)NOCc1ccc(-c2ncn(-c3ccc(OC(F)(F)F)cc3)n2)cc1] |
| P61 | 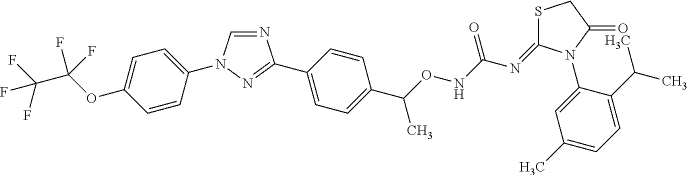 [SMILES: Cc1ccc(C(C)C)c(N2C(=O)CS/C2=N\C(=O)NOC(C)c2ccc(-c3ncn(-c4ccc(OC(F)(F)C(F)(F)F)cc4)n3)cc2)c1] |
| P62 | 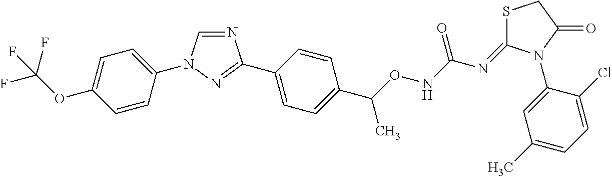 [SMILES: Cc1ccc(Cl)c(N2C(=O)CS/C2=N\C(=O)NOC(C)c2ccc(-c3ncn(-c4ccc(OC(F)(F)F)cc4)n3)cc2)c1] |
| P63 | 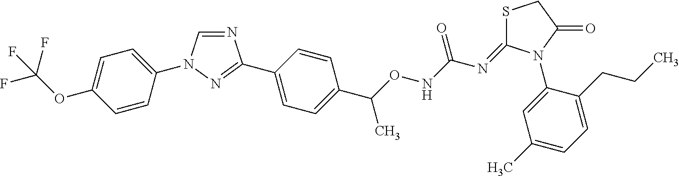 [SMILES: CCCc1ccc(C)cc1N1C(=O)CS/C1=N\C(=O)NOC(C)c1ccc(-c2ncn(-c3ccc(OC(F)(F)F)cc3)n2)cc1] |
| P64 | 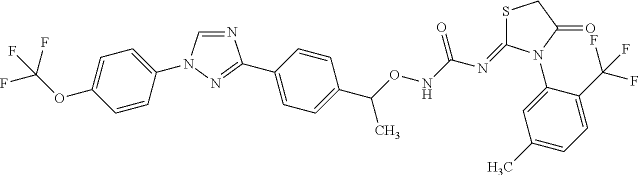 [SMILES: Cc1ccc(C(F)(F)F)c(N2C(=O)CS/C2=N\C(=O)NOC(C)c2ccc(-c3ncn(-c4ccc(OC(F)(F)F)cc4)n3)cc2)c1] |
| P65 | 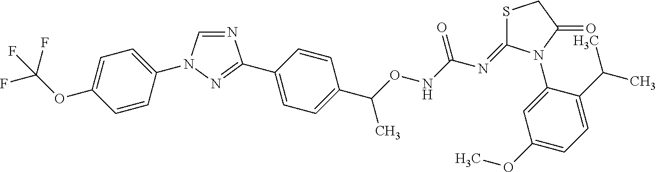 [SMILES: COc1ccc(C(C)C)c(N2C(=O)CS/C2=N\C(=O)NOC(C)c2ccc(-c3ncn(-c4ccc(OC(F)(F)F)cc4)n3)cc2)c1] |
| P66 | 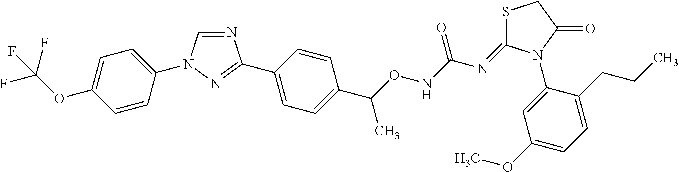 [SMILES: CCCc1ccc(OC)cc1N1C(=O)CS/C1=N\C(=O)NOC(C)c1ccc(-c2ncn(-c3ccc(OC(F)(F)F)cc3)n2)cc1] |
| P67 | 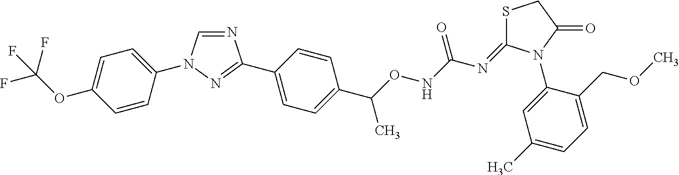 [SMILES: COCc1ccc(C)cc1N1C(=O)CS/C1=N\C(=O)NOC(C)c1ccc(-c2ncn(-c3ccc(OC(F)(F)F)cc3)n2)cc1] |
| P68 | 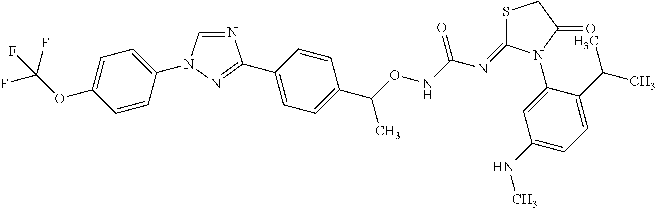 [SMILES: CNc1ccc(C(C)C)c(N2C(=O)CS/C2=N\C(=O)NOC(C)c2ccc(-c3ncn(-c4ccc(OC(F)(F)F)cc4)n3)cc2)c1] |
| P69 | 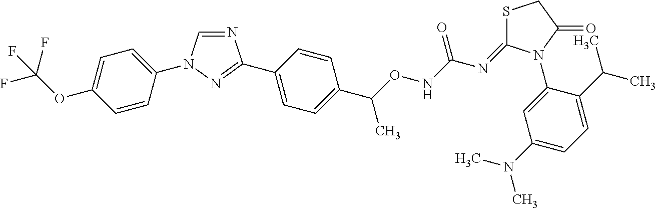 [SMILES: CC(C)c1ccc(N(C)C)cc1N1C(=O)CS/C1=N\C(=O)NOC(C)c1ccc(-c2ncn(-c3ccc(OC(F)(F)F)cc3)n2)cc1] |
| P70 | 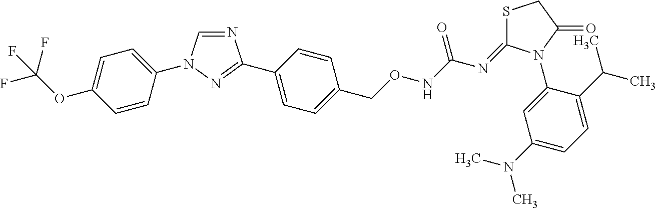 [SMILES: CC(C)c1ccc(N(C)C)cc1N1C(=O)CS/C1=N\C(=O)NOCc1ccc(-c2ncn(-c3ccc(OC(F)(F)F)cc3)n2)cc1] |
| P71 | 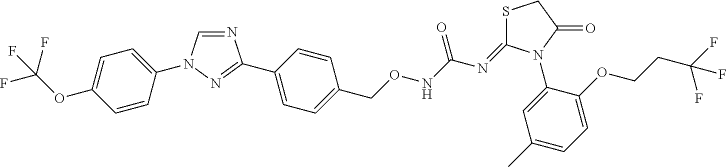 [SMILES: Cc1ccc(OCCC(F)(F)F)c(N2C(=O)CS/C2=N\C(=O)NOCc2ccc(-c3ncn(-c4ccc(OC(F)(F)F)cc4)n3)cc2)c1] |
| P72 | 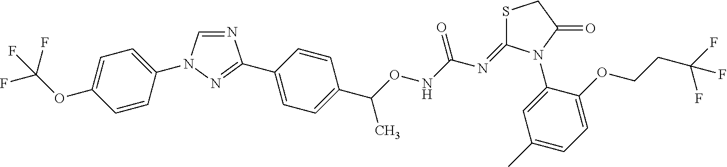 [SMILES: Cc1ccc(OCCC(F)(F)F)c(N2C(=O)CS/C2=N\C(=O)NOC(C)c2ccc(-c3ncn(-c4ccc(OC(F)(F)F)cc4)n3)cc2)c1] |
| P73 A39 | 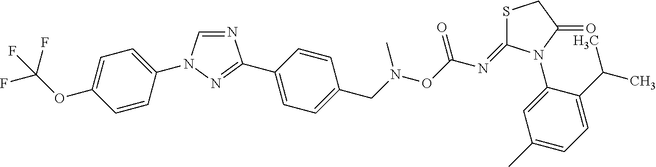 [SMILES: Cc1ccc(C(C)C)c(N2C(=O)CS/C2=N\C(=O)ON(C)Cc2ccc(-c3ncn(-c4ccc(OC(F)(F)F)cc4)n3)cc2)c1] |
| P74 A35 | 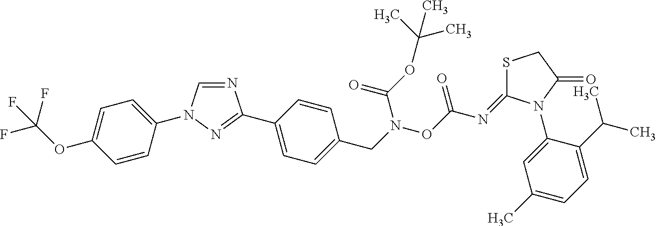 [SMILES: Cc1ccc(C(C)C)c(N2C(=O)CS/C2=N\C(=O)ON(Cc2ccc(-c3ncn(-c4ccc(OC(F)(F)F)cc4)n3)cc2)C(=O)OC(C)(C)C)c1] |
| P75 A40 | 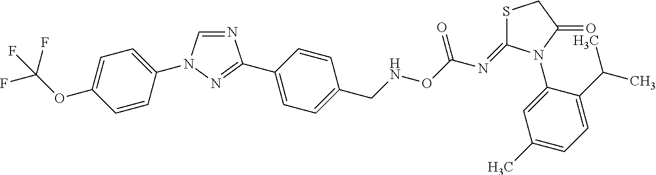 [SMILES: Cc1ccc(C(C)C)c(N2C(=O)CS/C2=N\C(=O)ONCc2ccc(-c3ncn(-c4ccc(OC(F)(F)F)cc4)n3)cc2)c1] |
| P76 | 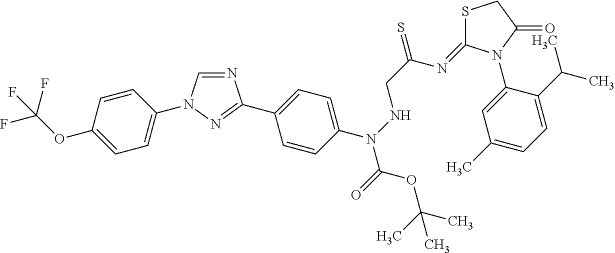 [SMILES: Cc1ccc(C(C)C)c(N2C(=O)CS/C2=N\C(=S)CNN(C(=O)OC(C)(C)C)c2ccc(-c3ncn(-c4ccc(OC(F)(F)F)cc4)n3)cc2)c1] |
| P77 | 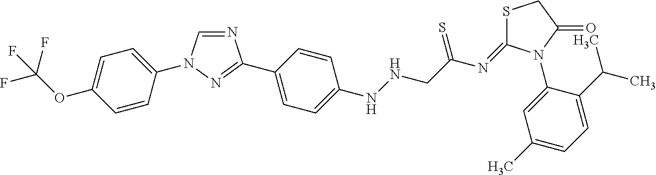 [SMILES: Cc1ccc(C(C)C)c(N2C(=O)CS/C2=N\C(=S)CNNc2ccc(-c3ncn(-c4ccc(OC(F)(F)F)cc4)n3)cc2)c1] |
| P78 | 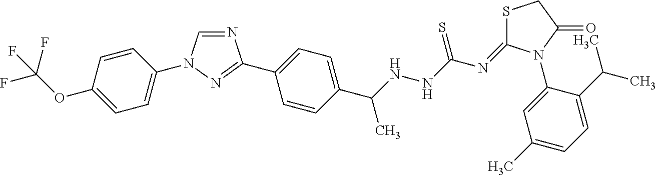 [SMILES: Cc1ccc(C(C)C)c(N2C(=O)CS/C2=N\C(=S)NNC(C)c2ccc(-c3ncn(-c4ccc(OC(F)(F)F)cc4)n3)cc2)c1] |
| P79 | 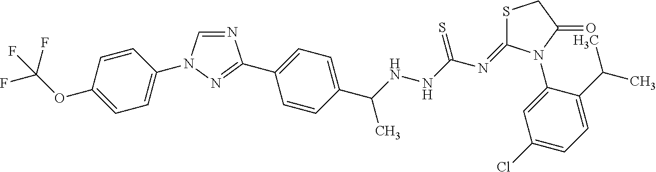 [SMILES: CC(C)c1ccc(Cl)cc1N1C(=O)CS/C1=N\C(=S)NNC(C)c1ccc(-c2ncn(-c3ccc(OC(F)(F)F)cc3)n2)cc1] |
| P80 | 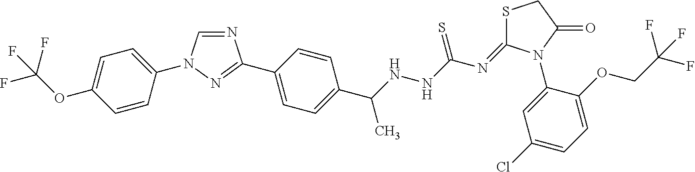 [SMILES: CC(NNC(=S)/N=C1\SCC(=O)N1c1cc(Cl)ccc1OCC(F)(F)F)c1ccc(-c2ncn(-c3ccc(OC(F)(F)F)cc3)n2)cc1] |
| P81 | 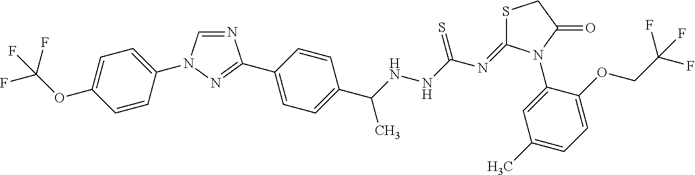 [SMILES: Cc1ccc(OCC(F)(F)F)c(N2C(=O)CS/C2=N\C(=S)NNC(C)c2ccc(-c3ncn(-c4ccc(OC(F)(F)F)cc4)n3)cc2)c1] |
| P82 | 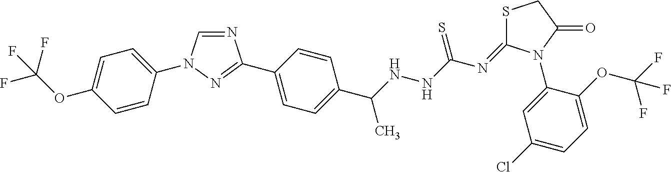 [SMILES: CC(NNC(=S)/N=C1\SCC(=O)N1c1cc(Cl)ccc1OC(F)(F)F)c1ccc(-c2ncn(-c3ccc(OC(F)(F)F)cc3)n2)cc1] |
| P83 | 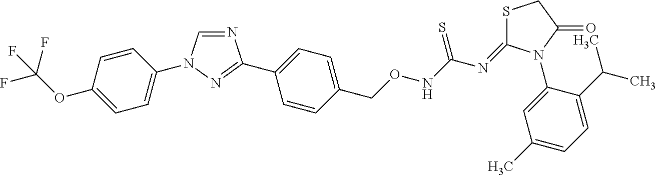 [SMILES: Cc1ccc(C(C)C)c(N2C(=O)CS/C2=N\C(=S)NOCc2ccc(-c3ncn(-c4ccc(OC(F)(F)F)cc4)n3)cc2)c1] |
| P84 | 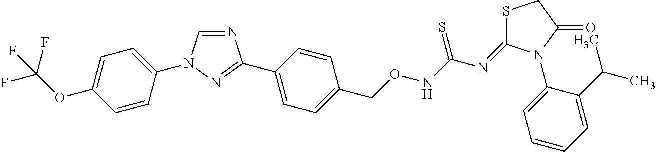 [SMILES: CC(C)c1ccccc1N1C(=O)CS/C1=N\C(=S)NOCc1ccc(-c2ncn(-c3ccc(OC(F)(F)F)cc3)n2)cc1] |
| P85 | 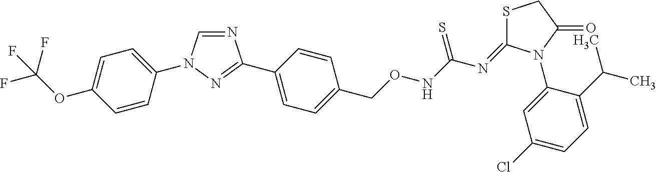 [SMILES: CC(C)c1ccc(Cl)cc1N1C(=O)CS/C1=N\C(=S)NOCc1ccc(-c2ncn(-c3ccc(OC(F)(F)F)cc3)n2)cc1] |
| P86 | 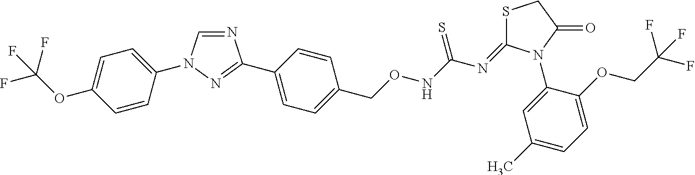 [SMILES: Cc1ccc(OCC(F)(F)F)c(N2C(=O)CS/C2=N\C(=S)NOCc2ccc(-c3ncn(-c4ccc(OC(F)(F)F)cc4)n3)cc2)c1] |
| P87 | 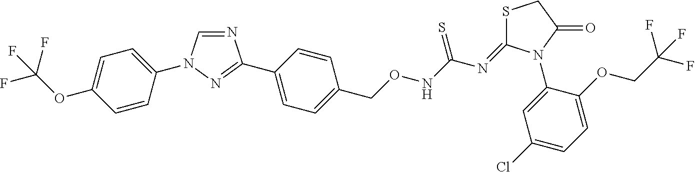 [SMILES: O=C1CS/C(=N\C(=S)NOCc2ccc(-c3ncn(-c4ccc(OC(F)(F)F)cc4)n3)cc2)N1c1cc(Cl)ccc1OCC(F)(F)F] |
| P88 | 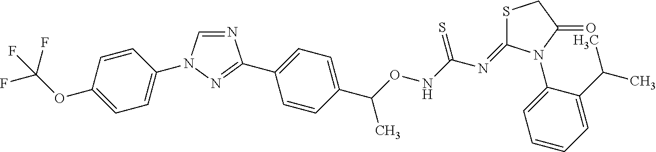 [SMILES: CC(C)c1ccccc1N1C(=O)CS/C1=N\C(=S)NOC(C)c1ccc(-c2ncn(-c3ccc(OC(F)(F)F)cc3)n2)cc1] |
| P89 | 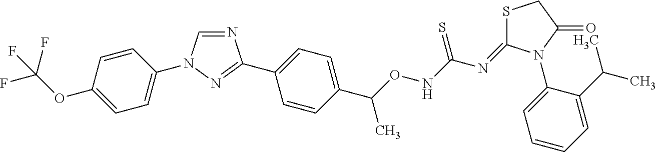 [SMILES: CC(C)c1ccccc1N1C(=O)CS/C1=N\C(=S)NOC(C)c1ccc(-c2ncn(-c3ccc(OC(F)(F)F)cc3)n2)cc1] |
| P90 | 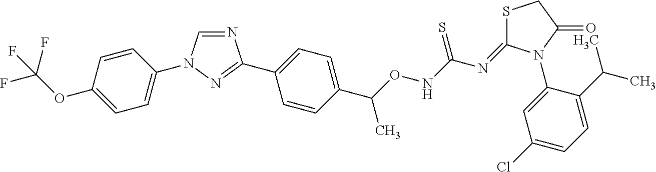 [SMILES: CC(C)c1ccc(Cl)cc1N1C(=O)CS/C1=N\C(=S)NOC(C)c1ccc(-c2ncn(-c3ccc(OC(F)(F)F)cc3)n2)cc1] |
| P91 | 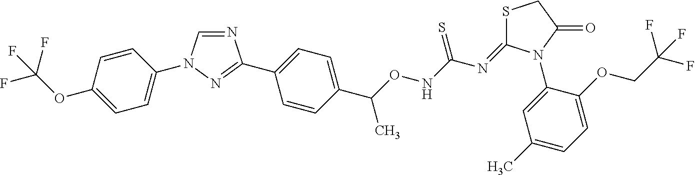 [SMILES: Cc1ccc(OCC(F)(F)F)c(N2C(=O)CS/C2=N\C(=S)NOC(C)c2ccc(-c3ncn(-c4ccc(OC(F)(F)F)cc4)n3)cc2)c1] |
| P92 | 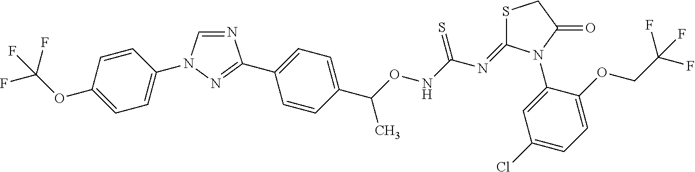 [SMILES: CC(ONC(=S)/N=C1\SCC(=O)N1c1cc(Cl)ccc1OCC(F)(F)F)c1ccc(-c2ncn(-c3ccc(OC(F)(F)F)cc3)n2)cc1] |
| P93 | 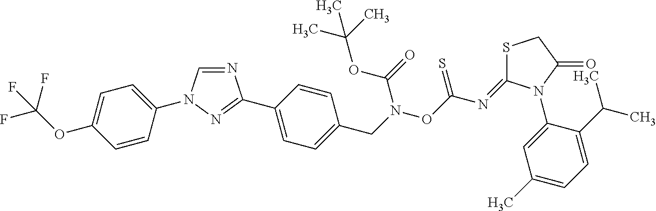 [SMILES: Cc1ccc(C(C)C)c(N2C(=O)CS/C2=N\C(=S)ON(Cc2ccc(-c3ncn(-c4ccc(OC(F)(F)F)cc4)n3)cc2)C(=O)OC(C)(C)C)c1] |
| P94 | 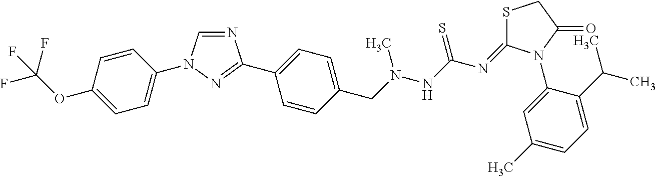 [SMILES: Cc1ccc(C(C)C)c(N2C(=O)CS/C2=N\C(=S)NN(C)Cc2ccc(-c3ncn(-c4ccc(OC(F)(F)F)cc4)n3)cc2)c1] |
| P95 | 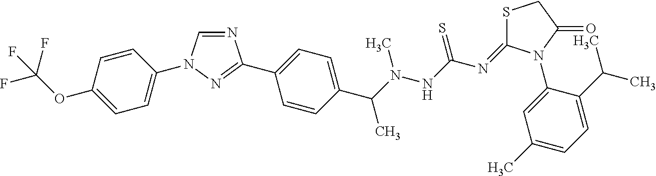 [SMILES: Cc1ccc(C(C)C)c(N2C(=O)CS/C2=N\C(=S)NN(C)C(C)c2ccc(-c3ncn(-c4ccc(OC(F)(F)F)cc4)n3)cc2)c1] |
| P96 | 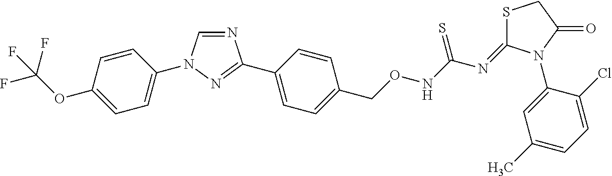 [SMILES: Cc1ccc(Cl)c(N2C(=O)CS/C2=N\C(=S)NOCc2ccc(-c3ncn(-c4ccc(OC(F)(F)F)cc4)n3)cc2)c1] |
| P97 | 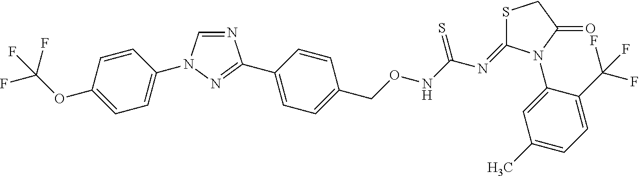 [SMILES: Cc1ccc(C(F)(F)F)c(N2C(=O)CS/C2=N\C(=S)NOCc2ccc(-c3ncn(-c4ccc(OC(F)(F)F)cc4)n3)cc2)c1] |
| P98 | 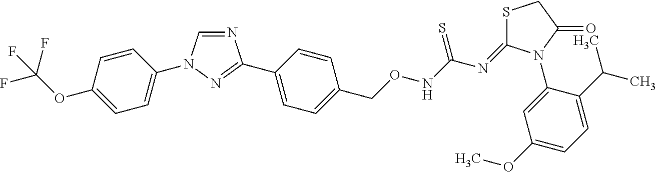 [SMILES: COc1ccc(C(C)C)c(N2C(=O)CS/C2=N\C(=S)NOCc2ccc(-c3ncn(-c4ccc(OC(F)(F)F)cc4)n3)cc2)c1] |
| P99 | 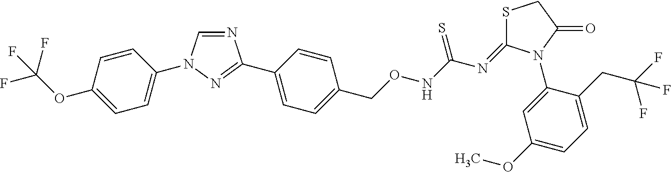 [SMILES: COc1ccc(CC(F)(F)F)c(N2C(=O)CS/C2=N\C(=S)NOCc2ccc(-c3ncn(-c4ccc(OC(F)(F)F)cc4)n3)cc2)c1] |
| P100 | 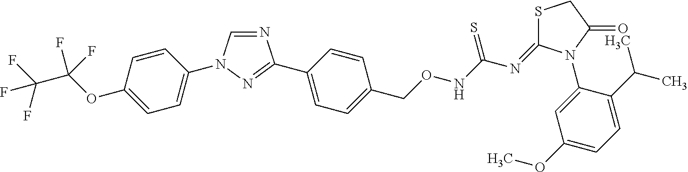 [SMILES: COc1ccc(C(C)C)c(N2C(=O)CS/C2=N\C(=S)NOCc2ccc(-c3ncn(-c4ccc(OC(F)(F)C(F)(F)F)cc4)n3)cc2)c1] |
| P101 | 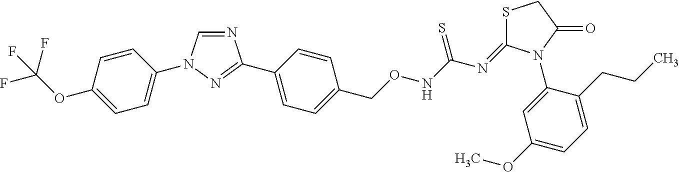 [SMILES: CCCc1ccc(OC)cc1N1C(=O)CS/C1=N\C(=S)NOCc1ccc(-c2ncn(-c3ccc(OC(F)(F)F)cc3)n2)cc1] |
| P102 | 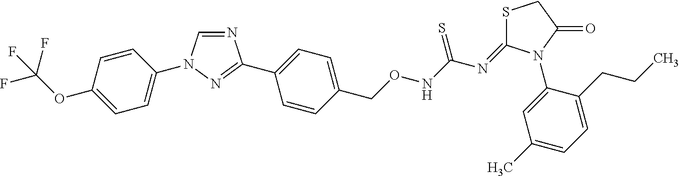 [SMILES: CCCc1ccc(C)cc1N1C(=O)CS/C1=N\C(=S)NOCc1ccc(-c2ncn(-c3ccc(OC(F)(F)F)cc3)n2)cc1] |
| P103 | 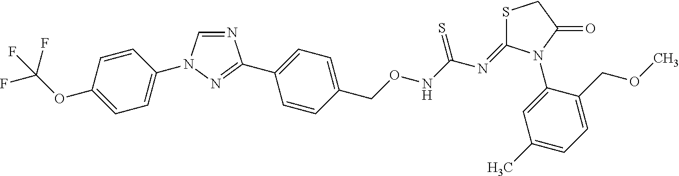 [SMILES: COCc1ccc(C)cc1N1C(=O)CS/C1=N\C(=S)NOCc1ccc(-c2ncn(-c3ccc(OC(F)(F)F)cc3)n2)cc1] |
| P104 | 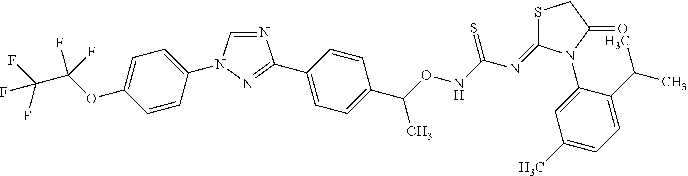 [SMILES: Cc1ccc(C(C)C)c(N2C(=O)CS/C2=N\C(=S)NOC(C)c2ccc(-c3ncn(-c4ccc(OC(F)(F)C(F)(F)F)cc4)n3)cc2)c1] |
| P105 | 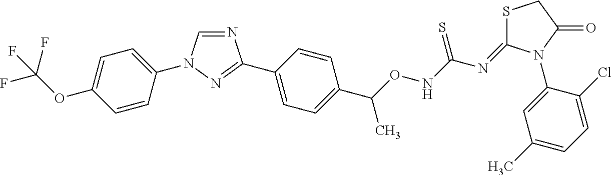 [SMILES: Cc1ccc(Cl)c(N2C(=O)CS/C2=N\C(=S)NOC(C)c2ccc(-c3ncn(-c4ccc(OC(F)(F)F)cc4)n3)cc2)c1] |
| P106 | 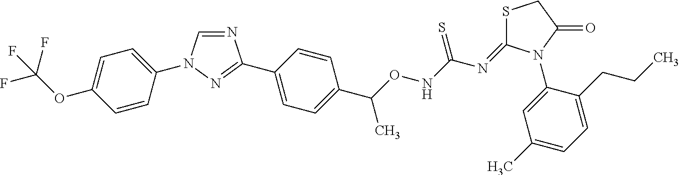 [SMILES: CCCc1ccc(C)cc1N1C(=O)CS/C1=N\C(=S)NOC(C)c1ccc(-c2ncn(-c3ccc(OC(F)(F)F)cc3)n2)cc1] |
| P107 | 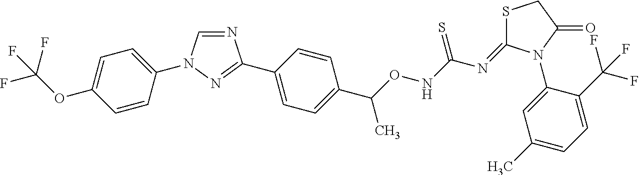 [SMILES: Cc1ccc(C(F)(F)F)c(N2C(=O)CS/C2=N\C(=S)NOC(C)c2ccc(-c3ncn(-c4ccc(OC(F)(F)F)cc4)n3)cc2)c1] |
| P108 | 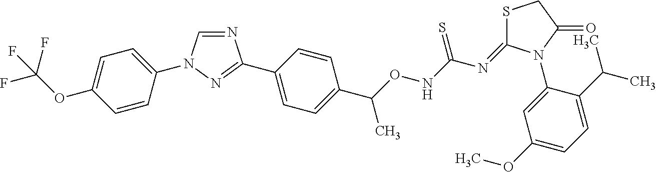 [SMILES: COc1ccc(C(C)C)c(N2C(=O)CS/C2=N\C(=S)NOC(C)c2ccc(-c3ncn(-c4ccc(OC(F)(F)F)cc4)n3)cc2)c1] |
| P109 | 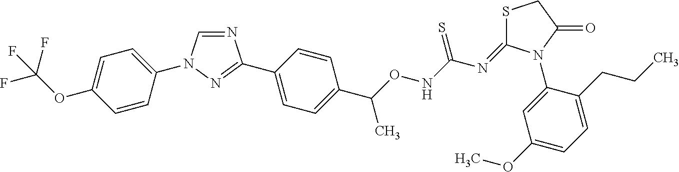 [SMILES: CCCc1ccc(OC)cc1N1C(=O)CS/C1=N\C(=S)NOC(C)c1ccc(-c2ncn(-c3ccc(OC(F)(F)F)cc3)n2)cc1] |
| P110 | 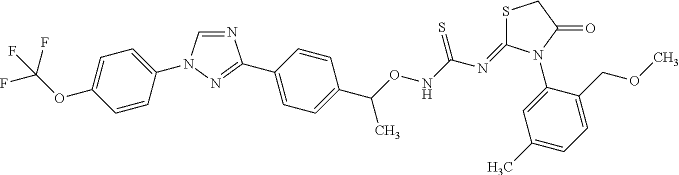 [SMILES: COCc1ccc(C)cc1N1C(=O)CS/C1=N\C(=S)NOC(C)c1ccc(-c2ncn(-c3ccc(OC(F)(F)F)cc3)n2)cc1] |
| P111 | 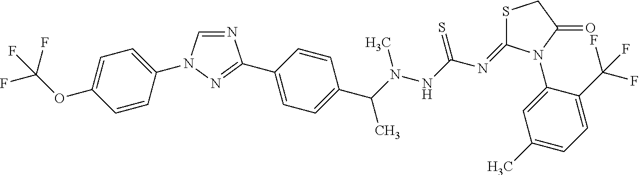 [SMILES: Cc1ccc(C(F)(F)F)c(N2C(=O)CS/C2=N\C(=S)NN(C)C(C)c2ccc(-c3ncn(-c4ccc(OC(F)(F)F)cc4)n3)cc2)c1] |
| P112 | 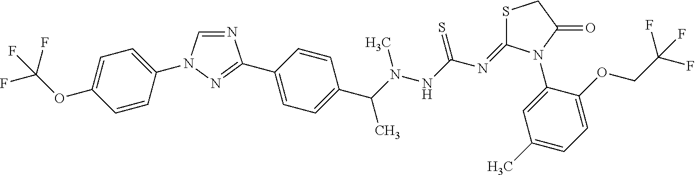 [SMILES: Cc1ccc(OCC(F)(F)F)c(N2C(=O)CS/C2=N\C(=S)NN(C)C(C)c2ccc(-c3ncn(-c4ccc(OC(F)(F)F)cc4)n3)cc2)c1] |
| P113 | 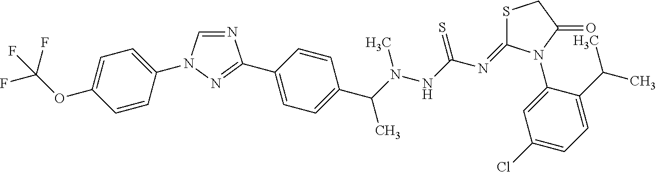 [SMILES: CC(C)c1ccc(Cl)cc1N1C(=O)CS/C1=N\C(=S)NN(C)C(C)c1ccc(-c2ncn(-c3ccc(OC(F)(F)F)cc3)n2)cc1] |
| P114 | 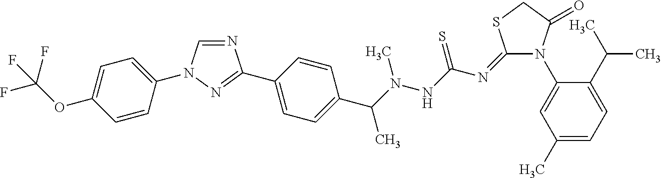 [SMILES: Cc1ccc(C(C)C)c(N2C(=O)CS/C2=N\C(=S)NN(C)C(C)c2ccc(-c3ncn(-c4ccc(OC(F)(F)F)cc4)n3)cc2)c1] |
| P115 | 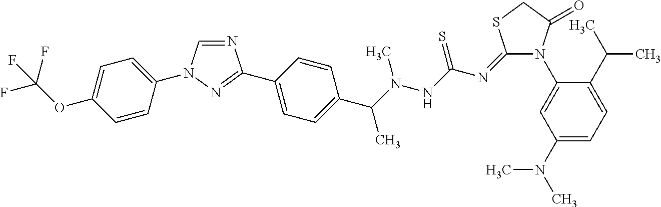 [SMILES: CC(C)c1ccc(N(C)C)cc1N1C(=O)CS/C1=N\C(=S)NN(C)C(C)c1ccc(-c2ncn(-c3ccc(OC(F)(F)F)cc3)n2)cc1] |
| P116 | 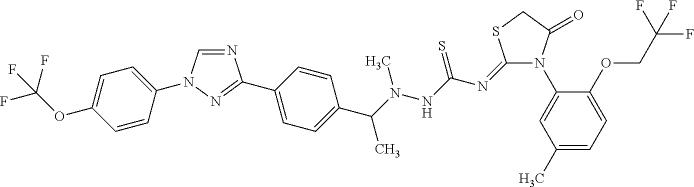 [SMILES: Cc1ccc(OCC(F)(F)F)c(N2C(=O)CS/C2=N\C(=S)NN(C)C(C)c2ccc(-c3ncn(-c4ccc(OC(F)(F)F)cc4)n3)cc2)c1] |
| P117 | 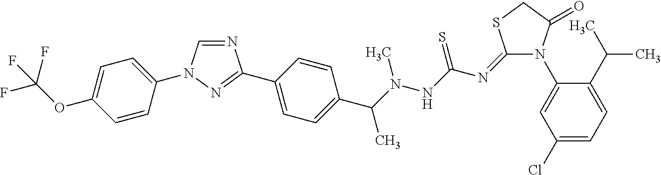 [SMILES: CC(C)c1ccc(Cl)cc1N1C(=O)CS/C1=N\C(=S)NN(C)C(C)c1ccc(-c2ncn(-c3ccc(OC(F)(F)F)cc3)n2)cc1] |
| P118 | 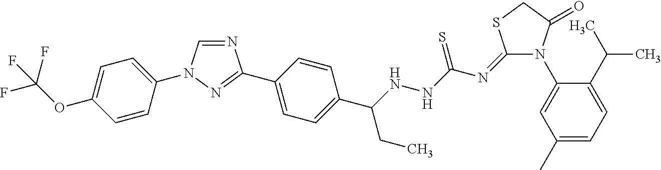 [SMILES: CCC(NNC(=S)/N=C1\SCC(=O)N1c1cc(C)ccc1C(C)C)c1ccc(-c2ncn(-c3ccc(OC(F)(F)F)cc3)n2)cc1] |
| P119 | 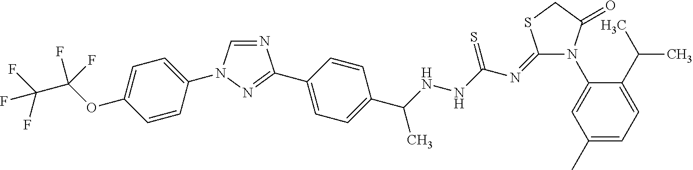 [SMILES: Cc1ccc(C(C)C)c(N2C(=O)CS/C2=N\C(=S)NNC(C)c2ccc(-c3ncn(-c4ccc(OC(F)(F)C(F)(F)F)cc4)n3)cc2)c1] |
| P120 | 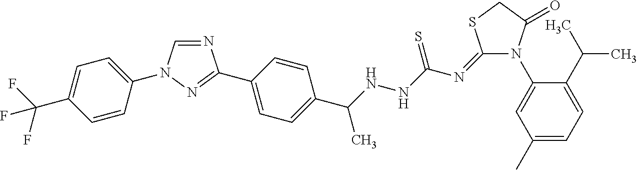 [SMILES: Cc1ccc(C(C)C)c(N2C(=O)CS/C2=N\C(=S)NNC(C)c2ccc(-c3ncn(-c4ccc(C(F)(F)F)cc4)n3)cc2)c1] |
| P121 | 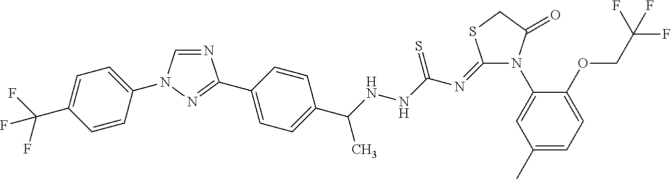 [SMILES: Cc1ccc(OCC(F)(F)F)c(N2C(=O)CS/C2=N\C(=S)NNC(C)c2ccc(-c3ncn(-c4ccc(C(F)(F)F)cc4)n3)cc2)c1] |
| P122 | 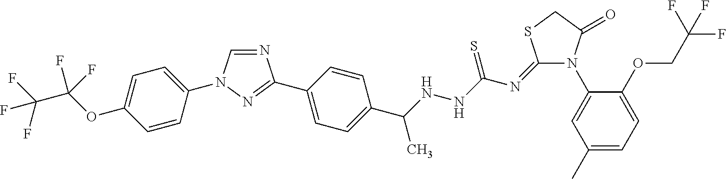 [SMILES: Cc1ccc(OCC(F)(F)F)c(N2C(=O)CS/C2=N\C(=S)NNC(C)c2ccc(-c3ncn(-c4ccc(OC(F)(F)C(F)(F)F)cc4)n3)cc2)c1] |
| P123 | 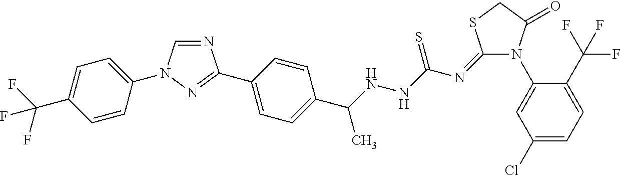 [SMILES: CC(NNC(=S)/N=C1\SCC(=O)N1c1cc(Cl)ccc1C(F)(F)F)c1ccc(-c2ncn(-c3ccc(C(F)(F)F)cc3)n2)cc1] |
| P124 | 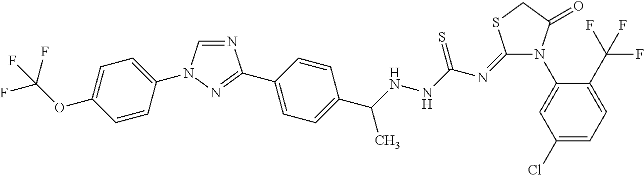 [SMILES: CC(NNC(=S)/N=C1\SCC(=O)N1c1cc(Cl)ccc1C(F)(F)F)c1ccc(-c2ncn(-c3ccc(OC(F)(F)F)cc3)n2)cc1] |
| P125 | 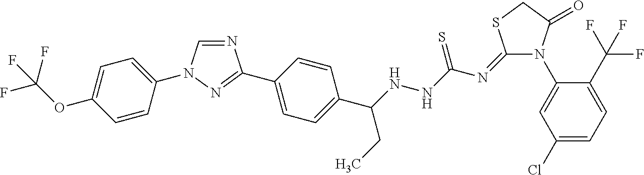 [SMILES: CCC(NNC(=S)/N=C1\SCC(=O)N1c1cc(Cl)ccc1C(F)(F)F)c1ccc(-c2ncn(-c3ccc(OC(F)(F)F)cc3)n2)cc1] |
| P126 | 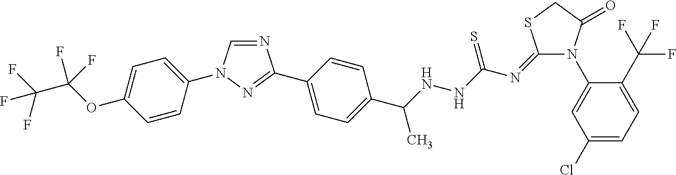 [SMILES: CC(NNC(=S)/N=C1\SCC(=O)N1c1cc(Cl)ccc1C(F)(F)F)c1ccc(-c2ncn(-c3ccc(OC(F)(F)C(F)(F)F)cc3)n2)cc1] |
| P127 | 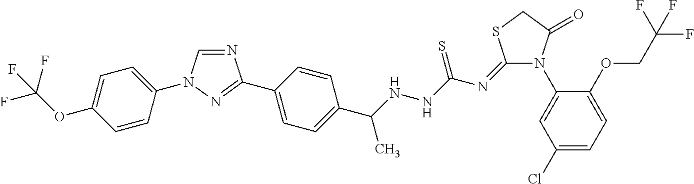 [SMILES: CC(NNC(=S)/N=C1\SCC(=O)N1c1cc(Cl)ccc1OCC(F)(F)F)c1ccc(-c2ncn(-c3ccc(OC(F)(F)F)cc3)n2)cc1] |
| P128 | 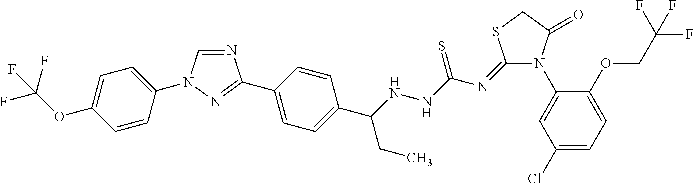 [SMILES: CCC(NNC(=S)/N=C1\SCC(=O)N1c1cc(Cl)ccc1OCC(F)(F)F)c1ccc(-c2ncn(-c3ccc(OC(F)(F)F)cc3)n2)cc1] |
| P129 | 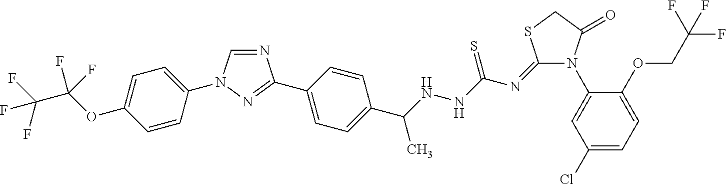 [SMILES: CC(NNC(=S)/N=C1\SCC(=O)N1c1cc(Cl)ccc1OCC(F)(F)F)c1ccc(-c2ncn(-c3ccc(OC(F)(F)C(F)(F)F)cc3)n2)cc1] |
| P130 | 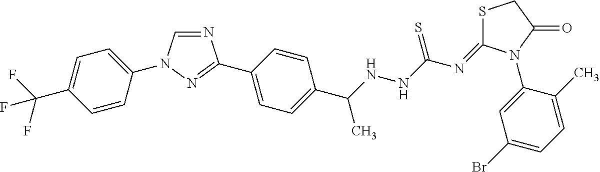 [SMILES: Cc1ccc(Br)cc1N1C(=O)CS/C1=N\C(=S)NNC(C)c1ccc(-c2ncn(-c3ccc(C(F)(F)F)cc3)n2)cc1] |
| P131 | 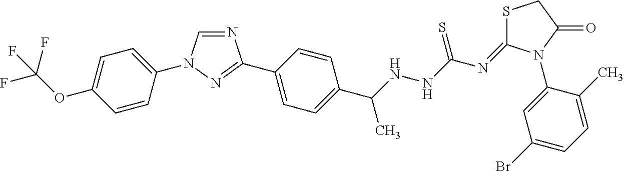 [SMILES: Cc1ccc(Br)cc1N1C(=O)CS/C1=N\C(=S)NNC(C)c1ccc(-c2ncn(-c3ccc(OC(F)(F)F)cc3)n2)cc1] |
| P132 | 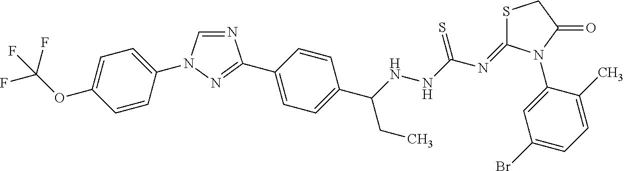 [SMILES: CCC(NNC(=S)/N=C1\SCC(=O)N1c1cc(Br)ccc1C)c1ccc(-c2ncn(-c3ccc(OC(F)(F)F)cc3)n2)cc1] |
| P133 | 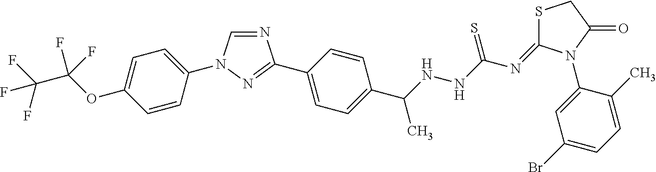 [SMILES: Cc1ccc(Br)cc1N1C(=O)CS/C1=N\C(=S)NNC(C)c1ccc(-c2ncn(-c3ccc(OC(F)(F)C(F)(F)F)cc3)n2)cc1] |
| P134 | 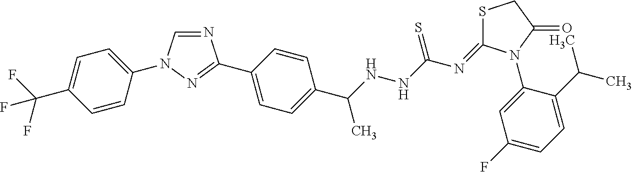 [SMILES: CC(C)c1ccc(F)cc1N1C(=O)CS/C1=N\C(=S)NNC(C)c1ccc(-c2ncn(-c3ccc(C(F)(F)F)cc3)n2)cc1] |
| P135 | 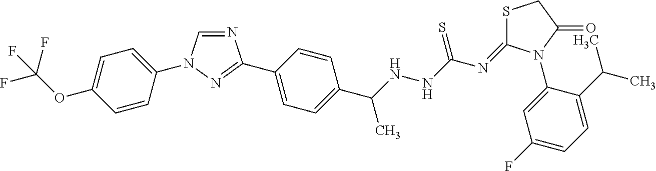 [SMILES: CC(C)c1ccc(F)cc1N1C(=O)CS/C1=N\C(=S)NNC(C)c1ccc(-c2ncn(-c3ccc(OC(F)(F)F)cc3)n2)cc1] |
| P136 | 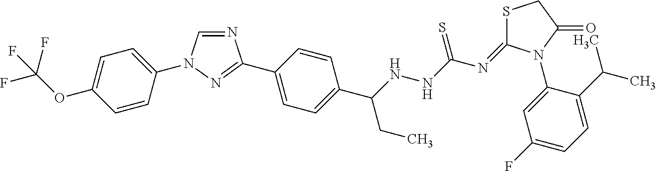 [SMILES: CCC(NNC(=S)/N=C1\SCC(=O)N1c1cc(F)ccc1C(C)C)c1ccc(-c2ncn(-c3ccc(OC(F)(F)F)cc3)n2)cc1] |
| P137 | 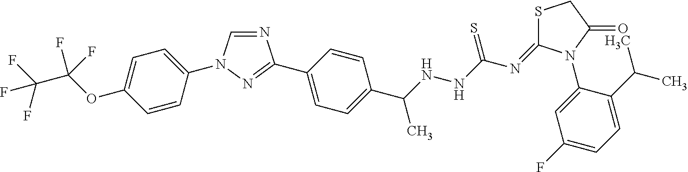 [SMILES: CC(C)c1ccc(F)cc1N1C(=O)CS/C1=N\C(=S)NNC(C)c1ccc(-c2ncn(-c3ccc(OC(F)(F)C(F)(F)F)cc3)n2)cc1] |
| P138 | 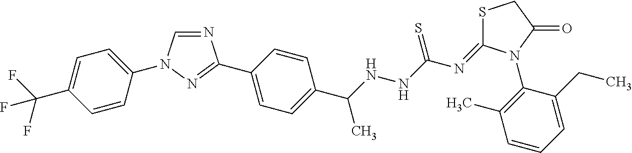 [SMILES: CCc1cccc(C)c1N1C(=O)CS/C1=N\C(=S)NNC(C)c1ccc(-c2ncn(-c3ccc(C(F)(F)F)cc3)n2)cc1] |
| P139 | 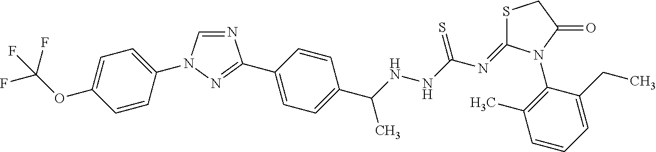 [SMILES: CCc1cccc(C)c1N1C(=O)CS/C1=N\C(=S)NNC(C)c1ccc(-c2ncn(-c3ccc(OC(F)(F)F)cc3)n2)cc1] |
| P140 | 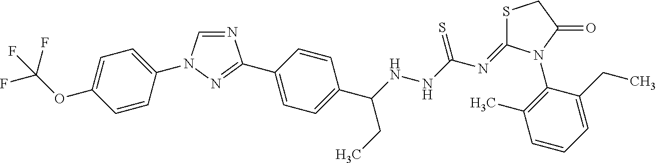 [SMILES: CCc1cccc(C)c1N1C(=O)CS/C1=N\C(=S)NNC(CC)c1ccc(-c2ncn(-c3ccc(OC(F)(F)F)cc3)n2)cc1] |
| P141 | 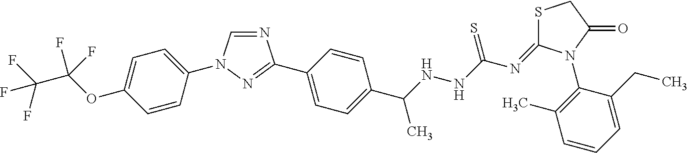 [SMILES: CCc1cccc(C)c1N1C(=O)CS/C1=N\C(=S)NNC(C)c1ccc(-c2ncn(-c3ccc(OC(F)(F)C(F)(F)F)cc3)n2)cc1] |
| P142 | 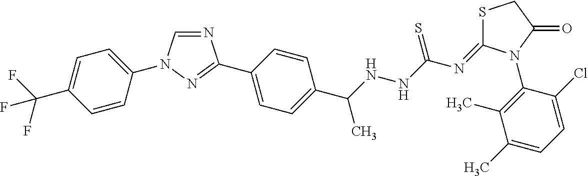 [SMILES: Cc1ccc(Cl)c(N2C(=O)CS/C2=N\C(=S)NNC(C)c2ccc(-c3ncn(-c4ccc(C(F)(F)F)cc4)n3)cc2)c1C] |
| P143 | 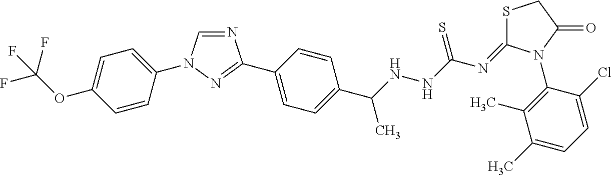 [SMILES: Cc1ccc(Cl)c(N2C(=O)CS/C2=N\C(=S)NNC(C)c2ccc(-c3ncn(-c4ccc(OC(F)(F)F)cc4)n3)cc2)c1C] |
| P144 | 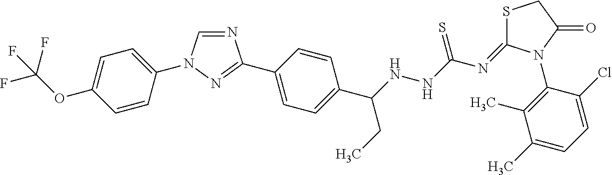 [SMILES: CCC(NNC(=S)/N=C1\SCC(=O)N1c1c(Cl)ccc(C)c1C)c1ccc(-c2ncn(-c3ccc(OC(F)(F)F)cc3)n2)cc1] |
| P145 | 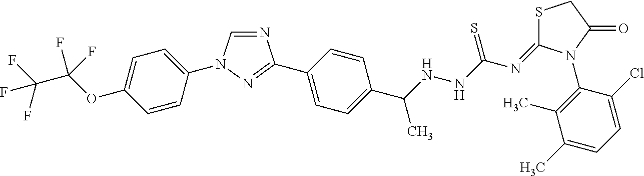 [SMILES: Cc1ccc(Cl)c(N2C(=O)CS/C2=N\C(=S)NNC(C)c2ccc(-c3ncn(-c4ccc(OC(F)(F)C(F)(F)F)cc4)n3)cc2)c1C] |
| P146 | 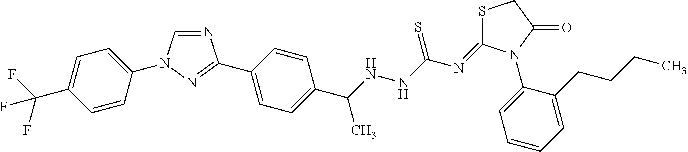 [SMILES: CCCCc1ccccc1N1C(=O)CS/C1=N\C(=S)NNC(C)c1ccc(-c2ncn(-c3ccc(C(F)(F)F)cc3)n2)cc1] |
| P147 | 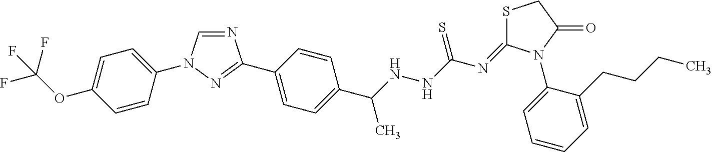 [SMILES: CCCCc1ccccc1N1C(=O)CS/C1=N\C(=S)NNC(C)c1ccc(-c2ncn(-c3ccc(OC(F)(F)F)cc3)n2)cc1] |
| P148 | 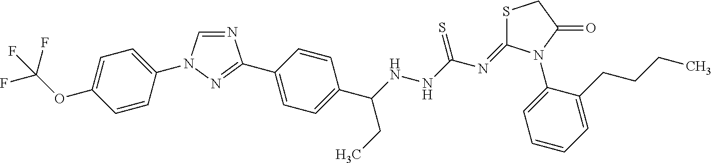 [SMILES: CCCCc1ccccc1N1C(=O)CS/C1=N\C(=S)NNC(CC)c1ccc(-c2ncn(-c3ccc(OC(F)(F)F)cc3)n2)cc1] |
| P149 | 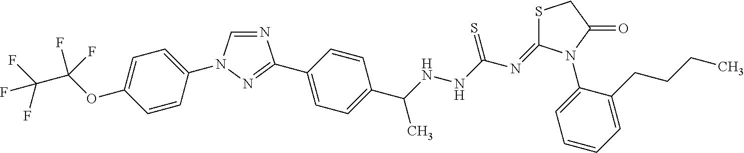 [SMILES: CCCCc1ccccc1N1C(=O)CS/C1=N\C(=S)NNC(C)c1ccc(-c2ncn(-c3ccc(OC(F)(F)C(F)(F)F)cc3)n2)cc1] |
| P150 | 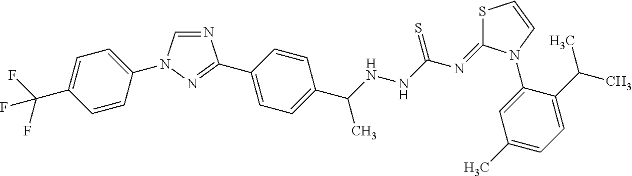 [SMILES: Cc1ccc(C(C)C)c(-n2ccs/c2=N\C(=S)NNC(C)c2ccc(-c3ncn(-c4ccc(C(F)(F)F)cc4)n3)cc2)c1] |
| P151 | 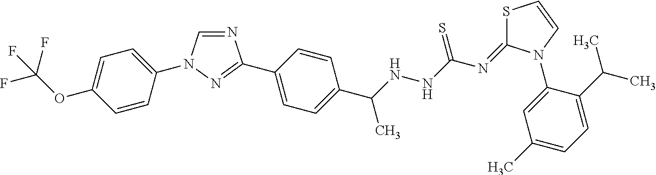 [SMILES: Cc1ccc(C(C)C)c(-n2ccs/c2=N\C(=S)NNC(C)c2ccc(-c3ncn(-c4ccc(OC(F)(F)F)cc4)n3)cc2)c1] |
| P152 | 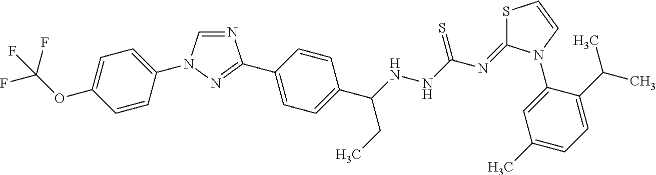 [SMILES: CCC(NNC(=S)/N=c1\sccn1-c1cc(C)ccc1C(C)C)c1ccc(-c2ncn(-c3ccc(OC(F)(F)F)cc3)n2)cc1] |
| P153 | 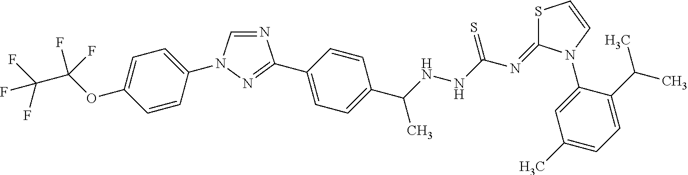 [SMILES: Cc1ccc(C(C)C)c(-n2ccs/c2=N\C(=S)NNC(C)c2ccc(-c3ncn(-c4ccc(OC(F)(F)C(F)(F)F)cc4)n3)cc2)c1] |
| P154 | 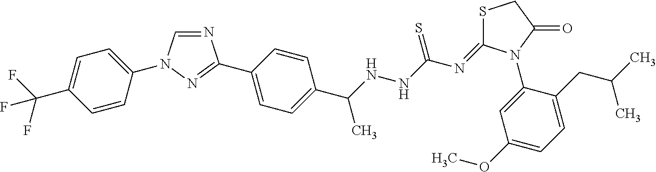 [SMILES: COc1ccc(CC(C)C)c(N2C(=O)CS/C2=N\C(=S)NNC(C)c2ccc(-c3ncn(-c4ccc(C(F)(F)F)cc4)n3)cc2)c1] |
| P155 | 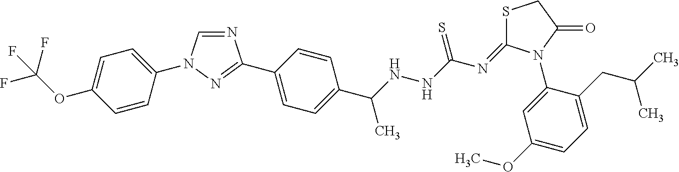 [SMILES: COc1ccc(CC(C)C)c(N2C(=O)CS/C2=N\C(=S)NNC(C)c2ccc(-c3ncn(-c4ccc(OC(F)(F)F)cc4)n3)cc2)c1] |
| P156 | 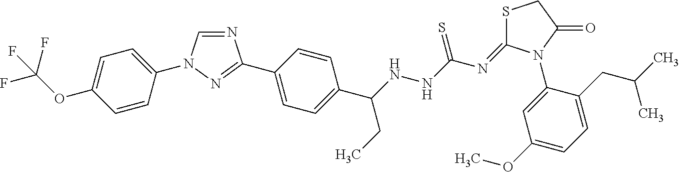 [SMILES: CCC(NNC(=S)/N=C1\SCC(=O)N1c1cc(OC)ccc1CC(C)C)c1ccc(-c2ncn(-c3ccc(OC(F)(F)F)cc3)n2)cc1] |
| P157 | 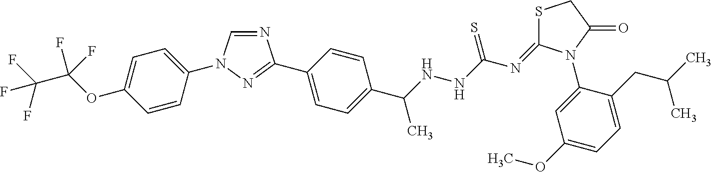 [SMILES: COc1ccc(CC(C)C)c(N2C(=O)CS/C2=N\C(=S)NNC(C)c2ccc(-c3ncn(-c4ccc(OC(F)(F)C(F)(F)F)cc4)n3)cc2)c1] |
| P158 | 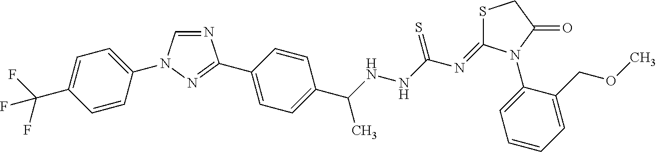 [SMILES: COCc1ccccc1N1C(=O)CS/C1=N\C(=S)NNC(C)c1ccc(-c2ncn(-c3ccc(C(F)(F)F)cc3)n2)cc1] |
| P159 | 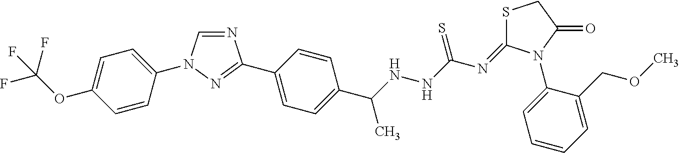 [SMILES: COCc1ccccc1N1C(=O)CS/C1=N\C(=S)NNC(C)c1ccc(-c2ncn(-c3ccc(OC(F)(F)F)cc3)n2)cc1] |
| P160 | 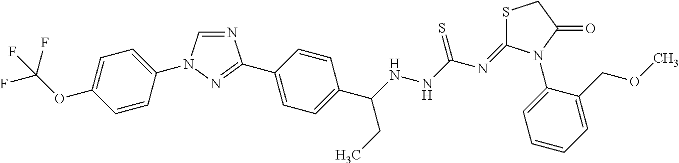 [SMILES: CCC(NNC(=S)/N=C1\SCC(=O)N1c1ccccc1COC)c1ccc(-c2ncn(-c3ccc(OC(F)(F)F)cc3)n2)cc1] |
| P161 | 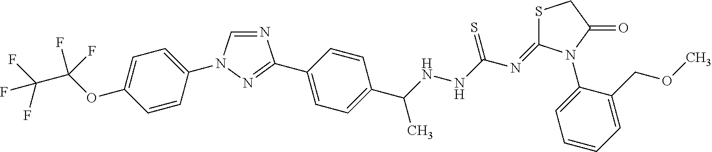 [SMILES: COCc1ccccc1N1C(=O)CS/C1=N\C(=S)NNC(C)c1ccc(-c2ncn(-c3ccc(OC(F)(F)C(F)(F)F)cc3)n2)cc1] |
| P162 | 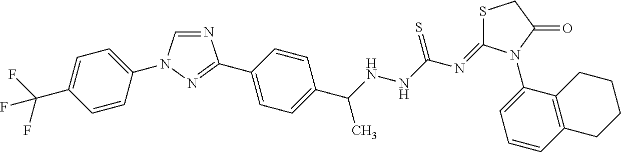 |
| P163 | 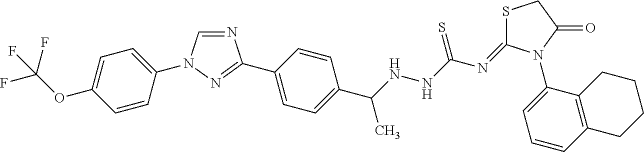 [SMILES: CC(NNC(=S)/N=C1\SCC(=O)N1c1cccc2c1CCCC2)c1ccc(-c2ncn(-c3ccc(OC(F)(F)F)cc3)n2)cc1] |
| P164 | 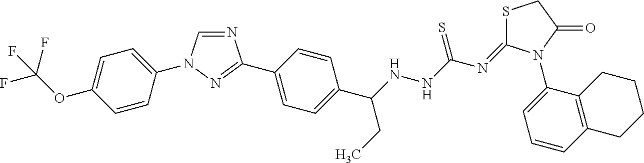 [SMILES: CCC(NNC(=S)/N=C1\SCC(=O)N1c1cccc2c1CCCC2)c1ccc(-c2ncn(-c3ccc(OC(F)(F)F)cc3)n2)cc1] |
| P165 | 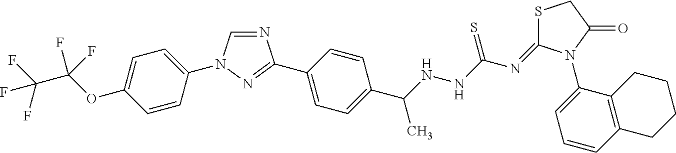 [SMILES: CC(NNC(=S)/N=C1\SCC(=O)N1c1cccc2c1CCCC2)c1ccc(-c2ncn(-c3ccc(OC(F)(F)C(F)(F)F)cc3)n2)cc1] |
| P166 | 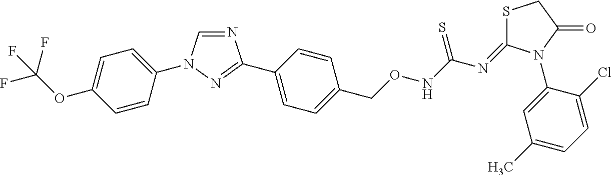 [SMILES: Cc1ccc(Cl)c(N2C(=O)CS/C2=N\C(=S)NOCc2ccc(-c3ncn(-c4ccc(OC(F)(F)F)cc4)n3)cc2)c1] |
| P167 | 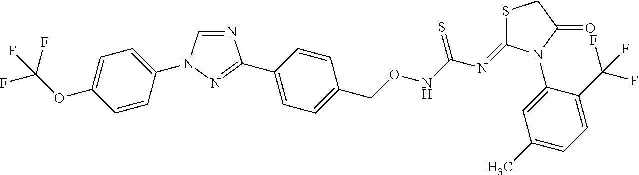 [SMILES: Cc1ccc(C(F)(F)F)c(N2C(=O)CS/C2=N\C(=S)NOCc2ccc(-c3ncn(-c4ccc(OC(F)(F)F)cc4)n3)cc2)c1] |
| P168 | 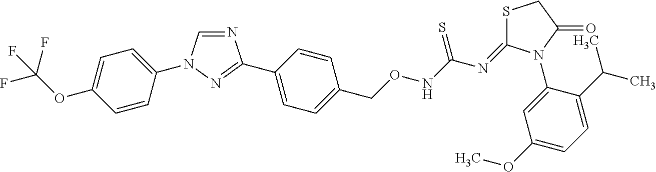 [SMILES: COc1ccc(C(C)C)c(N2C(=O)CS/C2=N\C(=S)NOCc2ccc(-c3ncn(-c4ccc(OC(F)(F)F)cc4)n3)cc2)c1] |
| P169 | 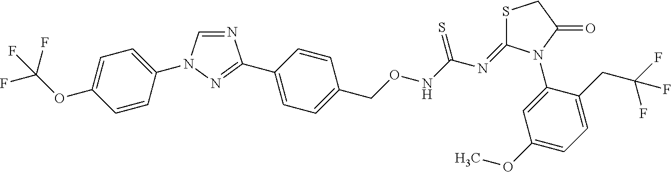 [SMILES: COc1ccc(CC(F)(F)F)c(N2C(=O)CS/C2=N\C(=S)NOCc2ccc(-c3ncn(-c4ccc(OC(F)(F)F)cc4)n3)cc2)c1] |
| P170 | 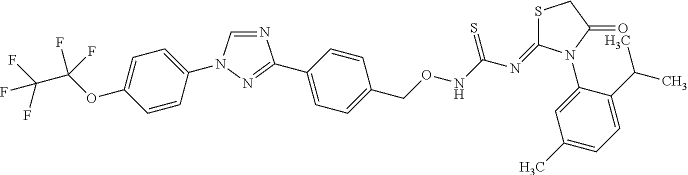 [SMILES: Cc1ccc(C(C)C)c(N2C(=O)CS/C2=N\C(=S)NOCc2ccc(-c3ncn(-c4ccc(OC(F)(F)C(F)(F)F)cc4)n3)cc2)c1] |
| P171 | 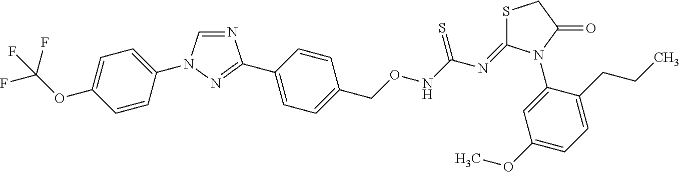 [SMILES: CCCc1ccc(OC)cc1N1C(=O)CS/C1=N\C(=S)NOCc1ccc(-c2ncn(-c3ccc(OC(F)(F)F)cc3)n2)cc1] |
| P172 | 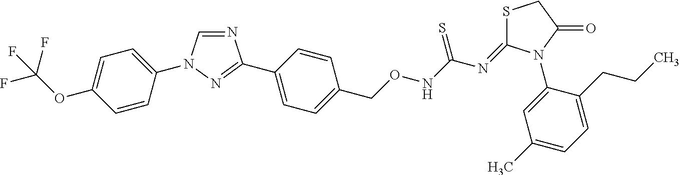 [SMILES: CCCc1ccc(C)cc1N1C(=O)CS/C1=N\C(=S)NOCc1ccc(-c2ncn(-c3ccc(OC(F)(F)F)cc3)n2)cc1] |
| P173 | 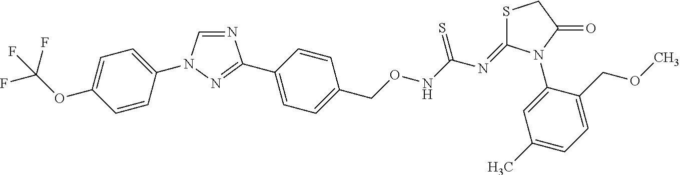 [SMILES: COCc1ccc(C)cc1N1C(=O)CS/C1=N\C(=S)NOCc1ccc(-c2ncn(-c3ccc(OC(F)(F)F)cc3)n2)cc1] |
| P174 | 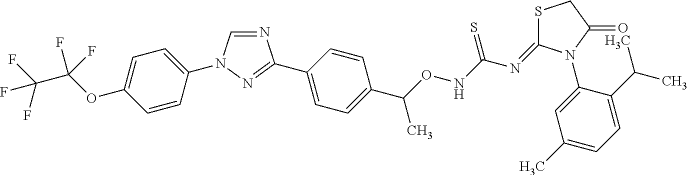 [SMILES: Cc1ccc(C(C)C)c(N2C(=O)CS/C2=N\C(=S)NOC(C)c2ccc(-c3ncn(-c4ccc(OC(F)(F)C(F)(F)F)cc4)n3)cc2)c1] |
| P175 | 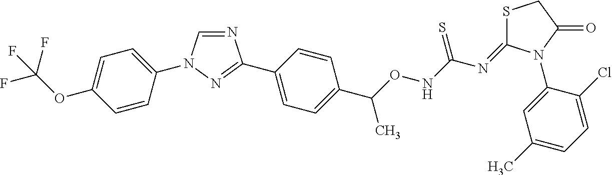 [SMILES: Cc1ccc(Cl)c(N2C(=O)CS/C2=N\C(=S)NOC(C)c2ccc(-c3ncn(-c4ccc(OC(F)(F)F)cc4)n3)cc2)c1] |
| P176 | 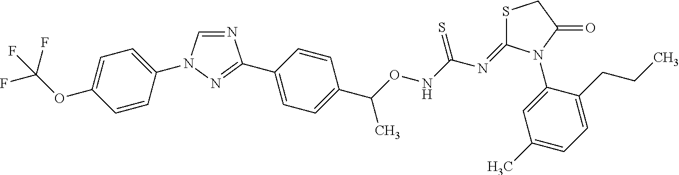 [SMILES: CCCc1ccc(C)cc1N1C(=O)CS/C1=N\C(=S)NOC(C)c1ccc(-c2ncn(-c3ccc(OC(F)(F)F)cc3)n2)cc1] |
| P177 | 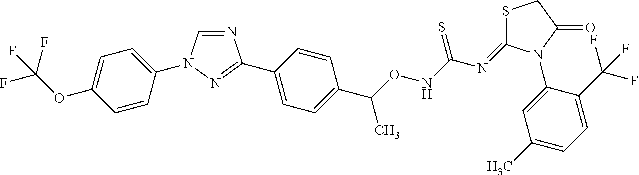 [SMILES: Cc1ccc(C(F)(F)F)c(N2C(=O)CS/C2=N\C(=S)NOC(C)c2ccc(-c3ncn(-c4ccc(OC(F)(F)F)cc4)n3)cc2)c1] |
| P178 | 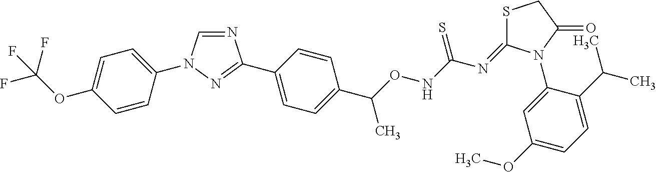 [SMILES: COc1ccc(C(C)C)c(N2C(=O)CS/C2=N\C(=S)NOC(C)c2ccc(-c3ncn(-c4ccc(OC(F)(F)F)cc4)n3)cc2)c1] |
| P179 | 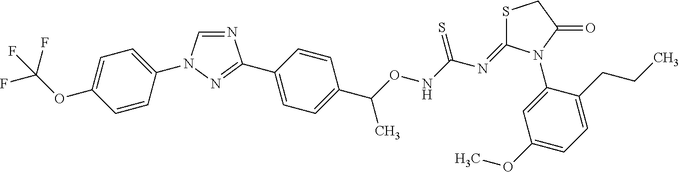 [SMILES: CCCc1ccc(OC)cc1N1C(=O)CS/C1=N\C(=S)NOC(C)c1ccc(-c2ncn(-c3ccc(OC(F)(F)F)cc3)n2)cc1] |
| P180 | 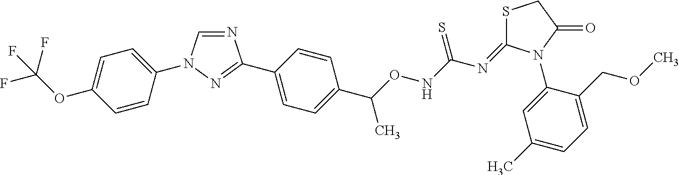 [SMILES: COCc1ccc(C)cc1N1C(=O)CS/C1=N\C(=S)NOC(C)c1ccc(-c2ncn(-c3ccc(OC(F)(F)F)cc3)n2)cc1] |
| P181 | 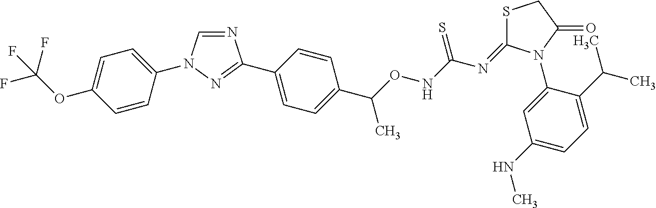 [SMILES: CNc1ccc(C(C)C)c(N2C(=O)CS/C2=N\C(=S)NOC(C)c2ccc(-c3ncn(-c4ccc(OC(F)(F)F)cc4)n3)cc2)c1] |
| P182 | 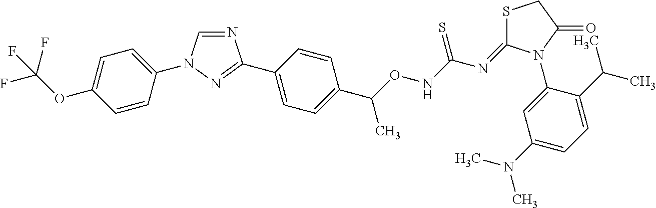 [SMILES: CC(C)c1ccc(N(C)C)cc1N1C(=O)CS/C1=N\C(=S)NOC(C)c1ccc(-c2ncn(-c3ccc(OC(F)(F)F)cc3)n2)cc1] |
| P183 | 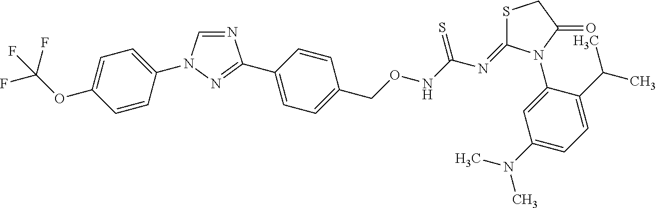 [SMILES: CC(C)c1ccc(N(C)C)cc1N1C(=O)CS/C1=N\C(=S)NOCc1ccc(-c2ncn(-c3ccc(OC(F)(F)F)cc3)n2)cc1] |
| P184 | 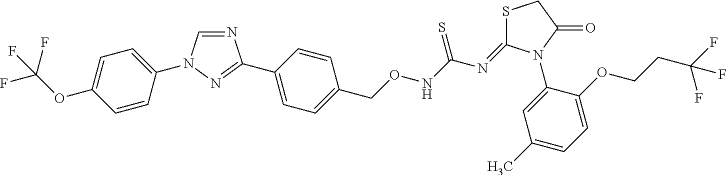 [SMILES: Cc1ccc(OCCC(F)(F)F)c(N2C(=O)CS/C2=N\C(=S)NOCc2ccc(-c3ncn(-c4ccc(OC(F)(F)F)cc4)n3)cc2)c1] |
| P185 | 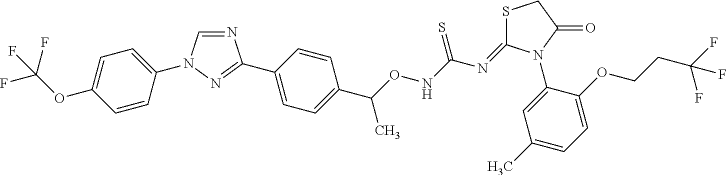 [SMILES: Cc1ccc(OCCC(F)(F)F)c(N2C(=O)CS/C2=N\C(=S)NOC(C)c2ccc(-c3ncn(-c4ccc(OC(F)(F)F)cc4)n3)cc2)c1] |
| P186 | 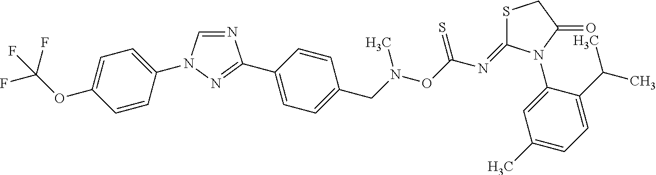 [SMILES: Cc1ccc(C(C)C)c(N2C(=O)CS/C2=N\C(=S)ON(C)Cc2ccc(-c3ncn(-c4ccc(OC(F)(F)F)cc4)n3)cc2)c1] |
| P187 | 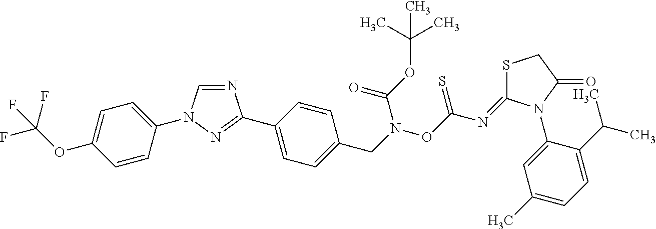 [SMILES: Cc1ccc(C(C)C)c(N2C(=O)CS/C2=N\C(=S)ON(Cc2ccc(-c3ncn(-c4ccc(OC(F)(F)F)cc4)n3)cc2)C(=O)OC(C)(C)C)c1] |
| P188 | 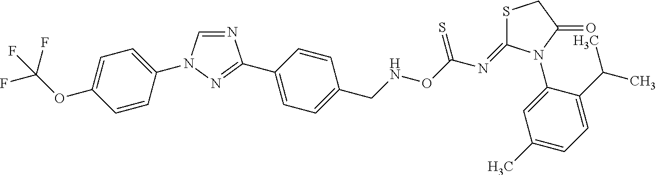 [SMILES: Cc1ccc(C(C)C)c(N2C(=O)CS/C2=N\C(=S)ONCc2ccc(-c3ncn(-c4ccc(OC(F)(F)F)cc4)n3)cc2)c1] |

TABLE 1
Structures for Compounds

| | |
|---|---|
| A3 | 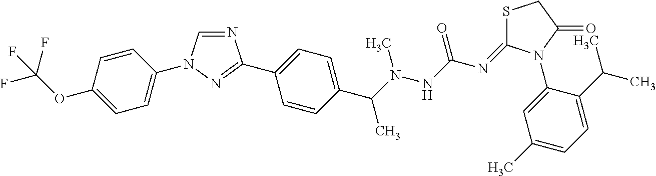 [SMILES: Cc1ccc(C(C)C)c(N2C(=O)CS/C2=N\C(=O)NN(C)C(C)c2ccc(-c3ncn(-c4ccc(OC(F)(F)F)cc4)n3)cc2)c1] |
| A4 | 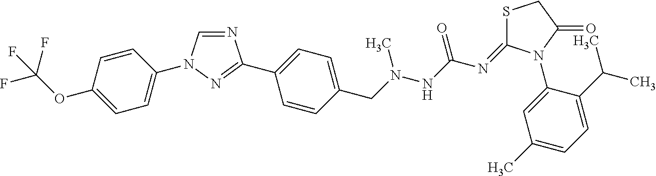 [SMILES: Cc1ccc(C(C)C)c(N2C(=O)CS/C2=N\C(=O)NN(C)Cc2ccc(-c3ncn(-c4ccc(OC(F)(F)F)cc4)n3)cc2)c1] |
| A5 | 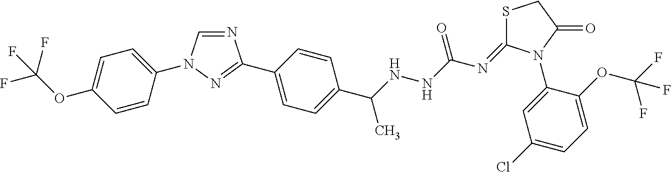 [SMILES: CC(NNC(=O)/N=C1\SCC(=O)N1c1cc(Cl)ccc1OC(F)(F)F)c1ccc(-c2ncn(-c3ccc(OC(F)(F)F)cc3)n2)cc1] |
| A6 | 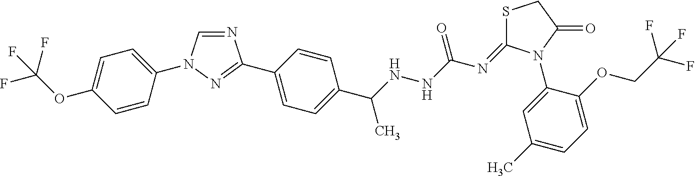 [SMILES: Cc1ccc(OCC(F)(F)F)c(N2C(=O)CS/C2=N\C(=O)NNC(C)c2ccc(-c3ncn(-c4ccc(OC(F)(F)F)cc4)n3)cc2)c1] |
| A7 | 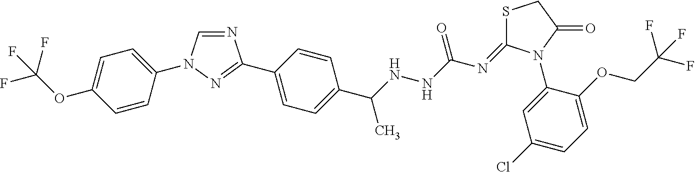 [SMILES: CC(NNC(=O)/N=C1\SCC(=O)N1c1cc(Cl)ccc1OCC(F)(F)F)c1ccc(-c2ncn(-c3ccc(OC(F)(F)F)cc3)n2)cc1] |
| A8 | 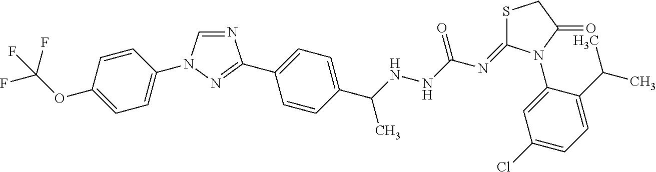 [SMILES: CC(C)c1ccc(Cl)cc1N1C(=O)CS/C1=N\C(=O)NNC(C)c1ccc(-c2ncn(-c3ccc(OC(F)(F)F)cc3)n2)cc1] |
| A9 | 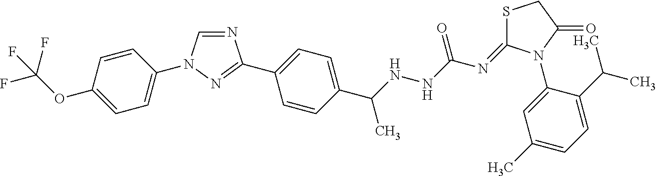 [SMILES: Cc1ccc(C(C)C)c(N2C(=O)CS/C2=N\C(=O)NNC(C)c2ccc(-c3ncn(-c4ccc(OC(F)(F)F)cc4)n3)cc2)c1] |
| A10 | 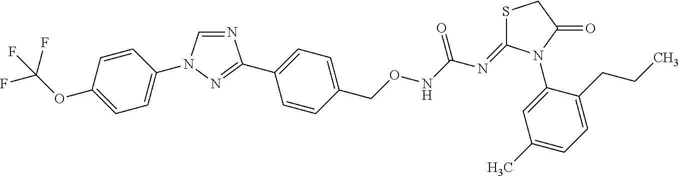 [SMILES: CCCc1ccc(C)cc1N1C(=O)CS/C1=N\C(=O)NOCc1ccc(-c2ncn(-c3ccc(OC(F)(F)F)cc3)n2)cc1] |
| A11 | 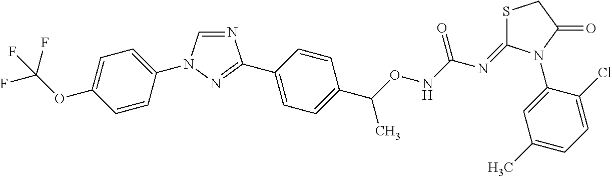 [SMILES: Cc1ccc(Cl)c(N2C(=O)CS/C2=N\C(=O)NOC(C)c2ccc(-c3ncn(-c4ccc(OC(F)(F)F)cc4)n3)cc2)c1] |
| A12 | 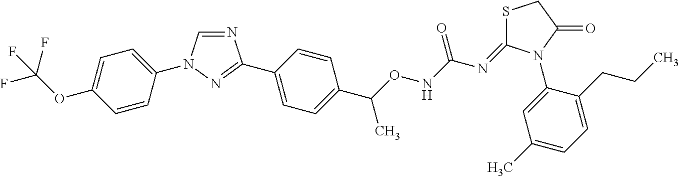 [SMILES: CCCc1ccc(C)cc1N1C(=O)CS/C1=N\C(=O)NOC(C)c1ccc(-c2ncn(-c3ccc(OC(F)(F)F)cc3)n2)cc1] |
| A13 | 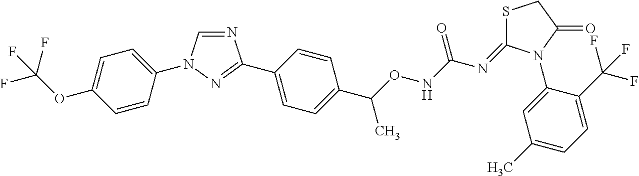 [SMILES: Cc1ccc(C(F)(F)F)c(N2C(=O)CS/C2=N\C(=O)NOC(C)c2ccc(-c3ncn(-c4ccc(OC(F)(F)F)cc4)n3)cc2)c1] |
| A14 | 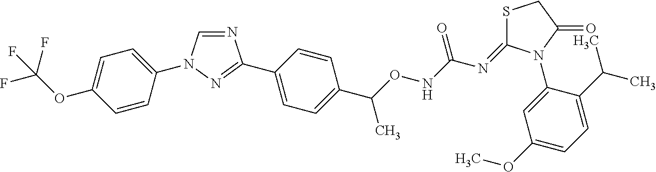 [SMILES: COc1ccc(C(C)C)c(N2C(=O)CS/C2=N\C(=O)NOC(C)c2ccc(-c3ncn(-c4ccc(OC(F)(F)F)cc4)n3)cc2)c1] |
| A15 | 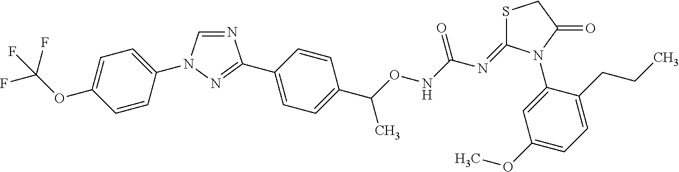 [SMILES: CCCc1ccc(OC)cc1N1C(=O)CS/C1=N\C(=O)NOC(C)c1ccc(-c2ncn(-c3ccc(OC(F)(F)F)cc3)n2)cc1] |
| A16 | 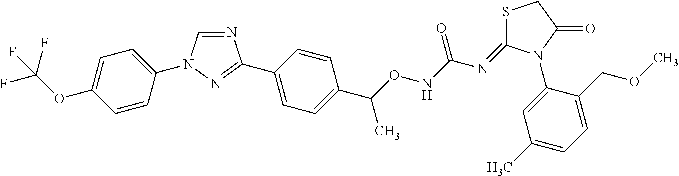 [SMILES: COCc1ccc(C)cc1N1C(=O)CS/C1=N\C(=O)NOC(C)c1ccc(-c2ncn(-c3ccc(OC(F)(F)F)cc3)n2)cc1] |
| A17 | 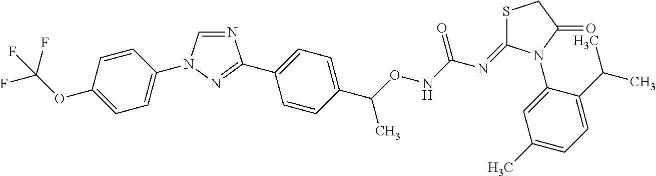 [SMILES: Cc1ccc(C(C)C)c(N2C(=O)CS/C2=N\C(=O)NOC(C)c2ccc(-c3ncn(-c4ccc(OC(F)(F)F)cc4)n3)cc2)c1] |
| A18 | 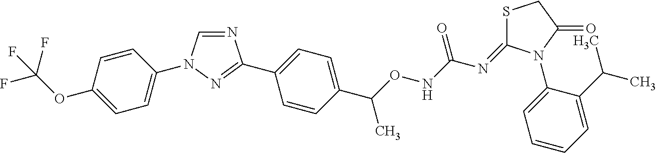 [SMILES: CC(C)c1ccccc1N1C(=O)CS/C1=N\C(=O)NOC(C)c1ccc(-c2ncn(-c3ccc(OC(F)(F)F)cc3)n2)cc1] |
| A19 | 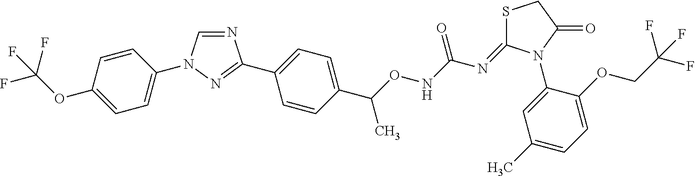 [SMILES: Cc1ccc(OCC(F)(F)F)c(N2C(=O)CS/C2=N\C(=O)NOC(C)c2ccc(-c3ncn(-c4ccc(OC(F)(F)F)cc4)n3)cc2)c1] |
| A20 | 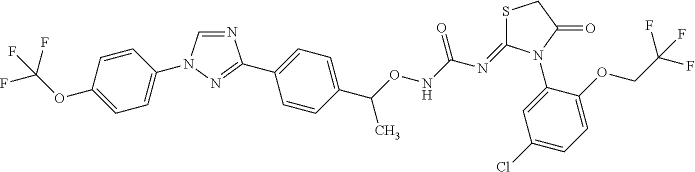 [SMILES: CC(ONC(=O)/N=C1\SCC(=O)N1c1cc(Cl)ccc1OCC(F)(F)F)c1ccc(-c2ncn(-c3ccc(OC(F)(F)F)cc3)n2)cc1] |
| A21 | 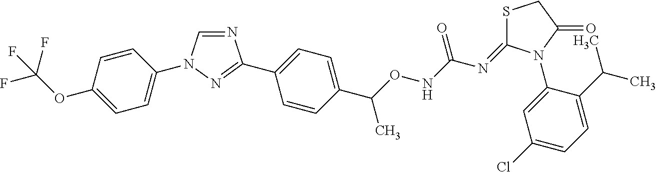 [SMILES: CC(C)c1ccc(Cl)cc1N1C(=O)CS/C1=N\C(=O)NOC(C)c1ccc(-c2ncn(-c3ccc(OC(F)(F)F)cc3)n2)cc1] |
| A22 | 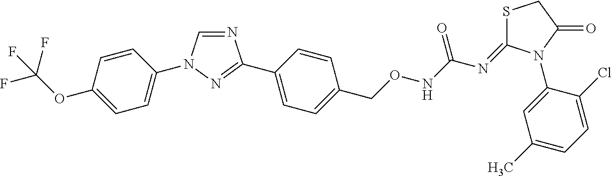 [SMILES: Cc1ccc(Cl)c(N2C(=O)CS/C2=N\C(=O)NOCc2ccc(-c3ncn(-c4ccc(OC(F)(F)F)cc4)n3)cc2)c1] |
| A23 | 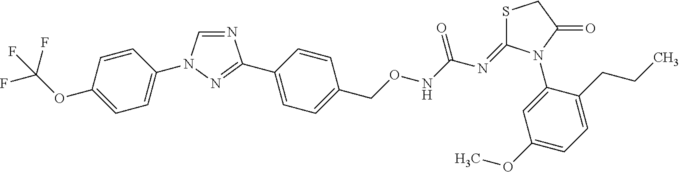 [SMILES: CCCc1ccc(OC)cc1N1C(=O)CS/C1=N\C(=O)NOCc1ccc(-c2ncn(-c3ccc(OC(F)(F)F)cc3)n2)cc1] |
| A24 | 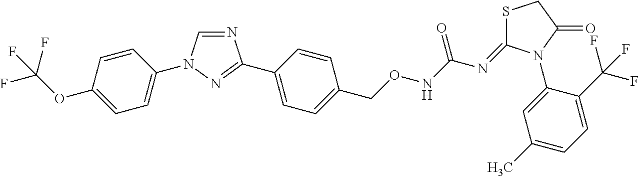 [SMILES: Cc1ccc(C(F)(F)F)c(N2C(=O)CS/C2=N\C(=O)NOCc2ccc(-c3ncn(-c4ccc(OC(F)(F)F)cc4)n3)cc2)c1] |
| A25 | 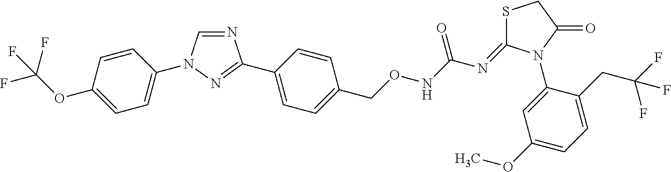 [SMILES: COc1ccc(CC(F)(F)F)c(N2C(=O)CS/C2=N\C(=O)NOCc2ccc(-c3ncn(-c4ccc(OC(F)(F)F)cc4)n3)cc2)c1] |
| A26 | 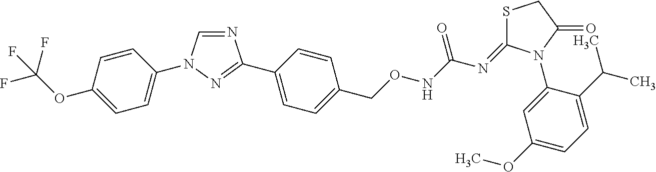 [SMILES: COc1ccc(C(C)C)c(N2C(=O)CS/C2=N\C(=O)NOCc2ccc(-c3ncn(-c4ccc(OC(F)(F)F)cc4)n3)cc2)c1] |
| A27 | 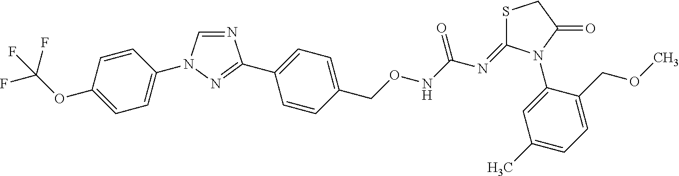 [SMILES: COCc1ccc(C)cc1N1C(=O)CS/C1=N\C(=O)NOCc1ccc(-c2ncn(-c3ccc(OC(F)(F)F)cc3)n2)cc1] |
| A28 | 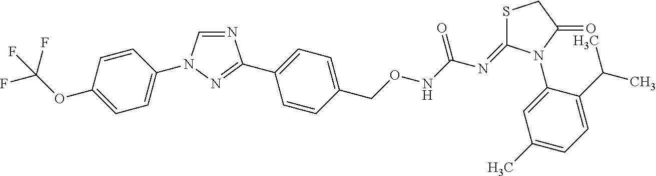 [SMILES: Cc1ccc(C(C)C)c(N2C(=O)CS/C2=N\C(=O)NOCc2ccc(-c3ncn(-c4ccc(OC(F)(F)F)cc4)n3)cc2)c1] |
| A29 | 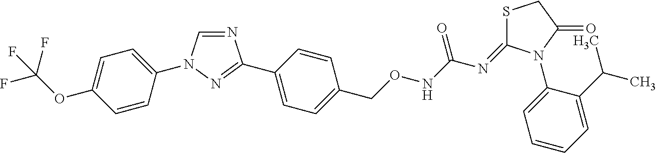 [SMILES: CC(C)c1ccccc1N1C(=O)CS/C1=N\C(=O)NOCc1ccc(-c2ncn(-c3ccc(OC(F)(F)F)cc3)n2)cc1] |
| A30 | 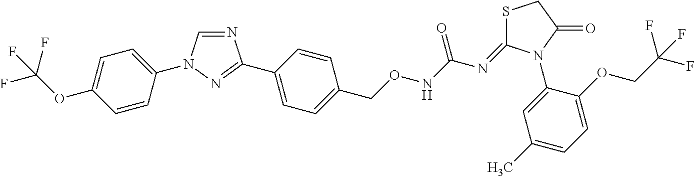 [SMILES: Cc1ccc(OCC(F)(F)F)c(N2C(=O)CS/C2=N\C(=O)NOCc2ccc(-c3ncn(-c4ccc(OC(F)(F)F)cc4)n3)cc2)c1] |
| A31 | 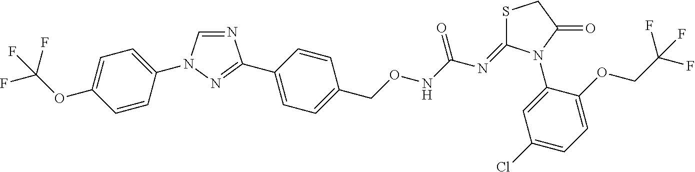 [SMILES: O=C(/N=C1\SCC(=O)N1c1cc(Cl)ccc1OCC(F)(F)F)NOCc1ccc(-c2ncn(-c3ccc(OC(F)(F)F)cc3)n2)cc1] |
| A32 | 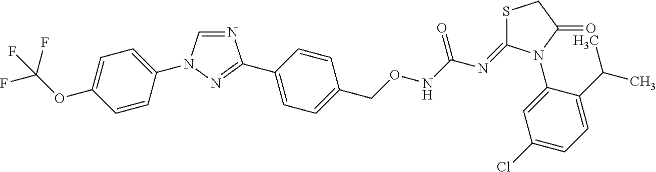 [SMILES: CC(C)c1ccc(Cl)cc1N1C(=O)CS/C1=N\C(=O)NOCc1ccc(-c2ncn(-c3ccc(OC(F)(F)F)cc3)n2)cc1] |
| A33 | 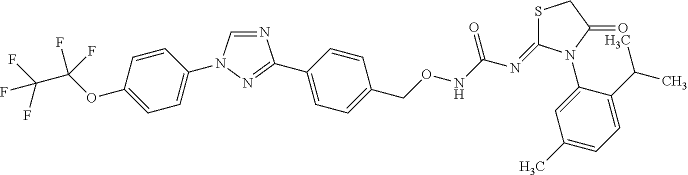 [SMILES: Cc1ccc(C(C)C)c(N2C(=O)CS/C2=N\C(=O)NOCc2ccc(-c3ncn(-c4ccc(OC(F)(F)C(F)(F)F)cc4)n3)cc2)c1] |
| A34 | 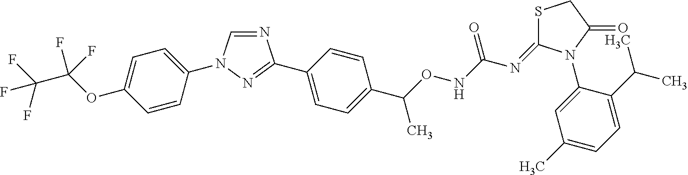 [SMILES: Cc1ccc(C(C)C)c(N2C(=O)CS/C2=N\C(=O)NOC(C)c2ccc(-c3ncn(-c4ccc(OC(F)(F)C(F)(F)F)cc4)n3)cc2)c1] |
| A35 | 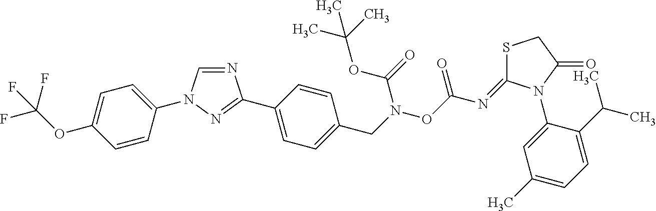 [SMILES: Cc1ccc(C(C)C)c(N2C(=O)CS/C2=N\C(=O)ON(Cc2ccc(-c3ncn(-c4ccc(OC(F)(F)F)cc4)n3)cc2)C(=O)OC(C)(C)C)c1] |
| A36 | 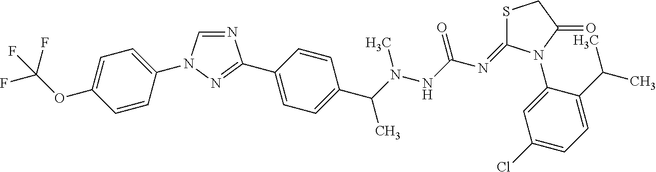 [SMILES: CC(C)c1ccc(Cl)cc1N1C(=O)CS/C1=N\C(=O)NN(C)C(C)c1ccc(-c2ncn(-c3ccc(OC(F)(F)F)cc3)n2)cc1] |
| A37 | 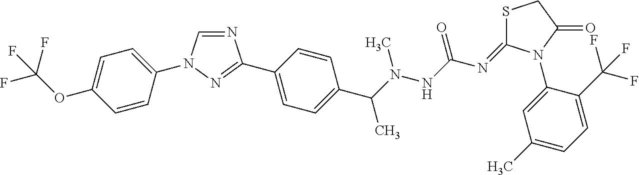 [SMILES: Cc1ccc(C(F)(F)F)c(N2C(=O)CS/C2=N\C(=O)NN(C)C(C)c2ccc(-c3ncn(-c4ccc(OC(F)(F)F)cc4)n3)cc2)c1] |
| A38 | 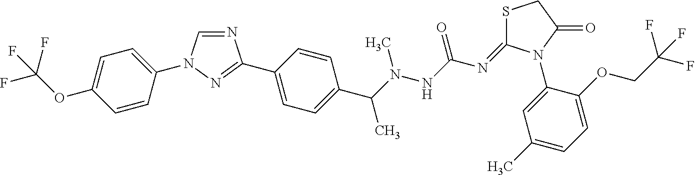 [SMILES: Cc1ccc(OCC(F)(F)F)c(N2C(=O)CS/C2=N\C(=O)NN(C)C(C)c2ccc(-c3ncn(-c4ccc(OC(F)(F)F)cc4)n3)cc2)c1] |
| A39 | 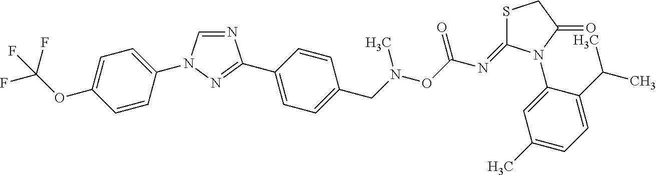 [SMILES: Cc1ccc(C(C)C)c(N2C(=O)CS/C2=N\C(=O)ON(C)Cc2ccc(-c3ncn(-c4ccc(OC(F)(F)F)cc4)n3)cc2)c1] |
| A40 | 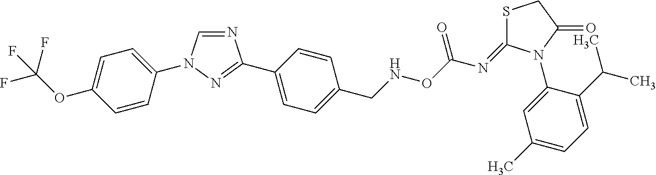 [SMILES: Cc1ccc(C(C)C)c(N2C(=O)CS/C2=N\C(=O)ONCc2ccc(-c3ncn(-c4ccc(OC(F)(F)F)cc4)n3)cc2)c1] |

TABLE 2
Analytical Data for Compounds in Table 1

| | Melting | | | |
|---|---|---|---|---|
| Cmpd. | Point | IR | | |
| No. | (° C.) | (cm⁻¹) | MASS SPEC | NMR |
| A3 | | | HRMS-ESI (m/z) | ¹H NMR (400 MHz, CDCl₃) δ 8.64 (s, |
| | | | [M + H]⁺ calcd for | 1H), 8.09 (d, J = 7.8 Hz, 2H), 7.81 (d, J = |
| | | | C₃₂H₃₂F₃N₇O₃S, | 8.8 Hz, 2H), 7.46-7.37 (m, 4H), |
| | | | 652.2312; found, | 7.36-7.28 (m, 1H), 6.84 (d, J = 8.0 |
| | | | 652.2319 | Hz, 1H), 6.28 (d, J = 28.1 Hz, 1H), |
| | | | | 4.00-3.87 (m, 3H), 2.84 (d, J = 4.0 |
| | | | | Hz, 1H), 2.60 (dd, J = 12.9, 6.2 Hz, |
| | | | | 1H), 2.51 (d, J = 1.6 Hz, 3H), 2.34 (d, J = |
| | | | | 8.2 Hz, 3H), 1.43 (dd, J = 6.7, 4.2 |
| | | | | Hz, 3H), 1.18-1.08 (m, 6H); |
| | | | | ¹⁹F NMR (376 MHz, CDCl₃) δ −58.02 |
| A4 | | (thin film) | HRMS-ESI (m/z) | ¹H NMR (400 MHz, CDCl₃) δ 8.63 (s, |
| | | 3119, 1701, | [M + H]⁺ calcd for | 1H), 8.09 (d, J = 7.9 Hz, 2H), 7.85- |
| | | 1515 | C₃₁H₃₀F₃N₇O₃S, | 7.78 (m, 2H), 7.41 (t, J = 7.7 Hz, 4H), |
| | | | 638.2156; found, | 7.33-7.20 (m, 2H), 6.83 (d, J = 1.8 |
| | | | 638.2162 | Hz, 1H), 6.42 (s, 1H), 4.07-3.94 (m, |
| | | | | 2H), 3.91 (d, J = 1.7 Hz, 2H), 2.66 (s, |
| | | | | 3H), 2.58 (q, J = 7.0 Hz, 1H), 2.33 (s, |
| | | | | 3H), 1.11 (dd, J = 12.5, 6.8 Hz, 6H); |
| | | | | ¹⁹F NMR (376 MHz, CDCl₃) δ −58.02 |
| A5 | | (thin film) | ESIMS m/z 700.0 | ¹H NMR (400 MHz, CDCl₃) δ 9.73 (s, |
| | | 2991, 1692, | ([M + H]⁺) | 1H), 8.60-8.51 (m, 1H), 8.21-8.12 |
| | | 1604, 1516, | | (m, 2H), 7.83-7.68 (m, 2H), 7.63- |
| | | 1416 | | 7.48 (m, 1H), 7.40 (dd, J = 12.7, 8.5 |
| | | | | Hz, 5H), 7.17-6.98 (m, 1H), 6.92 (dd, |
| | | | | J = 8.8, 2.5 Hz, 1H), 5.44-5.32 (m, |
| | | | | 1H), 4.00-3.79 (m, 2H), 1.93-1.73 |
| | | | | (m, 3H); |
| | | | | ¹⁹F NMR (376 MHz, CDCl₃) δ −57.98, |
| | | | | −58.03 |
| A6 | | (thin film) | ESIMS m/z 694.1 | ¹H NMR (400 MHz, CDCl₃) δ 8.56 (s, |
| | | 3284, 2928, | ([M + H]⁺) | 1H), 8.12 (dd, J = 8.2, 4.9 Hz, 2H), |
| | | 1737, 1650, | | 7.80 (d, J = 8.7 Hz, 2H), 7.45 (dd, J = |
| | | 1570, 1513 | | 8.3, 6.2 Hz, 2H), 7.39 (d, J = 8.5 Hz, |
| | | | | 3H), 7.22-7.15 (m, 1H), 6.99-6.95 |
| | | | | (m, 1H), 6.88 (dd, J = 15.8, 8.5 Hz, |
| | | | | 1H), 6.68 (d, J = 5.6 Hz, 1H), 4.34- |
| | | | | 4.19 (m, 3H), 3.98-3.85 (m, 2H), 2.31 |
| | | | | (d, J = 8.5 Hz, 3H), 1.37 (dd, J = 6.6, |
| | | | | 3.4 Hz, 3H); |
| | | | | ¹⁹F NMR (376 MHz, CDCl₃) δ −58.03, |
| | | | | −74.16 |
| A7 | | | ESIMS m/z 714.0 | ¹H NMR (400 MHz, CDCl₃) δ 8.55 (d, |
| | | | ([M + H]⁺) | J = 1.3 Hz, 1H), 8.15-8.09 (m, 2H), |
| | | | | 7.84-7.75 (m, 2H), 7.49-7.43 (m, |
| | | | | 2H), 7.43-7.32 (m, 3H), 7.18 (dd, J = |
| | | | | 4.1, 2.5 Hz, 1H), 6.91 (dd, J = 14.6, 8.9 |
| | | | | Hz, 1H), 6.78-6.70 (m, 1H), 4.55 (s, |
| | | | | 1H), 4.45-4.35 (m, 1H), 4.35-4.20 |
| | | | | (m, 2H), 3.94-3.89 (m, 2H), 1.33 (d, J = |
| | | | | 6.7 Hz, 3H); |
| | | | | ¹⁹F NMR (376 MHz, CDCl₃) δ −58.03, |
| | | | | −74.05, −74.07 |
| A8 | | (thin film) | ESIMS m/z 658.1 | ¹H NMR (400 MHz, CDCl₃) δ 8.59- |
| | | 1713, 1615, | ([M + H]⁺) | 8.54 (m, 1H), 8.23-8.08 (m, 2H), 7.86- |
| | | 1515¹ | | 7.76 (m, 3H), 7.55-7.27 (m, 6H), |
| | | | | 7.02 (dd, J = 5.3, 2.1 Hz, 1H), 6.71 (d, |
| | | | | J = 7.8 Hz, 1H), 4.26 (p, J = 6.6 Hz, |
| | | | | 1H), 4.03-3.91 (m, 2H), 2.58 (dq, J = |
| | | | | 12.7, 6.7 Hz, 1H), 1.37 (dd, J = 6.6, 5.3 |
| | | | | Hz, 3H), 1.17-1.09 (m, 3H), 1.01 (d, |
| | | | | J = 6.8 Hz, 3H); |
| | | | | ¹⁹F NMR (376 MHz, CDCl₃) δ −58.03 |
| A9 | | (thin film) | ESIMS m/z 638.3 | ¹H NMR (400 MHz, CDCl₃) δ 8.59- |
| | | 3290, 2965, | ([M + H]⁺) | 8.54 (m, 1H), 8.17-8.07 (m, 2H), 7.85- |
| | | 1732, 1648, | | 7.75 (m, 2H), 7.50-7.36 (m, 4H), |
| | | 1563, 1514 | | 7.24 (s, 2H), 6.81 (d, J = 6.1 Hz, 1H), |
| | | | | 6.74 (d, J = 8.1 Hz, 1H), 4.51 (d, J = |
| | | | | 27.2 Hz, 1H), 4.32-4.21 (m, 1H), 3.94 |
| | | | | (d, J = 1.5 Hz, 2H), 2.57 (dq, J = 12.9, |
| | | | | 6.8 Hz, 1H), 2.30 (d, J = 10.1 Hz, 3H), |
| | | | | 1.15-1.09 (m, 6H), 1.01 (d, J = 6.8 |
| | | | | Hz, 3H); |
| | | | | ¹⁹F NMR (376 MHz, CDCl₃) δ −58.03 |
| A10 | | | ESIMS m/z 625 | ¹H NMR (400 MHz, CDCl₃) δ 8.56 (s, |
| | | | ([M + H]⁺) | 1H), 8.16 (d, J = 7.8 Hz, 2H), 7.92 (s, |
| | | | | 1H), 7.83-7.75 (m, 2H), 7.48 (d, J = |
| | | | | 7.8 Hz, 2H), 7.39 (d, J = 8.6 Hz, 2H), |
| | | | | 7.21 (t, J = 7.3 Hz, 1H), 6.85 (s, 1H), |
| | | | | 4.98 (s, 2H), 3.96 (d, J = 2.4 Hz, 2H), |
| | | | | 2.32 (m, 5H), 1.52 (q, J = 7.6 Hz, 2H), |
| | | | | 0.89 (t, J = 7.3 Hz, 3H); |
| | | | | ¹⁹F NMR (376 MHz, CDCl₃) δ −58.03 |
| A11 | | | ESIMS m/z 631 | ¹H NMR (400 MHz, CDCl₃) δ 8.55 (d, |
| | | | ([M + H]⁺) | J = 1.0 Hz, 1H), 8.15 (d, J = 7.9 Hz, |
| | | | | 2H), 7.80 (dd, J = 8.9, 1.4 Hz, 2H), |
| | | | | 7.75 (d, J = 8.8 Hz, 1H), 7.44 (s, 3H), |
| | | | | 7.39 (d, J = 8.6 Hz, 2H), 7.15 (d, J = |
| | | | | 8.4 Hz, 1H), 6.98 (d, J = 6.2 Hz, 1H), |
| | | | | 5.10-5.01 (m, 1H), 4.03-3.87 (m, |
| | | | | 2H), 2.32 (d, J = 9.6 Hz, 3H), 1.58 (d, J = |
| | | | | 6.9 Hz, 3H); |
| | | | | ¹⁹F NMR (376 MHz, CDCl₃) δ −58.03 |
| A12 | | | ESIMS m/z 639 | ¹H NMR (400 MHz, CDCl₃) δ 8.56 (d, |
| | | | ([M + H]⁺) | J = 1.2 Hz, 1H), 8.15 (d, J = 7.6 Hz, |
| | | | | 2H), 7.83-7.77 (m, 2H), 7.73 (s, 1H), |
| | | | | 7.43 (d, J = 8.0 Hz, 2H), 7.39 (d, J = |
| | | | | 8.6 Hz, 2H), 7.17 (d, J = 9.9 Hz, 2H), |
| | | | | 6.82 (s, 1H), 5.07 (s, 1H), 3.94 (s, 2H), |
| | | | | 2.31 (t, J = 8.5 Hz, 5H), 1.58 (d, J = |
| | | | | 6.7 Hz, 5H), 0.88 (ddt, J = 20.3, 13.8, |
| | | | | 6.9 Hz, 3H); |
| | | | | ¹⁹F NMR (376 MHz, CDCl₃) δ −58.03 |
| A13 | | | ESIMS m/z 665 | ¹H NMR (400 MHz, CDCl₃) δ 8.56 (s, |
| | | | ([M + H]⁺) | 1H), 8.15 (d, J = 7.8 Hz, 2H), 7.80 (dd, |
| | | | | J = 9.0, 1.4 Hz, 2H), 7.68-7.56 (m, |
| | | | | 2H), 7.44-7.34 (m, 5H), 7.00 (d, J = |
| | | | | 5.0 Hz, 1H), 5.06 (s, 1H), 3.93 (q, J = |
| | | | | 18.0 Hz, 2H), 2.41 (d, J = 8.4 Hz, 3H), |
| | | | | 1.58 (s, 3H); |
| | | | | ¹⁹F NMR (376 MHz, CDCl₃) δ −58.03, |
| | | | | −61.22 |
| A14 | | | ESIMS m/z 655 | ¹H NMR (400 MHz, CDCl₃) δ 8.56 (d, |
| | | | ([M + H]⁺) | J = 1.4 Hz, 1H), 8.15 (t, J = 7.4 Hz, |
| | | | | 2H), 7.83-7.77 (m, 2H), 7.73 (s, 1H), |
| | | | | 7.47-7.36 (m, 5H), 6.96 (s, 1H), 6.50 |
| | | | | (s, 1H), 5.07 (s, 1H), 3.95 (s, 2H), 3.81- |
| | | | | 3.66 (m, 3H), 2.52 (s, 1H), 1.58 (s, |
| | | | | 3H), 1.15-0.95 (m, 6H); |
| | | | | ¹⁹F NMR (376 MHz, CDCl₃) δ −58.03 |
| A15 | | | ESIMS m/z 655 | ¹H NMR (400 MHz, CDCl₃) δ 8.56 (d, |
| | | | ([M + H]⁺) | J = 1.2 Hz, 1H), 8.15 (d, J = 7.7 Hz, |
| | | | | 2H), 7.80 (d, J = 8.8 Hz, 2H), 7.72 (s, |
| | | | | 1H), 7.42 (dd, J = 17.9, 8.2 Hz, 3H), |
| | | | | 6.90 (d, J = 8.9 Hz, 1H), 6.54 (s, 1H), |
| | | | | 5.07 (s, 1H), 3.94 (s, 2H), 3.81-3.64 |
| | | | | (m, 3H), 2.24 (s, 2H), 2.17 (s, 3H), |
| | | | | 1.59 (d, J = 6.4 Hz, 2H), 1.59-1.41 |
| | | | | (m, 2H), 0.84 (dt, J = 14.1, 7.3 Hz, |
| | | | | 3H); |
| | | | | ¹⁹F NMR (376 MHz, CDCl₃) δ −58.03 |
| A16 | | | ESIMS m/z 641 | ¹H NMR (400 MHz, CDCl₃) δ 8.56 (s, |
| | | | ([M + H]⁺) | 1H), 8.15 (d, J = 7.9 Hz, 2H), 7.80 (d, J = |
| | | | | 8.5 Hz, 2H), 7.68 (s, 1H), 7.46-7.35 |
| | | | | (m, 4H), 7.26-7.21 (m, 2H), 6.90 (s, |
| | | | | 1H), 5.07 (s, 1H), 4.21 (dd, J = 23.9, |
| | | | | 10.0 Hz, 2H), 3.91 (s, 2H), 3.25-3.09 |
| | | | | (m, 3H), 2.35 (t, J = 8.3 Hz, 3H), 1.58 |
| | | | | (m, 3H); |
| | | | | ¹⁹F NMR (376 MHz, CDCl₃) δ −58.03 |
| A17 | | | ESIMS m/z 369 | ¹H NMR (400 MHz, CDCl₃) δ 8.56 (d, |
| | | | ([M + H]⁺) | J = 1.2 Hz, 1H), 8.14 (t, J = 7.6 Hz, |
| | | | | 2H), 7.85-7.76 (m, 2H), 7.72 (d, J = |
| | | | | 6.2 Hz, 1H), 7.41 (t, J = 9.1 Hz, 4H), |
| | | | | 7.26-7.22 (m, 2H), 6.80 (s, 1H), 5.05 |
| | | | | (d, J = 8.3 Hz, 1H), 3.94 (s, 2H), 2.55 |
| | | | | (d, J = 7.2 Hz, 1H), 2.29 (d, J = 8.9 Hz, |
| | | | | 3H), 1.58 (s, 3H), 1.17-0.97 (m, 6H); |
| | | | | ¹⁹F NMR (376 MHz, CDCl₃) δ −58.03 |
| A18 | | | ESIMS m/z 625 | ¹H NMR (400 MHz, CDCl₃) δ 8.56 (d, |
| | | | ([M + H]⁺) | J = 1.5 Hz, 1H), 8.17-8.10 (m, 2H), |
| | | | | 7.83-7.77 (m, 2H), 7.69 (d, J = 5.9 |
| | | | | Hz, 1H), 7.39 (d, J = 8.8 Hz, 7H), 6.99 |
| | | | | (d, J = 6.9 Hz, 1H), 5.06 (s, 1H), 3.95 |
| | | | | (s, 2H), 2.60 (d, J = 6.5 Hz, 1H), 1.58 |
| | | | | (s, 3H), 1.13 (q, J = 7.5, 6.9 Hz, 6H); |
| | | | | ¹⁹F NMR (376 MHz, CDCl₃) δ −58.03 |
| A19 | | | ESIMS m/z 695 | ¹H NMR (400 MHz, CDCl₃) δ 8.56 (d, |
| | | | ([M + H]⁺) | J = 0.7 Hz, 1H), 8.17-8.11 (m, 2H), |
| | | | | 7.83-7.75 (m, 2H), 7.68 (s, 1H), 7.44 |
| | | | | (s, 2H), 7.39 (d, J = 8.6 Hz, 2H), 7.18 |
| | | | | (s, 1H), 6.95 (s, 1H), 6.87 (dd, J = 14.0, |
| | | | | 8.6 Hz, 1H), 5.07 (s, 1H), 4.25 (dt, J = |
| | | | | 22.5, 8.4 Hz, 2H), 3.99-3.83 (m, 2H), |
| | | | | 2.30 (d, J = 7.8 Hz, 3H), 1.58 (d, J = |
| | | | | 6.5 Hz, 3H); |
| | | | | ¹⁹F NMR (376 MHz, CDCl₃) δ −58.03, |
| | | | | −74.14, −74.18 |
| A20 | | | ESIMS m/z 715 | ¹H NMR (400 MHz, CDCl₃) δ 8.56 (d, |
| | | | ([M + H]⁺) | J = 1.0 Hz, 1H), 8.16 (dd, J = 8.1, 4.6 |
| | | | | Hz, 2H), 7.83-7.76 (m, 2H), 7.68 (s, |
| | | | | 1H), 7.49-7.32 (m, 5H), 7.17 (s, 1H), |
| | | | | 6.96-6.86 (m, 1H), 5.07 (s, 1H), 4.36- |
| | | | | 4.19 (m, 2H), 3.92 (d, J = 4.7 Hz, |
| | | | | 2H), 1.59 (d, J = 6.6 Hz, 3H); |
| | | | | ¹⁹F NMR (376 MHz, CDCl₃) δ −58.03, |
| | | | | −74.04, −74.05 |
| A21 | | | ESIMS m/z 659 | ¹H NMR (400 MHz, CDCl₃) δ 8.56 (d, |
| | | | ([M + H]⁺) | J = 1.7 Hz, 1H), 8.15 (t, J = 7.7 Hz, |
| | | | | 3H), 7.84-7.76 (m, 2H), 7.69 (s, 1H), |
| | | | | 7.38 (dt, J = 21.1, 10.5 Hz, 5H), 7.00 |
| | | | | (s, 1H), 5.07 (s, 1H), 3.95 (s, 2H), 2.56 |
| | | | | (s, 1H), 1.59 (s, 3H), 1.15-0.96 (m, |
| | | | | 6H); |
| | | | | ¹⁹F NMR (376 MHz, CDCl₃) δ −58.03 |
| A22 | | | ESIMS m/z 617 | ¹H NMR (400 MHz, CDCl₃) δ 8.56 (s, |
| | | | ([M + H]⁺) | 1H), 8.17 (d, J = 7.9 Hz, 2H), 7.89 (s, |
| | | | | 1H), 7.80 (d, J = 9.0 Hz, 2H), 7.49 (d, J = |
| | | | | 7.8 Hz, 2H), 7.39 (dd, J = 8.6, 3.2 |
| | | | | Hz, 3H), 7.20 (d, J = 8.2 Hz, 1H), 7.03 |
| | | | | (s, 1H), 4.99 (s, 2H), 4.07-3.87 (m, |
| | | | | 2H), 2.35 (s, 3H); |
| | | | | ¹⁹F NMR (376 MHz, CDCl₃) δ −58.03 |
| A23 | 170-172 | | ESIMS m/z 641 | ¹H NMR (400 MHz, CDCl₃) δ 8.56 (s, |
| | | | ([M + H]⁺) | 1H), 8.17 (d, J = 7.9 Hz, 2H), 7.90 (s, |
| | | | | 1H), 7.80 (d, J = 8.9 Hz, 2H), 7.49 (d, J = |
| | | | | 7.7 Hz, 2H), 7.39 (d, J = 8.6 Hz, 2H), |
| | | | | 7.22 (s, 1H), 6.94 (d, J = 8.5 Hz, 1H), |
| | | | | 6.57 (s, 1H), 4.98 (s, 2H), 3.96 (d, J = |
| | | | | 2.2 Hz, 2H), 3.77 (s, 3H), 2.36-2.20 |
| | | | | (m, 2H), 1.53-1.42 (m, 2H), 0.88 (t, J = |
| | | | | 7.3 Hz, 3H); |
| | | | | ¹⁹F NMR (376 MHz, CDCl₃) δ −58.03 |
| A24 | | | ESIMS m/z 651 | ¹H NMR (400 MHz, CDCl₃) δ 8.56 (s, |
| | | | ([M + H]⁺) | 1H), 8.16 (d, J = 7.9 Hz, 2H), 7.84- |
| | | | | 7.81 (m, 3H), 7.65 (d, J = 8.1 Hz, 1H), |
| | | | | 7.48 (d, J = 7.8 Hz, 2H), 7.39 (d, J = |
| | | | | 8.7 Hz, 3H), 7.04 (s, 1H), 4.97 (s, 2H), |
| | | | | 3.95 (q, J = 18.1 Hz, 2H), 2.43 (s, 3H); |
| | | | | ¹⁹F NMR (376 MHz, CDCl₃) δ −58.03, |
| | | | | −61.18 |
| A25 | | | ESIMS m/z 681 | ¹H NMR (400 MHz, CDCl₃) δ 8.56 (s, |
| | | | ([M + H]⁺) | 1H), 8.17 (d, J = 7.9 Hz, 2H), 7.88 (s, |
| | | | | 1H), 7.80 (d, J = 8.9 Hz, 2H), 7.49 (d, J = |
| | | | | 7.7 Hz, 2H), 7.39-7.35 (m, 2H), |
| | | | | 6.99 (d, J = 6.8 Hz, 1H), 6.66 (s, 1H), |
| | | | | 4.98 (s, 2H), 4.04-3.88 (m, 2H), 3.80 |
| | | | | (s, 3H), 3.24-3.03 (m, 3H); |
| | | | | ¹⁹F NMR (376 MHz, CDCl₃) δ −58.03, |
| | | | | −64.77 |
| A26 | | | ESIMS m/z 641 | ¹H NMR (400 MHz, CDCl₃) δ 8.56 (s, |
| | | | ([M + H]⁺) | 1H), 8.16 (d, J = 7.8 Hz, 2H), 7.90 (s, |
| | | | | 1H), 7.84-7.77 (m, 2H), 7.48 (d, J = |
| | | | | 7.7 Hz, 2H), 7.39 (d, J = 8.6 Hz, 2H), |
| | | | | 7.31 (d, J = 8.8 Hz, 1H), 6.99 (d, J = |
| | | | | 8.8 Hz, 1H), 6.53 (s, 1H), 4.97 (s, 2H), |
| | | | | 3.97 (d, J = 1.8 Hz, 2H), 3.76 (s, 3H), |
| | | | | 2.56 (t, J = 7.3 Hz, 1H), 1.12 (dd, J = |
| | | | | 12.8, 6.8 Hz, 6H); |
| | | | | ¹⁹F NMR (376 MHz, CDCl₃) δ −58.03 |
| A27 | | | ESIMS m/z 627 | ¹H NMR (400 MHz, CDCl₃) δ 8.56 (s, |
| | | | ([M + H]⁺) | 1H), 8.16 (d, J = 7.8 Hz, 2H), 7.88 (s, |
| | | | | 1H), 7.84-7.77 (m, 2H), 7.48 (d, J = |
| | | | | 7.8 Hz, 2H), 7.39 (d, J = 8.5 Hz, 2H), |
| | | | | 7.33 (d, J = 7.7 Hz, 1H), 7.23 (d, J = |
| | | | | 7.5 Hz, 1H), 6.93 (s, 1H), 4.98 (s, 2H), |
| | | | | 4.35-4.14 (m, 2H), 3.93 (d, J = 3.8 |
| | | | | Hz, 2H), 3.22 (s, 3H), 2.37 (s, 3H); |
| | | | | ¹⁹F NMR (376 MHz, CDCl₃) δ −58.03 |
| A28 | | | ESIMS m/z 625 | ¹H NMR (400 MHz, CDCl₃) δ 8.55 (s, |
| | | | ([M + H]⁺) | 1H), 8.15 (d, J = 7.9 Hz, 2H), 7.94 (s, |
| | | | | 1H), 7.83-7.75 (m, 2H), 7.47 (d, J = |
| | | | | 7.7 Hz, 2H), 7.42-7.37 (m, 2H), 7.29 |
| | | | | (d, J = 8.0 Hz, 1H), 7.23 (d, J = 8.2 Hz, |
| | | | | 1H), 6.82 (s, 1H), 4.96 (s, 2H), 3.96 (d, |
| | | | | J = 1.9 Hz, 2H), 2.63-2.52 (m, 1H), |
| | | | | 2.32 (s, 3H), 1.13 (dd, J = 9.8, 6.8 Hz, |
| | | | | 6H); |
| | | | | ¹⁹F NMR (376 MHz, CDCl₃) δ −58.02 |
| A29 | | | ESIMS m/z 611 | ¹H NMR (400 MHz, CDCl₃) δ 8.56 (s, |
| | | | ([M + H]⁺) | 1H), 8.16 (d, J = 7.9 Hz, 2H), 7.87 (s, |
| | | | | 1H), 7.83-7.75 (m, 2H), 7.53-7.34 |
| | | | | (m, 7H), 7.01 (d, J = 9.5 Hz, 1H), 4.97 |
| | | | | (s, 2H), 3.98 (d, J = 1.7 Hz, 2H), 2.64 |
| | | | | (s, 1H), 1.16 (dd, J = 11.8, 6.9 Hz, 6H); |
| | | | | ¹⁹F NMR (376 MHz, CDCl₃) δ −58.03 |
| A30 | | | ESIMS m/z 681 | ¹H NMR (400 MHz, CDCl₃) δ 8.56 (s, |
| | | | ([M + H]⁺) | 1H), 8.17 (d, J = 7.9 Hz, 2H), 7.84 (s, |
| | | | | 1H), 7.83-7.76 (m, 2H), 7.51 (d, J = |
| | | | | 9.0 Hz, 2H), 7.39 (d, J = 8.6 Hz, 2H), |
| | | | | 7.22 (d, J = 8.4 Hz, 1H), 6.99 (d, J = |
| | | | | 2.8 Hz, 1H), 6.91 (d, J = 8.4 Hz, 1H), |
| | | | | 4.98 (s, 2H), 4.29 (qd, J = 8.1, 1.3 Hz, |
| | | | | 2H), 4.00-3.85 (m, 2H), 2.33 (s, 3H); |
| | | | | ¹⁹F NMR (376 MHz, CDCl₃) δ −58.03, |
| | | | | −74.14 |
| A31 | 183-184 | | ESIMS m/z 701 | ¹H NMR (400 MHz, CDCl₃) δ 8.56 (s, |
| | | | ([M + H]⁺) | 1H), 8.17 (d, J = 7.9 Hz, 2H), 7.87 (s, |
| | | | | 1H), 7.83-7.76 (m, 2H), 7.51 (d, J = |
| | | | | 6.7 Hz, 2H), 7.43-7.36 (m, 3H), 7.21 |
| | | | | (s, 1H), 6.95 (d, J = 8.9 Hz, 1H), 4.98 |
| | | | | (s, 2H), 4.37-4.25 (m, 2H), 4.01- |
| | | | | 3.86 (m, 2H); |
| | | | | ¹⁹F NMR (376 MHz, CDCl₃) δ −58.03, |
| | | | | −74.02 |
| A32 | | | ESIMS m/z 645 | ¹H NMR (400 MHz, CDCl₃) δ 8.56 (s, |
| | | | ([M + H]⁺) | 1H), 8.17 (d, J = 7.8 Hz, 2H), 7.87 (s, |
| | | | | 1H), 7.80 (d, J = 9.0 Hz, 2H), 7.48 (d, J = |
| | | | | 8.0 Hz, 2H), 7.39 (d, J = 8.5 Hz, 3H), |
| | | | | 7.34 (d, J = 8.6 Hz, 1H), 7.04 (s, 1H), |
| | | | | 4.98 (s, 2H), 3.97 (d, J = 1.5 Hz, 2H), |
| | | | | 2.60 (s, 1H), 1.13 (dd, J = 14.5, 6.8 Hz, |
| | | | | 6H); |
| | | | | ¹⁹F NMR (376 MHz, CDCl₃) δ −58.03 |
| A33 | | | ESIMS m/z 675 | ¹H NMR (400 MHz, CDCl₃) δ 8.57 (s, |
| | | | ([M + H]⁺) | 1H), 8.16 (d, J = 7.8 Hz, 2H), 7.92 (s, |
| | | | | 1H), 7.84-7.76 (m, 2H), 7.48 (d, J = |
| | | | | 7.7 Hz, 2H), 7.40 (d, J = 8.9 Hz, 2H), |
| | | | | 7.26 (m, 2H), 6.83 (s, 1H), 4.97 (s, |
| | | | | 2H), 3.96 (d, J = 2.0 Hz, 2H), 2.66- |
| | | | | 2.53 (m, 1H), 2.32 (s, 3H), 1.13 (dd, J = |
| | | | | 9.7, 6.8 Hz, 6H); |
| | | | | ¹⁹F NMR (376 MHz, CDCl₃) δ −85.89, |
| | | | | −87.85 |
| A34 | 125-140 | | ESIMS m/z 689 | ¹H NMR (400 MHz, CDCl₃) δ 8.57 (d, |
| | | | ([M + H]⁺) | J = 1.2 Hz, 1H), 8.15 (t, J = 7.6 Hz, |
| | | | | 2H), 7.81 (dd, J = 8.9, 1.8 Hz, 2H), |
| | | | | 7.72 (d, J = 6.1 Hz, 1H), 7.41 (t, J = |
| | | | | 7.7 Hz, 4H), 7.23 (m, 2H), 6.80 (s, |
| | | | | 1H), 5.07 (s, 1H), 3.94 (s, 2H), 2.55 (d, |
| | | | | J = 8.8 Hz, 1H), 2.29 (d, J = 8.8 Hz, |
| | | | | 3H), 1.58 (s, 3H), 1.13-1.09 (m, 6H); |
| | | | | ¹⁹F NMR (376 MHz, CDCl₃) δ −85.89, |
| | | | | −87.85 |
| A35 | | | ESIMS m/z 725 | ¹H NMR (400 MHz, CDCl₃) δ 8.55 (s, |
| | | | ([M + H]⁺) | 1H), 8.13-8.07 (m, 2H), 7.82-7.74 |
| | | | | (m, 2H), 7.39 (t, J = 7.9 Hz, 4H), 7.29 |
| | | | | (d, J = 8.0 Hz, 1H), 7.23 (dd, J = 8.1, |
| | | | | 1.8 Hz, 1H), 6.84-6.78 (m, 1H), 4.78 |
| | | | | (d, J = 5.0 Hz, 2H), 4.01 (d, J = 1.1 Hz, |
| | | | | 2H), 2.52 (p, J = 6.8 Hz, 1H), 2.31 (s, |
| | | | | 3H), 1.45 (s, 9H), 1.13 (d, J = 6.8 Hz, |
| | | | | 6H); |
| | | | | ¹⁹F NMR (376 MHz, CDCl₃) δ −58.02 |
| A36 | | | HRMS-ESI (m/z) | ¹H NMR (400 MHz, CDCl₃) δ 8.56 (s, |
| | | | [M + H]⁺ calcd for | 1H), 8.12 (dd, J = 8.3, 2.8 Hz, 2H), |
| | | | C₃₁H₂₉ClF₃N₇O₃, | 7.84-7.75 (m, 2H), 7.45-7.35 (m, |
| | | | 671.1693; found, | 6H), 7.05 (dd, J = 8.9, 2.1 Hz, 1H), |
| | | | 671.1703 | 6.18 (d, J = 23.1 Hz, 1H), 4.01-3.93 |
| | | | | (m, 1H), 3.92 (d, J = 1.8 Hz, 2H), 2.68- |
| | | | | 2.56 (m, 1H), 2.52 (s, 3H), 1.42 (dd, |
| | | | | J = 6.7, 3.3 Hz, 3H), 1.17-1.06 (m, |
| | | | | 6H); |
| | | | | ¹⁹F NMR (376 MHz, CDCl₃) δ −58.03 |
| A37 | | | HRMS-ESI (m/z) | ¹H NMR (400 MHz, CDCl₃) δ 8.56 (d, |
| | | | [M + H]⁺ calcd for | J = 0.7 Hz, 1H), 8.15-8.08 (m, 2H), |
| | | | C₃₀H₂₅F₆N₇O₃S, | 7.84-7.76 (m, 2H), 7.68 (t, J = 8.3 |
| | | | 677.1644; found, | Hz, 1H), 7.44-7.35 (m, 5H), 7.05 (d, |
| | | | 677.1646 | J = 7.5 Hz, 1H), 6.13 (d, J = 6.8 Hz, |
| | | | | 1H), 4.03-3.83 (m, 3H), 2.50 (d, J = |
| | | | | 3.9 Hz, 3H), 2.46 (d, J = 9.2 Hz, 3H), |
| | | | | 1.42 (d, J = 6.6 Hz, 3H); |
| | | | | ¹⁹F NMR (376 MHz, CDCl₃) δ −58.03, |
| | | | | −61.18, −61.21 |
| A38 | | | HRMS-ESI (m/z) | ¹H NMR (400 MHz, CDCl₃) δ 8.55 (s, |
| | | | [M + H]⁺ calcd for | 1H), 8.14-8.10 (m, 2H), 7.84-7.77 |
| | | | C₃₁H₂₇F₆N₇O₄S, | (m, 2H), 7.45-7.35 (m, 4H), 7.23 (dd, |
| | | | 707.1749; found, | J = 8.5, 2.2 Hz, 1H), 7.00 (dd, J = 7.5, |
| | | | 707.1752 | 2.2 Hz, 1H), 6.93 (t, J = 8.1 Hz, 1H), |
| | | | | 6.21 (d, J = 2.1 Hz, 1H), 4.35-4.26 |
| | | | | (m, 2H), 4.08-3.81 (m, 3H), 2.51 (d, J = |
| | | | | 1.4 Hz, 3H), 2.34 (d, J = 8.0 Hz, 3H), |
| | | | | 1.42 (dd, J = 6.7, 1.5 Hz, 3H); |
| | | | | ¹⁹F NMR (376 MHz, CDCl₃) δ −58.03, |
| | | | | −74.04, −74.09 |
| A39 | | | HRMS-ESI (m/z) | ¹H NMR (400 MHz, CDCl₃) δ 8.56 (s, |
| | | | [M + H]⁺ calcd for | 1H), 8.14-8.07 (m, 2H), 7.86-7.74 |
| | | | C₃₁H₂₉F₃N₆O₄S, | (m, 2H), 7.50-7.42 (m, 2H), 7.42- |
| | | | 639.1996; found, | 7.36 (m, 2H), 7.30 (d, J = 8.1 Hz, 1H), |
| | | | 639.1999 | 7.24 (s, 1H), 6.85-6.78 (m, 1H), 4.04 |
| | | | | (t, J = 12.9 Hz, 2H), 3.94 (s, 2H), 2.83 |
| | | | | (s, 3H), 2.51 (p, J = 6.8 Hz, 1H), 2.33 |
| | | | | (s, 3H), 1.10 (t, J = 6.9 Hz, 6H); |
| | | | | ¹⁹F NMR (376 MHz, CDCl₃) δ −58.02 |
| A40 | | | ESIMS m/z 624 | ¹H NMR (400 MHz, CDCl₃) δ 8.55 (s, |
| | | | ([M]⁺) | 1H), 8.16-8.07 (m, 2H), 7.83-7.74 |
| | | | | (m, 2H), 7.47-7.34 (m, 4H), 7.30 (d, J = |
| | | | | 8.1 Hz, 1H), 7.25-7.21 (m, 1H), |
| | | | | 6.83 (d, J = 2.0 Hz, 2H), 4.61 (s, 1H), |
| | | | | 4.10-3.99 (m, 2H), 3.95 (d, J = 1.6 |
| | | | | Hz, 2H), 2.60 (p, J = 6.8 Hz, 1H), 2.32 |
| | | | | (s, 3H), 1.13 (dd, J = 8.7, 6.8 Hz, 6H); |
| | | | | ¹⁹F NMR (376 MHz, CDCl₃) δ −58.03 |

### Example: Bioassays

Insecticidal test for beet armyworm (Spodoptera exigua, LAPHEG) (“BAW”)

Bioassays on beet armyworm (BAW; Spodoptera exigua: Lepidoptera) are conducted using a 128-well diet tray assay. One to five second instar BAW larvae are placed in each well (3 mL) of the diet tray that had been previously filled with 1 mL of artificial diet to which 50 μg/cm² of the test compound (dissolved in 50 μL of 90:10 acetone-water mixture) had been applied (to each of eight wells) and then allowed to dry. Trays are covered with a clear self-adhesive cover, vented to allow gas exchange, and held at 25° C., 14:10 light-dark for five to seven days. Percent mortality is recorded for the larvae in each well; activity in the eight wells is then averaged. The results are indicated in Table 3.

### Insecticidal Test for Cabbage Looper (Trichloplusia ni, TRIPNI) (“CL”)

Bioassays on cabbage looper (CL; Trichloplusia ni: Lepidoptera) are conducted using a 128-well diet tray assay. One to five second instar CL larvae are placed in each well (3 mL) of the diet tray that had been previously filled with 1 mL of artificial diet to which 50 μg/cm² of the test compound (dissolved in 50 μL of 90:10 acetone-water mixture) had been applied (to each of eight wells) and then allowed to dry. Trays are covered with a clear self-adhesive cover, vented to allow gas exchange, and held at 25° C., 14:10 light-dark for five to seven days. Percent mortality is recorded for the larvae in each well; activity in the eight wells is then averaged. The results are indicated in Table 3.

Insecticidal Test for Yellow Fever Mosquito (Aedes aegypti, AEDSAE) (“YFM”)

Master plates containing 400 μg of a molecule dissolved in 100 μL of dimethyl sulfoxide (DMSO) (equivalent to a 4000 ppm solution) are used. A master plate of assembled molecules contains 15 μL per well. To this plate, 135 μL of a 90:10 water/acetone mixture is added to each well. A robot (Biomek® NXP Laboratory Automation Workstation) is programmed to dispense 15 μL aspirations from the master plate into an empty 96-well shallow plate (“daughter” plate). There are 6 reps (“daughter” plates) created per master. The created “daughter” plates are then immediately infested with YFM larvae.

The day before plates are to be treated, mosquito eggs are placed in Millipore water containing liver powder to begin hatching (4 g. into 400 mL). After the “daughter” plates are created using the robot, they are infested with 220 μL of the liver powder/larval mosquito mixture (about 1 day-old larvae). After plates are infested with mosquito larvae, a non-evaporative lid is used to cover the plate to reduce drying. Plates are held at room temperature for 3 days prior to grading. After 3 days, each well is observed and scored based on mortality. The results are indicated in Table 3.

BAW & CL Rating Table

| % Control (or Mortality) | Rating |
|---|---|
| 50-100 | A |
| More than 0 - Less than 50 | B |
| Not Tested | C |
| No activity noticed in this bioassay | D |

YFM Rating Table

| % Control (or Mortality) | Rating |
|---|---|
| 80-100 | A |
| More than 0 - Less than 80 | B |
| Not Tested | C |
| No activity noticed in this bioassay | D |

TABLE 3
Bioassay Activities of Compounds

| Cmpd No | BAW | CL | YFM |
|---|---|---|---|
| A3 | A | A | C |
| A4 | A | A | C |
| A5 | A | A | C |
| A6 | A | A | C |
| A7 | C | C | C |
| A8 | A | A | C |
| A9 | A | A | C |
| A10 | A | A | C |
| A11 | A | A | C |
| A12 | A | A | C |
| A13 | A | A | C |
| A14 | A | A | C |
| A15 | A | A | C |
| A16 | A | A | C |
| A17 | A | A | C |
| A18 | A | A | C |
| A19 | A | A | C |
| A20 | A | A | C |
| A21 | A | A | C |
| A22 | A | A | C |
| A23 | A | A | C |
| A24 | A | A | C |
| A25 | A | A | C |
| A26 | A | A | C |
| A27 | A | A | C |
| A28 | A | A | C |
| A29 | A | A | C |
| A30 | A | A | C |
| A31 | A | A | C |
| A32 | A | A | C |
| A33 | A | A | C |
| A34 | A | A | C |
| A35 | D | D | C |
| A36 | A | A | C |
| A37 | A | A | C |
| A38 | A | A | C |
| A39 | A | A | C |
| A40 | A | A | C |

We claim:

1. A process to control a lepidopteran pest comprising:
applying one or more of the following molecules, to an area to control said lepidopteran pest, in an amount sufficient to control said lepidopteran pest
TABLE 1

| | |
|---|---|
| A3 | 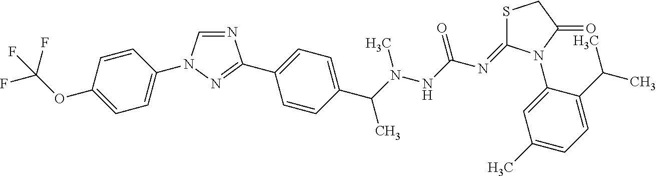 |
| A4 | 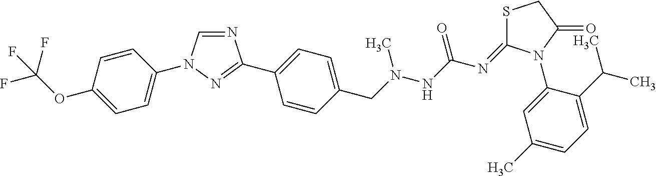 |
| A5 | 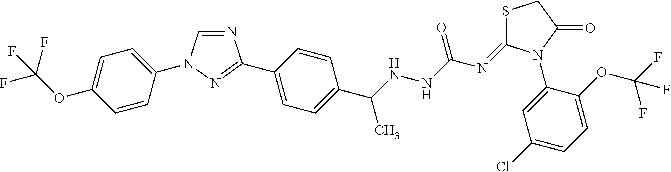 |
| A6 | 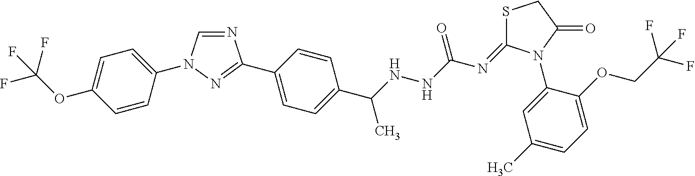 |
| A7 | 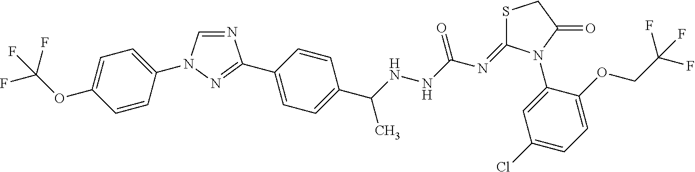 |
| A8 | 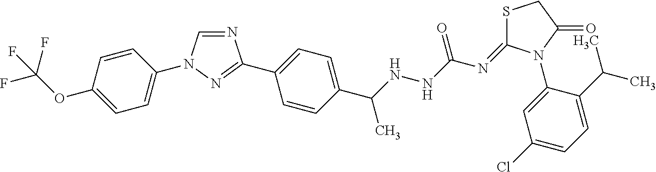 |
| A9 | 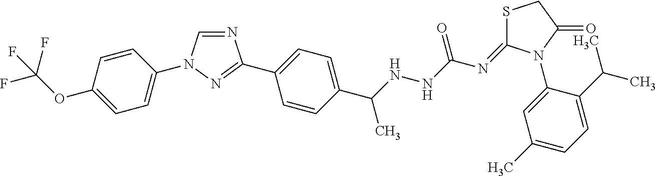 |
| A10 | 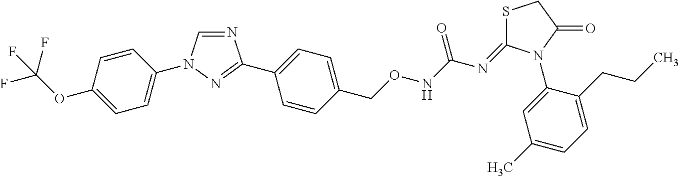 |
| A11 | 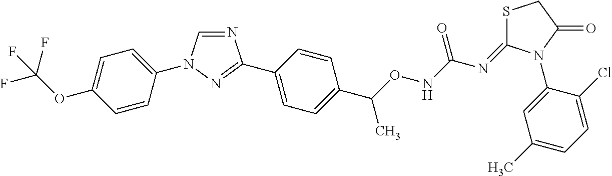 |
| A12 | 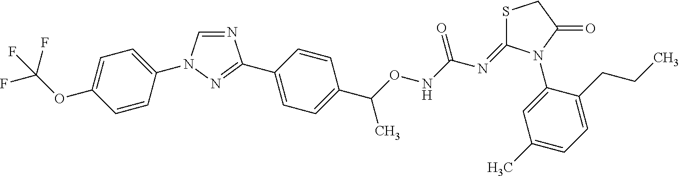 |
| A13 | 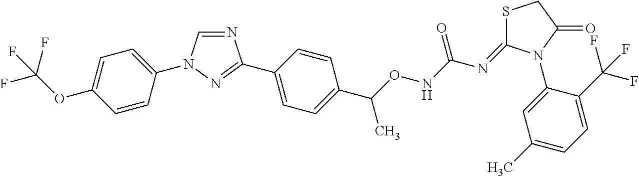 |
| A14 | 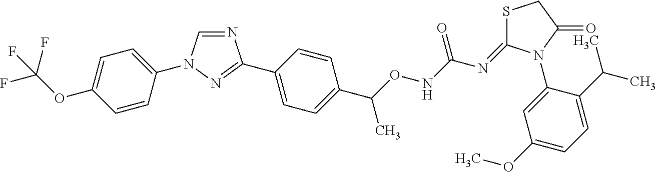 |
| A15 | 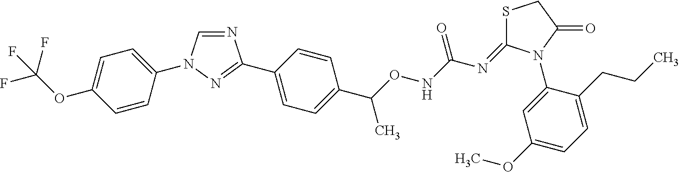 |
| A16 | 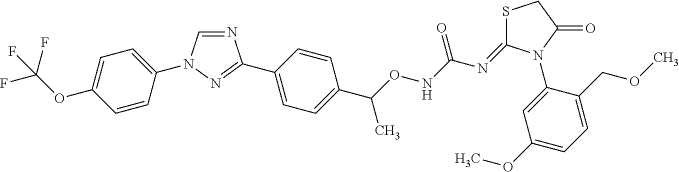 |
| A17 | 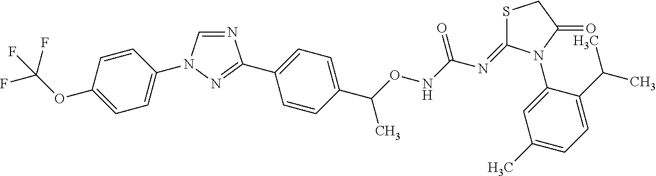 |
| A18 | 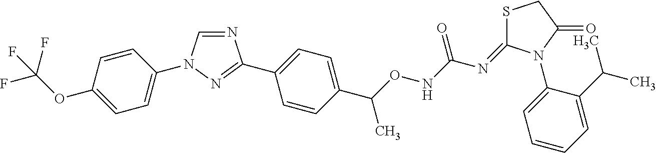 |
| A19 | 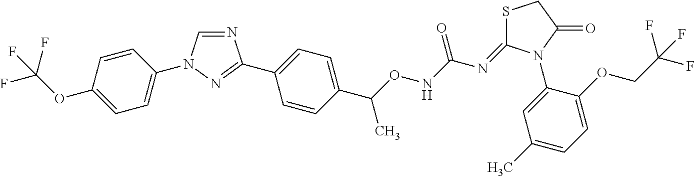 |
| A20 | 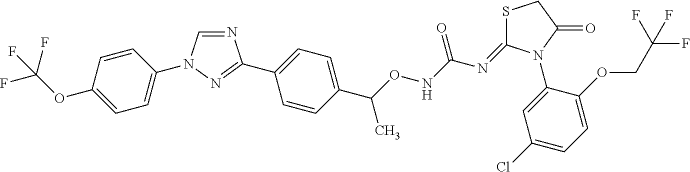 |
| A21 | 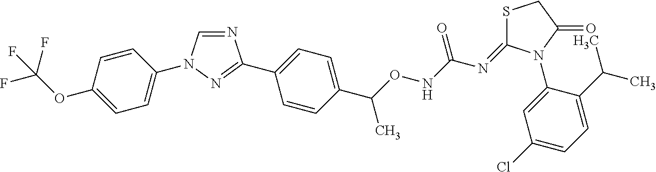 |
| A22 | 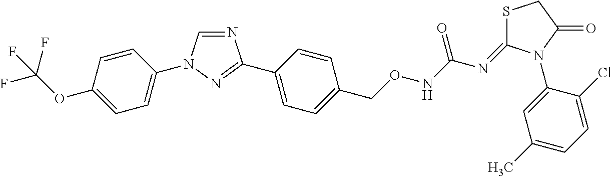 |
| A23 | 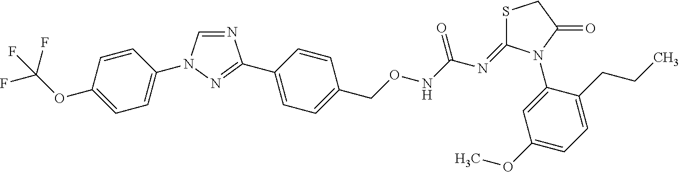 |
| A24 | 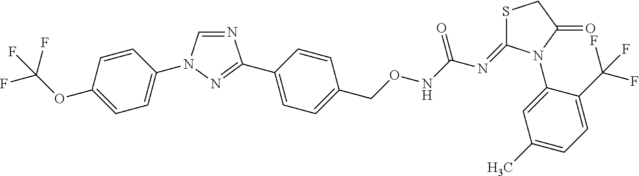 |
| A25 | 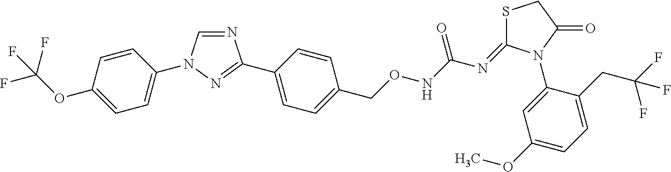 |
| A26 | 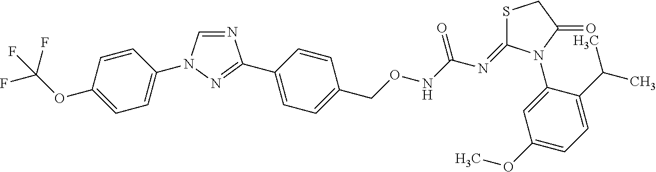 |
| A27 | 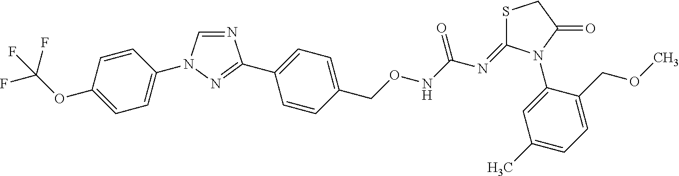 |
| A28 | 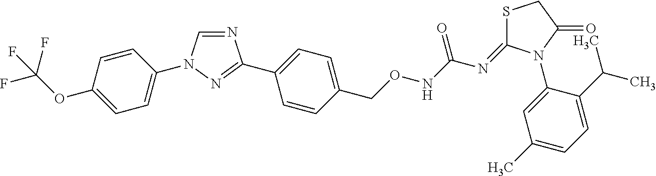 |
| A29 | 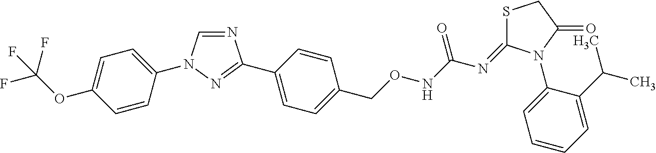 |
| A30 | 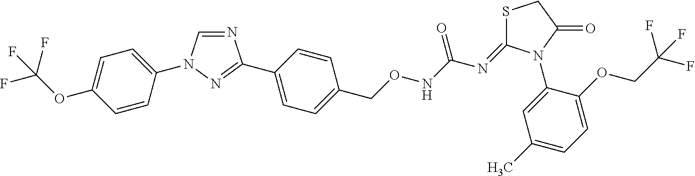 |
| A31 | 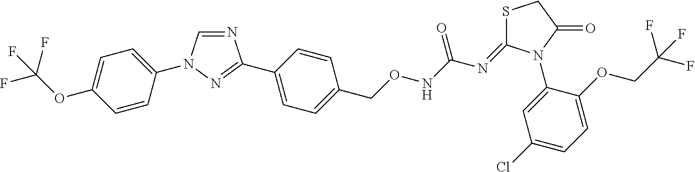 |
| A32 | 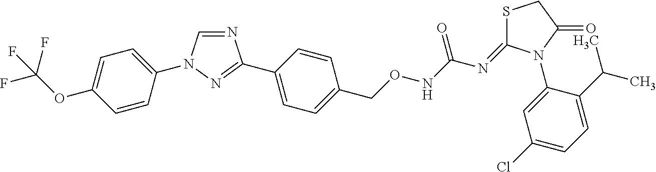 |
| A33 | 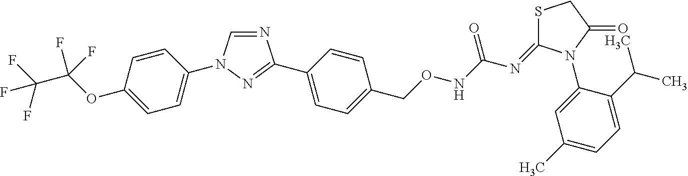 |
| A34 | 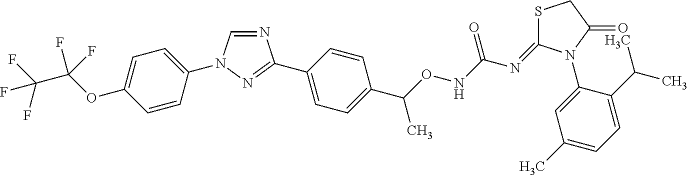 |
| A36 | 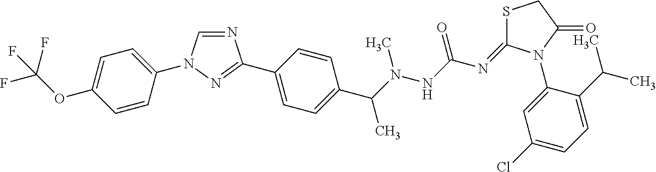 |
| A37 | 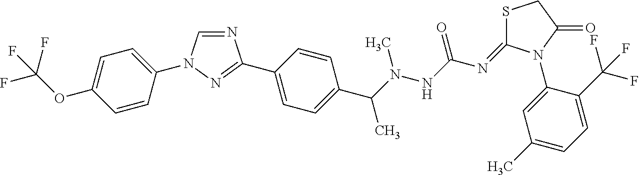 |
| A38 | 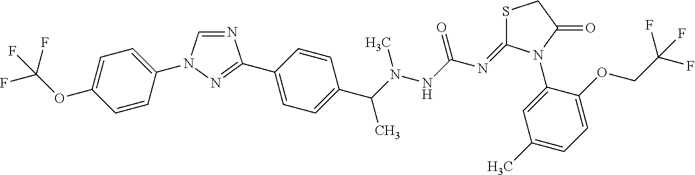 |
| A39 | 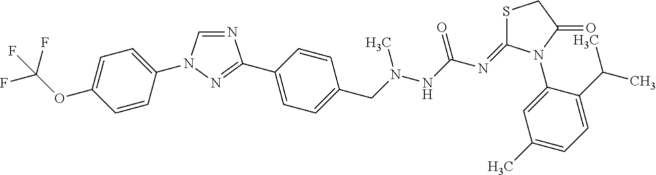 |
| A40 | 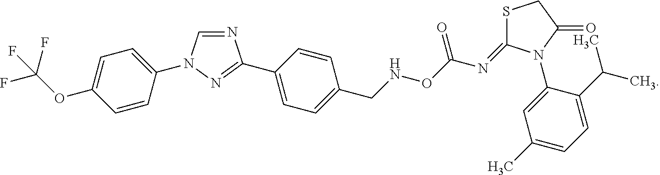 |

2. The process according to claim 1, wherein said lepidopteran pest is a beet armyworm (BAW).

3. The process according to claim 1, wherein said lepidopteran pest is a cabbage looper (CL).

4. The process according to claim 1, wherein said molecule is

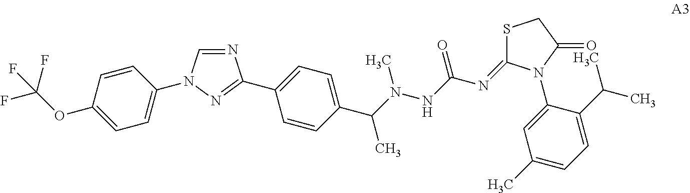 [SMILES: Cc1ccc(C(C)C)c(N2C(=O)CS/C2=N\C(=O)NN(C)C(C)c2ccc(-c3ncn(-c4ccc(OC(F)(F)F)cc4)n3)cc2)c1]

5. The process according to claim 1, wherein said molecule is

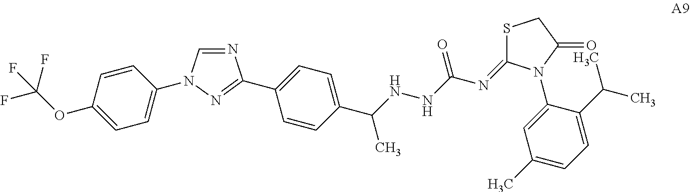 [SMILES: Cc1ccc(C(C)C)c(N2C(=O)CS/C2=N\C(=O)NNC(C)c2ccc(-c3ncn(-c4ccc(OC(F)(F)F)cc4)n3)cc2)c1]

6. The process according to claim 1, wherein said molecule is 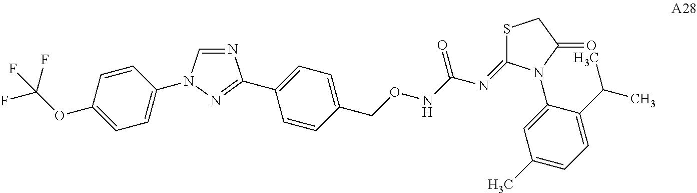 [SMILES: Cc1ccc(C(C)C)c(N2C(=O)CS/C2=N\C(=O)NOCc2ccc(-c3ncn(-c4ccc(OC(F)(F)F)cc4)n3)cc2)c1]
